SEQUENCE LISTING (171 sequences)

SEQ ID NO: 1 - RNA, 18 nt, Artificial Sequence
  Synthetic
agggguccacauggcaac

SEQ ID NO: 2 - RNA, 73 nt, Artificial Sequence
  Synthetic
ggugucgagaagaggagaacaauaugcuaaauguuguucucgucuccucgacaccagggguccacauggcaac

SEQ ID NO: 3 - RNA, 25 nt, Artificial Sequence
  Synthetic
ggugucgagaagaggagaacaauau

SEQ ID NO: 4 - RNA, 25 nt, Artificial Sequence
  Synthetic
auguuguucucgucuccucgacacc

SEQ ID NO: 5 - RNA, 24 nt, Artificial Sequence
  Synthetic
ggguggaagaguagaacaauaugc

SEQ ID NO: 6 - RNA, 24 nt, Artificial Sequence
  Synthetic
gcauauuguucuagucucccaccc

SEQ ID NO: 7 - RNA, 24 nt, Artificial Sequence
  Synthetic
ggguggaacaguacaacaauaugc

SEQ ID NO: 8 - RNA, 45 nt, Artificial Sequence
  Synthetic
guggaauaguauaacaauaugcuaaauguuguuauagugucccac

SEQ ID NO: 9 - DNA, 11 bp, Artificial Sequence
  Synthetic
atatatagcca

SEQ ID NO: 10 - DNA, 11 bp, Artificial Sequence
  Synthetic
tggccatatat

SEQ ID NO: 11 - RNA, 43 nt, Artificial Sequence
  Synthetic
auguuguucucgucuccucgacaccagggguccacauggcaac

SEQ ID NO: 12 - RNA, 42 nt, Artificial Sequence
  Synthetic
gcauauuguucuagucucccacccagggguccacauggcaac

SEQ ID NO: 13 - RNA, 42 nt, Artificial Sequence
  Synthetic
gcauauuguuguagugucccacccagggguccacauggcaac

SEQ ID NO: 14 - RNA, 20 nt, Artificial Sequence
  Synthetic
ggugaagaggagaacaauau

SEQ ID NO: 15 - RNA, 38 nt, Artificial Sequence
  Synthetic
auguuguucucgucuccaccagggguccacauggcaac

SEQ ID NO: 16 - RNA, 20 nt, Artificial Sequence
  Synthetic
ggugaauaguauaacaauau

SEQ ID NO: 17 - RNA, 38 nt, Artificial Sequence
  Synthetic
auguuguuauaguauccaccagggguccacauggcaac

SEQ ID NO: 18 - RNA, 24 nt, Artificial Sequence
  Synthetic
ggguggaauaguauaacaauaugc

SEQ ID NO: 19 - RNA, 42 nt, Artificial Sequence
  Synthetic
gcauauuguuauaguaucccacccagggguccacauggcaac

SEQ ID NO: 20 - RNA, 24 nt, Artificial Sequence
  Synthetic
ggguggaauaguacaacaauaugc

SEQ ID NO: 21 - RNA, 42 nt, Artificial Sequence
  Synthetic
gcauauuguuguaguaucccacccagggguccacauggcaac

SEQ ID NO: 22 - RNA, 22 nt, Artificial Sequence
  Synthetic
ggguggaauaguauaacaauau

SEQ ID NO: 23 - RNA, 40 nt, Artificial Sequence
  Synthetic
auauuguuauaguaucccacccagggguccacauggcaac

SEQ ID NO: 24 - RNA, 22 nt, Artificial Sequence
  Synthetic
ggguggaauaguauaacaaugc

SEQ ID NO: 25 - RNA, 40 nt, Artificial Sequence
  Synthetic
gcauuguuauaguaucccacccagggguccacauggcaac

SEQ ID NO: 26 - RNA, 22 nt, Artificial Sequence
  Synthetic
ggguggaauaguacaacaaugc

SEQ ID NO: 27 - RNA, 40 nt, Artificial Sequence
  Synthetic
gcauuguuguaguaucccacccagggguccacauggcaac

SEQ ID NO: 28 - RNA, 25 nt, Artificial Sequence
  Synthetic
gguggguggaauaguauaacaauau

SEQ ID NO: 29 - RNA, 43 nt, Artificial Sequence
  Synthetic
auguuguuauaguaucccacccaccagggguccacauggcaac

SEQ ID NO: 30 - RNA, 25 nt, Artificial Sequence
  Synthetic
gguggguggaagaguauaacaaugc

SEQ ID NO: 31 - RNA, 43 nt, Artificial Sequence
  Synthetic
gcguuguuauagucucccacccaccagggguccacauggcaac

SEQ ID NO: 32 - RNA, 25 nt, Artificial Sequence
  Synthetic
gguggguggaacaguauaacaaugc

SEQ ID NO: 33 - RNA, 43 nt, Artificial Sequence
  Synthetic
gcguuguuauagugucccacccaccagggguccacauggcaac

SEQ ID NO: 34 - RNA, 27 nt, Artificial Sequence
  Synthetic
gguggguggaauaguauaacaauaugc

SEQ ID NO: 35 - RNA, 45 nt, Artificial Sequence
  Synthetic
gcauguuguuauaguaucccacccaccagggguccacauggcaac

SEQ ID NO: 36 - RNA, 27 nt, Artificial Sequence
  Synthetic
gguggguggaauaguacaacaauaugc

SEQ ID NO: 37 - RNA, 45 nt, Artificial Sequence
  Synthetic
gcauguuguuguaguaucccacccaccagggguccacauggcaac

SEQ ID NO: 38 - RNA, 42 nt, Artificial Sequence
  Synthetic
gcauguuguucuagucucccacccagggguccacauggcaac

SEQ ID NO: 39 - RNA, 22 nt, Artificial Sequence
  Synthetic
ggguggaagaguagaacaaugc

SEQ ID NO: 40 - RNA, 40 nt, Artificial Sequence
  Synthetic
gcguuguucuagucucccacccagggguccacauggcaac

SEQ ID NO: 41 - RNA, 24 nt, Artificial Sequence
  Synthetic
ggguguaagaguagaacaauaugc

SEQ ID NO: 42 - RNA, 42 nt, Artificial Sequence
  Synthetic
gcauauuguucuagucucacacccagggguccacauggcaac

SEQ ID NO: 43 - RNA, 42 nt, Artificial Sequence
  Synthetic
gcauguuguucuagucucacacccagggguccacauggcaac

SEQ ID NO: 44 - RNA, 24 nt, Artificial Sequence
  Synthetic
ggguggaagaguacaacaauaugc

SEQ ID NO: 45 - RNA, 42 nt, Artificial Sequence
  Synthetic
gcauauuguuguagucucccacccagggguccacauggcaac

SEQ ID NO: 46 - RNA, 42 nt, Artificial Sequence
  Synthetic
gcauguuguuguagucucccacccagggguccacauggcaac

SEQ ID NO: 47 - RNA, 24 nt, Artificial Sequence
  Synthetic
ggguggaagaguauaacaauaugc

SEQ ID NO: 48 - RNA, 42 nt, Artificial Sequence
  Synthetic
gcauguuguuauagucucccacccagggguccacauggcaac

SEQ ID NO: 49 - RNA, 42 nt, Artificial Sequence
  Synthetic
gcauauuguuauagucucccacccagggguccacauggcaac

SEQ ID NO: 50 - RNA, 27 nt, Artificial Sequence
  Synthetic
ggugucgagaagaggagaacaauaugc

SEQ ID NO: 51 - RNA, 45 nt, Artificial Sequence
  Synthetic
gcauguuguucucgucuccucgacaccagggguccacauggcaac

SEQ ID NO: 52 - RNA, 45 nt, Artificial Sequence
  Synthetic
gcauauuguucucgucuccucgacaccagggguccacauggcaac

SEQ ID NO: 53 - RNA, 21 nt, Artificial Sequence
  Synthetic
ggugucgagaagaggagaaca

SEQ ID NO: 54 - RNA, 39 nt, Artificial Sequence
  Synthetic
uguucucgucuccucgacaccagggguccacauggcaac

SEQ ID NO: 55 - RNA, 21 nt, Artificial Sequence
  Synthetic
ggugucgagaagaggagaacg

SEQ ID NO: 56 - RNA, 39 nt, Artificial Sequence
  Synthetic
cguucucgucuccucgacaccagggguccacauggcaac

SEQ ID NO: 57 - RNA, 18 nt, Artificial Sequence
  Synthetic
ggguggaagaggagaacg

SEQ ID NO: 58 - RNA, 36 nt, Artificial Sequence
  Synthetic
cguucucgucucccacccagggguccacauggcaac

SEQ ID NO: 59 - RNA, 18 nt, Artificial Sequence
  Synthetic
ggguggaagaguagaccg

SEQ ID NO: 60 - RNA, 36 nt, Artificial Sequence
  Synthetic
cggucuagucucccacccagggguccacauggcaac

SEQ ID NO: 61 - RNA, 18 nt, Artificial Sequence
  Synthetic
ggguggaagaggagacca

SEQ ID NO: 62 - RNA, 36 nt, Artificial Sequence
  Synthetic
uggucucgucucccacccagggguccacauggcaac

SEQ ID NO: 63 - RNA, 16 nt, Artificial Sequence
  Synthetic
guggaagaggagaaca

SEQ ID NO: 64 - RNA, 34 nt, Artificial Sequence
  Synthetic
uguucucgucucccacagggguccacauggcaac

SEQ ID NO: 65 - RNA, 16 nt, Artificial Sequence
  Synthetic
guggaagaguagacca

SEQ ID NO: 66 - RNA, 34 nt, Artificial Sequence
  Synthetic
uggucuagucucccacagggguccacauggcaac

SEQ ID NO: 67 - RNA, 16 nt, Artificial Sequence
  Synthetic
guggaagaggagaacg

SEQ ID NO: 68 - RNA, 34 nt, Artificial Sequence
  Synthetic
cguucucgucucccacagggguccacauggcaac

SEQ ID NO: 69 - RNA, 16 nt, Artificial Sequence
  Synthetic
guggaagaguagaccg

SEQ ID NO: 70 - RNA, 34 nt, Artificial Sequence
  Synthetic
cggucuagucucccacagggguccacauggcaac

SEQ ID NO: 71 - RNA, 18 nt, Artificial Sequence
  Synthetic
guggaagaggagaacaac

SEQ ID NO: 72 - RNA, 36 nt, Artificial Sequence
  Synthetic
guuguucucgucucccacagggguccacauggcaac

SEQ ID NO: 73 - RNA, 18 nt, Artificial Sequence
  Synthetic
guggaagaggagaacagc

SEQ ID NO: 74 - RNA, 36 nt, Artificial Sequence
  Synthetic
gcuguucucgucucccacagggguccacauggcaac

SEQ ID NO: 75 - RNA, 20 nt, Artificial Sequence
  Synthetic
guggaagaggagaacaacgc

SEQ ID NO: 76 - RNA, 38 nt, Artificial Sequence
  Synthetic
gcguuguucucgucucccacagggguccacauggcaac

SEQ ID NO: 77 - RNA, 20 nt, Artificial Sequence
  Synthetic
guggaagaggagaacaaugc

SEQ ID NO: 78 - RNA, 38 nt, Artificial Sequence
  Synthetic
gcauuguucucgucucccacagggguccacauggcaac

SEQ ID NO: 79 - RNA, 17 nt, Artificial Sequence
  Synthetic
guggaagaguagaccac

SEQ ID NO: 80 - RNA, 35 nt, Artificial Sequence
  Synthetic
guggucuagucucccacagggguccacauggcaac

SEQ ID NO: 81 - RNA, 17 nt, Artificial Sequence
  Synthetic
guggaagaggagaacgc

SEQ ID NO: 82 - RNA, 35 nt, Artificial Sequence
  Synthetic
gcguucucgucucccacagggguccacauggcaac

SEQ ID NO: 83 - RNA, 43 nt, Artificial Sequence
  Synthetic
auguuguucucgucuccucgacaccagggguccacauggaaac

SEQ ID NO: 84 - RNA, 21 nt, Artificial Sequence
  Synthetic
gguggguggaagaggagaaca

SEQ ID NO: 85 - RNA, 39 nt, Artificial Sequence
  Synthetic
uguucucgucucccacccaccagggguccacauggcaac

SEQ ID NO: 86 - RNA, 21 nt, Artificial Sequence
  Synthetic
gguggguggaagaggagaacg

SEQ ID NO: 87 - RNA, 39 nt, Artificial Sequence
  Synthetic
cguucucgucucccacccaccagggguccacauggcaac

SEQ ID NO: 88 - RNA, 27 nt, Artificial Sequence
  Synthetic
gguggguggaacaggacaacaauaugc

SEQ ID NO: 89 - RNA, 45 nt, Artificial Sequence
  Synthetic
gcauauuguugucgugucccacccaccagggguccacauggcaac

SEQ ID NO: 90 - RNA, 21 nt, Artificial Sequence
  Synthetic
gguggguggaacaggagaaca

SEQ ID NO: 91 - RNA, 39 nt, Artificial Sequence
  Synthetic
uguucucgugucccacccaccagggguccacauggcaac

SEQ ID NO: 92 - RNA, 21 nt, Artificial Sequence
  Synthetic
gguggguggaacaggagaacg

SEQ ID NO: 93 - RNA, 39 nt, Artificial Sequence
  Synthetic
cguucucgugucccacccaccagggguccacauggcaac

SEQ ID NO: 94 - RNA, 27 nt, Artificial Sequence
  Synthetic
gguggguggaagaggagaacaauaugc

SEQ ID NO: 95 - RNA, 45 nt, Artificial Sequence
  Synthetic
gcauguuguucucgucucccacccaccagggguccacauggaaac

SEQ ID NO: 96 - RNA, 45 nt, Artificial Sequence
  Synthetic
gcauauuguucucgucucccacccaccagggguccacauggaaac

SEQ ID NO: 97 - RNA, 39 nt, Artificial Sequence
  Synthetic
uguucucgucucccacccaccagggguccacauggaaac

SEQ ID NO: 98 - RNA, 39 nt, Artificial Sequence
  Synthetic
cguucucgucucccacccaccagggguccacauggaaac

SEQ ID NO: 99 - RNA, 42 nt, Artificial Sequence
  Synthetic
gcauauuguucuagucucccacccagggguccacauggaaac

SEQ ID NO: 100 - RNA, 45 nt, Artificial Sequence
  Synthetic
gcauauuguugucgugucccacccaccagggguccacauggaaac

SEQ ID NO: 101 - RNA, 39 nt, Artificial Sequence
  Synthetic
uguucucgugucccacccaccagggguccacauggaaac

SEQ ID NO: 102 - RNA, 39 nt, Artificial Sequence
  Synthetic
cguucucgugucccacccaccagggguccacauggaaac

SEQ ID NO: 103 - RNA, 24 nt, Artificial Sequence
  Synthetic
ggguggaagaggagaacaauaugc

SEQ ID NO: 104 - RNA, 42 nt, Artificial Sequence
  Synthetic
gcauauuguucucgucucccacccagggguccacauggcaac

SEQ ID NO: 105 - RNA, 42 nt, Artificial Sequence
  Synthetic
gcguauuguucuagucucccacccagggguccacauggcaac

SEQ ID NO: 106 - RNA, 42 nt, Artificial Sequence
  Synthetic
gcguauuguucucgucucccacccagggguccacauggcaac

SEQ ID NO: 107 - RNA, 21 nt, Artificial Sequence
  Synthetic
ggguggaagaggagaacaaua

SEQ ID NO: 108 - RNA, 39 nt, Artificial Sequence
  Synthetic
uauuguucucgucucccacccagggguccacauggcaac

SEQ ID NO: 109 - RNA, 21 nt, Artificial Sequence
  Synthetic
ggguggaagaggagaacagcg

SEQ ID NO: 110 - RNA, 39 nt, Artificial Sequence
  Synthetic
cgcuguucucgucucccacccagggguccacauggcaac

SEQ ID NO: 111 - RNA, 21 nt, Artificial Sequence
  Synthetic
guguggaagaggagaacagug

SEQ ID NO: 112 - RNA, 39 nt, Artificial Sequence
  Synthetic
cacuguucucgucucccacccagggguccacauggcaac

SEQ ID NO: 113 - RNA, 18 nt, Artificial Sequence
  Synthetic
agggguccacauggaaac

SEQ ID NO: 114 - RNA, 18 nt, Artificial Sequence
  Synthetic
ggcuccccaggccccucc

SEQ ID NO: 115 - RNA, 21 nt, Artificial Sequence
  Synthetic
uguucucgucucccacccacc

SEQ ID NO: 116 - RNA, 21 nt, Artificial Sequence
  Synthetic
uguucucgucuccucgacacc

SEQ ID NO: 117 - RNA, 27 nt, Artificial Sequence
  Synthetic
gcauauuguucucgucuccucgacacc

SEQ ID NO: 118 - RNA, 22 nt, Artificial Sequence
  Synthetic
uuguucucgucucccacccacc

SEQ ID NO: 119 - RNA, 27 nt, Artificial Sequence
  Synthetic
gcauguuguucucgucuccucgacacc

SEQ ID NO: 120 - RNA, 21 nt, Artificial Sequence
  Synthetic
cguucucgucuccucgacacc

SEQ ID NO: 121 - RNA, 24 nt, Artificial Sequence
  Synthetic
gcauguuguucuagucucccaccc

SEQ ID NO: 122 - RNA, 24 nt, Artificial Sequence
  Synthetic
gcguauuguucuagucucccaccc

SEQ ID NO: 123 - RNA, 22 nt, Artificial Sequence
  Synthetic
gcguuguucuagucucccaccc

SEQ ID NO: 124 - RNA, 22 nt, Artificial Sequence
  Synthetic
auauuguuauaguaucccaccc

SEQ ID NO: 125 - RNA, 24 nt, Artificial Sequence
  Synthetic
gcauauuguuauaguaucccaccc

SEQ ID NO: 126 - RNA, 25 nt, Artificial Sequence
  Synthetic
auguuguuauaguaucccacccacc

SEQ ID NO: 127 - RNA, 27 nt, Artificial Sequence
  Synthetic
gcauguuguuauaguaucccacccacc

SEQ ID NO: 128 - RNA, 24 nt, Artificial Sequence
  Synthetic
gcauauuguuguaguaucccaccc

SEQ ID NO: 129 - RNA, 27 nt, Artificial Sequence
  Synthetic
gcauguuguuguaguaucccacccacc

SEQ ID NO: 130 - RNA, 24 nt, Artificial Sequence
  Synthetic
gcauauuguuguagugucccaccc

SEQ ID NO: 131 - RNA, 16 nt, Artificial Sequence
  Synthetic
cguucucgucucccac

SEQ ID NO: 132 - RNA, 17 nt, Artificial Sequence
  Synthetic
gcguucucgucucccac

SEQ ID NO: 133 - RNA, 18 nt, Artificial Sequence
  Synthetic
cguucucgucucccaccc

SEQ ID NO: 134 - RNA, 21 nt, Artificial Sequence
  Synthetic
cguucucgucucccacccacc

SEQ ID NO: 135 - RNA, 16 nt, Artificial Sequence
  Synthetic
cggucuagucucccac

SEQ ID NO: 136 - RNA, 18 nt, Artificial Sequence
  Synthetic
cggucuagucucccaccc

SEQ ID NO: 137 - RNA, 16 nt, Artificial Sequence
  Synthetic
uggucuagucucccac

SEQ ID NO: 138 - RNA, 17 nt, Artificial Sequence
  Synthetic
guggucuagucucccac

SEQ ID NO: 139 - RNA, 16 nt, Artificial Sequence
  Synthetic
uguucucgucucccac

SEQ ID NO: 140 - RNA, 18 nt, Artificial Sequence
  Synthetic
guuguucucgucucccac

SEQ ID NO: 141 - RNA, 18 nt, Artificial Sequence
  Synthetic
gcuguucucgucucccac

SEQ ID NO: 142 - RNA, 20 nt, Artificial Sequence
  Synthetic
gcguuguucucgucucccac

SEQ ID NO: 143 - RNA, 20 nt, Artificial Sequence
  Synthetic
gcauuguucucgucucccac

SEQ ID NO: 144 - RNA, 21 nt, Artificial Sequence
  Synthetic
cacuguucucgucucccaccc

SEQ ID NO: 145 - RNA, 21 nt, Artificial Sequence
  Synthetic
cgcuguucucgucucccaccc

SEQ ID NO: 146 - RNA, 27 nt, Artificial Sequence
  Synthetic
gcauguuguucucgucucccacccacc

SEQ ID NO: 147 - RNA, 27 nt, Artificial Sequence
  Synthetic
gcauauuguucucgucucccacccacc

SEQ ID NO: 148 - RNA, 21 nt, Artificial Sequence
  Synthetic
uauuguucucgucucccaccc

SEQ ID NO: 149 - RNA, 24 nt, Artificial Sequence
  Synthetic
gcauauuguucucgucucccaccc

SEQ ID NO: 150 - RNA, 24 nt, Artificial Sequence
  Synthetic
gcguauuguucucgucucccaccc

SEQ ID NO: 151 - RNA, 24 nt, Artificial Sequence
  Synthetic
gcauauuguucuagucucacaccc

SEQ ID NO: 152 - RNA, 24 nt, Artificial Sequence
  Synthetic
gcauguuguucuagucucacaccc

SEQ ID NO: 153 - RNA, 24 nt, Artificial Sequence
  Synthetic
gcauauuguuguagucucccaccc

SEQ ID NO: 154 - RNA, 24 nt, Artificial Sequence
  Synthetic
gcauguuguuguagucucccaccc

SEQ ID NO: 155 - RNA, 24 nt, Artificial Sequence
  Synthetic
gcauguuguuauagucucccaccc

SEQ ID NO: 156 - RNA, 24 nt, Artificial Sequence
  Synthetic
gcauauuguuauagucucccaccc

SEQ ID NO: 157 - RNA, 25 nt, Artificial Sequence
  Synthetic
gcguuguuauagucucccacccacc

SEQ ID NO: 158 - RNA, 20 nt, Artificial Sequence
  Synthetic
auguuguucucgucuccacc

SEQ ID NO: 159 - RNA, 20 nt, Artificial Sequence
  Synthetic
auguuguuauaguauccacc

SEQ ID NO: 160 - RNA, 18 nt, Artificial Sequence
  Synthetic
uggucucgucucccaccc

SEQ ID NO: 161 - RNA, 27 nt, Artificial Sequence
  Synthetic
gcauauuguugucgugucccacccacc

SEQ ID NO: 162 - RNA, 21 nt, Artificial Sequence
  Synthetic
uguucucgugucccacccacc

SEQ ID NO: 163 - RNA, 21 nt, Artificial Sequence
  Synthetic
cguucucgugucccacccacc

SEQ ID NO: 164 - RNA, 22 nt, Artificial Sequence
  Synthetic
gcauuguuauaguaucccaccc

SEQ ID NO: 165 - RNA, 22 nt, Artificial Sequence
  Synthetic
gcauuguuguaguaucccaccc

SEQ ID NO: 166 - RNA, 20 nt, Artificial Sequence
  Synthetic
gguggguggaagaggagaac

SEQ ID NO: 167 - RNA, 20 nt, Artificial Sequence
  Synthetic
guucucgucucccacccacc

SEQ ID NO: 168 - RNA, 25 nt, Artificial Sequence
  Synthetic
auauuguucucgucuccucgacacc

SEQ ID NO: 169 - DNA, 16 bp, Artificial Sequence
  Synthetic
gccatgtagacccctt

SEQ ID NO: 170 - DNA, 16 bp, Artificial Sequence
  Synthetic
gccatgtggacccctt

SEQ ID NO: 171 - DNA, 19 bp, Artificial Sequence
  Synthetic
ggaggggcctagggagcct

(19) United States
(12) Patent Application Publication
(10) Pub. No.: US 2023/0272391 A1
(43) Pub. Date: Aug. 31, 2023

(54) ADAR DEPENDENT EDITING COMPOSITIONS AND METHODS OF USE THEREOF

(71) Applicant: ADARx Pharmaceutical, Inc., San Diego, CA (US)

(72) Inventors: Zhen Li, San Diego, CA (US); Rui Zhu, San Diego, CA (US); Jean da Silva Correia, San Diego, CA (US); Kimberly Fultz, San Diego, CA (US); Sean Studer, San Diego, CA (US); Sam Lear, San Diego, CA (US)

(73) Assignee: ADARx Pharmaceutical, Inc., San Diego, CA (US)

(21) Appl. No.: 18/018,379

(22) PCT Filed: Jul. 30, 2021
(86) PCT No.: PCT/US2021/044074

§ 371 Date: Jan. 27, 2023

Related U.S. Application Data
(60) Provisional application No. 63/059,084, filed on Jul. 30, 2020.
(60) Provisional application No. 63/127,839, filed on Dec. 18, 2020.

(51) Int. Cl. C12N 15/113; C12N 15/85

(52) U.S. Cl. CPC C12N 15/1137; C12N 15/85; C12Y 102/01012; C12N 2310/321; C12N 2310/315; C12N 2320/33; C12N 2310/531

(57) ABSTRACT

Disclosed herein are compositions and methods for recruiting and using ADAR. In some aspects, compositions and methods for targeting ADAR activity to a desired region, sequence, or nucleotide are disclosed. In some aspects, compositions and methods for increasing the efficiency of directed ADAR editing are disclosed.

### CROSS-REFERENCE TO RELATED APPLICATIONS

This application claims priority under 35 U.S.C. § 119(e) to U.S. Provisional Patent Application No. 63/127,839, filed Dec. 18, 2020, and U.S. Provisional Patent Application No. 63/059,084, filed Jul. 30, 2020, each of which is hereby incorporated by reference in its entirety.

### BACKGROUND

Many diseases, disorders, and conditions are associated with one or more genetic mutations. Treatments based on replacing or correcting genetic mutations are being developed by harnessing naturally occurring enzymes and processes to supplement, substitute, and/or modify mutant intracellular DNA and/or RNA molecules.

Ribonucleic acid (RNA) editing is a natural process through which eukaryotic cells alter the sequence of their RNA molecules, often in a site-specific and precise way. This molecular process allows cells to make discrete changes (e.g., insertions, deletions, substitutions, etc.) to specific nucleotide sequences within an RNA molecule (e.g., mRNA, tRNA, rRNA, miRNA, etc.), after transcription by RNA polymerase. RNA editing is known to occur in all living organisms and is believed to be a highly conserved property of RNA.

RNA editing has several advantages including the ability to modulate the proportion of RNA that is edited and/or the timing of editing in contrast to DNA editing which has permanent and long-term effects.

### SUMMARY

The present disclosure relates to methods and compositions for editing nucleic acids and RNA molecules in particular. Methods and compositions can be used to change the sequence of one or more RNA molecules at one or more positions and either correct unwanted mutations or introduce changes that may have beneficial therapeutic or other effects.

In some aspects, compositions comprise one or more features that are effective for recruiting intracellular RNA editing enzymes (e.g., an endogenous adenosine deaminase acting on RNA (ADAR) enzyme). In some aspects, compositions comprise one or more features that are effective for increasing the local concentration (e.g., the concentration proximal to a composition as compared to the location absent a composition) of an RNA editing enzyme. In some embodiments, compositions comprise one or more features to promote binding of an intracellular RNA editing enzyme. In some aspects, compositions comprise one or more features that are effective for guiding RNA editing enzymes to a particular target nucleic acid sequence. In some aspects, compositions comprise one or more features that are effective for guiding RNA editing enzymes to a particular target nucleotide. In some aspects, compositions comprise one or more features that are effective for increasing the local concentration (e.g., the concentration proximal to a target nucleic acid sequence and/or target nucleotide as compared to the location absent the composition) of an RNA editing enzyme to a target nucleic acid sequence and/or target nucleotide.

In some aspects, a composition is a recruiting molecule that recruits (e.g., attracts) editing enzymes for use in editing a nucleic acid. In some aspects, a composition is a targeting molecule which targets a nucleic acid sequence for editing by an editing enzyme. In some embodiments, a nucleic acid is a ribonucleic acid (RNA). In some embodiments, a nucleic acid is a duplexed nucleic acid (e.g., comprising two strands). In some embodiments, a nucleic acid is a duplexed RNA.

In some aspects, the disclosure relates to an adenosine deaminase acting on ribonucleic acid (RNA) (ADAR) recruiting molecule comprising a double-stranded RNA duplex, wherein the double-stranded RNA duplex comprises two strands of RNA of an equal number of nucleotides, which two RNA strands are not connected to one another by means of a hairpin, wherein: (a) the 5′ nucleotide of each RNA strand is complementary to the 3′ nucleotide of the other RNA strand; and (b) at least one RNA strand of the double-stranded RNA duplex comprises at least one nucleoside modification and/or at least one backbone modification.

In some aspects, the disclosure relates to an adenosine deaminase acting on ribonucleic acid (RNA) (ADAR) recruiting molecule comprising a double-stranded RNA duplex, wherein the double-stranded RNA duplex comprises two strands of RNA of a different number of nucleotides, which two RNA strands are not connected to one another by means of a hairpin, wherein: (a) the 5′ nucleotide of each RNA strand is complementary to the 3′ nucleotide of the other RNA strand; and (b) at least one RNA strand of the double-stranded RNA duplex comprises at least one nucleoside modification and/or at least one backbone modification.

For example, in some aspects, the disclosure provides an ADAR recruiting molecule comprising a double-stranded RNA duplex, wherein the double-stranded RNA duplex comprises two RNA strands hybridized to form a duplex, wherein: (a) the two RNA strands are not connected to one another by means of a hairpin; (b) each of the two RNA strands forming the duplex has a 5′ terminal nucleotide that is complementary to a 3′ terminal nucleotide of the other RNA strand; (c) the double-stranded RNA duplex comprises at least one base pair mismatch, wherein the mismatch does not occur between the 5′ and 3′ terminal nucleotides; and (d) at least one of the two RNA strands forming the duplex comprises at least one nucleoside modification and/or at least one backbone modification. In some embodiments, the two RNA strands forming the duplex are of an equal number of nucleotides. In some embodiments, the two RNA strands forming the duplex are of a different number of nucleotides.

In some embodiments, the double-stranded RNA duplex comprises at least one base pair mismatch, wherein the mismatch is not positioned at either terminal nucleotide base pair of the double-stranded RNA duplex.

In some embodiments, an ADAR recruiting molecule, further comprises a single-stranded guide nucleic acid.

In some embodiments, a double-stranded RNA duplex comprises at least one nucleoside modification and at least one backbone modification.

In some embodiments, the at least one nucleoside modification comprises a 2′-O-Methyl, a 2′-O-methoxyethyl (2′-O-MOE), or a 2′-Fluoro modification.

In some embodiments, the at least one backbone modification of a double-stranded RNA duplex comprises a phosphorothioate modification.

In some embodiments, the at least one backbone modification of a double-stranded RNA duplex is positioned within 1-5 nucleotides of the terminal nucleotide of the RNA strand on which it is located. In some embodiments, the at least one backbone modification of a double-stranded RNA duplex is positioned within 1-3 nucleotides of the terminal nucleotide of the RNA strand on which it is located. In some embodiments, the at least one backbone modification of a double-stranded RNA duplex is positioned within 1 nucleotide of the terminal nucleotide of the RNA strand on which it is located.

In some embodiments, a double-stranded RNA duplex comprises more than one nucleoside modification. In some embodiments, a double-stranded RNA duplex comprises more than two nucleoside modifications. In some embodiments, more than 25% of the nucleosides in a double-stranded RNA duplex comprise a nucleoside modification. In some embodiments, more than 50% of the nucleosides in a double-stranded RNA duplex comprise a nucleoside modification. In some embodiments, more than 75% of the nucleosides in a double-stranded RNA duplex comprise a nucleoside modification.

In some embodiments, a double-stranded RNA duplex comprises more than one backbone modification. In some embodiments, a double-stranded RNA duplex comprises more than two backbone modifications. In some embodiments, a double-stranded RNA duplex comprises more than three backbone modifications. In some embodiments, more than 25% of the internucleoside linkages of a double-stranded RNA duplex comprise a modification. In some embodiments, more than 50% of the internucleoside linkages of a double-stranded RNA duplex comprise a modification. In some embodiments, more than 75% of the internucleoside linkages of a double-stranded RNA duplex comprise a modification.

In some embodiments, an ADAR recruiting molecule further comprises nucleotides attached to the 3′ end or 5′ end of at least one of the RNA strands of a double-stranded RNA duplex creating a 3′ and/or 5′ end overhang.

In some embodiments, an ADAR recruiting molecule further comprises an additional moiety.

In some embodiments, an ADAR recruiting molecule further comprises a linker.

In some embodiments, a single-stranded guide nucleic acid is guide Ribonucleic Acid (gRNA).

In some embodiments, a single-stranded guide nucleic acid comprises at least one nucleoside modification. In some embodiments, a single-stranded guide nucleic acid comprises at least one backbone modification. In some embodiments, a single-stranded guide nucleic acid comprises at least one nucleoside modification and at least one backbone modification. In some embodiments, a single-stranded guide nucleic acid comprises at least two nucleoside modifications. In some embodiments, a single-stranded guide nucleic acid comprises at least three nucleoside modifications. In some embodiments, more than 25% of the nucleosides of a single-stranded guide nucleic acid comprise a nucleoside modification. In some embodiments, more than 50% of the nucleosides in a single-stranded guide nucleic acid comprise a nucleoside modification. In some embodiments, more than 75% of the nucleosides in a single-stranded guide nucleic acid comprise a nucleoside modification.

In some embodiments, a single-stranded nucleic acid comprises at least one backbone modification. In some embodiments, a single-stranded nucleic acid comprises at least two backbone modifications. In some embodiments, a single-stranded nucleic acid comprises at least three backbone modifications. In some embodiments, more than 25% of the internucleoside linkages in a single-stranded guide nucleic acid comprise a phosphate modification. In some embodiments, more than 50% of the internucleoside linkages in a single-stranded guide nucleic acid comprise a phosphate modification. In some embodiments, more than 75% of the internucleoside linkages in a single-stranded guide nucleic acid comprise a phosphate modification.

In some embodiments, a single-stranded guide nucleic acid comprises sufficient complementarity to hybridize with a target sequence.

In some embodiments, a single-stranded guide nucleic acid comprises three consecutive non-modified nucleotides. In some embodiments, at least one of the three consecutive non-modified nucleotides pairs with a nucleotide adjacent to a target adenosine in the target sequence. In some embodiments, the middle nucleotide of the three consecutive non-modified nucleotides is opposite the target adenosine. In some embodiments, the middle nucleotide of the three consecutive non-modified nucleotides comprises cytosine (C). In some embodiments, a nucleotide opposite a target adenosine comprises: (a) cytosine (C); (b) a natural or modified nucleotide which does not base pair with adenosine (A); and/or (c) a natural or modified nucleotide which base pairs with guanine (G) or inosine (I).

In some embodiments, each RNA strand of a double-stranded RNA duplex is at least 5 nucleotides in length. In some embodiments, each RNA strand of a double-stranded RNA duplex is fewer than or equal to 100 nucleotides in length. In some embodiments, each RNA strand of a double-stranded RNA duplex is about 5 to about 80 nucleotides in length. In some embodiments, each RNA strand of a double-stranded RNA duplex is about 5 to about 60 nucleotides in length. In some embodiments, each RNA strand of a double-stranded RNA duplex is about 5 to about 40 nucleotides in length. In some embodiments, each RNA strand of a double-stranded RNA duplex is about 5 to about 30 nucleotides in length. In some embodiments, each RNA strand of a double-stranded RNA duplex is about 5 to about 20 nucleotides in length. In some embodiments, each RNA strand of a double-stranded RNA duplex is about 5 to about 10 nucleotides in length.

In some embodiments, a single-stranded guide nucleic acid is at least 5 nucleotides in length. In some embodiments, a single-stranded guide nucleic acid is fewer than or equal to 100 nucleotides in length. In some embodiments, a single-stranded guide nucleic acid is about 5 to about 80 nucleotides in length. In some embodiments, a single-stranded guide nucleic acid is about 5 to about 60 nucleotides in length. In some embodiments, a single-stranded guide nucleic acid is about 5 to about 40 nucleotides in length. In some embodiments, a single-stranded guide nucleic acid is about 5 to about 30 nucleotides in length. In some embodiments, a single-stranded guide nucleic acid is about 5 to about 20 nucleotides in length. In some embodiments, a single-stranded guide nucleic acid is about 5 to about 10 nucleotides in length. In some embodiments, a single-stranded guide nucleic acid is about 10 to about 30 nucleotides in length. In some embodiments, a single-stranded guide nucleic acid is about 15 to about 25 nucleotides in length. In some embodiments, a single-stranded guide nucleic acid is about 15 to about 20 nucleotides in length. In some embodiments, a single-stranded guide nucleic acid is about 17 to about 19 nucleotides in length.

In some embodiments, a single-stranded guide nucleic acid comprises at least 50% complementarity with a target sequence. In some embodiments, a single-stranded guide nucleic acid comprises at least 70% complementarity with a target sequence. In some embodiments, a single-stranded guide nucleic acid comprises at least 80% complementarity with a target sequence. In some embodiments, a single-stranded guide nucleic acid comprises at least 90% complementarity with a target sequence. In some embodiments, a single-stranded guide nucleic acid comprises at least 95% complementarity with a target sequence.

In some aspects, the disclosure relates to an RNA targeting molecule comprising: (a) a double-stranded RNA duplex, wherein a double-stranded RNA duplex comprises, two strands of RNA of an equal number of nucleotides, which two RNA strands are not connected to one another by means of a hairpin, wherein the 5′ nucleotide of each RNA strand is complementary to the 3′ nucleotide of the other RNA strand a double-stranded RNA duplex comprises at least one base pair mismatch, wherein the mismatch is not positioned at either terminal nucleotide base pair of a double-stranded RNA duplex; and (b) a single-stranded guide nucleic acid.

In some embodiments, a double-stranded RNA duplex comprises at least one nucleoside modification, and/or at least one backbone modification. In some embodiments, a double-stranded RNA duplex comprises at least one nucleoside modification and at least one backbone modification. In some embodiments, wherein the at least one nucleoside modification comprises a 2′-O-Methyl, a 2′-O-methoxyethyl (2′O-MOE), or a 2′-Fluoro modification. In some embodiments, wherein the at least one backbone modification comprises a phosphorothioate modification.

In some embodiments, the at least one backbone modification of a double-stranded RNA duplex is positioned within 1-5 nucleotides of the terminal nucleotide of the RNA strand on which it is located. In some embodiments, the at least one backbone modification of a double-stranded RNA duplex is positioned within 1-3 nucleotides of the terminal nucleotide of the RNA strand on which it is located. In some embodiments, the at least one backbone modification of a double-stranded RNA duplex is positioned within 1 nucleotide of the terminal nucleotide of the RNA strand on which it is located.

In some embodiments, a double-stranded RNA duplex comprises more than one nucleoside modification. In some embodiments, a double-stranded RNA duplex comprises more than two nucleoside modifications. In some embodiments, more than 25% of the nucleosides in a double-stranded RNA duplex comprise a nucleoside modification. In some embodiments, more than 50% of the nucleosides in a double-stranded RNA duplex comprise a nucleoside modification. In some embodiments, more than 75% of the nucleosides in a double-stranded RNA duplex comprise a nucleoside modification.

In some embodiments, a double-stranded RNA duplex comprises more than one backbone modification. In some embodiments, a double-stranded RNA duplex comprises more than two backbone modification. In some embodiments, a double-stranded RNA duplex comprises more than three backbone modification. In some embodiments, more than 25% of the internucleoside linkages of a double-stranded RNA duplex comprise a modification. In some embodiments, more than 50% of the internucleoside linkages of a double-stranded RNA duplex comprise a modification. In some embodiments, more than 75% of the internucleoside linkages of a double-stranded RNA duplex comprise a modification.

In some embodiments, an RNA targeting molecule further comprises nucleotides attached to the 3′ end or 5′ end of at least one of the RNA strands of a double-stranded RNA duplex creating a 3′ and/or 5′ end overhang.

In some embodiments, an RNA targeting molecule further comprises an additional moiety.

In some embodiments, an RNA targeting molecule further comprises a linker.

In some embodiments, a single-stranded guide nucleic acid is guide ribonucleic acid (gRNA).

In some embodiments, a single-stranded guide nucleic acid comprises at least one nucleoside modification. In some embodiments, a single-stranded guide nucleic acid comprises at least one backbone modification. In some embodiments, a single-stranded guide nucleic acid comprises at least one nucleoside modification and at least one backbone modification. In some embodiments, a single-stranded guide nucleic acid comprises at least two nucleoside modifications. In some embodiments, a single-stranded guide nucleic acid comprises at least three nucleoside modifications. In some embodiments, more than 25% of the nucleosides of a single-stranded guide nucleic acid comprise a nucleoside modification. In some embodiments, more than 50% of the nucleosides in a single-stranded guide nucleic acid comprise a nucleoside modification. In some embodiments, more than 75% of the nucleosides in a single-stranded guide nucleic acid comprise a nucleoside modification.

In some embodiments, a single-stranded guide nucleic acid comprises at least one backbone modification. In some embodiments, a single-stranded guide nucleic acid comprises at least two backbone modifications. In some embodiments a single-stranded guide nucleic acid comprises at least three backbone modifications. In some embodiments, more than 25% of the internucleoside linkages in a single-stranded guide nucleic acid comprise a phosphate modification. In some embodiments, more than 50% of the internucleoside linkages in a single-stranded guide nucleic acid comprise a phosphate modification. In some embodiments, more than 75% of the internucleoside linkages in a single-stranded guide nucleic acid comprise a phosphate modification.

In some embodiments, a single-stranded guide nucleic acid comprises sufficient complementarity to hybridize with a target sequence.

In some embodiments, a single-stranded guide nucleic acid comprises three consecutive non-modified nucleotides. In some embodiments, at least one of the three consecutive non-modified nucleotides of a single-stranded guide nucleic acid is complementary to a nucleotide adjacent to a target adenosine in the target sequence. In some embodiments, the middle nucleotide of the three consecutive non-modified nucleotides is opposite the target adenosine. In some embodiments, the middle nucleotide of the three consecutive non-modified nucleotides comprises cytosine (C). In some embodiments, a nucleotide opposite a target adenosine comprises: (a) cytosine (C); (b) a natural or modified nucleotide which does not base pair with adenosine (A); and/or (c) a natural or modified nucleotide which base pairs with guanine (G) or inosine (I).

In some embodiments, each RNA strand of a double-stranded RNA duplex is at least 5 nucleotides in length. In some embodiments, each RNA strand of a double-stranded RNA duplex is fewer than or equal to 100 nucleotides in length. In some embodiments, each RNA strand of a double-stranded RNA duplex is about 5 to about 80 nucleotides in length. In some embodiments, each RNA strand of a double-stranded RNA duplex is about 5 to about 60 nucleotides in length. In some embodiments, each RNA strand of a double-stranded RNA duplex is about 5 to about 40 nucleotides in length. In some embodiments, each RNA strand of a double-stranded RNA duplex is about 5 to about 30 nucleotides in length. In some embodiments, each RNA strand of a double-stranded RNA duplex is about 5 to about 20 nucleotides in length. In some embodiments, each RNA strand of a double-stranded RNA duplex is about 5 to about 10 nucleotides in length. In some embodiments, each RNA strand of a double-stranded RNA duplex is about 10 to about 30 nucleotides in length. In some embodiments, each RNA strand of a double-stranded RNA duplex is about 15 to about 25 nucleotides in length. In some embodiments, each RNA strand of a double-stranded RNA duplex is about 16 to about 23 nucleotides in length. In some embodiments, each RNA strand of a double-stranded RNA duplex is about 18 to about 22 nucleotides in length. In some embodiments, each RNA strand of a double-stranded RNA duplex is about 20 to about 22 nucleotides in length.

In some embodiments, a single-stranded guide nucleic acid is at least 5 nucleotides in length. In some embodiments, a single-stranded guide nucleic acid is fewer than or equal to 100 nucleotides in length. In some embodiments, a single-stranded guide nucleic acid is about 5 to about 80 nucleotides in length. In some embodiments, a single-stranded guide nucleic acid is about 5 to about 60 nucleotides in length. In some embodiments, a single-stranded guide nucleic acid is about 5 to about 40 nucleotides in length. In some embodiments, a single-stranded guide nucleic acid is about 5 to about 30 nucleotides in length. In some embodiments, a single-stranded guide nucleic acid is about 5 to about 20 nucleotides in length. In some embodiments, a single-stranded guide nucleic acid is about 5 to about 10 nucleotides in length.

In some embodiments, a single-stranded guide nucleic acid comprises at least 50% complementarity with a target sequence. In some embodiments, a single-stranded guide nucleic acid comprises at least 70% complementarity with a target sequence. In some embodiments, a single-stranded guide nucleic acid comprises at least 80% complementarity with a target sequence. In some embodiments, a single-stranded guide nucleic acid comprises at least 90% complementarity with a target sequence. In some embodiments, the single-stranded guide nucleic acid comprises at least 95% complementarity with a target sequence.

In some aspects, this disclosure relates to a method of deaminating a target nucleic acid in a subject, comprising, administering an effective amount of any of the ADAR recruiting molecules of the disclosure, and/or any of the RNA targeting molecules of the disclosure, wherein an ADAR recruiting molecule and/or an RNA targeting molecule comprises a single-stranded guide nucleic acid comprising a sequence which is sufficiently complementary to a target sequence to hybridize with the target sequence.

In some embodiments, the target sequence comprises a target adenosine.

In some aspects, the disclosure relates to a method of treating a subject, comprising administering any of the ADAR recruiting molecules of the disclosure, and/or any of the RNA targeting molecules of the disclosure, wherein an ADAR recruiting molecule and/or an RNA targeting molecule comprises a single-stranded guide nucleic acid comprising a sequence which is sufficiently complementary to a target sequence to hybridize with the target sequence.

In some embodiments, the target sequence comprises a target adenosine.

In some embodiments, the target adenosine is related to a disease or disorder, wherein the deamination of the target adenosine treats the disease or disorder.

In some embodiments, the disease or disorder is related to a point mutation wherein the point mutation is part of a nucleic acid encoding a protein related to the disorder. In some embodiments, the disease or disorder is related to a point mutation wherein the point mutation is part of a nucleic acid encoding a protein related to the disorder and the point mutation causes the protein to become inactive. In some embodiments, the disease or disorder is related to a point mutation wherein the point mutation is part of a nucleic acid encoding a protein related to the disorder and the point mutation causes the protein to have an altered activity as compared to the wild-type protein. In some embodiments, the disease or disorder is related to a point mutation wherein the point mutation is part of a nucleic acid encoding a protein related to the disorder and the point mutation causes the protein to have an altered activity as compared to the wild-type protein and the activity is detrimental to the subject. In some embodiments, the disease or disorder is related to a point mutation wherein the point mutation is part of a nucleic acid encoding a protein related to the disorder and the point mutation causes the protein to have an altered activity as compared to the wild-type protein and loses its wild-type activity. In some embodiments, the disease or disorder is related to a point mutation wherein the point mutation is part of a nucleic acid encoding a protein related to the disorder and the point mutation causes the otherwise inactive protein (e.g., the wild-type state of the protein is to be inactive) to become active. In some embodiments, the disease or disorder is related to a point mutation wherein the point mutation is part of a nucleic acid encoding a protein related to the disorder and the point mutation causes the protein to have decreased activity as compared to the wild-type. In some embodiments, the disease or disorder is related to a point mutation wherein the point mutation is part of a nucleic acid encoding a protein related to the disorder and the point mutation causes the protein to have increased activity as compared to the wild-type. In some embodiments, the disease or disorder is related to a point mutation wherein the editing of a target adenosine to an inosine alters the function of protein encoded by the nucleic acid harboring the target adenosine, thereby ameliorating the disease or disorder.

In some embodiments, the disease or disorder is selected from: Cystic fibrosis, Hurler Syndrome, alpha-1-antitrypsin (A1AT) deficiency, Parkinson's disease, Alzheimer's disease, albinism, Amyotrophic lateral sclerosis, Asthma, beta-thalassemia (β-thalassemia), Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B, and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, Sturge-Weber Syndrome, and cancer.

In some aspect, the disclosure relates to an RNA targeting molecule comprising: (a) a double-stranded RNA duplex comprising two RNA strands; (b) a single-stranded guide nucleic acid; and (c) a linker; wherein the double-stranded RNA duplex is connected to the single-stranded guide nucleic acid via the linker.

In some embodiments, a linker is connected to the 5′ terminal nucleotide of one of the two strands of the RNA duplex. In some embodiments, a linker is connected to the 3′ terminal nucleotide of one of the two strands of the RNA duplex. In some embodiments, a linker is connected to a nucleotide located between the 5′ terminal nucleotide and the 3′ terminal nucleotide of one of the two strands of the RNA duplex. In some embodiments, a linker is connected to the 5′ nucleotide of the guide nucleic acid. In some embodiments, a linker is connected to the 3′ nucleotide of the guide nucleic acid. In some embodiments, a linker is connected to a nucleotide located between the 5′ terminal nucleotide and the 3′ terminal nucleotide the guide nucleic acid. In some embodiments, a linker is connected to a sugar of the 5′ terminal nucleotide, or a 3′ hydroxyl or sugar of the 3′ terminal nucleotide of one strand of the RNA duplex. In some embodiments, a linker is connected to a sugar of the 5′ terminal nucleotide, or a 3′ hydroxyl or sugar of the 3′ terminal nucleotide of the guide nucleic acid. In some embodiments, a linker is connected to an internucleoside linkage of one RNA strand of the RNA duplex. In some embodiments, a linker is connected to an internucleoside linkage of the guide nucleic acid. In some embodiments, a linker is connected to a nucleoside sugar of one RNA strand of the RNA duplex. In some embodiments, a linker is connected to a nucleoside sugar of the guide nucleic acid.

In some embodiments, a linker connects the 3′ end of the guide nucleic acid to the 5′ end of one RNA strand of the RNA duplex. In some embodiments, a linker connects the 5′ end of the guide nucleic acid to the 3′ end of one RNA strand of the RNA duplex. In some embodiments, a linker connects the 3′ end of the guide nucleic acid to the 3′ end of one RNA strand of the RNA duplex. In some embodiments, a linker connects the 5′ end of the guide nucleic acid to the 5′ end of one RNA strand of the RNA duplex. In some embodiments, a linker connects the 3′ end of the guide nucleic acid to a nucleotide located between the 5′ terminal nucleotide and the 3′ terminal nucleotide of one RNA strand of the RNA duplex.

In some embodiments, a linker is connected to an internucleoside linkage or a nucleoside sugar of one RNA strand of the RNA duplex.

In some embodiments, a linker connects the 5′ end of the guide nucleic acid to a nucleotide located between the 5′ terminal nucleotide and the 3′ terminal nucleotide of one RNA strand of the RNA duplex.

In some embodiments, a linker is connected to an internucleoside linkage or a nucleoside sugar of one RNA strand of the RNA duplex. In some embodiments, a linker connects the 3′ end of one RNA strand of the RNA duplex to a nucleotide located between the 5′ terminal nucleotide and the 3′ terminal nucleotide of the guide nucleic acid. In some embodiments, a linker is connected to an internucleoside linkage or a nucleoside sugar of the guide nucleic acid.

In some embodiments, a linker connects the 5′ end of one RNA strand of the RNA duplex to a nucleotide located between the 5′ terminal nucleotide and the 3′ terminal nucleotide of the guide nucleic acid. In some embodiments, a linker is connected to an internucleoside linkage or a nucleoside sugar of the guide nucleic acid.

In some embodiments, a linker is an unbranched linker. In some embodiments, a linker is a branched linker.

In some embodiments, a linker is a non-covalent linker comprising a first binding partner covalently attached to one strand of the double-stranded RNA duplex, and a second binding partner covalently attached to the single-stranded guide nucleic acid. In some embodiments, a first binding partner is a receptor and a second binding partner is a ligand specific for the receptor. In some embodiments, a second binding partner is a receptor and a first binding partner is a ligand specific for the receptor. In some embodiments, a first binding partner is biotin and a second binding partner is streptavidin. In some embodiments, a first binding partner is streptavidin and a second binding partner is biotin.

In some embodiments, a linker is a covalent linker.

In some embodiments, at least one linker is greater than or equal to 4 atoms in length. In some embodiments, at least one linker is fewer than or equal to 180 atoms in length.

In some embodiments, a linker comprises an alkyl, alkenyl, alkynyl, substituted alkyl, substituted alkenyl, substituted alkynyl, repeated ethylene glycol group, ether, thioether, urea, carbonate, amine, amide, maleimide-thioether, disulfide, phosphodiester, sulfonamide linkage, a product of a click reaction, a triazole from the azide-alkyne cycloaddition, carbamate, a cleavable linker such as, a redox cleavable linker such as a reductively cleavable linker, a disulfide group, an acid cleavable linker, a hydrazone group, an ester group, an acetal group, or a ketal group, an esterase cleavable linker, an ester group, a phosphatase cleavable linker, a phosphate group, or a peptidase cleavable linker, a peptide bond, a bio-cleavable linker, DNA, RNA, disulfide, amide, functionalized monosaccharides, or oligosaccharides of galactosamine.

In some embodiments, a linker comprises a moiety derived from a click chemistry reaction. In some embodiments, a linker is a triazole, diazole, diazine, sulfide bond, maleimide ring, succinimide ring, ester, or amide. In some embodiments, a linker comprises one or more amino acids.

In some embodiments, a linker comprises an organic molecule, group, polymer, or chemical domain. In some embodiments, a chemical domain comprises an amide, urea, carbamate, carbonate, ester, acetal, ketal, phosphoramidite, hydrazone, imine, oxime, disulfide, silyl, hydrazine, hydrazone, thiol, imidazole, carbon-carbon bond, carbon-heteroatom bond, or azo domain.

In some embodiments, a linker is polymeric. In some embodiments, a polymeric linker comprises polyethylene, polyethylene glycol, polyamide, polyester, or polyether.

In some embodiments, the linker comprises any one of Formula (I)-Formula (VII).

In some embodiments, the linker does not comprise a nucleotide or nucleoside. In some embodiments, the linker is a non-nucleic acid linker.

In some aspects, the disclosure relates to a double-stranded RNA duplex comprising: (a) an RNA strand comprising a sequence with at least 70% identity to Strand Ref.: 24 or 27; and (b) an RNA strand comprising a sequence with at least 70% identity to Strand Ref.: 25 or 28; and wherein the single-stranded guide nucleic acid comprises a sequence with at least 70% identity to Strand Ref.: 23 or 26.

In some aspects, the disclosure relates to a double-stranded RNA duplex comprising: (a) an RNA strand comprising a sequence according to Strand Ref.: 24 or 27; and (b) an RNA strand comprising a sequence according to Strand Ref.: 25 or 28; and wherein, the at least one single-stranded guide nucleic acid comprises a sequence according to Strand Ref.: 23 or 26.

In some aspects, the disclosure relates to a double-stranded RNA duplex comprising: (a) an RNA strand comprising a sequence with at least 70% identity to Strand Ref.: 353 or 355; and (b) an RNA strand comprising a sequence with at least 70% identity to Strand Ref.: 815 or 818. In some aspects, the disclosure relates to a double-stranded RNA duplex comprising: (a) an RNA strand comprising a sequence according to Strand Ref.: 353 or 355; and (b) an RNA strand comprising a sequence according to Strand Ref.: 815 or 818.

In some aspects, the disclosure relates to a double-stranded RNA duplex comprising: (a) an RNA strand comprising a sequence with at least 70% identity to Strand Ref.: 641 or 643; and (b) an RNA strand comprising a sequence with at least 70% identity to Strand Ref.: 841 or 869. In some aspects, the disclosure relates to a double-stranded RNA duplex comprising: (a) an RNA strand comprising a sequence according to Strand Ref.: 641 or 643; and (b) an RNA strand comprising a sequence according to Strand Ref.: 841 or 869.

In some embodiments, an RNA targeting molecule comprises two or more double-stranded RNA duplexes. In some embodiments, an RNA targeting molecule comprises two or more single-stranded guide nucleic acids.

In some embodiments, an RNA targeting molecule comprises 2-10 double-stranded RNA duplexes. In some embodiments, an RNA targeting molecule comprises 2-10 single-stranded guide nucleic acids. In some embodiments, an RNA targeting molecule comprises 2-5 double-stranded RNA duplexes. In some embodiments, an RNA targeting molecule comprises 2-5 single-stranded guide nucleic acids.

In some embodiments, one strand of a double-stranded RNA duplex is not covalently connected to the other strand of the RNA duplex. In some embodiments, a double-stranded RNA duplex does not comprise a hairpin connecting one strand of the RNA duplex to the other strand of the RNA duplex. In some embodiments, a double-stranded RNA duplex comprises two RNA strands having an equal number of nucleotides. In some embodiments, a double-stranded RNA duplex comprises two RNA strands having a different number of nucleotides.

In some aspects, the disclosure relates to an RNA targeting molecule comprising: (a) a first double-stranded RNA duplex comprising two RNA strands; (b) a second double-stranded RNA duplex comprising two RNA strands; (b) a single-stranded guide nucleic acid; and (c) a linker; wherein the first double-stranded RNA duplex is connected to the second double-stranded RNA duplex via the linker.

In some aspects, the disclosure relates to an RNA targeting molecule comprising: (a) a double-stranded RNA duplex comprising two RNA strands; (b) a first single-stranded guide nucleic acid; (c) a second single-stranded guide nucleic acid; and (c) a linker; wherein the first single-stranded guide nucleic acid is connected to the second single-stranded guide nucleic acid via the linker.

In some embodiments, an RNA targeting molecule comprises a double-stranded RNA duplex comprising at least one mismatch. In some embodiments, an RNA targeting molecule comprises a single-stranded guide nucleic acid comprising at least two mismatches.

These and other aspects and embodiments will be described in greater detail herein. The description of some exemplary embodiments of the disclosure are provided for illustration purposes only and not meant to be limiting. Additional compositions and methods are also embraced by this disclosure.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, Drawings, Examples, and Claims.

### BRIEF DESCRIPTIONS OF THE DRAWINGS

Further aspects of the disclosure will be readily appreciated upon review of the Detailed Description of its various aspects and embodiments, described below, when taken in conjunction with the accompanying Drawings.

FIGS. 1A-1B show non-limiting components and examples of an RNA Recruiting Molecule and/or an RNA Targeting Molecule. FIG. 1A shows a double-stranded RNA duplex 100, where two strands of RNA are depicted running anti-parallel to one another with the terminal nucleotides aligned (e.g., non-overhanging, without 5′ and/or 3′ overhang). Arrow heads depict the opposite directionality of the two RNA strands, wherein the “tip” of the arrow (e.g., the arrow head's direction) indicates the 3′ end of the RNA strand and the opposite end (e.g., the tail, end opposite the end carrying the arrow head) indicates the 5′ end of the RNA strand. Also shown is a single-stranded guide nucleic acid 102. Arrow heads depict the opposite directionality of the nucleic acid, wherein the “tip” of the arrow (e.g., the arrow head's direction) indicates the 3′ end of the nucleic acid and the opposite end (e.g., the tail, end opposite the end carrying the arrow head) indicates the 5′ end of the nucleic acid. Further shown is an illustration of a linker 104 as further described in the instant disclosure. The general structures as depicted in FIG. 1A (e.g., open shapes: double-stranded RNA duplex; stippled shapes: single-stranded guide nucleic acid; and wavy lines: linker) shall continue and apply throughout these FIGS. unless context requires otherwise. FIG. 1B shows non-limiting examples of double-stranded RNA duplexes of the instant disclosure where one strand may be longer than the other. As shown, the terminal nucleotides are aligned (e.g., ends are blunt with no overhang), but the excess length comprises a hairpin (e.g., stem-loop construct) within one or both strands of a double-stranded duplex. The top structures shows an example wherein the top strand is longer and comprises a bulge without 110 or with 112 a hairpin, but the bottom strand does not comprise a bulge. The bottom structures show examples wherein each strand comprises a hairpin. As shown, the hairpins may be of the same 114 or different 116 lengths, the top and bottom strand may further be of equal 114 or different 116 lengths, but the terminal nucleotides are still aligned (e.g., ends are blunt with no overhang).

FIGS. 2A-2G show non-limiting examples of several embodiments as described herein. In FIG. 2A, a double-stranded RNA duplex is shown connected to a single-stranded guide nucleic acid at the 5′ end (200) and 3′ end (202) of a double-stranded RNA duplex. Further embodiments are depicted using a linker positioned between the double-stranded RNA duplex and the single-stranded guide nucleic acid (204 and 206). Further, an additional moiety 280 (e.g., a delivery moiety, tag or marker) is depicted attached to the end of the double-stranded RNA duplex distal to the connection of the double-stranded RNA duplex to the single-stranded guide nucleic acid. The additional moiety (e.g., a delivery moiety, tag or marker) is shown attached to a different RNA strand of the double-stranded RNA duplex as the single-stranded guide nucleic acid (208) and the same RNA strand of the double-stranded RNA duplex as the single-stranded guide nucleic acid (210). FIG. 2B shows non-limiting examples of several embodiments as described herein. A single-stranded guide nucleic acid is shown connected to a linker which is connected to a double-stranded RNA duplex. As shown, the configuration may be the linker connected to either the 5′ end (212 and 218) or the 3′ end (214 and 216) of the single-stranded guide nucleic acid, and similarly the linker may be connected to either the 5′ end (216 and 218) or the 3′ end (212 and 214) of either RNA strand of the double-stranded RNA duplex. A double-stranded RNA duplex is shown connected to a linker which is connected to a single-stranded guide nucleic acid, the configuration may be the linker connected to either the 5′ or the 3′ end of the single-stranded guide nucleic acid, and similarly the linker may be the connected to either the 5′ or the 3′ end of either RNA strand of the double-stranded RNA duplex. FIG. 2C shows different example configurations of a double-stranded RNA duplex connected to a linker which is connected to a single-stranded guide nucleic acid. As shown, the configuration may be the linker connected to either the 5′ or the 3′ end of the single-stranded guide nucleic acid, and similarly the linker may be connected to either the 5′ or the 3′ end of either RNA strand of the double-stranded RNA duplex. In structures 220, 222, 224, and 226, the linker and the single-stranded guide nucleic acid are shown in parentheses to illustrate that there may be multiple modules of the linker-single-stranded guide nucleic acid complex (e.g., more than one linker, more than one single-stranded guide nucleic acid) attached to the double-stranded RNA duplex (indicated by the subscript “n,” where n is greater than or equal to 1). In structures 228, 230, 232, and 234, the linker and the double-stranded RNA duplex are shown in parentheses to illustrate that there may be multiple modules of the linker-double-stranded RNA duplex complex (e.g., more than one linker, more than one double-stranded RNA duplex) attached to the single-stranded guide nucleic acid (indicated by the subscript “n,” where n is greater than or equal to 1). If there are multiple modules of the linker-single-stranded guide nucleic acid complex or linker-double-stranded RNA duplex complex, they may be sequential (e.g., linker-single-stranded guide nucleic acid-linker-single-stranded guide nucleic acid . . . n), or they may branch in various configurations (e.g., branching linker with multiple single-stranded guide nucleic acids, or linkers attached to a single single-stranded guide nucleic acid); there is no requirement that they each attach to the end of the prior component (e.g., linker or single-stranded guide nucleic acid), and they are shown attaching through the terminal nucleotides of each of the double-stranded RNA duplex and the single-stranded guide nucleic acid. The connection may be through any portion (e.g., through the nucleobase, five carbon sugar, and/or phosphate, or as further described herein). FIGS. 2D-2F show a single-stranded guide nucleic acid connected to a linker which is connected to a double-stranded RNA duplex in various configurations. The linker and the double-stranded RNA duplex, or the linker and the single-stranded guide nucleic acid, are shown in parentheses to illustrate that there may be multiple modules of the linker-double-stranded RNA duplex or linker-single-stranded guide nucleic acid complex, and these multiple modules are as described for FIG. 2C ((e.g., more than one linker, more than one double-stranded RNA duplex, more than one single-stranded guide nucleic acid) attached to the single-stranded guide nucleic acid or double-stranded RNA duplex (indicated by the subscript “n,” where n is greater than or equal to 1)). If there are multiple modules of the linker-double-stranded RNA duplex or linker-single-stranded guide nucleic acid complex, they may be sequential (e.g., linker-double-stranded RNA duplex-linker-double-stranded RNA duplex . . . n), or they may branch in various configurations (e.g., branching linker with multiple double-stranded RNA duplexes, or linkers attached to a single double-stranded RNA duplex), e.g., there is no requirement that they each attach to the end of the prior component (e.g., linker or double-stranded RNA duplex), and they are shown attaching through a point between the terminal nucleotides of one or each of the double-stranded RNA duplex and the single-stranded guide nucleic acid. The connection may be through any portion (e.g., through the nucleobase, five carbon sugar, and/or phosphate, or as further described herein). FIG. 2D shows example configurations in which a linker connects a terminal end of the single-stranded guide nucleic acid to the double-stranded RNA duplex at a position between the terminal nucleotides (e.g., not at the 5′ or 3′ ends) of any of the strands comprised by the double-stranded RNA duplex. FIG. 2E shows example configurations in which a linker connects a terminal end of the double-stranded RNA duplex to the single-stranded guide nucleic acid at a position between the terminal nucleotides (e.g., not at the 5′ or 3′ ends) of the single-stranded guide nucleic acid. FIG. 2F shows non-limiting examples of a double-stranded RNA duplex connected to a linker connected to a single-stranded guide nucleic acid, wherein the linker is connected to each component between the terminal nucleotides (e.g., not at the 5′ or 3′ ends) of any of the strands comprised by the double-stranded RNA duplex and single-stranded guide nucleic acid. FIG. 2G shows example configurations in which a double-stranded RNA duplex is attached to multiple single-stranded guide nucleic acids.

FIGS. 3A-3B show editing by deamination of a target adenosine in the 3′ untranslated region (UTR) of Glyceraldehyde 3-phosphate dehydrogenase (GAPDH) by RD0016 and RD0034 against RL0079 (Negative Control) and RH0001 (Positive Control; RH0001 strand). An asterisk marks the peak of detected editing at the target adenosine. FIG. 3A shows editing in HeLa cells in the 3′ UTR of GAPDH in RL0079, RH0001, and RD0016. FIG. 3B shows editing in the 3′ UTR of GAPDH in RL0079, RH0001, and RD0034. Editing is shown by calculated change of a target base based upon sequencing data.

FIGS. 4A-4E show editing by deamination of a target adenosine (boxed in) in the 3′ untranslated region (UTR) of Glyceraldehyde 3-phosphate dehydrogenase (GAPDH) by RL0079 (Negative Control), RH0001 (Positive Control; RH0001 strand), RD0016, RD0034, and RD0037, in the absence (top panel of each of FIGS. 4A-4E) and presence (bottom panel of each of FIGS. 4A-4E) of interferon alpha (IFNα; 1200U) in HeLa cells. FIG. 4A shows editing in the 3′ UTR of GAPDH by RL0079 without IFNα (top) and with 1200U IFNα (bottom) in HeLa cells. FIG. 4B shows editing in the 3′ UTR of GAPDH by RH0001 without IFNα (top) and with 1200U IFNα (bottom) in HeLa cells. FIG. 4C shows editing in the 3′ UTR of GAPDH by RD0016 without IFNα (top) and with 1200U IFNα (bottom) in HeLa cells. FIG. 4D shows editing in the 3′ UTR of GAPDH by RD0034 without IFNα (top) and with 1200U IFNα (bottom) in HeLa cells. FIG. 4E shows editing in the 3′ UTR of GAPDH by RD0037 without IFNα (top) and with 1200U IFNα (bottom) in HeLa cells. Editing is shown by calculated change of a target base based upon sequencing data. Sequences shown:

(SEQ ID NO: 169)
GCCATGTAGACCCCTT
and
(SEQ ID NO: 170)
GCCATGTGGACCCCTT.

FIGS. 5A-5E show editing by deamination of a target adenosine (boxed in) in the 3′ untranslated region (UTR) of Glyceraldehyde 3-phosphate dehydrogenase (GAPDH) by RL0079 (Negative Control), RH0001 (Positive Control; RH0001 strand), RD0016, RD0034, and RD0037, in the absence (top panel of each of FIGS. 5A-5E) and presence (bottom panel of each of FIGS. 5A-5E) of interferon alpha (IFNα; 1200U) in U-2 OS cells. FIG. 5A shows editing in the 3′ UTR of GAPDH by RL0079 without IFNα (top) and with 1200U IFNα (bottom) in U-2 OS cells. FIG. 5B shows editing in the 3′ UTR of GAPDH by RH0001 without IFNα (top) and with 1200U IFNα (bottom) in U-2 OS cells. FIG. 5C shows editing in the 3′ UTR of GAPDH by RD0016 without IFNα (top) and with 1200U IFNα (bottom) in U-2 OS cells. FIG. 5D shows editing in the 3′ UTR of GAPDH by RD0034 without IFNα (top) and with 1200U IFNα (bottom) in U-2 OS cells. FIG. 5E shows editing in the 3′ UTR of GAPDH by RD0037 without IFNα (top) and with 1200U IFNα (bottom) in U-2 OS cells. Editing is shown by calculated change of a target base based upon sequencing data. Sequences shown:

(SEQ ID NO: 169)
GCCATGTAGACCCCTT
and
(SEQ ID NO: 170)
GCCATGTGGACCCCTT.

FIGS. 6A-6E show editing by deamination of a target adenosine (boxed in) in the 3′ untranslated region (UTR) of Glyceraldehyde 3-phosphate dehydrogenase (GAPDH) by RL0079 (Negative Control), RH0001 (Positive Control; RH0001 strand), RD0016, RD0034, and RD0037, in the absence (top panel of each of FIGS. 6A-6E) and presence (bottom panel of each of FIGS. 6A-6E) of interferon alpha (IFNα; 1200U) in NCI-H1395 cells. FIG. 6A shows editing in the 3′ UTR of GAPDH by RL0079 without IFNα (top) and with 1200U IFNα (bottom) in NCI-H1395 cells. FIG. 6B shows editing in the 3′ UTR of GAPDH by RH0001 without IFNα (top) and with 1200U IFNα (bottom) in NCI-H1395 cells. FIG. 6C shows editing in the 3′ UTR of GAPDH by RD0016 with 1200U IFNα in NCI-H1395 cells. FIG. 6D shows editing in the 3′ UTR of GAPDH by RD0034 without IFNα (top) and with 1200U IFNα (bottom) in NCI-H1395 cells. FIG. 6E shows editing in the 3′ UTR of GAPDH by RD0037 without IFNα (top) and with 1200U IFNα (bottom) in NCI-H1395 cells. Editing is shown by calculated change of a target base based upon sequencing data. Sequences shown:

(SEQ ID NO: 169)
GCCATGTAGACCCCTT
and
(SEQ ID NO: 170)
GCCATGTGGACCCCTT.

FIGS. 7A-7E show editing by deamination of a target adenosine (boxed in) in the 3′ untranslated region (UTR) of Glyceraldehyde 3-phosphate dehydrogenase (GAPDH) by RL0079 (Negative Control), RH0001 (Positive Control; RH0001 strand), RD0016, RD0034, and RD0037, in the absence (top panel of each of FIGS. 7A-7E) and presence (bottom panel of each of FIGS. 7A-7E) of interferon alpha (IFNα; 1200U) in NCI-H1993 cells. FIG. 7A shows editing in the 3′ UTR of GAPDH by RL0079 without IFNα (top) and with 1200U IFNα (bottom) in NCI-H1993 cells. FIG. 7B shows editing in the 3′ UTR of GAPDH by RH0001 without IFNα (top) and with 1200U IFNα (bottom) in NCI-H1993 cells. FIG. 7C shows editing in the 3′ UTR of GAPDH by RD0016 without IFNα (top) and with 1200U IFNα (bottom) in NCI-H1993 cells. FIG. 7D shows editing in the 3′ UTR of GAPDH by RD0034 without IFNα (top) and with 1200U IFNα (bottom) in NCI-H1993 cells. FIG. 7E shows editing in the 3′ UTR of GAPDH by RD0037 without IFNα (top) and with 1200U IFNα (bottom) in NCI-H1993 cells. Editing is shown by calculated change of a target base based upon sequencing data. Sequences shown:

(SEQ ID NO: 169)
GCCATGTAGACCCCTT
and
(SEQ ID NO: 170)
GCCATGTGGACCCCTT.

FIGS. 8A-8E show editing by deamination of a target adenosine (boxed in) in the 3′ untranslated region (UTR) of Glyceraldehyde 3-phosphate dehydrogenase (GAPDH) by RL0079 (Negative Control), RH0001 (Positive Control; RH0001 strand), RD0016, RD0034, and RD0037, in the absence (top panel of each of FIGS. 8A-8E) and presence (bottom panel of each of FIGS. 8A-8E) of interferon alpha (IFNα; 1200U) in Hep G2 cells. FIG. 8A shows editing in the 3′ UTR of GAPDH by RL0079 without IFNα (top) and with 1200U IFNα (bottom) in Hep G2 cells. FIG. 8B shows editing in the 3′ UTR of GAPDH by RH0001 without IFNα (top) and with 1200U IFNα (bottom) in Hep G2 cells. FIG. 8C shows editing in the 3′ UTR of GAPDH by RD0016 without IFNα (top) and with 1200U IFNα (bottom) in Hep G2 cells. FIG. 8D shows editing in the 3′ UTR of GAPDH by RD0034 without IFNα (top) and with 1200U IFNα (bottom) in Hep G2 cells. FIG. 8E shows editing in the 3′ UTR of GAPDH by RD0037 without IFNα (top) and with 1200U IFNα (bottom) in Hep G2 cells. Editing is shown by calculated change of a target base based upon sequencing data.

Sequences shown:

(SEQ ID NO: 169)
GCCATGTAGACCCCTT
and
(SEQ ID NO: 170)
GCCATGTGGACCCCTT.

FIGS. 9A-9E show editing by deamination of a target adenosine (boxed in) in the 3′ untranslated region (UTR) of Glyceraldehyde 3-phosphate dehydrogenase (GAPDH) by RL0079 (Negative Control), RH0001 (Positive Control; RH0001 strand), RD0016, RD0034, and RD0037, in the absence (top panel of each of FIGS. 9A-9E) and presence (bottom panel of each of FIGS. 9A-9E) of interferon alpha (IFNα; 1200U) in SK-BR-3 cells. FIG. 9A shows editing in the 3′ UTR of GAPDH by RL0079 without IFNα (top) and with 1200U IFNα (bottom) in SK-BR-3 cells. FIG. 9B shows editing in the 3′ UTR of GAPDH by RH0001 without IFNα (top) and with 1200U IFNα (bottom) in SK-BR-3 cells. FIG. 9C shows editing in the 3′ UTR of GAPDH by RD0016 without IFNα (top) and with 1200U IFNα (bottom) in SK-BR-3 cells. FIG. 9D shows editing in the 3′ UTR of GAPDH by RD0034 without IFNα (top) and with 1200U IFNα (bottom) in SK-BR-3 cells. FIG. 9E shows editing in the 3′ UTR of GAPDH by RD0037 without IFNα (top) and with 1200U IFNα (bottom) in SK-BR-3 cells. Editing is shown by calculated change of a target base based upon sequencing data. Sequences shown:

(SEQ ID NO: 169)
GCCATGTAGACCCCTT
and
(SEQ ID NO: 170)
GCCATGTGGACCCCTT.

FIGS. 10A-10E show editing by deamination of a target adenosine (boxed in) in the 3′ untranslated region (UTR) of Glyceraldehyde 3-phosphate dehydrogenase (GAPDH) by RL0079 (Negative Control), RH0001 (Positive Control; RH0001 strand), RD0016, RD0034, and RD0037, in the absence (top panel of each of FIGS. 10A-10E) and presence (bottom panel of each of FIGS. 10A-10E) of interferon alpha (IFNα; 1200U) in MCF-7 cells. FIG. 10A shows editing in the 3′ UTR of GAPDH by RL0079 without IFNα (top) and with 1200U IFNα (bottom) in MCF-7 cells. FIG. 10B shows editing in the 3′ UTR of GAPDH by RH0001 without IFNα (top) and with 1200U IFNα (bottom) in MCF-7 cells. FIG. 10C shows editing in the 3′ UTR of GAPDH by RD0016 without IFNα (top) and with 1200U IFNα (bottom) in MCF-7 cells. FIG. 10D shows editing in the 3′ UTR of GAPDH by RD0034 without IFNα (top) and with 1200U IFNα (bottom) in MCF-7 cells. FIG. 10E shows editing in the 3′ UTR of GAPDH by RD0037 without IFNα (top) and with 1200U IFNα (bottom) in MCF-7 cells. Editing is shown by calculated change of a target base based upon sequencing data. Sequences shown:

(SEQ ID NO: 169)
GCCATGTAGACCCCTT
and
(SEQ ID NO: 170)
GCCATGTGGACCCCTT.

FIGS. 11A-11C show editing by deamination of a target adenosine (boxed in) in the 3′ untranslated region (UTR) of Glyceraldehyde 3-phosphate dehydrogenase (GAPDH) by RD0016 and RD0034, each figure represents testing performed in duplicate (top and bottom panel). FIG. 11A shows editing in the 3′ UTR of GAPDH by RD0016 without IFNα in NCI-H1623 cells. FIG. 11B shows editing in the 3′ UTR of GAPDH by RD0016 with 240U IFNα in NCI-H1623 cells. FIG. 11C shows editing in the 3′ UTR of GAPDH by RD0034 without IFNα in NCI-H1623 cells. Editing is shown by calculated change of a target base based upon sequencing data. Sequences shown:

(SEQ ID NO: 169)
GCCATGTAGACCCCTT
and
(SEQ ID NO: 170)
GCCATGTGGACCCCTT.

FIGS. 12A-12G show non-limiting examples of linkers and configurations thereof. FIGS. 12A-12G correspond to Formula (I)-Formula (VII), as detailed herein.

FIGS. 13A-13C show editing by deamination of a target adenosine in the 3′ untranslated region (UTR) of SK-BR3 cells. FIG. 13A shows percent editing (% editing; y-axis) against concentration of two editing constructs: RD0209 and RP0001-PEG2-RLE0001 (an RNA targeting molecule with a PEG linker; RLE0001—Strand Ref.: 23; RP0001—comprised of Strand Ref.s: 24-25). FIG. 13B shows editing graphs across nucleotides at various concentrations of the editing compositions RD0209 (top row) and RP0001-PEG2-RLE0001 (bottom row). FIG. 13C shows an editing graph of RL0079 at 100 nanomolar (nM) concentration.

FIGS. 14A-14G show editing by deamination in SK-BR-3 human breast cancer cells of a target adenosine in the 3′ UTR of GAPDH. Percent editing is shown on the x-axis with the editing construct shown on the y-axis.

FIG. 15 shows editing by deamination in murine primary hepatocytes of a target adenosine in the 3′ UTR of mouse GAPDH. The editing compound and corresponding editing efficiency are indicated on each graph. Sequence shown:

(SEQ ID NO: 171)
GGAGGGGCCTAGGGAGCCT.

FIGS. 16A-16B show editing by deamination in primary monkey liver fibroblasts of a target adenosine in the 3′ UTR of monkey GAPDH. The editing compound and corresponding concentration and editing efficiency are indicated for each graph of FIG. 16A. The bar chart in FIG. 16B summarizes the results by showing the editing efficiencies on the target adenosine of GAPDH on the y-axis while the compounds and their concentrations are indicated on the x-axis.

FIGS. 17A-17F show editing by deamination in transgenic mouse hepatocytes of a target adenosine in the 3′ UTR of a human gene comprising a mutation that results in a protein mutational variant. The editing compound and corresponding editing efficiency is shown for each graph. Unless otherwise indicated, the concentration of compound used is 100 nM.

### DETAILED DESCRIPTION

The instant disclosure relates, at least in part, to methods and compositions useful for editing nucleic acids, preferably ribonucleic acids (RNA). By exploiting these compositions and practicing these methods, the data contained in the sequences of nucleic acids may be changed (e.g., altered, modified, corrected). For example, without limitation, point mutations may be changed to wild-type nucleotides. Furthermore, proteins coded by the nucleic acids may be modified through nucleotide (e.g., nucleotide, nucleoside, nucleobase) modifications, which may affect subsequent translation. Accordingly, the compositions and methods of the instant disclosure are useful, at least in part, for therapeutic and/or diagnostic effects.

For example, without limitation, in some aspects, compositions comprise one or more features that are effective for recruiting intracellular RNA editing (e.g., modifying) enzymes, and methods of using the same. In some aspects, compositions comprise one or more features that are effective for increasing the local concentration (e.g., the concentration proximal to a composition as compared to the location absent a composition) of an RNA editing enzyme, and methods of practicing the same. These recruiting molecules (e.g., compositions) may be useful in modifying a given nucleotide and/or nucleic acid sequence (for example, without limitation, by deaminating a target nucleotide). In some aspects, compositions comprise one or more features that are effective for guiding RNA editing enzymes to a particular target nucleic acid sequence, and methods of using the same. In some aspects, compositions comprise one or more features that are effective for guiding RNA editing enzymes to a particular target nucleotide, and methods of practicing the same. In some aspects, compositions comprise one or more features that are effective for increasing the local concentration (e.g., the concentration proximal to a target nucleic acid sequence and/or target nucleotide as compared to the location absent the composition) of an RNA editing enzyme to a target nucleic acid sequence and/or target nucleotide, and methods of using the same.

RNA editing enzymes have been observed in eukaryotic species throughout the animal and plant kingdoms, as well as viruses, archaea, and prokaryotes and can occur in the cytosol and nucleus of cells, as well as in the mitochondria and plastids of plants. Various examples of RNA editing exist, but includes nucleoside editing by deamination by enzymes. These enzymes (e.g., adenosine deaminase, cytidine deaminase) act on nucleosides and result in adenosine (A)-to-inosine (I) and cytidine (C)-to-uridine (U) conversions. These conversions can have far reaching effects within an organism. For example, an A-to-I conversion may result in a translational change as I will be interpreted as a guanosine (G) during translation, effectively making an A-to-I conversion an A-to-G conversion with respect to the informational content of the RNA. Thus, an A-to-I conversion in an mRNA or pre-mRNA, has the possibility to alter the protein coding ability or message of the RNA molecule. The adenosine deaminase enzyme (e.g., Adenosine Deaminase Acting on RNA (ADAR)) is a multi-domain protein comprising a recognition domain and a catalytic domain. The recognition domain recognizes a specific double-stranded RNA (dsRNA) sequence and/or conformation, whereas the catalytic domain deaminates (e.g., converts) an A into an I at a relatively close position to the recognition site on a target RNA.

Adenosine deaminases acting on RNA (ADAR) are a group of enzymes responsible for binding to double stranded RNA (dsRNA) and post-transcriptionally converting adenosine (A) to inosine (I) by deamination. In humans, there are a variety of ADAR enzymes known, for example, hADAR1, hADAR2, and hADAR3, and the deaminating activity of these enzymes has been a focus of many areas of biological research. For example, the manipulation of these enzymes for directed deamination of target nucleic acids (e.g., RNA), and even more specifically target nucleotides, has been investigated for a number of years.

ADARs catalyze the reaction from A to I by use of an activated water molecule for a nucleophilic attack (e.g., hydrolytic deamination). Since inosine is structurally similar to guanine (G), post-conversion the deaminated nucleotide (e.g., I) will pair with cytosine (C). Inosine further typically is interpreted as guanosine during translation, a characteristic that can generate codon changes which subsequently can affect protein translation, as well as other mechanisms as described elsewhere herein.

Editing (e.g., deamination) may also occur in non-coding sequences of a target RNA (e.g., untranslated regions (UTR), introns). For example, editing or conversions in the 5′ UTR may result in the creation of a non-native translational start site upstream of the native (e.g., original, wild-type (wt)) start site, which gives rise to proteins with additional residues at the amino-terminus (i.e., N-terminus). Editing events in the 3′ UTRs may affect 3′ UTR binding or processing (e.g., miRNA-based regulation, polyadenylation), or editing of introns may affect splicing, thereby changing the final protein by exon skipping.

As discussed hereinabove, ADAR enzymes direct editing (e.g., by deamination) in a general fashion, meaning that they do not edit at a specific and finely controlled location, but instead are influenced by a variety of factors which impact which nucleotide(s) is edited. For example, such factors can relate to, including without limitation, the target sequence, the sequence of the second RNA strand (e.g., gRNA), location of the target nucleotide, the degree of complementarity of the second RNA strand (e.g., gRNA), the degree and type of modifications in the RNA (e.g., gRNA modifications (e.g., nucleoside modification, linkage or backbone modification)), and length of the nucleic acids (e.g., the target nucleic acid, gRNA).

Accordingly, disclosed herein are ADAR recruiting and targeting molecules which can direct efficient use of ADAR for editing of target sequences and nucleotides (e.g., comprising double-stranded RNA duplex (e.g., recruiting domain) linked to a single-stranded guide nucleic acid (e.g., editing domain, targeting domain)).

### ADAR Recruiting and Targeting Molecules

The present disclosure, at least in part, relates to compositions for use in editing nucleic acids. In some aspects, a composition is used to attract an editing enzyme to the molecule. In doing so, the recruiting molecule may increase the concentration of the editing enzyme in the vicinity (e.g., location, position, proximity) of a target, thereby increasing the likelihood of the enzyme editing the target (e.g., nucleic acid, nucleotide). In some aspects, a composition is or comprises a targeting molecule which targets a nucleic acid sequence for editing by an editing enzyme. A targeting molecule, in some aspects, creates a binding point for an editing enzyme, and directs it by means of a single-stranded guide nucleic acid to a target sequence, thereby increasing the likelihood that an editing enzyme will be present near a target editing site. In some embodiments, a targeting molecule comprises a nucleic acid. For example, in some embodiments, a nucleic acid (e.g., of a targeting molecule) is a ribonucleic acid (RNA). In some embodiments, a nucleic acid is a duplexed nucleic acid (e.g., comprising two strands). In some embodiments, a nucleic acid is a duplexed RNA.

In some aspects, the disclosure relates to an adenosine deaminase acting on ribonucleic acid (RNA) (ADAR) recruiting molecule comprising a double-stranded RNA duplex, wherein the double-stranded RNA duplex comprises two strands of RNA (also referred to herein as “two RNA strands” and/or generally as the “RNA strands” of the double-stranded RNA duplex). In some embodiments, a double-stranded RNA duplex comprises two RNA strands, wherein the terminal nucleotides of one strand of RNA align with the terminal nucleotides of the other strand of RNA comprised by the double-stranded RNA duplex. In some embodiments, the two RNA strands have an equal number of nucleotides. In some embodiments, the two RNA strands do not have an equal number of nucleotides.

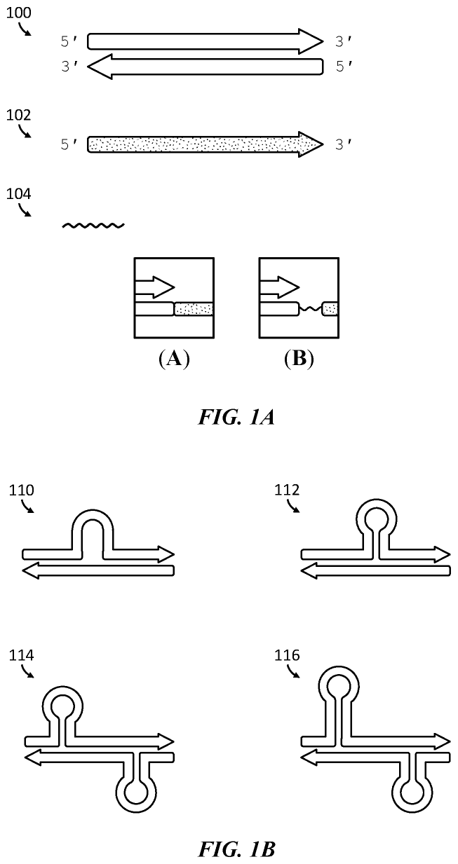

FIG. 1A shows an example structure of a double-stranded RNA duplex 100 having a top strand, which is shown oriented 5′ to 3′, and a bottom strand, which is shown oriented anti-parallel to the top strand. As described herein, in some embodiments, a terminal nucleotide of the top strand aligns with a terminal nucleotide of the bottom strand. For example, where the 5′ terminal nucleotide of the top strand aligns with the 3′ terminal nucleotide of the bottom strand, the left end of duplex 100 can be described as being blunt-ended and/or as not containing an overhang. In some embodiments, both terminal nucleotides of the top strand align with the terminal nucleotides of the bottom strand. Continuing from the previous example, where the top strand further aligns at its 3′ terminal nucleotide with the 5′ terminal nucleotide of the bottom strand, both ends of duplex 100 can be described as being blunt-ended and/or as not containing an overhang.

In some embodiments, an end of a double-stranded RNA duplex may be characterized herein as being blunt-ended or as containing an overhang. It should be appreciated that such characterizations may be used to particularly describe the relationship between strands of the duplex. Accordingly, such characterizations may not account for any additional components (e.g., single-stranded guide nucleic acid, linker) which may be attached to the duplex. For example, referring again to FIG. 1A, in some aspects, the disclosure provides RNA targeting molecules comprising double-stranded RNA duplex 100 attached to a single-stranded guide nucleic acid 102, optionally through a linker 104.

As shown in panel (A), in some embodiments, one strand of duplex 100 is attached directly to single-stranded guide 102 (e.g., through an internucleoside linkage). While it may be said that the RNA targeting molecule shown in panel (A) contains an overhang provided by the single-stranded guide portion (stippled shape), the duplex portion (open shapes) may or may not contain an overhang as relating to the top and bottom strands of the duplex portion. Similarly, panel (B) depicts an example in which one strand of duplex 100 is attached to single-stranded guide 102 through linker 104, and the duplex portion of the RNA targeting molecule may or may not contain an overhang as relating to the top and bottom strands of the duplex portion.

Accordingly, referring once again to the RNA targeting molecule shown in panel (A) of FIG. 1A, it should be appreciated that, in some embodiments, the 3′ terminal nucleotide of the top strand of the duplex portion aligns with the 5′ terminal nucleotide of the bottom strand of the duplex portion. Thus, the disclosure may refer to a terminal nucleotide of a double-stranded RNA duplex, where the terminal nucleotide is defined with respect to the duplex portion of a molecule. As the molecule may include additional nucleic acid components (e.g., one or more additional nucleotides, a linker, and/or a single-stranded guide nucleic acid), it is to be understood that a terminal nucleotide of the duplex portion is not necessarily the terminal nucleotide of the strand on which it is located.

In instances where the two RNA strands do not have an equal number of nucleotides, and the terminal nucleotides of each RNA strand comprising the double-stranded RNA duplex are aligned (e.g., do not hang past the terminal nucleotide of the opposite strand), the duplex will comprise a bulge due to the fact that the terminal nucleotides of each RNA strand comprising the double-stranded RNA duplex are aligned (e.g., do not hang past the terminal nucleotide of the opposite strand). The term “bulge,” as may be used herein, refers to the property of a strand of a nucleic acid to be not parallel to its partner (e.g., complementary) strand of nucleic acid, such that it forms a shape which is aberrant from a strand which shares 100% complementarity and/or is comprised of the same number nucleotides. A bulge may simply be illustrated as a non-linked segment of a duplex (e.g., nucleotide) which is spatially further away from its opposite strand than those segments (e.g., nucleotides) which are base paired. A bulge may also be illustrated as a floating single-stranded segment of the nucleic acid, wherein it may form additional shapes (e.g., hairpins). In some embodiments, an RNA strand of the double-stranded RNA duplex comprises a bulge. In some embodiments, the bulge is not at the 5′ or 3′ end of the RNA strand.

FIG. 1B shows examples of double-stranded RNA duplexes having at least one bulge. In some embodiments, a bulge is an unpaired segment of nucleotides located within one strand of a duplex, as illustrated by duplex 110. In some embodiments, where the unpaired segment includes non-contiguous portions capable of self-hybridizing within the strand, the bulge is a hairpin (e.g., as illustrated by duplexes 112, 114, and 116). In some embodiments, a double-stranded RNA duplex comprises a hairpin in one RNA strand, as shown for duplex 112. In some embodiments, an RNA strand comprises more than one bulge. In some embodiments, a double-stranded RNA duplex comprises two RNA strands, wherein each of the RNA strands of the double-stranded RNA duplex comprises at least one bulge. In some embodiments, each strand of a duplex comprises a bulge formed by segments of the same number of nucleotides, as shown for duplex 114. In some embodiments, each strand of a duplex comprises a bulge formed by segments of a different number of nucleotides, as shown for duplex 116.

In some embodiments, an ADAR recruiting molecule comprises: (a) a double-stranded RNA duplex; and (b) at least one RNA strand of the double-stranded RNA duplex comprises at least one nucleoside modification and/or at least one backbone modification. In some embodiments, an ADAR recruiting molecule comprises: (a) a double-stranded RNA duplex, wherein the double-stranded RNA duplex comprises two strands of RNA, which two RNA strands are not connected to one another by means of a hairpin, wherein: (a) the 5′ nucleotide of each RNA strand is complementary to the 3′ nucleotide of the other RNA strand; and (b) at least one RNA strand of the double-stranded RNA duplex comprises at least one nucleoside modification and/or at least one backbone modification.

In some aspects, the disclosure relates to an adenosine deaminase acting on ribonucleic acid (RNA) (ADAR) recruiting molecule comprising a double-stranded RNA duplex, wherein the double-stranded RNA duplex comprises two strands of RNA of an equal number of nucleotides, which two RNA strands are not connected to one another by means of a hairpin, wherein: (a) the 5′ nucleotide of each RNA strand is complementary to the 3′ nucleotide of the other RNA strand; and (b) at least one RNA strand of the double-stranded RNA duplex comprises at least one nucleoside modification and/or at least one backbone modification.

In some embodiments, the double-stranded RNA duplex comprises at least one base pair mismatch, wherein the mismatch is not positioned at either terminal nucleotide base pair of the double-stranded RNA duplex. In some embodiments, the double-stranded RNA duplex comprises 1-5 mismatches. In some embodiments, at least one (e.g., 1, 2, or 3) of the mismatches are wobble base pairs. However, in some embodiments, none of the mismatched base pairs are wobble base pairs.

In some aspects, the disclosure relates to an RNA targeting molecule comprising: (a) a double-stranded RNA duplex, wherein the double-stranded RNA duplex comprises two strands of RNA of an equal number of nucleotides, which two RNA strands are not connected to one another by means of a hairpin, wherein the 5′ nucleotide of each RNA strand is complementary to the 3′ nucleotide of the other RNA strand; and (b) a single-stranded guide nucleic acid. In some embodiments, one strand of a double-stranded RNA duplex is directly connected to a single-stranded guide nucleic acid (e.g., in the form of a long RNA strand that is longer than the complementary RNA strand of the RNA duplex). However, in some embodiments, a double-stranded RNA duplex is connected to a single-stranded guide nucleic acid via a linker (e.g., a covalent linker, for example a polymeric or other synthetic linker).

In some embodiments, a double-stranded RNA duplex of an RNA targeting molecule comprises at least one base pair mismatch, wherein the mismatch is not positioned at either terminal nucleotide base pair of the double-stranded RNA duplex. In some embodiments, the double-stranded RNA duplex of an RNA targeting molecule comprises 1-5 mismatches. In some embodiments, at least one (e.g., 1, 2, or 3) of the mismatches are wobble base pairs. However, in some embodiments, none of the mismatched base pairs are wobble base pairs.

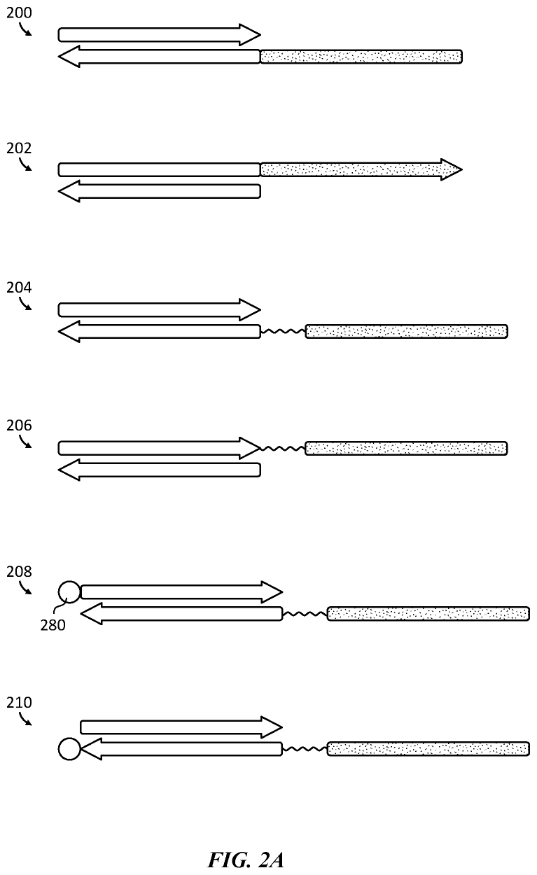

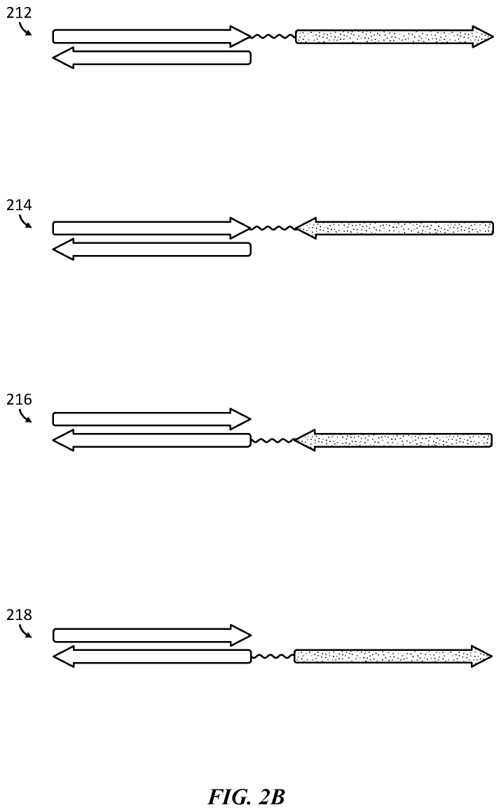

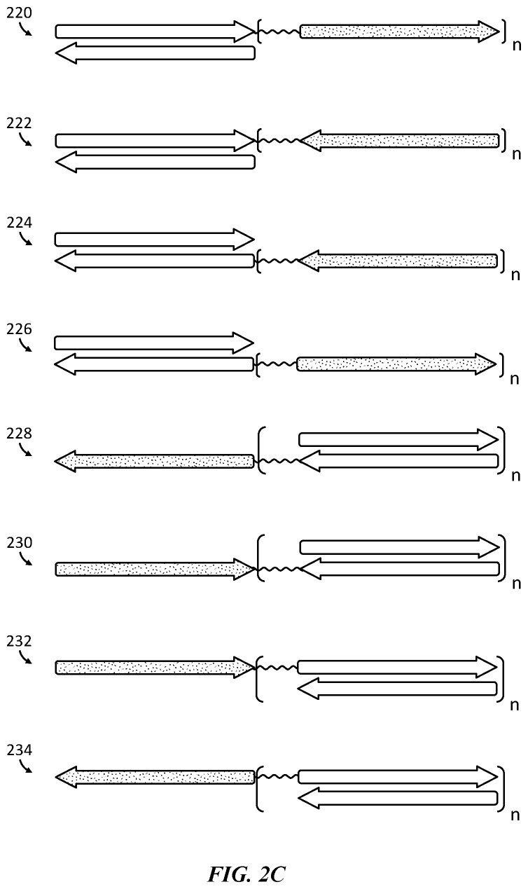

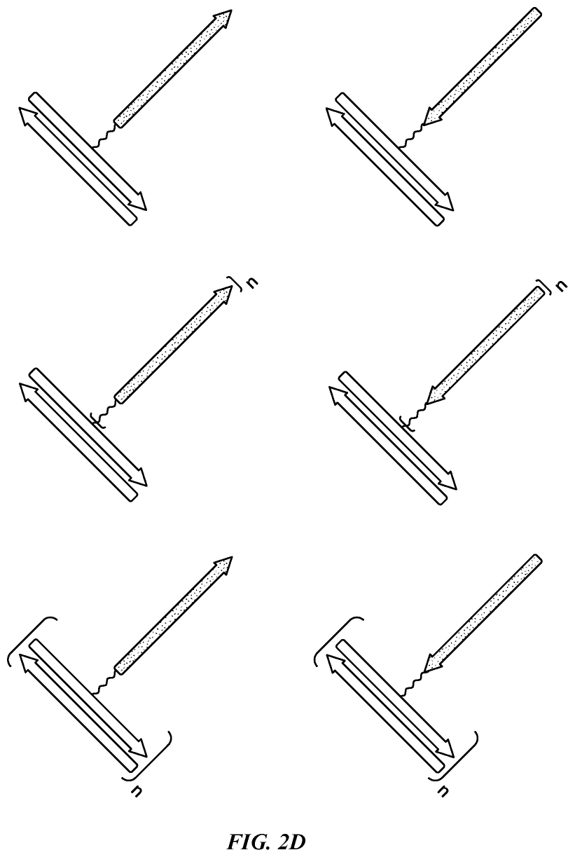

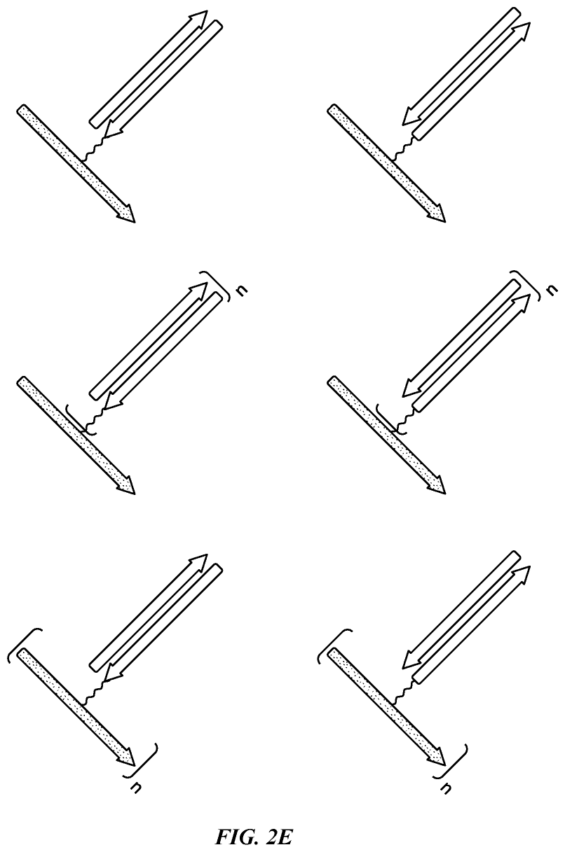

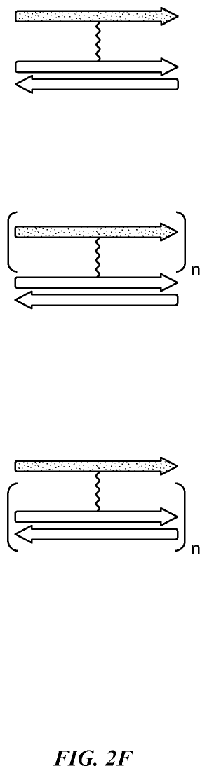

FIG. 2A depicts non-limiting examples of different arrangements of portions of an RNA targeting molecule, including the double-stranded RNA duplex (open shapes) and the single-stranded guide nucleic acid (stippled shapes), and optional components including linkers (wavy shapes) and additional moieties (including tags or markers, and delivery moieties).

In some embodiments, an RNA targeting molecule comprises a duplex portion and a single-stranded guide portion, where one strand of the duplex portion is attached to the single-stranded guide portion through an internucleoside linkage. For example, RNA targeting molecule 200 shows an example in which the 3′ terminal nucleotide of the single-stranded guide portion is attached to the 5′ terminal nucleotide of one strand of the duplex portion through an internucleoside linkage. RNA targeting molecule 202 shows an example in which the 5′ terminal nucleotide of the single-stranded guide portion is attached to the 3′ terminal nucleotide of one strand of the duplex portion through an internucleoside linkage. As generally illustrated by RNA targeting molecules 200 and 202, one strand of an RNA targeting molecule can include a contiguous stretch of nucleotides comprising the single-stranded guide portion and one strand of the duplex portion.

In some embodiments, an RNA targeting molecule comprises a duplex portion and a single-stranded guide portion, where one strand of the duplex portion is attached to the single-stranded guide portion through a linker. Referring again to FIG. 2A, RNA targeting molecule 204 shows an example in which the 3′ terminal nucleotide of the single-stranded guide portion is attached to the 5′ terminal nucleotide of one strand of the duplex portion through a linker. RNA targeting molecule 206 shows an example in which the 3′ terminal nucleotide of the single-stranded guide portion is attached to the 3′ terminal nucleotide of one strand of the duplex portion through a linker. In some embodiments, an RNA targeting molecule comprises an additional moiety 280, as described elsewhere herein. For example, RNA targeting molecules 208 and 210 show examples in which an additional moiety is present on RNA targeting molecules 204 and 206, respectively.

FIG. 2B depicts further examples of RNA targeting molecules comprising a linker. RNA targeting molecule 212 shows an example in which the 5′ terminal nucleotide of the single-stranded guide portion is attached to the 3′ terminal nucleotide of one strand of the duplex portion. RNA targeting molecule 214 shows an example in which the 3′ terminal nucleotide of the single-stranded guide portion is attached to the 3′ terminal nucleotide of one strand of the duplex portion. RNA targeting molecule 216 shows an example in which the 3′ terminal nucleotide of the single-stranded guide portion is attached to the 5′ terminal nucleotide of one strand of the duplex portion. RNA targeting molecule 218 shows an example in which the 5′ terminal nucleotide of the single-stranded guide portion is attached to the 5′ terminal nucleotide of one strand of the duplex portion.

Other configurations of the different components (e.g., with different relative positions of the double-stranded RNA duplex, single-stranded guide nucleic acid, and one or more optional components) also can be used as described in more detail in this application.

In some aspects, the disclosure relates to an RNA targeting molecule comprising: (a) at least one double-stranded RNA duplex; and (b) at least one single-stranded guide nucleic acid. In some embodiments, an RNA targeting molecule comprises more than one double-stranded RNA duplex (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more). In some embodiments, an RNA targeting molecule comprises at least two double-stranded RNA duplexes. In some embodiments, an RNA targeting molecule comprises at least three double-stranded RNA duplexes. In some embodiments, an RNA targeting molecule comprises more than one single-stranded guide nucleic acid (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more). In some embodiments, an RNA targeting molecule comprises at least two single-stranded guide nucleic acids. In some embodiments, an RNA targeting molecule comprises at least three single-stranded guide nucleic acids.

FIG. 2C shows different example configurations of a double-stranded RNA duplex attached to a single-stranded guide nucleic acid through a linker. In some embodiments, a double-stranded RNA duplex is attached to one or more single-stranded guide nucleic acids, as illustrated by RNA targeting molecules 220, 222, 224, and 226. In some embodiments, a single-stranded guide nucleic acid is attached to one or more double-stranded RNA duplexes, as illustrated by RNA targeting molecules 228, 230, 232, and 234. The example configurations shown in FIG. 2C depict either the single-stranded guide portion or the duplex portion in parentheses to illustrate that there may be multiple modules of either portion attached to the other (indicated by the subscript “n,” where n is greater than or equal to 1). The different attachment configurations are as described for the corresponding structures depicted in FIGS. 2A and 2B.

The RNA targeting molecule configurations shown in FIGS. 2A-2C provide example in which a linker is attached to a terminal nucleotide of each of the duplex portion and the single-stranded guide portion of the molecule. However, in some embodiments, a terminal nucleotide of a single-stranded guide portion is attached to an internal (e.g., non-terminal) nucleotide of one strand of a duplex portion, as illustrated by the example configurations shown in FIG. 2D. In some embodiments, a terminal nucleotide of one strand of a duplex portion is attached to an internal nucleotide of a single-stranded guide portion, as illustrated by the example configurations shown in FIG. 2E. In some embodiments, a linker is attached to an internal nucleotide of each of the duplex portion and the single-stranded guide portion of an RNA targeting molecule, as illustrated by the example configurations shown in FIG. 2F. Additional example configurations are illustrated in FIG. 2G.

In some embodiments, a double-stranded RNA duplex of an RNA targeting molecule comprises at least one base pair mismatch, wherein the mismatch is not positioned at either terminal nucleotide base pair of the double-stranded RNA duplex.

The term “nucleoside,” as may be used herein, refers to glycosylamines (e.g., N-glycosides) that are generally known to be nucleotides without a phosphate group. A nucleoside consists of a nucleobase (e.g., a nitrogenous base (e.g., nucleobase)) and a pentose sugar (e.g., ribose). The pentose sugar can be either ribose or deoxyribose. Nucleosides are the biochemical precursors of nucleotides, which are the constituent components of RNA and DNA. The term “nucleotide,” as may be used herein, refers to a nucleobase and a pentose sugar (i.e., nucleoside), and one or more phosphate groups. In a nucleoside, the anomeric carbon is linked through a glycosidic bond to the N9 of a purine or the N1 of a pyrimidine. Examples of nucleosides and nucleobases include, without limitation, cytidine (C), uridine (U), adenosine (A), guanosine (G), thymidine (T), and inosine (I), however it is also to be understood that the term describes nucleosides which result from modification (as such term is defined herein) as they contain a nucleobase and a pentose sugar. For example, nucleosides include, natural nucleosides (e.g., deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, O(6) methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages), xanthine, hypoxanthine, nubularine, isoguanisine, tubercidine, 2-aminopurine, 2,6-diaminopurine, 3-deazaadenosine, 7-deazaadenosine, 7-methyladenosine, 8-azidoadenosine, 8-methyladenosine, 5-hydroxymethylcytosine, 5-methylcytidine, Pyrrolocytidine, 7-aminomethyl-7-deazaguanosine, 7-deazaguanosine, 7-methylguanosine, 8-aza-7-deazaguanosine, thienoguanosine, inosine, 4-thio-uridine, 5-methoxyuridine, dihydrouridine, and pseudouridine. The terms “nucleic acid,” “nucleotide sequence,” “polynucleotide,” “oligonucleotide,” and “polymer of nucleotides” as may be used interchangeably herein, refer to a string of at least two, base-sugar-phosphate combinations (e.g., nucleotides, nucleosides and a phosphate group) and includes, among others, single-stranded and double-stranded DNA, DNA that is a mixture of single-stranded and double-stranded regions, single-stranded and double-stranded RNA, and RNA that is mixture of single-stranded and double-stranded regions, hybrid molecules comprising DNA and RNA that may be single-stranded or, more typically, double-stranded or a mixture of single-stranded and double-stranded regions. In addition, the terms (e.g., nucleic acid, et al.) as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often referred to as an oligonucleotide.

The terms (e.g., nucleic acid, et al.) also encompass such chemically, enzymatically, or metabolically modified forms of nucleic acids, as well as the chemical forms of DNA and RNA characteristic of viruses and cells, including simple and complex cells. For instance, the terms (e.g., nucleic acid, et al.) as used herein can include DNA or RNA as described herein that contain one or more modified bases. The nucleic acids may comprise natural and synthetic or modified nucleotides, nucleosides, and/or nucleobases. Thus, DNA or RNA including unusual bases, such as inosine, or modified bases, such as tritylated bases, to name just two examples, are nucleic acids as the term is used herein. The terms (e.g., nucleic acid, et al.) also includes peptide nucleic acids (PNAs), phosphorothioates, and other variants of the phosphate backbone of native nucleic acids. Natural nucleic acids have a phosphate backbone, artificial nucleic acids can contain other types of backbones, but contain the same bases. Thus, DNA or RNA with backbones modified for stability or for other reasons are nucleic acids as that term is intended herein.

The terms “backbone modification,” “internucleoside linkage modification,” “modifications to backbone linkages,” and variations of these phrases as context may require, as may be used interchangeably herein, refer to a modification (e.g., chemical, structural, as is further described below) to the phosphate of the phosphate-sugar backbone or linkages of a nucleic acid (e.g., the phosphate which connects the nucleosides). In other words, when referring to a backbone modification herein, it is to be understood that the phosphate of a nucleic acid has been modified (e.g., altered) from its natural or wild-type state. For example, a phosphorothioate modification to a phosphate group may be referred to more generally herein as a backbone modification, or a modification to the internucleoside linkage.

In some embodiments, a double-stranded RNA duplex described herein comprises one or more modifications. For example, in some embodiments, one or both RNA strands of a double-stranded RNA duplex comprises one or more modifications. In some embodiments, a single-stranded guide nucleic acid described herein comprises one or more modifications. “Modifications,” as the term may be used herein, refer to modifications of, on, or to a nucleotide, nucleoside, nucleobase, an oligonucleotide, phosphate backbone, or the constituent portions or linkages thereof (i.e., the nitrogenous base (e.g., nucleobase), sugar, or phosphate group). Additionally, it is to be noted that a modified nucleic acid may further be used in the construction of an ADAR recruiting molecule and/or an RNA targeting molecule of the disclosure, and do not necessarily need to be modified as part of the creation of the molecules. For example, without limitation, an ADAR recruiting molecule and/or an RNA targeting containing a modification as described herein may be the result of a modification to a nucleotide (or any of its constituent parts or equivalents), or may result from using a nucleotide (or any of its constituent parts or equivalents) that was created in its modified state. Modifications may be introduced for a variety of reasons, often to increase stability, reduce off-target effects, increase hybridization (e.g., binding) properties, or to reduce toxicity.

In some embodiments, one or both RNA strands of a double-stranded RNA duplex described herein comprises at least one nucleoside modification. In some embodiments, a single-stranded guide nucleic acid described herein comprises at least one nucleoside modification. For example, in some embodiments, a double-stranded RNA duplex and/or a single-stranded guide nucleic acid comprises at least one nucleobase modification. Purine and/or pyrimidine nucleobases may be modified, for example by amination or deamination of the heterocyclic rings. Further, modified sugars, such as 2′-O substitutions (e.g., modifications) to the sugar (e.g., ribose), including without limitation, 2′-O-methoxyethyl sugar, a 2′-fluoro sugar modification (2′-Fluoro), a 2′-O-methyl sugar (2′-O-Methyl), 2′-O-ethyl sugar, 2′-Cl, 2′-SH and substitutions thereof (e.g., 2′-SCH₃) a bicyclic sugar moiety, or substitutions such as a 2′-O moiety with a lower alkyl, cycloalkyl, aryl, aralkyl, heteroaryl or substitutions thereof (e.g., —CH₃, —CF₃), 2′-amino or substitutions thereof, 2′,3′-seco nucleotide mimic, 2′-F-arabino nucleotide, inverted nucleotides, inverted 2′-O-Methyl nucleotide, polyethyleneglycols (PEG), alkylamino nucleotide, dialkylamino nucleotide, heterocyclyl nucleotide, arylamino nucleotide, diaryl amino nucleotide, heteroaryl amino nucleotide, diheteroaryl amino nucleotide, ethylene diamine nucleotide, polyamino nucleotide, aminoalkoxy nucleotide, 2′-O-deoxy nucleotide, cyano nucleotide, mercapto nucleotide, alkyl-thio-alkyl nucleotide, thioalkoxy nucleotide, an alkenyl, an alkynyl, a cycloalkyl, an aryl, a methoxyethyl (2′-O-MOE), 2′-O-allyl, 2′-C-allyl, 2′-fluoro, an —H (as in DNA), or other substituent may be introduced. Ribose mimics are also contemplated, such as, without limitation, morpholino, Glycol nucleic acid (GNA), UNA, cyclohexenyl nucleic acid (CeNA).

Other examples include 2′-4′ sugar bridged variants, such as locked-nucleic acids (LNAs), and 2′-O, 4′-C-ethylene-bridged nucleic acid (ENA). Locked nucleic acids are modified RNA nucleotides in which the ribose sugar is modified by means of a bridge connecting the 2′ oxygen and 4′ carbon (often seen as a methylene bridge between the 2′ oxygen and 4′ carbon). This bridge operably “locks” the ribose in the 3′-endo conformation. The locked ribose sugar conformation can enhance base stacking and backbone pre-organization, which can affect (e.g., increase) its hybridization properties (e.g., thermal stability and hybridization specificity). Locked nucleic acids can be inserted into both RNA and DNA oligonucleotides to hybridize with DNA or RNA according to typical Watson-Crick base-pairing rules (i.e., complementarity).

Other chemistries and modification are known in the field of oligonucleotides that can be readily used in accordance with the disclosure and are encompassed within the definition of a nucleic acid modification, for example, the term modification shall further include any alteration, change, or manipulation, which results in the formation of any nucleoside other than the natural nucleosides.

In some embodiments, one or both RNA strands of a double-stranded RNA duplex described herein comprises at least one backbone modification. In some embodiments, a single-stranded guide nucleic acid described herein comprises at least one backbone modification. For example, in some embodiments, a double-stranded RNA duplex and/or a single-stranded guide nucleic acid comprises at least one modified internucleoside linkage. Linkages between the nucleotides may be modified by means of thioation of the phosphodiester bonds which can be used to yield phosphorothioate esters or phosphorodithioate esters, and cholesterol. Further modification to the linkages include amidation and peptide linkers. Other examples include, phosphodiester, phosphotriester, phosphoro(di)thioate, methylphosphonate, phosphor-amidate linkers, phosphonates, 3′-methylenephosphonate, 5′-methylenephosphonate, Boranophosphate, 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (e.g. RP(OH)(O)—O-5′-, (OH)₂(O)P-5′-CH₂—, R=alkyl, methyl, ethyl, isopropyl, propyl, etc.), 5′-alkyletherphosphonates (e.g. RP(OH)(O)—O-5′-, R=alkylether, methoxymethyl (MeOCH₂—), ethoxymethyl, etc.) and the like. Further, the chirality of the isomers may be modified (e.g., Rp and Sp).

### Double-Stranded RNA Duplex

In some aspects the disclosure relates to components of an ADAR recruiting molecule and/or an RNA targeting molecule. In some embodiments, an ADAR recruiting molecule and/or an RNA targeting molecule of the disclosure comprise a double-stranded RNA duplex. In some embodiments, a double-stranded RNA duplex comprises two nucleic acids (e.g., two RNA strands, two DNA strands, a combination of DNA and/or RNA, etc.) which are hybridized together to form a duplex, wherein the two RNA strands consist of equal numbers of nucleotides. The double-stranded RNA duplex may be of any length to effectuate the purpose of an ADAR recruiting molecule and/or an RNA targeting molecule of the disclosure. In some embodiments, a double-stranded RNA duplex is at least 2 base pairs in length, in other words, since each RNA strand of a double-stranded RNA duplex is equal in length (e.g., consists of an equal number of nucleotides), each RNA strand of a double-stranded RNA duplex comprises at least 2 nucleotides. In this sense, the length of a double-stranded RNA duplex can be referred to by the length of an RNA strand of which it is comprised. In some embodiments, an RNA strand of a double-stranded RNA duplex is at least 5 nucleotides in length (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nucleotides). In some embodiments, an RNA strand of a double-stranded RNA duplex is about 5 to about 100 nucleotides in length. In some embodiments, an RNA strand of a double-stranded RNA duplex is about 5 to about 90 nucleotides in length. In some embodiments, an RNA strand of a double-stranded RNA duplex is about 5 to about 80 nucleotides in length. In some embodiments, an RNA strand of a double-stranded RNA duplex is about 5 to about 70 nucleotides in length. In some embodiments, an RNA strand of a double-stranded RNA duplex is about 5 to about 60 nucleotides in length. In some embodiments, an RNA strand of a double-stranded RNA duplex is about 5 to about 50 nucleotides in length. In some embodiments, an RNA strand of a double-stranded RNA duplex is about 5 to about 40 nucleotides in length. In some embodiments, an RNA strand of a double-stranded RNA duplex is about 5 to about 30 nucleotides in length. In some embodiments, an RNA strand of a double-stranded RNA duplex is about 5 to about 20 nucleotides in length. In some embodiments, an RNA strand of a double-stranded RNA duplex is about 10 to about 30 nucleotides in length. In some embodiments, an RNA strand of a double-stranded RNA duplex is about 15 to about 27 nucleotides in length. In some embodiments, an RNA strand of a double-stranded RNA duplex is about 16 to about 26 nucleotides in length. In some embodiments, each RNA strand of a double-stranded RNA duplex is about 10 to about 30 nucleotides in length. In some embodiments, each RNA strand of a double-stranded RNA duplex is about 15 to about 25 nucleotides in length. In some embodiments, each RNA strand of a double-stranded RNA duplex is about 16 to about 23 nucleotides in length. In some embodiments, each RNA strand of a double-stranded RNA duplex is about 18 to about 22 nucleotides in length. In some embodiments, each RNA strand of a double-stranded RNA duplex is about 20 to about 22 nucleotides in length.

In some embodiments, at least one of the RNA strands of a double-stranded RNA duplex comprises at least one nucleotide modification. A modification may be to any part of the nucleotide. For example, without limitation, a nucleotide modification may be in the phosphate group, the pentose sugar group (e.g., ribose), or the nucleobase. In some embodiments, a nucleotide modification is in the phosphate group. In some embodiments, a nucleotide modification is in the pentose sugar group. In some embodiments, a nucleotide modification is in the nucleobase group. In some embodiments, a double-stranded RNA duplex comprises at least one nucleoside modification. A modification may be to any part of the nucleoside. For example, without limitation, a nucleoside modification may be in the pentose sugar group (e.g., ribose) or the nucleobase. In some embodiments, a nucleoside modification is in the pentose sugar group. In some embodiments, a nucleoside modification is in the nucleobase group. In some embodiments, the at least one nucleoside modification is a 2′-O-Methyl modification. In some embodiments, the at least one nucleoside modification is a 2′-O-methoxyethyl (2′-O-MOE). In some embodiments, the at least one nucleoside modification is a 2′-Fluoro modification. In some embodiments, a double-stranded duplex comprises at least one nucleobase modification. A nucleoside modification may occur at point throughout a double-stranded RNA duplex. A nucleoside modification may occur on either strand of a double-stranded RNA duplex. In some embodiments, a nucleoside modification occurs on only one strand of a double-stranded RNA duplex. In some embodiments, a nucleoside modification occurs in both strands of a double-stranded RNA duplex. In some embodiments, a nucleoside modification is positioned toward the ends of a given strand of a double-stranded RNA duplex (e.g., toward the 5′ or 3′ end, or both in the case of multiple modifications). In some embodiments, a nucleoside modification is positioned toward the middle of a given strand of a double-stranded RNA duplex. As one of ordinary skill in the art will appreciate, as discussed herein, when referring to a nucleoside modification, such terminology will encompass modifications to the nucleoside, or a component thereof individually and collectively. In other words, a modification of a nucleobase and/or 5 carbon sugar (in the context of being part of a nucleoside/nucleotide) in a nucleic acid will inherently also be a nucleoside modification.

In some embodiments, a double-stranded RNA duplex comprises more than one nucleoside modification. In some embodiments, a double-stranded RNA duplex comprises more than two nucleoside modifications. In some embodiments, more than 25%, but less than or equal to 100%, of the nucleosides in a double-stranded RNA duplex comprise a nucleoside modification. In some embodiments, more than 50% of the nucleosides in a double-stranded RNA duplex comprise a nucleoside modification. In some embodiments, more than 75%, but less than or equal to 100%, of the nucleosides in a double-stranded RNA duplex comprise a nucleoside modification. In some embodiments, at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the nucleosides in a double-stranded RNA duplex comprise a nucleoside modification. In some embodiments, at least 95%, but less than or equal to 100%, of the nucleosides in a double-stranded RNA duplex comprise a nucleoside modification.

In some embodiments, a double-stranded RNA duplex comprises at least one backbone modification. In some embodiments, the at least one backbone modification of a double-stranded RNA duplex comprises a phosphorothioate modification (e.g., a substitution of a phosphodiester bond with a phosphorothioate bond). A backbone modification may occur at any point throughout a double-stranded RNA duplex. A backbone modification may occur on either strand of a double-stranded RNA duplex. In some embodiments, a backbone modification occurs on only one strand of a double-stranded RNA duplex. In some embodiments, a backbone modification occurs in both strands of a double-stranded RNA duplex. In some embodiments, a backbone modification is positioned toward the ends of a given strand of a double-stranded RNA duplex (e.g., toward the 5′ or 3′ end, or both in the case of multiple modifications). In some embodiments, a backbone modification is positioned toward the middle of a given strand of a double-stranded RNA duplex. In some embodiments, the at least one backbone modification of a double-stranded RNA duplex is positioned within 1-5 nucleotides of the terminal nucleotide of the RNA strand on which it is located. In some embodiments, the at least one backbone modification of a double-stranded RNA duplex is positioned within 1-3 nucleotides of the terminal nucleotide of the RNA strand on which it is located. In some embodiments, the at least one backbone modification of a double-stranded RNA duplex is positioned within 1 nucleotide of the terminal nucleotide of the RNA strand on which it is located.

In some embodiments, a double-stranded RNA duplex comprises at least one backbone modification. In some embodiments, a double-stranded RNA duplex comprises more than one backbone modification. In some embodiments, a double-stranded RNA duplex comprises more than two backbone modifications. In some embodiments, a double-stranded RNA duplex comprises more than three backbone modifications. In some embodiments, more than 25% of the internucleoside linkages of a double-stranded RNA duplex comprise a modification. In some embodiments, more than 50% of the internucleoside linkages of a double-stranded RNA duplex comprise a modification. In some embodiments, more than 75% of the internucleoside linkages of a double-stranded RNA duplex comprise a modification. In some embodiments, at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the internucleoside linkages in a double-stranded RNA duplex comprise a modification. In some embodiments, at least 95% of the internucleoside linkages of a double-stranded RNA duplex comprise a modification.

In some embodiments, a double-stranded RNA duplex comprises at least one nucleoside modification and at least one backbone modification. In some embodiments, a double-stranded RNA duplex comprises more than one nucleoside modification and at least one backbone modification. In some embodiments, a double-stranded RNA duplex comprises at least one nucleoside modification and more than one backbone modification. In some embodiments, a double-stranded RNA duplex comprises more than one nucleoside modification and more than one backbone modification.

In some embodiments the double-stranded RNA duplex further comprises at least one mismatched base pair of the duplex (e.g., nucleotides which do not base pair according to customary Watson-Crick base pairing rules). When every base in one strand of a pair of nucleic acid strands is found opposite its complementary base pair, such strand is considered fully complementary to the sequence of the other strand. When a base of such a strand is found in a position where it is opposite any other base excepting its complementary base of the pair, that base is considered “mis-matched” (also referred to as a mismatch) and the strand is considered partially complementary. Accordingly, strands can be varying degrees of partially complementary, until no bases align, at which point they are non-complementary. Other non-standard nucleotides (e.g., 5-methylcytosine, 5-hydroxymethylcytosine) are known in the art and their properties and complementarity will be readily apparent to the skilled artisan. Methods and calculations are known in the art to determine percent complementarity.

In some embodiments, a double-stranded RNA duplex contains more than one mismatch. In some embodiments, a double-stranded RNA duplex contains fewer than 30 mismatches. In some embodiments, a double-stranded RNA duplex contains more than one mismatch, but fewer than 30 mismatches. In some embodiments, a double-stranded RNA duplex contains at least one, but fewer than 11 mismatches. In some embodiments, a double-stranded RNA duplex contains at least one, but fewer than 6 mismatches. In some embodiments, a double-stranded RNA duplex contains at least one, but fewer than 4 mismatches. In some embodiments, where a double-stranded RNA duplex contains more than one mismatch, the mismatches are consecutive (e.g., adjacent) in the nucleic acid. In some embodiments, where a double-stranded RNA duplex contains more than one mismatch, the mismatches are non-consecutive (e.g., not adjacent) in the nucleic acid. In some embodiments, where a double-stranded RNA duplex contains more than two mismatches, there is at least one grouping of two or more mismatches adjacent to one another. In some embodiments, where a double-stranded RNA duplex contains more than two mismatches, there are no two or more mismatches adjacent to one another. In some embodiments, the double-stranded RNA duplex does not comprise a mismatch.

In some embodiments, one or more of the mismatches are wobble base pairs. In some embodiments, a double-stranded RNA duplex comprises a wobble base pair. The term “wobble,” as may be used in the context of a base pairing herein, refers to a term of art generally known in the field to refer to a base pairing of specific nucleotides (e.g., a wobble base pair), which are non-canonical in that they are not Watson-Crick base pairs (e.g., are a form of, or subset of, mismatched base pairs). Specifically, the term wobble is used as a term to describe base pairings of hypoxanthine (inosine (I)) and uracil (U) (I/U); guanine (G) and U (G/U); I and adenine (A) (I/A); and I and cytosine (C) (I/C). In some embodiments, a double-stranded RNA duplex comprises at least 1 (e.g., 1, 2, 3, 4, 5, 6, 7, 8, 9, 10, 11, 12, 13, 14, 15, 16, 17, 18, 19, 20, or more) wobble base pair. In some embodiments, a double-stranded RNA duplex comprises at least 2 wobble base pairs. In some embodiments, a double-stranded RNA duplex comprises at least 3 wobble base pairs. In some embodiments, a double-stranded RNA duplex comprises at least 4 wobble base pairs. In some embodiments, a double-stranded RNA duplex comprises at least 5 wobble base pairs. In some embodiments, a double-stranded RNA duplex comprises at least 10 wobble base pairs. In some embodiments, a double-stranded RNA duplex comprises at least 20 wobble base pairs.

In some embodiments, a double-stranded RNA duplex comprises fewer than 20 wobble base pairs. In some embodiments, a double-stranded RNA duplex comprises fewer than 10 wobble base pairs. In some embodiments, a double-stranded RNA duplex comprises fewer than 5 wobble base pairs. In some embodiments, a double-stranded RNA duplex comprises fewer than 4 wobble base pairs. In some embodiments, a double-stranded RNA duplex comprises fewer than 3 wobble base pairs. In some embodiments, a double-stranded RNA duplex comprises fewer than 2 wobble base pairs.

In some embodiments, a double-stranded RNA duplex comprises at least 1 wobble base pair, but fewer than or equal to 20 wobble base pairs. In some embodiments, a double-stranded RNA duplex comprises at least 1 wobble base pair, but fewer than or equal to 10 wobble base pairs. In some embodiments, a double-stranded RNA duplex comprises at least 1 wobble base pair, but fewer than or equal to 7 wobble base pairs. In some embodiments, a double-stranded RNA duplex comprises at least 1 wobble base pair, but fewer than or equal to 5 wobble base pairs. In some embodiments, a double-stranded RNA duplex comprises at least 1 wobble base pair, but fewer than or equal to 4 wobble base pairs. In some embodiments, a double-stranded RNA duplex comprises at least 1 wobble base pair, but fewer than or equal to 3 wobble base pairs. In some embodiments, a double-stranded RNA duplex comprises at least 2 wobble base pairs, but fewer than or equal to 20 wobble base pairs. In some embodiments, a double-stranded RNA duplex comprises at least 2 wobble base pairs, but fewer than or equal to 10 wobble base pairs. In some embodiments, a double-stranded RNA duplex comprises at least 2 wobble base pairs, but fewer than or equal to 7 wobble base pairs. In some embodiments, a double-stranded RNA duplex comprises at least 2 wobble base pairs, but fewer than or equal to 5 wobble base pairs. In some embodiments, a double-stranded RNA duplex comprises at least 2 wobble base pairs, but fewer than or equal to 4 wobble base pairs.

In some embodiments, a wobble base pair is within 8 nucleotides of a terminal nucleotide of at least one of the RNA strands of a double-stranded RNA duplex. In some embodiments, a wobble base pair is within 7 nucleotides of a terminal nucleotide of at least one of the RNA strands of a double-stranded RNA duplex. In some embodiments, a wobble base pair is within 6 nucleotides of a terminal nucleotide of at least one of the RNA strands of a double-stranded RNA duplex. In some embodiments, a wobble base pair is within 5 nucleotides of a terminal nucleotide of at least one of the RNA strands of a double-stranded RNA duplex. In some embodiments, a wobble base pair is within 4 nucleotides of a terminal nucleotide of at least one of the RNA strands of a double-stranded RNA duplex. In some embodiments, a wobble base pair is within 3 nucleotides of a terminal nucleotide of at least one of the RNA strands of a double-stranded RNA duplex. In some embodiments, a wobble base pair is within 2 nucleotides of a terminal nucleotide of at least one of the RNA strands of a double-stranded RNA duplex. In some embodiments, a wobble base pair is within 1 nucleotide of a terminal nucleotide of at least one of the RNA strands of a double-stranded RNA duplex.

In some embodiments, the double-stranded RNA duplex does not comprise a wobble base pair.

In some embodiments, a mismatched base pair is within 8 nucleotides of the terminal nucleotide of at least one of the RNA strands of the double-stranded RNA duplex. In some embodiments, a mismatched base pair is within 7 nucleotides of the terminal nucleotide of at least one of the RNA strands of the double-stranded RNA duplex. In some embodiments, a mismatched base pair is within 6 nucleotides of the terminal nucleotide of at least one of the RNA strands of the double-stranded RNA duplex. In some embodiments, a mismatched base pair is within 5 nucleotides of the terminal nucleotide of at least one of the RNA strands of the double-stranded RNA duplex. In some embodiments, a mismatched base pair is within 4 nucleotides of the terminal nucleotide of at least one of the RNA strands of the double-stranded RNA duplex. In some embodiments, a mismatched base pair is within 3 nucleotides of the terminal nucleotide of at least one of the RNA strands of the double-stranded RNA duplex. In some embodiments, a mismatched base pair is within 2 nucleotides of the terminal nucleotide of at least one of the RNA strands of the double-stranded RNA duplex. In some embodiments, a mismatched base pair is within 1 nucleotide of the terminal nucleotide of at least one of the RNA strands of the double-stranded RNA duplex.

Linkers, as used in the compositions of the present disclosure (e.g., nucleic acid editing molecules, ADAR recruiting molecules, RNA targeting molecules, etc.) may attach to the components of such compositions (e.g., nucleic acids, double-stranded RNA duplex, single-stranded guide nucleic acid, etc.) at any point which facilitates the use of the compositions. Such placement and methods of attachment will be readily appreciated by the skilled artisan. For example, without limitation, a linker may be attached (e.g., connected, placed, linked, associated with) to a nucleobase, sugar, phosphate (any of which may be modified or unmodified). Further, without limitation, the placement may be at a 5′ end of a nucleic acid, 3′ end of a nucleic acid, or somewhere in between. Additionally, without limitation, the linker may be attached by integration or use of other components to facilitate the attachment, for example, attachment at the 3′ or 5′ hydroxyl via incorporation of an amine-, thiol-, alkyne-, azide-containing amidite (for conjugation post-cleavage in solution), or active ester-containing amidite (such as NHS ester), further, amino, alkyne and thiol groups may also be incorporated internally, for example, attached to an exocyclic amino group of a nucleobase, or a sugar hydroxyl group at the 2′ position.

In some embodiments, an ADAR recruiting molecule further comprises nucleotides attached to the 3′ end or 5′ end of at least one of the RNA strands of a double-stranded RNA duplex creating a 3′ and/or 5′ end overhang. Any number of additional nucleotides may be attached to a double-stranded RNA duplex (or a strand thereof). For example, in some embodiments, at least one nucleotide (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nucleotides) is attached to the 5′ end of one of the strands of a double-stranded RNA duplex. In some embodiments, at least one nucleotide (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nucleotides) is attached to the 3′ end of one of the strands of a double-stranded RNA duplex. In some embodiments, at least one nucleotide (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nucleotides) is attached to the 5′ end of both of the strands of a double-stranded RNA duplex. In some embodiments, at least one nucleotide (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nucleotides) is attached to the 3′ end of both of the strands of a double-stranded RNA duplex. In some embodiments, the at least one nucleotide is not comprised by a single-stranded guide nucleic acid. In some embodiments, the at least one nucleotide is not comprised by a linker. In some embodiments, the at least one nucleotide is not comprised by a single-stranded guide nucleic acid or a linker.

In some embodiments, nucleotides are attached to both strands of a double-stranded RNA duplex at the same end (e.g., that nucleotides are attached to the 3′ end of one of the strands and nucleotides are attached to the 5′ end of the other strand). In some embodiments, when nucleotides are attached to both strands of a double-stranded RNA duplex at the same end, the number of nucleotides attached to each strand may be the same or may be different. In some embodiments, when nucleotides are attached to both strands of a double-stranded RNA duplex at the same end, the number of nucleotides attached to each strand is the same. In some embodiments, when nucleotides are attached to both strands of a double-stranded RNA duplex at the same end, the number of nucleotides attached to each strand is different. This scenario, where nucleotides are attached to both strands of a double-stranded RNA duplex at the same end, may occur at one or both ends of a double-stranded RNA duplex. In some embodiments, the nucleotides attached to the 3′ and/or 5′ end are modified (e.g., contain a modification as described herein (e.g., sugar, phosphate modifications). In some embodiments, one or more nucleotides attached to the 3′ and/or 5′ end are attached to an additional moiety such as a delivery moiety.

In some embodiments, a double-stranded RNA duplex comprises one or more (e.g., 1, 2, 3, 4, 5, or more) mismatches. In some embodiments, a double-stranded RNA duplex comprises one or more (e.g., 1, 2, 3, 4, 5, or more) mismatches, of which none or some may be wobble base pairs. In some embodiments, a double-stranded RNA duplex comprises one or more (e.g., 1, 2, 3, 4, 5, or more) mismatches, of which none are wobble base pairs. In some embodiments, a double-stranded RNA duplex comprises one or more (e.g., 1, 2, 3, 4, 5, or more) mismatches, of which at least 1 (e.g., 1, 2, 3, 4, 5, or more) comprises a wobble base pair. In some embodiments, a double-stranded RNA duplex comprises one or more (e.g., 1, 2, 3, 4, 5, or more) mismatches, of which all comprise wobble base pairs.

In some embodiments, a double-stranded RNA duplex comprises one or more (e.g., 1, 2, 3, 4, 5, or more) mismatches, of which at least 1 (e.g., 1, 2, 3, 4, 5, or more) mismatches are within 7 or fewer (e.g., 7, 6, 5, 4, 3, 2, or 1) nucleotides of the terminal nucleotide base pair of the double-stranded RNA duplex. In some embodiments, a double-stranded RNA duplex comprises one or more (e.g., 1, 2, 3, 4, 5, or more) mismatches, of which none or some may be wobble base pairs, of which at least 1 (e.g., 1, 2, 3, 4, 5, or more) wobble base pair is within 7 or fewer (e.g., 7, 6, 5, 4, 3, 2, or 1) nucleotides of the terminal nucleotide base pair of the double-stranded RNA duplex. In some embodiments, a double-stranded RNA duplex comprises one or more (e.g., 1, 2, 3, 4, 5, or more) mismatches, of which none are wobble base pairs, of which at least 1 (e.g., 1, 2, 3, 4, 5, or more) mismatch is within 7 or fewer (e.g., 7, 6, 5, 4, 3, 2, or 1) nucleotides of the terminal nucleotide base pair of the double-stranded RNA duplex. In some embodiments, a double-stranded RNA duplex comprises one or more (e.g., 1, 2, 3, 4, 5, or more) mismatches, of which at least 1 (e.g., 1, 2, 3, 4, 5, or more) comprises a wobble base pair, of which at least 1 (e.g., 1, 2, 3, 4, 5, or more) wobble base pair is within 7 or fewer (e.g., 7, 6, 5, 4, 3, 2, or 1) nucleotides of the terminal nucleotide base pair of the double-stranded RNA duplex. In some embodiments, a double-stranded RNA duplex comprises one or more (e.g., 1, 2, 3, 4, 5, or more) mismatches, of which all comprise wobble base pairs, of which at least 1 (e.g., 1, 2, 3, 4, 5, or more) wobble base pair is within 7 or fewer (e.g., 7, 6, 5, 4, 3, 2, or 1) nucleotides of the terminal nucleotide base pair of the double-stranded RNA duplex.

In some embodiments, a double-stranded RNA duplex comprises two RNA strands having sequences selected from Table A. Table A provides examples of double-stranded RNA duplexes, or recruiting domain portions, of ADAR recruiting molecule sequences provided elsewhere herein in Table 8. The two RNA strands of each duplex in Table A are shown oriented anti-parallel to one another, with base pair mismatches shown emphasized.

TABLE 1
Examples of Double-Stranded RNA Duplexes

| | SEQ ID | |
|---|---|---|
| RNA Duplex Strand Sequences | NOs. | Example Compounds |
| 5-GGUGUCGAGAAGAGGAGAACA-3 | 53 | RD0226 |
| 3-CCACAGCUCCUCUGCUCUUGU-5 | 116 | |
| 5-GGUGUCGAGAAGAGGAGAACAAUAU-3 | 3 | RD0001, RD0016, |
| 3-CCACAGCUCCUCUGCUCUUGUUGUA-5 | 4 | RD0178, RD0441 |
| 5-GGUGUCGAGAAGAGGAGAACAAUAUGC-3 | 50 | RD0179 |
| 3-CCACAGCUCCUCUGCUCUUGUUGUACG-5 | 119 | |
| 5-GGUGUCGAGAAGAGGAGAACAAUAUGC-3 | 50 | RD0225 |
| 3-CCACAGCUCCUCUGCUCUUGUUAUACG-5 | 117 | |
| 5-GGUGUCGAGAAGAGGAGAACG-3 | 55 | RD0227 |
| 3-CCACAGCUCCUCUGCUCUUGC-5 | 120 | |
| 5-GGGUGGAAGAGUAGAACAAUAUGC-3 | 5 | RD0034, RD033, RD0164, |
| 3-CCCACCCUCUGAUCUUGUUAUACG-5 | 6 | RD0169, RD0465, |
| | | RD0473 |
| 5-GGGUGGAAGAGUAGAACAAUAUGC-3 | 5 | RD0160 |
| 3-CCCACCCUCUGAUCUUGUUGUACG-5 | 121 | |
| 5-GGGUGGAAGAGUAGAACAAUAUGC-3 | 5 | RD0480 |
| 3-CCCACCCUCUGAUCUUGUUAUGCG-5 | 122 | |
| 5-GGGUGGAAGAGUAGAACAAUGC-3 | 39 | RD0163 |
| 3-CCCACCCUCUGAUCUUGUUGCG-5 | 123 | |
| 5-GGGUGGAAUAGUAUAACAAUAU-3 | 22 | RD0042, RD0057 |
| 3-CCCACCCUAUGAUAUUGUUAUA-5 | 124 | |
| 5-GGGUGGAAUAGUAUAACAAUAUGC-3 | 18 | RD0027 |
| 3-CCCACCCUAUGAUAUUGUUAUACG-5 | 125 | |
| 5-GGUGGGUGGAAUAGUAUAACAAUAU-3 | 28 | RD0060 |
| 3-CCACCCACCCUAUGAUAUUGUUGUA-5 | 126 | |
| 5-GGUGGGUGGAAUAGUAUAACAAUAUGC-3 | 34 | RD0072 |
| 3-CCACCCACCCUAUGAUAUUGUUGUACG-5 | 127 | |
| 5-GGGUGGAAUAGUACAACAAUAUGC-3 | 20 | RD0039 |
| 3-CCCACCCUAUGAUGUUGUUAUACG-5 | 128 | |
| 5-GGUGGGUGGAAUAGUACAACAAUAUGC-3 | 36 | RD0074 |
| 3-CCACCCACCCUAUGAUGUUGUUGUACG-5 | 129 | |
| 5-GGGUGGAACAGUACAACAAUAUGC-3 | 7 | RD0036 |
| 3-CCCACCCUGUGAUGUUGUUAUACG-5 | 130 | |
| 5-GUGGAAGAGGAGAACG-3 | 67 | RD0233 |
| 3-CACCCUCUGCUCUUGC-5 | 131 | |
| 5-GUGGAAGAGGAGAACGC-3 | 81 | RD0240 |
| 3-CACCCUCUGCUCUUGCG-5 | 132 | |
| 5-GGGUGGAAGAGGAGAACG-3 | 57 | RD0228 |
| 3-CCCACCCUCUGCUCUUGC-5 | 133 | |
| 5-GGUGGGUGGAAGAGGAGAACG-3 | 86 | RD0453 |
| 3-CCACCCACCCUCUGCUCUUGC-5 | 134 | |
| 5-GGUGGGUGGAAGAGGAGAACG-3 | 86 | RD0464 |
| 3-CCACCCACCCUCUGCUCUUGC-5 | 134 | |
| 5-GUGGAAGAGUAGACCG-3 | 69 | RD0234 |
| 3-CACCCUCUGAUCUGGC-5 | 135 | |
| 5-GGGUGGAAGAGUAGACCG-3 | 59 | RD0229 |
| 3-CCCACCCUCUGAUCUGGC-5 | 136 | |
| 5-GUGGAAGAGUAGACCA-3 | 65 | RD0232 |
| 3-CACCCUCUGAUCUGGU-5 | 137 | |
| 5-GUGGAAGAGUAGACCAC-3 | 79 | RD0239 |
| 3-CACCCUCUGAUCUGGUG-5 | 138 | |
| 5-GUGGAAGAGGAGAACA-3 | 63 | RD0231 |
| 3-CACCCUCUGCUCUUGU-5 | 139 | |
| 5-GUGGAAGAGGAGAACAAC-3 | 71 | RD0235 |
| 3-CACCCUCUGCUCUUGUUG-5 | 140 | |
| 5-GUGGAAGAGGAGAACAGC-3 | 73 | RD0236 |
| 3-CACCCUCUGCUCUUGUCG-5 | 141 | |
| 5-GUGGAAGAGGAGAACAACGC-3 | 75 | RD0237 |
| 3-CACCCUCUGCUCUUGUUGCG-5 | 142 | |
| 5-GUGGAAGAGGAGAACAAUGC-3 | 77 | RD0238 |
| 3-CACCCUCUGCUCUUGUUACG-5 | 143 | |
| 5-GUGUGGAAGAGGAGAACAGUG-3 | 111 | RD0484 |
| 3-CCCACCCUCUGCUCUUGUCAC-5 | 144 | |
| 5-GUGUGGAAGAGGAGAACAGUG-3 | 111 | RD0485 |
| 3-CCCACCCUCUGCUCUUGUCGC-5 | 145 | |
| 5-GGUGGGUGGAAGAGGAGAACA-3 | 84 | RD0452, RD0463 |
| 3-CCACCCACCCUCUGCUCUUGU-5 | 115 | |
| 5-GGUGGGUGGAAGAGGAGAACAAUAUGC-3 | 94 | RD0459 |
| 3-CCACCCACCCUCUGCUCUUGUUGUACG-5 | 146 | |
| 5-GGUGGGUGGAAGAGGAGAACAAUAUGC-3 | 94 | RD0461 |
| 3-CCACCCACCCUCUGCUCUUGUUAUACG-5 | 147 | |
| 5-GGGUGGAAGAGGAGAACAAUA-3 | 107 | RD0482 |
| 3-CCCACCCUCUGCUCUUGUUAU-5 | 148 | |
| 5-GGGUGGAAGAGGAGAACAAUAUGC-3 | 103 | RD0479 |
| 3-CCCACCCUCUGCUCUUGUUAUACG-5 | 149 | |
| 5-GGGUGGAAGAGGAGAACAAUAUGC-3 | 103 | RD0481 |
| 3-CCCACCCUCUGCUCUUGUUAUGCG-5 | 150 | |
| 5-GGGUGGAAGAGGAGAACAGCG-3 | 109 | RD0483 |
| 3-CCCACCCUCUGCUCUUGUCGC-5 | 145 | |
| 5-GGGUGUAAGAGUAGAACAAUAUGC-3 | 41 | RD0172 |
| 3-CCCACACUCUGAUCUUGUUAUACG-5 | 151 | |
| 5-GGGUGUAAGAGUAGAACAAUAUGC-3 | 41 | RD0173 |
| 3-CCCACACUCUGAUCUUGUUGUACG-5 | 152 | |
| 5-GGGUGGAAGAGUACAACAAUAUGC-3 | 44 | RD0174 |
| 3-CCCACCCUCUGAUGUUGUUAUACG-5 | 153 | |
| 5-GGGUGGAAGAGUACAACAAUAUGC-3 | 44 | RD0175 |
| 3-CCCACCCUCUGAUGUUGUUGUACG-5 | 154 | |
| 5-GGGUGGAAGAGUAUAACAAUAUGC-3 | 47 | RD0176 |
| 3-CCCACCCUCUGAUAUUGUUGUACG-5 | 155 | |
| 5-GGGUGGAAGAGUAUAACAAUAUGC-3 | 47 | RD0177 |
| 3-CCCACCCUCUGAUAUUGUUAUACG-5 | 156 | |
| 5-GGUGGGUGGAAGAGUAUAACAAUGC-3 | 30 | RD0064 |
| 3-CCACCCACCCUCUGAUAUUGUUGCG-5 | 157 | |
| 5-GGUGAAGAGGAGAACAAUAU-3 | 14 | RD0020 |
| 3-CCACCUCUGCUCUUGUUGUA-5 | 158 | |
| 5-GGUGAAUAGUAUAACAAUAU-3 | 16 | RD0025 |
| 3-CCACCUAUGAUAUUGUUGUA-5 | 159 | |
| 5-GGGUGGAAGAGGAGACCA-3 | 61 | RD0230 |
| 3-CCCACCCUCUGCUCUGGU-5 | 160 | |
| 5-GGUGGGUGGAACAGGACAACAAUAUGC-3 | 88 | RD0455, RD0466 |
| 3-CCACCCACCCUGUGCUGUUGUUAUACG-5 | 161 | |
| 5-GGUGGGUGGAACAGGAGAACA-3 | 90 | RD0456, RD0467 |
| 3-CCACCCACCCUGUGCUCUUGU-5 | 162 | |
| 5-GGUGGGUGGAACAGGAGAACG-3 | 92 | RD0457, RD0468 |
| 3-CCACCCACCCUGUGCUCUUGC-5 | 163 | |
| 5-GGGUGGAAUAGUAUAACAAUGC-3 | 24 | RD0046 |
| 3-CCCACCCUAUGAUAUUGUUACG-5 | 164 | |
| 5-GGGUGGAAUAGUACAACAAUGC-3 | 26 | RD0049 |
| 3-CCCACCCUAUGAUGUUGUUACG-5 | 165 | |
| 5-GGUGGGUGGAAGAGGAGAAC-3 | 166 | RD1017 |
| 3-CCACCCACCCUCUGCUCUUGU-5 | 115 | |
| 5-GGUGGGUGGAAGAGGAGAACA-3 | 84 | RD1018 |
| 3-CCACCCACCCUCUGCUCUUGUU-5 | 118 | |
| 5-GGUGGGUGGAAGAGGAGAACA-3 | 84 | RD1019 |
| 3-CCACCCACCCUCUGCUCUUG-5 | 167 | |

As illustrated by the examples in Table A, in some embodiments, a double-stranded RNA duplex comprises at least two mismatches. In some embodiments, the at least two mismatches include two mismatches separated by 1-5 (e.g., 1, 2, 3, 4, or 5) complementary base pairs. In some embodiments, the two mismatches are separated by three complementary base pairs. In some embodiments, the two mismatches include one or both of AC and GG. In some embodiments, the two mismatches include both of AC and GG, where the AC and GG are separated by 1-5 (e.g., 1, 2, 3, 4, or 5) complementary base pairs. In some embodiments, the two mismatches include both of AC and GG, where the AC and GG are separated by three complementary base pairs. In some embodiments, each of the two mismatches is separated from one of the terminal ends of the duplex region by at least three (e.g., 3, 4, 5, 3-10, 5-15, 10-20, or more) nucleotides. In some embodiments, the at least two mismatches include the two mismatches as described, and further include a third mismatch of UG. In some embodiments, the third mismatch is within five nucleotides (e.g., 1, 2, 3, 4, or 5) of one of the terminal ends of the duplex region.

In some embodiments, at least one strand of a double-stranded RNA duplex comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to any one of Strand Ref.: 3-4, 6-7, 15-16, 18-19, and 24-25. In some embodiments, at least one strand of a double-stranded RNA duplex comprises a sequence comprising any one of Strand Ref.: 3-4, 6-7, 15-16, 18-19, and 24-25. The terms “percent identity,” “sequence identity,” “% identity,” “% sequence identity,” and “% identical,” as they may be interchangeably used herein, refer to a quantitative measurement of the similarity between two sequences (e.g., nucleic acid or amino acid). The percent identity of genomic DNA sequence, intron and exon sequence, and amino acid sequence between humans and other species varies by species type, with chimpanzee having the highest percent identity with humans of all species in each category.

Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F. et al., J. Molec. Biol., 215, 403 (1990)).

When a percent identity is stated, or a range thereof (e.g., at least, more than, etc.), unless otherwise specified, the endpoints shall be inclusive, and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (e.g., 0.1%), hundredths of a percent (e.g., 0.01%), etc.). In some embodiments, at least one strand of a double-stranded RNA duplex comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 3. In some embodiments, at least one of the strands in a double-stranded RNA duplex comprises a sequence of Strand Ref.: 3. In some embodiments, at least one strand of a double-stranded RNA duplex comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 4. In some embodiments, at least one of the strands in a double-stranded RNA duplex comprises a sequence of Strand Ref.: 4. In some embodiments, at least one strand of a double-stranded RNA duplex comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 6. In some embodiments, at least one of the strands in a double-stranded RNA duplex comprises a sequence of Strand Ref.: 6. In some embodiments, at least one strand of a double-stranded RNA duplex comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 7. In some embodiments, at least one of the strands in a double-stranded RNA duplex comprises a sequence of Strand Ref.: 7. In some embodiments, at least one strand of a double-stranded RNA duplex comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 15. In some embodiments, at least one of the strands in a double-stranded RNA duplex comprises a sequence of Strand Ref.: 15. In some embodiments, at least one strand of a double-stranded RNA duplex comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 16. In some embodiments, at least one of the strands in a double-stranded RNA duplex comprises a sequence of Strand Ref.: 16. In some embodiments, at least one strand of a double-stranded RNA duplex comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 18. In some embodiments, at least one of the strands in a double-stranded RNA duplex comprises a sequence of Strand Ref.: 18. In some embodiments, at least one strand of a double-stranded RNA duplex comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 19. In some embodiments, at least one of the strands in a double-stranded RNA duplex comprises a sequence of Strand Ref.: 19. In some embodiments, at least one strand of a double-stranded RNA duplex comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 24. In some embodiments, at least one of the strands in a double-stranded RNA duplex comprises a sequence of Strand Ref.: 24. In some embodiments, at least one strand of a double-stranded RNA duplex comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 25. In some embodiments, at least one of the strands in a double-stranded RNA duplex comprises a sequence of Strand Ref.: 25. In some embodiments, at least one strand of a double-stranded RNA duplex comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 27. In some embodiments, at least one of the strands in a double-stranded RNA duplex comprises a sequence of Strand Ref.: 27. In some embodiments, at least one strand of a double-stranded RNA duplex comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 28. In some embodiments, at least one of the strands in a double-stranded RNA duplex comprises a sequence of Strand Ref.: 28.

In some embodiments, a double-stranded RNA duplex comprises an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 3 and an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 4. In some embodiments, a double-stranded RNA duplex comprises an RNA strand comprising the sequence of Strand Ref.: 3 and an RNA strand comprising the sequence of Strand Ref.: 4. In some embodiments, a double-stranded RNA duplex comprises an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 6 and an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 7. In some embodiments, a double-stranded RNA duplex comprises an RNA strand comprising the sequence of Strand Ref.: 6 and an RNA strand comprising the sequence of Strand Ref.: 7. In some embodiments, a double-stranded RNA duplex comprises an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 15 and an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 16. In some embodiments, a double-stranded RNA duplex comprises an RNA strand comprising the sequence of Strand Ref.: 15 and an RNA strand comprising the sequence of Strand Ref.: 16. In some embodiments, a double-stranded RNA duplex comprises an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 18 and an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 19. In some embodiments, a double-stranded RNA duplex comprises an RNA strand comprising the sequence of Strand Ref.: 18 and an RNA strand comprising the sequence of Strand Ref.: 19. In some embodiments, a double-stranded RNA duplex comprises an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 24 and an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 25. In some embodiments, a double-stranded RNA duplex comprises an RNA strand comprising the sequence of Strand Ref.: 24 and an RNA strand comprising the sequence of Strand Ref.: 25. In some embodiments, a double-stranded RNA duplex comprises an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 27 and an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 28. In some embodiments, a double-stranded RNA duplex comprises an RNA strand comprising the sequence of Strand Ref.: 27 and an RNA strand comprising the sequence of Strand Ref.: 28.

In some embodiments, the double-stranded RNA duplex does not contain a hairpin. When referring herein to the absence (e.g., not containing) of a hairpin, the molecule being discussed or which is the subject of the description, does not contain a “stem-loop” configuration such that a single nucleic acid is folded back up on itself and base pairs a portion of its distal ends such that there is a loop of (e.g., stretch of, segment of, portion of) single-stranded nucleotides of the nucleic acid that are unpaired and thus form a “loop” or “hairpin.” In some embodiments, the RNA strands of a double-stranded RNA duplex are distinct in that they are wholly separable from one another. In some embodiments, the only bonds holding the RNA strands of double-stranded RNA duplex together are hydrogen bonds of the paired nucleotides.

In some embodiments, an ADAR recruiting molecule or an RNA targeting molecule further comprises at least one additional moiety. An additional moiety may be attached to the 5′ or 3′ end of at least one of the RNA strands of a double-stranded RNA duplex, and/or to the 5′ or 3′ end of a single-stranded guide nucleic acid. Additionally, an ADAR recruiting molecule or an RNA targeting molecule may comprise an additional moiety within one or more strands of the RNA strands of the double-stranded RNA duplex or single-stranded guide nucleic acid. Further, an ADAR recruiting molecule or an RNA targeting molecule may comprise more than one additional moiety. The additional moieties may be attached to the same or different ends of a double-stranded RNA duplex or single-stranded guide nucleic acid. An additional moiety may further be attached to the double-stranded RNA duplex or single-stranded guide nucleic acid in any configuration by means of additional nucleotides as described hereinabove, or by a linker. In some embodiments, an additional moiety may be a protein, or fragment thereof. In some embodiments, an additional moiety may be a nucleic acid, or fragment thereof (e.g., nucleotides). In some embodiments, an additional moiety is a therapeutic. In some embodiments, an additional moiety is a diagnostic.

In some embodiments, an additional moiety may be a delivery moiety. A “delivery moiety,” as may be used herein, refers to a moiety which influences (e.g., facilitates, targets, increases the likelihood it, or the composition (e.g., molecule) it is attached to, will be found) the position, migration, localization, or direction of the delivery moiety. For example, in some embodiments, a delivery moiety increases the likelihood that it, or the molecule to which it is attached, will localize to a desired target. In some embodiments, a delivery moiety helps target the molecule to one or more cell or tissue types of interest (e.g., a target tissue for a treatment). In some embodiments, a delivery moiety helps the molecule enter one or more cells or tissues (e.g., by promoting cell membrane and/or nuclear membrane penetration). In some embodiments, a delivery moiety may be a protein known to associate with a particular cell and/or receptor, thus influencing targeting capabilities of the composition (e.g., molecule) to which it is attached (e.g., associated). Various types of delivery moieties are known in the art, for example without limitation, proteins, oligonucleotides, and lipids. In some embodiments, a delivery moiety targets a specific cell type. In some embodiments, a delivery moiety targets a specific receptor. In some embodiments, a delivery moiety targets a specific biological component or cellular component (e.g., structure, molecule, tissue).

Accordingly, in some embodiments, an ADAR recruiting molecule or an RNA targeting molecule comprises a delivery moiety. For example, in some embodiments, an ADAR recruiting molecule or an RNA targeting molecule comprises a lipid (e.g., a delivery moiety comprising a lipid, such as cholesterol or a fatty acid). In some embodiments, an ADAR recruiting molecule or an RNA targeting molecule comprises a peptide, a protein, an antibody, or an antibody fragment (e.g., glucagon-like peptide 1, anti-CD71 antibody or a fragment thereof, anti-CD22 antibody or a fragment thereof). In some embodiments, an ADAR recruiting molecule or an RNA targeting molecule comprises an aptamer. In some embodiments, an ADAR recruiting molecule or an RNA targeting molecule comprises a sugar. For example, in some embodiments, an ADAR recruiting molecule or an RNA targeting molecule comprises N-acetylgalactosamine (GalNAc). In some embodiments, an ADAR recruiting molecule or an RNA targeting molecule comprises a trivalent GalNAc. In some embodiments, an ADAR recruiting molecule or an RNA targeting molecule comprises folate. Additional examples of suitable delivery moieties, as well as methods of designing and obtaining delivery moieties based on a desired target are known in the art.

In some embodiments, an additional moiety may be a marker or tag. A “marker” or “tag,” as used herein, refers to a molecule (e.g., nucleic acid, protein, etc.) which can be used to identify the molecule in vitro and/or in vivo. Markers or tags may be any composition or molecule (e.g., nucleic acid, amino acid, peptide). Examples of markers include nucleic acids, proteins (e.g., glycosylated proteins, oxine), fluorescent proteins (e.g., green and/or red fluorescent protein), proteins having functional structures (e.g., tetracysteine loops, epitopes), any of which may be natural or synthetic (e.g., synthetic nucleic acids, amino acids, peptides, etc.), which may be detected in vivo, in vitro, ex vivo, visually, or by exploitation of a property of the tag (e.g., fluorescence, magnetism, radioactivity, size, affinity, enzyme activity, etc.). For example, in some embodiments, a marker is a fluorescent protein, including without limitation, green fluorescent protein (GFP). As used herein, the term “green fluorescent protein” (GFP) refers to a protein originally isolated from the jellyfish Aequorea victoria that fluoresces green when exposed to blue light or a derivative of such a protein (e.g., an enhanced or wavelength-shifted version of the protein). In some embodiments, an additional moiety is GFP.

### Linkers

In some embodiments, an ADAR recruiting molecule and/or RNA targeting molecule further comprises a linker. The term “linker,” as used herein, refers to a chemical moiety linking two molecules or moieties, e.g., a double-stranded RNA duplex and a single-stranded guide nucleic acid. Typically, the linker is positioned between, or flanked by, two groups, molecules, or other moieties and connected to each one via a covalent bond, thus connecting the two. In some embodiments, the linker comprises an amino acid or a plurality of amino acids (e.g., a peptide or protein). In some embodiments, the linker comprises a nucleotide (e.g., DNA or RNA) or a plurality of nucleotides (e.g., a nucleic acid, (e.g., oligonucleotide)). In some embodiments, the linker is an organic molecule, functional group, group, polymer, or other chemical moiety. In some embodiments, the linker is a cleavable linker, e.g., the linker comprises a bond that can be cleaved upon exposure to, for example, UV light or a hydrolytic enzyme, such as a protease or esterase. In some embodiments, the linker is any stretch of amino acids or nucleotides having at least 1, at least 2, at least 3, at least 4, at least 5, at least 6, at least 7, at least 8, at least 9, at least 10, at least 15, at least 20, at least 25, at least 30, at least 40, at least 50, or more amino acids or nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more). In other embodiments, the linker is a chemical bond (e.g., a covalent bond, amide bond, disulfide bond, ester bond, carbon-carbon bond, carbon heteroatom bond). The length of a linker may be any suitable length to achieve the goal of linking the moieties as described herein, such assessments for determining a length and the knowledge for performing such assessments are known in the art and are readily appreciated by the skilled artisan. For example, without limitation, linkers may be greater than or equal to 1 atom in length (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, or more atoms in length). In some embodiments, a linker is fewer than or equal to 200 atoms in length (e.g., 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). In some embodiments, a linker is fewer than or equal to 200 atoms in length (e.g., 1-200, 10-150, 25-125, 1-100, 25-75, 20-60, 1-50, 20-40, 10-30, 5-25, 1-20, 10-15, 1-10, 5-50, 1-10, 100-200, 150-200, 100-150, or 50-150 atoms in length).

In some embodiments, a linker may be at least 4 atoms in length. In some embodiments, a linker may be at least 5 atoms in length. In some embodiments, a linker may be at least 6 atoms in length. In some embodiments, a linker may be at least 7 atoms in length. In some embodiments, a linker may be at least 8 atoms in length. In some embodiments, a linker may be at least 9 atoms in length. In some embodiments, a linker may be at least 10 atoms in length. In some embodiments, a linker may be at least 15 atoms in length. In some embodiments, a linker may be at least 20 atoms in length. In some embodiments, a linker may be at least 30 atoms in length. In some embodiments, a linker may be at least 40 atoms in length. In some embodiments, a linker may be at least 50 atoms in length. In some embodiments, a linker may be at least 75 atoms in length. In some embodiments, a linker may be at least 100 atoms in length. In some embodiments, a linker may be at least 150 atoms in length. In some embodiments, a linker may be at least 100 atoms in length. In some embodiments, a linker may be at least 180 atoms in length.

In some embodiments, a linker may be fewer than or equal to 180 atoms in length. In some embodiments, a linker may be fewer than or equal to 150 atoms in length. In some embodiments, a linker may be fewer than or equal to 100 atoms in length. In some embodiments, a linker may be fewer than or equal to 75 atoms in length. In some embodiments, a linker may be fewer than or equal to 50 atoms in length. In some embodiments, a linker may be fewer than or equal to 40 atoms in length. In some embodiments, a linker may be fewer than or equal to 30 atoms in length. In some embodiments, a linker may be fewer than or equal to 20 atoms in length. In some embodiments, a linker may be fewer than or equal to 15 atoms in length. In some embodiments, a linker may be fewer than or equal to 10 atoms in length. In some embodiments, a linker may be fewer than or equal to 9 atoms in length. In some embodiments, a linker may be fewer than or equal to 8 atoms in length. In some embodiments, a linker may be fewer than or equal to 7 atoms in length. In some embodiments, a linker may be fewer than or equal to 6 atoms in length. In some embodiments, a linker may be fewer than or equal to 5 atoms in length. In some embodiments, a linker may be fewer than or equal to 4 atoms in length.

In some embodiments, a linker may be about 4 atoms in length to about 200 atoms in length. In some embodiments, a linker may be about 5 atoms in length to about 180 atoms in length. In some embodiments, a linker may be about 6 atoms in length to about 150 atoms in length. In some embodiments, a linker may be about 7 atoms in length to about 140 atoms in length. In some embodiments, a linker may be about 8 atoms in length to about 130 atoms in length. In some embodiments, a linker may be about 9 atoms in length to about 120 atoms in length. In some embodiments, a linker may be about 10 atoms in length to about 110 atoms in length. In some embodiments, a linker may be about 15 atoms in length to about 100 atoms in length. In some embodiments, a linker may be about 20 atoms in length to about 100 atoms in length. In some embodiments, a linker may be about 30 atoms in length to about 150 atoms in length. In some embodiments, a linker may be about 40 atoms in length to about 150 atoms in length. In some embodiments, a linker may be about 50 atoms in length to about 150 atoms in length. In some embodiments, a linker may be about 75 atoms in length to about 150 atoms in length. In some embodiments, a linker may be about 100 atoms in length to about 150 atoms in length. In some embodiments, a linker may be about 150 atoms in length to about 200 atoms in length.

As described above, in some embodiments, a linker of the disclosure generally refers to a chemical moiety linking two molecules. In the context of a linker, in some embodiments, x atoms in length refers to the fewest number of contiguous atoms separating the two molecules, where the fewest number of contiguous atoms can be determined by counting the number of atoms in the linker as traced along the shortest path from one molecule to the other. By way of example, an amino acid linker may be of a general formula: Y—NH—CR—CO—Z, where Y and Z are two molecules joined by the amino acid linker, and R is the amino acid side chain. In accordance with the disclosure, the amino acid linker can be described as being 3 atoms in length, as the shortest path from Y to Z can be traced through the atoms N, C, and C of the amino acid linker.

Where reference is made herein to an “atom” in length, it should be appreciated that the actual length (e.g., in Angstroms) of a linker can depend on a variety of factors. For example, the actual linker length may depend on environmental factors and on the properties of a linker, including the types of chemical bonds, the substituents, and the particular atoms present in the linker, as each type of chemical element has a different size (e.g., atomic radius). Thus, it shall be understood that a linker described as having x atoms in length provides a useful structural descriptor that represents an approximate size (e.g., that may vary slightly depending on which atoms are in a linker). Accordingly, while the term atom, as a term of art, could imply differences in atomic radius, which is generally understood to reflect the different lengths of atoms of different elements, and encompass the same, such variations are not relevant to a measure of x atoms in length referred to herein. Thus, for illustration only, and in no way limiting, although 1 atom length (e.g., atomic radius) of helium will differ from 1 atom length of magnesium, either one could represent 1 atom in length in the context of a linker.

Without limitation, a linker may comprise an alkyl, alkenyl, alkynyl, substituted alkyl, substituted alkenyl, substituted alkynyl, repeated ethylene glycol groups, ether, thioether, urea, carbonate, amine, amide, maleimide-thioether, disulfide, phosphodiester, sulfonamide linkage, a product of a click reaction (e.g., a triazole from the azide-alkyne cycloaddition), and/or carbamate. Additionally, a linker may be a cleavable linker, e.g., redox cleavable linker (such as a reductively cleavable linker; e.g., a disulfide group), an acid cleavable linker (e.g., a hydrazone group, an ester group, an acetal group, and/or a ketal group), an esterase cleavable linker (e.g., an ester group), a phosphatase cleavable linker (e.g., a phosphate group), or a peptidase cleavable linker (e.g., a peptide bond), a bio-cleavable linker, e.g., comprising DNA, RNA, disulfide, amide, functionalized monosaccharides or oligosaccharides of galactosamine.

In some embodiments, a linker may comprise acetamido linkages, for example, bromoacetamido thiol-reactive chemistry instead of maleimide (for example, without limitation, Bromoacetamido-PEG2-NHS ester). In some embodiments, a linker may exploit other chemical properties, for example, as will be readily appreciated by the skilled artisan as applied to linking moieties (e.g., bioconjugation), without limitation, include: Diels-Alder; Reverse electron-demand Diels-Alder; Copper-catalyzed (CuAAC) Huisgen azide-alkyne 1,3-dipolar cycloaddition; Copper-free strained alkyne-azide (SPAAC); Staudinger ligation; Active esters including NHS, OPfp, OTfp esters; Native chemical ligation; and Chemoenzymatic ligation.

In some embodiments, a linker may comprise a conjugation or binding system. A conjugation or binding system comprises binding partners (e.g., nucleic acids, proteins, chemicals, vitamins, etc.) which have an affinity for one another, such that when exposed to each other they will bind readily to one another (e.g., covalently, non-covalently). For example, without limitation, biotin binding systems (e.g., biotin-avidin, biotin-streptavidin, biotin-neutravidin). Conjugation or binding systems such as these are readily known to the skilled artisan and are envisioned as being encompassed by the present disclosure. In some embodiments, a binding system comprises binding partners which bind covalently. In some embodiments, a binding system comprises binding partners which bind non-covalently. In some embodiments, a binding system comprises binding partners, wherein at least one binding partner comprises biotin. In some embodiments, a binding system comprises binding partners, wherein at least one binding partner comprises avidin. In some embodiments, a binding system comprises binding partners, wherein at least one binding partner comprises streptavidin. In some embodiments, a binding system comprises binding partners, wherein at least one binding partner comprises neutravidin. In some embodiments, a binding system comprises binding partners, wherein the binding partners comprise biotin and one of avidin, streptavidin, or neutravidin. In some embodiments, a binding system comprises binding partners, wherein the binding partners comprise biotin and streptavidin.

In some embodiments, binding partners may be non-covalent complexes between an antibody-cationic polymer (protamine) conjugate and (anionic) siRNA (for example, without limitation as shown in the art, see Bäumer et al., Clin. Cancer Res., 2015, 21, 1383). In some embodiments, non-covalent binding (e.g., of binding partners) may also include binding through hydrogen binding, such as Watson and Crick pairing such as with a short oligomer, RNA, DNA, PNA duplex.

In some embodiments, the constituent components of a linker are modified, for example without limitation, in the event a linker comprises nucleotides, the nucleosides and/or backbone thereof may be modified (e.g., contain a modification as described herein (e.g., sugar, phosphate modifications)). In some embodiments, a linker is attached at the 5′ end of at least one of the RNA strands of the double-stranded RNA duplex. In some embodiments, a linker is attached at the 3′ end of at least one of the RNA strands of the double-stranded RNA duplex. In some embodiments, a linker is attached at the 5′ end of at least one of the RNA strands of the double-stranded RNA duplex and the 3′ end of at least one of the RNA strands of the double-stranded RNA duplex. In some embodiments, a linker is attached at both 5′ ends of the RNA strands of the double-stranded RNA duplex. In some embodiments, a linker is attached at both 3′ ends of the RNA strands of the double-stranded RNA duplex. In some embodiments, a linker is attached through at least one (e.g., 1, 2, 3, 4) end of the RNA strands of a double-stranded RNA duplex. In some embodiments, a linker connects the double-stranded RNA duplex to a single-stranded guide nucleic acid. In some embodiments, a linker may be selected from any one of the following: polyethylene glycol (PEG), Carbon (C) (e.g., C₍ₙ₊₂₎, phosphate (Ph), Ph-C₄, cyclohexane (Cy), Cy-C₆, and/or C₃C₆. For example, without limitation, the following formulae are illustrative of the linkers mentioned above:

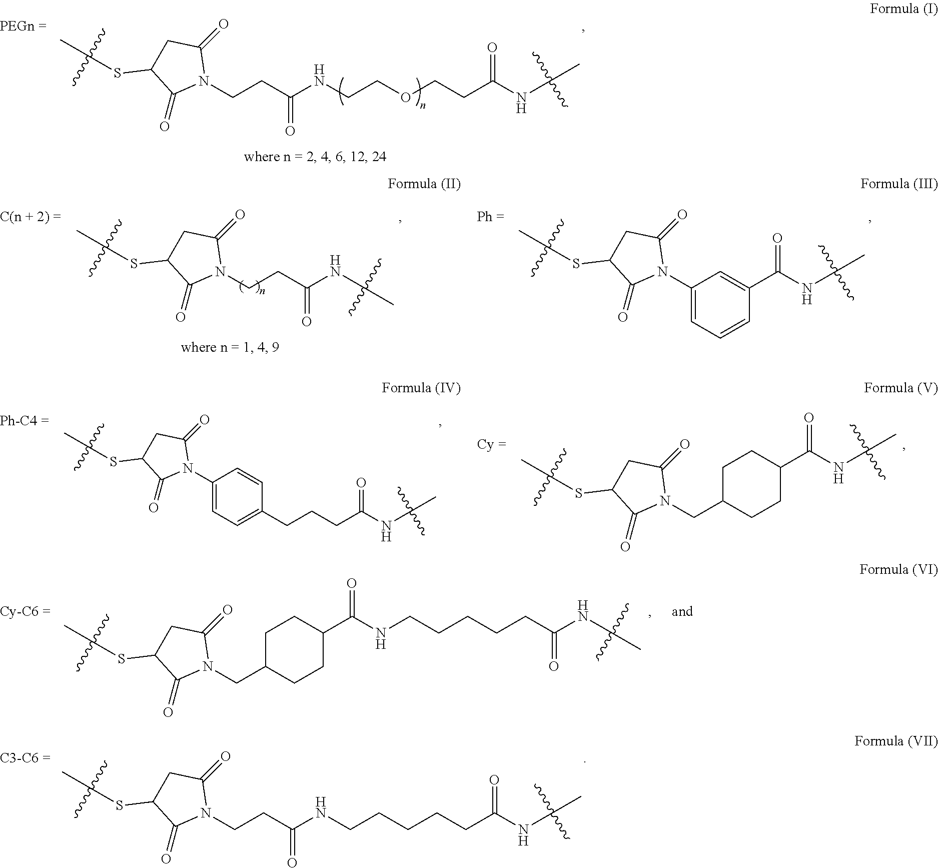

In some embodiments, an RNA strand of a double-stranded RNA duplex or single-stranded guide nucleic acid may also comprise at least one terminal moiety. In some embodiments, a terminal moiety comprises Formula (X₁):

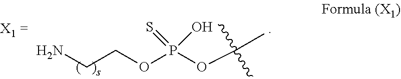

In some embodiments, a terminal moiety comprises Formula (X₂):

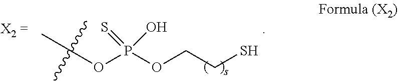

In some embodiments, a terminal moiety may be reacted to bond with a linker as described herein. As used herein, the term “reacted,” refers to the term of art understood by the skilled artisan to encompass chemical manipulations (e.g., reactions, derivatizations, covalent bonding) to change (e.g., modify, alter) the composition (e.g., terminal moiety) to allow for additional linkage (e.g., for example, without limitation, to a linker). For example, without limitation, X₁ and/or X₂ may be reacted to release the terminal amino group hydrogen (H; in the case of X₁) or the terminal thiol group H (in the case of X₂), to allow for linkage via the Nitrogen (N; in the case of X₁) or Sulfur (S; in the case of X₂). In some embodiments, a terminal moiety is reacted and joins (e.g., links, bonds) any of the linkers as described herein (e.g., for example, without limitation, those as in any of the compositions as described by the sequences exemplified in Table 8, Formulae (I)-(VII), as described elsewhere in this disclosure). As will be immediately appreciated by the skilled artisan, as used herein, Formula X₁ and/or X₂, may be used to refer to a terminal moiety (e.g., unreacted and containing the hydrogens as described hereinabove) and/or may be used to describe the reacted derivatives thereof when used to describe components joining at least two molecules.

A linker may be a branched or unbranched linker. For example, without limitation, a linker may be a tridentate branched linker of the form [X-PEGy-(Z)3], where X is a maleimide or protected amine, and Z are 3×active esters, such as Pfp or Tfp ester.

A branched linker may be of any size or number of branches suitable for the application in the compositions of the present disclosure. For example, a branched linker may have at least 2 (e.g., 2, 3, 4, 5, 6, 7, 8, 9, 10, or more) branches. In some embodiments, a branched linker has fewer than or equal to 10 (e.g., 10, 9, 8, 7, 6, 5, 4, 3, 2, 1) branches. In some embodiments, a branched linker has between 2 and 15 branches. In some embodiments, a branched linker has between 2 and 10 branches. In some embodiments, a branched linker has between 2 and 5 branches. In some embodiments, a linker can be PEG or other chemistry as described herein.

### Single-Stranded Guide Nucleic Acid

In some aspects the disclosure relates to components of an ADAR recruiting molecule and/or an RNA targeting molecule. In some embodiments, an ADAR recruiting molecule and/or an RNA targeting molecule of the disclosure comprise a single-stranded guide nucleic acid. The terms “single-stranded guide nucleic acid” and “guide strand,” as may be used interchangeably herein, refers to a nucleic acid comprised of nucleotides. A single-stranded guide nucleic acid may comprise RNA and/or DNA nucleotides. For example, without limitation, see PCT/EP2015/080347 (WO2016/097212); PCT/EP2017/065467 (WO2017/220751); PCT/EP2017/071912 (WO2018/041973); PCT/EP2018/051202 (WO2018/134301); PCT/EP2019/053291 (WO2019/158475); PCT/EP2019/062163 (WO2019/219581); PCT/DE2016/000309 (WO 2017/050306); PCT/EP2018/067718 (WO 2020/001793); and Ser. No. 15/744,771 (US 2018/0208924), which describe in part, nucleic acids which may comprise a single-stranded guide nucleic acid as contemplated herein. In some embodiments, a single-stranded guide nucleic acid comprises DNA. In some embodiments, a single-stranded guide nucleic acid comprises RNA.

The single-stranded guide nucleic acid may comprise modifications to the nucleosides and/or backbone. In some embodiments, a single-stranded guide nucleic acid comprises at least one nucleotide modification. A modification may be to any part of the nucleotide. For example, without limitation, a nucleotide modification may be in the phosphate group, the pentose sugar group (e.g., ribose), or the nucleobase. In some embodiments, a nucleotide modification is in the phosphate group. In some embodiments, a nucleotide modification is in the pentose sugar group. In some embodiments, a nucleotide modification is in the nucleobase group. In some embodiments, a single-stranded guide nucleic acid comprises at least one nucleoside modification. A modification may be to any part of the nucleoside. For example, without limitation, a nucleoside modification may be in the pentose sugar group (e.g., ribose) or the nucleobase. In some embodiments, a nucleoside modification is in the pentose sugar group. In some embodiments, a nucleoside modification is in the nucleobase group. In some embodiments, the at least one nucleoside modification is a 2′-O-Methyl modification. In some embodiments, the at least one nucleoside modification is a 2′-Fluoro modification. In some embodiments, the at least one nucleoside modification is a 2′-O-methoxyethyl (2′O-MOE). In some embodiments, a single-stranded guide nucleic acid comprises at least one nucleobase modification. A nucleoside modification may occur at point throughout a single-stranded guide nucleic acid. In some embodiments, a nucleoside modification is positioned toward the ends of a given single-stranded guide nucleic acid (e.g., toward the 5′ or 3′ end, or both in the case of multiple modifications). In some embodiments, a nucleoside modification is positioned toward the middle of a given single-stranded guide nucleic acid. As one of ordinary skill in the art will appreciate, as discussed herein, when referring to a nucleoside modification, such terminology will encompass modifications to the nucleoside, or a component thereof individually and collectively. In other words, a modification of a nucleobase and/or 5 carbon sugar (in the context of being part of a nucleoside/nucleotide) in a nucleic acid will inherently also be a nucleoside modification.

In some embodiments, a single-stranded guide nucleic acid comprises at least one backbone modification. In some embodiments, a single-stranded guide nucleic acid comprises at least two backbone modifications. In some embodiments, a single-stranded guide nucleic acid comprises at least three backbone modifications. In some embodiments, more than 25% of the internucleoside linkages in a single-stranded guide nucleic acid comprise a phosphate modification. In some embodiments, more than 50% of the internucleoside linkages in a single-stranded guide nucleic acid comprise a phosphate modification. In some embodiments, more than 75% of the internucleoside linkages in a single-stranded guide nucleic acid comprise a phosphate modification. A backbone modification may occur at any point throughout a single-stranded guide nucleic acid. In some embodiments, a backbone modification is positioned toward the ends of a given strand of a single-stranded guide nucleic acid (e.g., toward the 5′ or 3′ end, or both in the case of multiple modifications). In some embodiments, a nucleoside modification is positioned toward the middle of a given strand of a single-stranded guide nucleic acid. In some embodiments, the at least one backbone modification of a single-stranded guide nucleic acid is positioned within 1-5 nucleotides of the terminal nucleotide of a single-stranded guide nucleic acid. In some embodiments, the at least one backbone modification of a single-stranded guide nucleic acid is positioned within 1-3 nucleotides of the terminal nucleotide of a single-stranded guide nucleic acid. In some embodiments, the at least one backbone modification of a single-stranded guide nucleic acid is positioned within 1 nucleotide of the terminal nucleotide of a single-stranded guide nucleic acid. In some embodiments, the backbone modification is a phosphorothioate modification.

In some embodiments, a single-stranded guide nucleic acid comprises at least one nucleoside modification. In some embodiments, a single-stranded guide nucleic acid comprises at least one backbone modification. In some embodiments, a single-stranded guide nucleic acid comprises at least one nucleoside modification and at least one backbone modification. In some embodiments, a single-stranded guide nucleic acid comprises at least two nucleoside modifications. In some embodiments, a single-stranded guide nucleic acid comprises at least three nucleoside modifications. In some embodiments, more than 25% of the nucleosides of a single-stranded guide nucleic acid comprise a nucleoside modification. In some embodiments, more than 50% of the nucleosides in a single-stranded guide nucleic acid comprise a nucleoside modification. In some embodiments, more than 75% of the nucleosides in a single-stranded guide nucleic acid comprise a nucleoside modification.

In some embodiments, a single-stranded guide nucleic acid comprises sufficient complementarity to hybridize with a target sequence. The terms “complementary” and “complementarity,” as may be used interchangeably herein, refer a property of a nucleotide (e.g., A, C, G, T, U) in a nucleic acid (e.g., RNA, DNA, oligonucleotide) strand to pair with another particular nucleotide in a nucleic acid strand of the opposite orientation (e.g., strands running parallel, but in the reverse direction (i.e., 5′-3′ aligns with 3′-5′, and 3′-5′ with 5′-3′)) (i.e., Watson-Crick base-pairing rules). With respect to deoxyribonucleic acids (DNA) the base pairings which are complementary are adenine (A) and thymine (T) (e.g., A with T, T with A) and guanine (G) and Cytosine (C) (e.g., G with C, C with G) and with respect to ribonucleic acid (RNA) the base pairings which are complementary are A and uracil (U) (e.g., A with U, U with A) and G and C (e.g., G with C, C with G). This occurs because of the ability of each base pair to form an equivalent number of hydrogen bonds with its complementary base (e.g., A-T/U, T/U-A, C-G, G-C), for example the bond between guanine and cytosine shares three hydrogen bonds compared to the A-T/U bond which always shares two hydrogen bonds.

As discussed herein above, when every base in a strand of a pair of nucleic acid strands is found opposite its complementary base pair, such strand is considered fully complementary to the sequence of the other strand. When a base of such a strand is found in a position where it is opposite any other base excepting its complementary base of the pair, that base is considered “mis-matched” (also referred to as a mismatch) and the strand is considered partially complementary. Accordingly, strands can be varying degrees of partially complementary, until no bases align, at which point they are non-complementary. Other non-standard nucleotides (e.g., 5-methylcytosine, 5-hydroxymethylcytosine) are known in the art and their properties and complementarity will be readily apparent to the skilled artisan. Methods and calculations are known in the art to determine percent complementarity.

In some embodiments, a single-stranded guide nucleic acid comprises sufficient complementarity with a target sequence (e.g., sufficient to promote editing of the target sequence). In some embodiments, a single-stranded guide nucleic acid comprises one or more modifications that can help stabilize interactions between the single-stranded guide nucleic acid and a target sequence. In some embodiments, the single-stranded guide nucleic acid comprises modifications which intercalate with a target sequence (e.g., of a DNA or RNA duplex) stabilizing hybridization (e.g., molecules which are not 100% complementary). In some embodiments, a single-stranded guide nucleic acid comprises at least 25% complementarity with a target sequence. In some embodiments, a single-stranded guide nucleic acid comprises at least 50% complementarity with a target sequence. In some embodiments, a single-stranded guide nucleic acid comprises at least 70% complementarity with a target sequence. In some embodiments, a single-stranded guide nucleic acid comprises at least 80% complementarity with a target sequence. In some embodiments, a single-stranded guide nucleic acid comprises at least 90% complementarity with a target sequence. In some embodiments, a single-stranded guide nucleic acid comprises at least 95% complementarity with a target sequence.

In some embodiments, a single-stranded guide nucleic acid comprises a sequence complementary to a target sequence, wherein all nucleotides are complementary except the nucleotide opposite the target nucleotide to be edited. In some embodiments, the single-stranded guide nucleic acid comprises additional mismatches other than the nucleotide opposite the target nucleotide and the target nucleotide pairing. As used herein, the terms “nucleotide to be edited” or “target nucleotide,” as may be interchangeably used, refer to the nucleoside (e.g., inclusive of the nucleobase and pentose sugar) attached to a phosphate, which is intended or desired to be edited (e.g., deaminated). In some embodiments, the target nucleotide comprises an adenosine (A). The term “opposite the target nucleotide,” as may be used herein, refers to the nucleotide which is contained on the sequence which is to hybridize with the nucleic acid comprising the sequence containing the target nucleotide, which is oriented or positioned in the same pair (even though it may not base pair according to traditional Watson-Crick base pairing rules) as the target nucleotide. In other words, the nucleotide which resides in the corollary position in a nucleic acid which hybridizes with the target sequence and which would pair with the target nucleotide if it is of base which pairs with the target nucleotide. By way of example, and in no way limiting, in the sequences 5′-ATATAT [symbol] GCCA-3′ (Strand Ref.: 11) (SEQ ID NO: 9) and 5′-TGGC [symbol] ATATAT-3′ (Strand Ref.: 12) (SEQ ID NO: 10), the fifth nucleotide in Strand Ref.: 12 (i.e., C, which is highlighted, italicized, and underlined) is a nucleotide opposite a target nucleotide in the seventh position of Strand Ref.: 11 (i.e., A, which is highlighted, italicized, and underlined) even though they do not base pair and create a “mismatch” according to traditional Watson-Crick base pairing rules.

In some embodiments, a single-stranded guide nucleic acid comprises at least one non-modified nucleotide (e.g., nucleobase, pentose sugar, phosphate combination). In some embodiments, a single-stranded guide nucleic acid comprises at least one non-modified nucleotide, wherein the non-modified nucleotide is opposite the nucleotide to be edited. In some embodiments, a single-stranded guide nucleic acid comprises more than one non-modified nucleotide. In some embodiments, a single-stranded guide nucleic acid comprises more than one non-modified nucleotide adjacent to the nucleotide opposite the target nucleotide. In some embodiments, a single-stranded guide nucleic acid comprises three consecutive non-modified nucleotides. In some embodiments, at least one of the three consecutive non-modified nucleotides pairs with a nucleotide adjacent to a target adenosine in the target sequence. In some embodiments, the middle nucleotide of the three consecutive non-modified nucleotides is opposite the target adenosine. In some embodiments, the middle nucleotide of the three consecutive non-modified nucleotides comprises guanine (G). In some embodiments, the middle nucleotide of the three consecutive non-modified nucleotides comprises cytosine (C). In some embodiments, the nucleotide opposite the target nucleotide comprises a modification. In some embodiments, the nucleotides adjacent to the nucleotide opposite the target nucleotide comprises a modification. In some embodiments, the nucleotide opposite the target nucleotide comprises any natural, synthetic, or modified nucleotide which does not base pair with adenosine (A). In some embodiments, the nucleotide opposite the target nucleotide comprises any natural, synthetic, or modified nucleotide which base pairs with guanine (G). In some embodiments, the nucleotide opposite the target nucleotide comprises any natural, synthetic, or modified nucleotide which base pairs with inosine (I).

In some embodiments, a single-stranded guide nucleic acid comprises two or more mismatches relative to a target sequence, where only one of the two or more mismatches on the single-stranded guide nucleic acid comprises a non-modified nucleotide. In some embodiments, the non-modified nucleotide of the two or more mismatches is opposite the nucleotide to be edited.

In some embodiments, a single-stranded guide nucleic acid is at least 5 nucleotides in length. In some embodiments, a single-stranded guide nucleic acid is fewer than or equal to 100 nucleotides in length. In some embodiments, a single-stranded guide nucleic acid is about 5 to about 80 nucleotides in length. In some embodiments, a single-stranded guide nucleic acid is about 5 to about 60 nucleotides in length. In some embodiments, a single-stranded guide nucleic acid is about 5 to about 40 nucleotides in length. In some embodiments, a single-stranded guide nucleic acid is about 5 to about 30 nucleotides in length. In some embodiments, a single-stranded guide nucleic acid is about 5 to about 20 nucleotides in length. In some embodiments, a single-stranded guide nucleic acid is about 5 to about 10 nucleotides in length. In some embodiments, a single-stranded guide nucleic acid is about 10 to about 30 nucleotides in length. In some embodiments, a single-stranded guide nucleic acid is about 15 to about 27 nucleotides in length. In some embodiments, a single-stranded guide nucleic acid is about 16 to about 26 nucleotides in length. In some embodiments, a single-stranded guide nucleic acid is about 10 to about 30 nucleotides in length. In some embodiments, a single-stranded guide nucleic acid is about 15 to about 25 nucleotides in length. In some embodiments, a single-stranded guide nucleic acid is about 15 to about 20 nucleotides in length. In some embodiments, a single-stranded guide nucleic acid is about 17 to about 19 nucleotides in length.

In some embodiments, a single-stranded guide nucleic acid comprises a wobble base pair. As used herein, when a single-stranded guide nucleic acid is described as comprising a wobble base pair and/or a mismatch, such description shall be understood to refer to a property of such single-stranded guide nucleic acid when bound to a target nucleic acid having a target nucleotide sequence. For example, without limitation, a single-stranded guide nucleic acid may be designed (e.g., engineered by the skilled artisan) to comprise less than 100% (e.g., incomplete, imperfect) complementarity to the target sequence (e.g., the nucleotide sequence of the nucleic acid to which a single-stranded guide nucleic acid is intended to bind (e.g., anneal, hybridize)). By designing a single-stranded guide nucleic acid as such, a single-stranded guide nucleic acid may be engineered to create (e.g., form), upon annealing or hybridization, wobble base pairs and/or mismatches between the single-stranded guide nucleic acid and the nucleic acid having the target nucleotide sequence (e.g., target nucleic acid, oligonucleotide). Accordingly, where the disclosure describes a single-stranded guide nucleic acid as comprising a mismatch and/or wobble base pair, it is to be understood that these are relative properties which define a relationship between the single-stranded guide nucleic acid and a target nucleic acid.

In some embodiments, a single-stranded guide nucleic acid comprises at least 1 (e.g., 1, 2, 3, 4, 5, 6, 7, 8, 9, 10, 11, 12, 13, 14, 15, 16, 17, 18, 19, 20, or more) mismatch. In some embodiments, a single-stranded guide nucleic acid comprises at least 2 mismatches. In some embodiments, a single-stranded guide nucleic acid comprises at least 3 mismatches. In some embodiments, a single-stranded guide nucleic acid comprises at least 4 mismatches. In some embodiments, a single-stranded guide nucleic acid comprises at least 5 mismatches. In some embodiments, a single-stranded guide nucleic acid comprises at least 10 mismatches. In some embodiments, a single-stranded guide nucleic acid comprises at least 20 mismatches.

In some embodiments, a single-stranded guide nucleic acid comprises fewer than 20 mismatches. In some embodiments, a single-stranded guide nucleic acid comprises fewer than 10 mismatches. In some embodiments, a single-stranded guide nucleic acid comprises fewer than 5 mismatches. In some embodiments, a single-stranded guide nucleic acid comprises fewer than 4 mismatches. In some embodiments, a single-stranded guide nucleic acid comprises fewer than 3 mismatches. In some embodiments, a single-stranded guide nucleic acid comprises fewer than 2 mismatches.

In some embodiments, a single-stranded guide nucleic acid comprises at least 1 mismatch, but fewer than or equal to 20 mismatches. In some embodiments, a single-stranded guide nucleic acid comprises at least 1 mismatch, but fewer than or equal to 10 mismatches. In some embodiments, a single-stranded guide nucleic acid comprises at least 1 mismatch, but fewer than or equal to 7 mismatches. In some embodiments, a single-stranded guide nucleic acid comprises at least 1 mismatch, but fewer than or equal to 5 mismatches. In some embodiments, a single-stranded guide nucleic acid comprises at least 1 mismatch, but fewer than or equal to 4 mismatches. In some embodiments, a single-stranded guide nucleic acid comprises at least 1 mismatch, but fewer than or equal to 3 mismatches. In some embodiments, a single-stranded guide nucleic acid comprises at least 2 mismatches, but fewer than or equal to 20 mismatches. In some embodiments, a single-stranded guide nucleic acid comprises at least 2 mismatches, but fewer than or equal to 10 mismatches. In some embodiments, a single-stranded guide nucleic acid comprises at least 2 mismatches, but fewer than or equal to 7 mismatches. In some embodiments, a single-stranded guide nucleic acid comprises at least 2 mismatches, but fewer than or equal to 5 mismatches. In some embodiments, a single-stranded guide nucleic acid comprises at least 2 mismatches, but fewer than or equal to 4 mismatches.

In some embodiments, a mismatch is within 8 nucleotides of a terminal nucleotide of a single-stranded guide nucleic acid. In some embodiments, a mismatch is within 7 nucleotides of a terminal nucleotide of a single-stranded guide nucleic acid. In some embodiments, a mismatch is within 6 nucleotides of a terminal nucleotide of a single-stranded guide nucleic acid. In some embodiments, a mismatch is within 5 nucleotides of a terminal nucleotide of a single-stranded guide nucleic acid. In some embodiments, a mismatch is within 4 nucleotides of a terminal nucleotide of a single-stranded guide nucleic acid. In some embodiments, a mismatch is within 3 nucleotides of a terminal nucleotide of a single-stranded guide nucleic acid. In some embodiments, a mismatch is within 2 nucleotides of a terminal nucleotide of a single-stranded guide nucleic acid. In some embodiments, a mismatch is within 1 nucleotide of a terminal nucleotide of a single-stranded guide nucleic acid.

In some embodiments, a single-stranded guide nucleic acid comprises at least 1 (e.g., 1, 2, 3, 4, 5, 6, 7, 8, 9, 10, 11, 12, 13, 14, 15, 16, 17, 18, 19, 20, or more) wobble base pair. In some embodiments, a single-stranded guide nucleic acid comprises at least 2 wobble base pairs. In some embodiments, a single-stranded guide nucleic acid comprises at least 3 wobble base pairs. In some embodiments, a single-stranded guide nucleic acid comprises at least 4 wobble base pairs. In some embodiments, a single-stranded guide nucleic acid comprises at least 5 wobble base pairs. In some embodiments, a single-stranded guide nucleic acid comprises at least 10 wobble base pairs. In some embodiments, a single-stranded guide nucleic acid comprises at least 20 wobble base pairs.

In some embodiments, a single-stranded guide nucleic acid comprises fewer than 20 wobble base pairs. In some embodiments, a single-stranded guide nucleic acid comprises fewer than 10 wobble base pairs. In some embodiments, a single-stranded guide nucleic acid comprises fewer than 5 wobble base pairs. In some embodiments, a single-stranded guide nucleic acid comprises fewer than 4 wobble base pairs. In some embodiments, a single-stranded guide nucleic acid comprises fewer than 3 wobble base pairs. In some embodiments, a single-stranded guide nucleic acid comprises fewer than 2 wobble base pairs.

In some embodiments, a single-stranded guide nucleic acid comprises at least 1 wobble base pair, but fewer than or equal to 20 wobble base pairs. In some embodiments, a single-stranded guide nucleic acid comprises at least 1 wobble base pair, but fewer than or equal to 10 wobble base pairs. In some embodiments, a single-stranded guide nucleic acid comprises at least 1 wobble base pair, but fewer than or equal to 7 wobble base pairs. In some embodiments, a single-stranded guide nucleic acid comprises at least 1 wobble base pair, but fewer than or equal to 5 wobble base pairs. In some embodiments, a single-stranded guide nucleic acid comprises at least 1 wobble base pair, but fewer than or equal to 4 wobble base pairs. In some embodiments, a single-stranded guide nucleic acid comprises at least 1 wobble base pair, but fewer than or equal to 3 wobble base pairs. In some embodiments, a single-stranded guide nucleic acid comprises at least 2 wobble base pairs, but fewer than or equal to 20 wobble base pairs. In some embodiments, a single-stranded guide nucleic acid comprises at least 2 wobble base pairs, but fewer than or equal to 10 wobble base pairs. In some embodiments, a single-stranded guide nucleic acid comprises at least 2 wobble base pairs, but fewer than or equal to 7 wobble base pairs. In some embodiments, a single-stranded guide nucleic acid comprises at least 2 wobble base pairs, but fewer than or equal to 5 wobble base pairs. In some embodiments, a single-stranded guide nucleic acid comprises at least 2 wobble base pairs, but fewer than or equal to 4 wobble base pairs.

In some embodiments, a wobble base pair is within 8 nucleotides of a terminal nucleotide of a single-stranded guide nucleic acid. In some embodiments, a wobble base pair is within 7 nucleotides of a terminal nucleotide of a single-stranded guide nucleic acid. In some embodiments, a wobble base pair is within 6 nucleotides of a terminal nucleotide of a single-stranded guide nucleic acid. In some embodiments, a wobble base pair is within 5 nucleotides of a terminal nucleotide of a single-stranded guide nucleic acid. In some embodiments, a wobble base pair is within 4 nucleotides of a terminal nucleotide of a single-stranded guide nucleic acid. In some embodiments, a wobble base pair is within 3 nucleotides of a terminal nucleotide of a single-stranded guide nucleic acid. In some embodiments, a wobble base pair is within 2 nucleotides of a terminal nucleotide of a single-stranded guide nucleic acid. In some embodiments, a wobble base pair is within 1 nucleotide of a terminal nucleotide of a single-stranded guide nucleic acid.

In some embodiments, a single-stranded guide nucleic acid further comprises at least one additional moiety. An additional moiety may be attached to the 5′ or 3′ end of a single-stranded guide nucleic acid. An additional moiety may be attached to the single-stranded guide nucleic acid at a point (e.g., nucleotide) between the 5′ and 3′ ends of the single-stranded guide nucleic acid. Further, a single-stranded guide nucleic acid may comprise more than one additional moiety. An additional moiety may further be attached to a single-stranded guide nucleic acid in any configuration by means of additional nucleotides as described hereinabove, or by a linker.

In some embodiments, a single-stranded guide nucleic acid further comprises a linker. In some embodiments, a linker is attached at the 5′ end of a single-stranded guide nucleic acid. In some embodiments, a linker is attached at the 3′ end of a single-stranded guide nucleic acid. In some embodiments, a linker is attached at the 5′ end and the 3′ end of a single-stranded guide nucleic acid. In some embodiments, a linker may be attached to the single-stranded guide nucleic acid at a point (e.g., nucleotide) between the 5′ and 3′ ends of the single-stranded guide nucleic acid. In some embodiments, a linker connects a double-stranded RNA duplex to the single-stranded guide nucleic acid.

In some embodiments, a single-stranded guide nucleic acid is connected through its 5′ end to a double-stranded RNA duplex (e.g., the 3′ end of the single-stranded guide nucleic acid remains free, is distal to the double-stranded RNA duplex, is distal to the double-stranded RNA duplex but connected to another moiety and/or linker). In some embodiments, a single-stranded guide nucleic acid is connected through its 3′ end to a double-stranded RNA duplex (e.g., the 5′ end of the single-stranded guide nucleic acid remains free, is distal to the double-stranded RNA duplex, is distal to the double-stranded RNA duplex but connected to another moiety and/or linker). In some embodiments, a single-stranded guide nucleic acid is connected to a double-stranded RNA duplex at a point (e.g., nucleotide) between the 5′ and 3′ ends of the single-stranded guide nucleic acid (e.g., both the 5′ and 3′ ends of the single-stranded guide nucleic acid remain free).

In some embodiments, a single-stranded guide nucleic acid is connected to more than one double-stranded RNA duplex (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more). In some embodiments, the more than one double-stranded RNA duplexes are connected to the single-stranded guide nucleic acid at points (e.g., nucleotides) between the 5′ and 3′ ends of the single-stranded guide nucleic acid (e.g., both the 5′ and 3′ ends of the single-stranded guide nucleic acid remain free of a double-stranded RNA duplex (e.g., may be connected to another moiety or component)). In some embodiments, the more than one double-stranded RNA duplexes are connected to the single-stranded guide nucleic acid such that at least one double-stranded RNA duplex is connected at a point (e.g., nucleotide) between the 5′ and 3′ ends of the single-stranded guide nucleic acid and at least one double-stranded RNA duplex is connected to the single-stranded guide nucleic acid at either the 5′ or 3′ end of the single-stranded guide nucleic acid, with at least one of the 5′ or 3′ end of the single-stranded guide nucleic acid remaining free of a double-stranded RNA duplex (e.g., may be connected to another moiety or component). In some embodiments, the more than one double-stranded RNA duplexes are connected to the single-stranded guide nucleic acid such that at least one double-stranded RNA duplex is connected at a point (e.g., nucleotide) between the 5′ and 3′ ends of the single-stranded guide nucleic acid, and at least one double-stranded RNA duplex is connected to the single-stranded guide nucleic acid at each of the 5′ and 3′ end of the single-stranded guide nucleic acid, such that neither the 5′ or 3′ end of the single-stranded guide nucleic acid is free. In some embodiments, the more than one double-stranded RNA duplexes are connected to the single-stranded guide nucleic acid such that at least one double-stranded RNA duplex is connected to the single-stranded guide nucleic acid at each of the 5′ and 3′ end of the single-stranded guide nucleic acid, without any double-stranded RNA duplexes connected to the single-stranded guide nucleic acid at points (e.g., nucleotides) between the 5′ and 3′ ends of the single-stranded guide nucleic acid.

In some embodiments, a double-stranded RNA duplex is connected to more than one single-stranded guide nucleic acid (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more). In some embodiments, the more than one single-stranded guide nucleic acids are connected to the double-stranded RNA duplex at points (e.g., nucleotides) between the 5′ and 3′ ends of the double-stranded RNA duplex (e.g., both the 5′ and 3′ ends of each of the RNA strands of the double-stranded RNA duplex remain free from connections with single-stranded guide nucleic acids).

In some embodiments, the more than one single-stranded guide nucleic acids are connected to the double-stranded RNA duplex such that at least one single-stranded guide nucleic acid is connected at a point (e.g., nucleotide) between the 5′ and 3′ ends of the double-stranded RNA duplex and at least one single-stranded guide nucleic acid is connected to the double-stranded RNA duplex at either the 5′ or 3′ end of at least one of the RNA strands of the double-stranded RNA duplex, with at least one of the 5′ or 3′ end of at least one of the RNA strands of the double-stranded RNA duplex remaining free from connections with single-stranded guide nucleic acids.

In some embodiments, the more than one single-stranded guide nucleic acids are connected to the double-stranded RNA duplex such that at least two single-stranded guide nucleic acids are connected at points (e.g., nucleotide) between the 5′ and 3′ ends of the double-stranded RNA duplex, one on each RNA strand of the double-stranded RNA duplex, and at least one single-stranded guide nucleic acid is connected to the double-stranded RNA duplex at either the 5′ or 3′ end of at least one of the RNA strands of the double-stranded RNA duplex, with at least one of the 5′ or 3′ end of at least one of the RNA strands of the double-stranded RNA duplex remaining free from connections with single-stranded guide nucleic acids.

In some embodiments, the more than one single-stranded guide nucleic acids are connected to the double-stranded RNA duplex such that at least one single-stranded guide nucleic acid is connected at a point (e.g., nucleotide) between the 5′ and 3′ ends of the double-stranded RNA duplex and at least one single-stranded guide nucleic acid is connected to the double-stranded RNA duplex at each of the 5′ and 3′ ends of each of the RNA strands of the double-stranded RNA duplex.

In some embodiments, the more than one single-stranded guide nucleic acids are connected to the double-stranded RNA duplex such that at least two single-stranded guide nucleic acids are connected at points (e.g., nucleotide) between the 5′ and 3′ ends of the double-stranded RNA duplex, one connected to each RNA strand of the double-stranded RNA duplex, and at least one single-stranded guide nucleic acid is connected to the double-stranded RNA duplex at each of the 5′ and 3′ ends of each of the RNA strands of the double-stranded RNA duplex.

In some embodiments, the more than one single-stranded guide nucleic acids are connected to the double-stranded RNA duplex such that at least one single-stranded guide nucleic acid is connected to the double-stranded RNA duplex at each of the 5′ and 3′ ends of the double-stranded RNA duplex, without any single-stranded guide nucleic acids connected to the double-stranded RNA duplex in between the 5′ and 3′ ends of the RNA strands.

In some embodiments, in any of the configurations disclosed herein, wherein one or more double-stranded RNA duplex is connected to one or more double-stranded RNA duplex (e.g., another/other double-stranded RNA duplex) it is within the scope of the present disclosure that such configurations also embody the connection of such double-stranded RNA duplex to such other double-stranded RNA duplex by means of (e.g., linked via, connected by, etc.) a linker as described herein. It is further envisioned that configurations connecting the 5′ end to the 5′ end and/or 3′ end to 3′ end of nucleic acids (e.g., DNA, RNA, modified DNA, modified RNA, each RNA strand of a double-stranded RNA, single-stranded guide nucleic acid, etc.) is possible by use of linkers and moieties as described herein, such configurations are embodied by this disclosure.

In some embodiments, in any of the configurations disclosed herein, wherein one or more single-stranded guide nucleic acid is connected to one or more single-stranded guide nucleic acid (e.g., another/other single-stranded guide nucleic acid) it is within the scope of the present disclosure that such configurations also embody the connection of such single-stranded guide nucleic acid to such other single-stranded guide nucleic acid by means of (e.g., linked via, connected by, etc.) a linker as described herein. It is further envisioned that configurations connecting the 5′ end to the 5′ end and/or 3′ end to 3′ end of nucleic acids (e.g., DNA, RNA, modified DNA, modified RNA, each RNA strand of a double-stranded RNA, single-stranded guide nucleic acid, etc.) is possible by use of linkers and moieties as described herein, such configurations are embodied by this disclosure.

In some embodiments, in any of the configurations disclosed herein, wherein one or more double-stranded RNA duplex is connected to one or more single-stranded guide nucleic acid it is within the scope of the present disclosure that such configurations also embody the connection of such double-stranded RNA duplex to such single-stranded guide nucleic acid by means of (e.g., linked via, connected by, etc.) a linker as described herein. It is further envisioned that configurations connecting the 5′ end to the 5′ end and/or 3′ end to 3′ end of nucleic acids (e.g., DNA, RNA, modified DNA, modified RNA, each RNA strand of a double-stranded RNA duplex, single-stranded guide nucleic acid, etc.) is possible by use of linkers and moieties as described herein, such configurations are embodied by this disclosure.

In some embodiments, where there is more than one double-stranded RNA duplex connected to at least one single-stranded guide nucleic acid, the double-stranded RNA duplexes may all be uniform (e.g., have the same sequences in both RNA strands of each double-stranded RNA duplex). In some embodiments, where there is more than one double-stranded RNA duplex connected to at least one single-stranded guide nucleic acid, the double-stranded RNA duplexes may be not uniform, in that at least one RNA strand of at least one double-stranded RNA duplex is distinct from the others (e.g., distinct in terms of sequence, modifications, and/or additional moieties). In some embodiments, where there is more than one double-stranded RNA duplex connected to at least one single-stranded guide nucleic acid, the double-stranded RNA duplexes may be distinct, in that at least one RNA strand of each double-stranded RNA duplex is distinct from the RNA strands of the other double-stranded RNA duplexes.

In some embodiments, where there is more than one single-stranded guide nucleic acid connected to at least one double-stranded RNA duplex, the single-stranded guide nucleic acids may all be uniform (e.g., all share the same sequence). For example, in some embodiments, the single-stranded guide nucleic acids have the same sequence with the same modifications (e.g., nucleoside modification, internucleoside linkage modification). In some embodiments, the single-stranded guide nucleic acids have the same sequence, but not all nucleic acids have the same modifications (e.g., nucleoside modification, internucleoside linkage modification). In some embodiments, where there is more than one single-stranded guide nucleic acid connected to at least one double-stranded RNA duplex, the single-stranded guide nucleic acids may all be not uniform (e.g., at least one single-stranded guide nucleic acid has a sequence distinct from the other single-stranded guide nucleic acids). In some embodiments, where there is more than one single-stranded guide nucleic acid connected to at least one double-stranded RNA duplex, the single-stranded guide nucleic acids may distinct (e.g., all of the single-stranded guide nucleic acids have distinct sequences).

As can be appreciated by the skilled artisan, such degree of similarity and distinctness may be of varying degrees and varying similarity and the embodiments above illustrated a few instances across such a spectrum. All such combinations and permutations are envisioned and to be interpreted within the scope of the present disclosure.

In some embodiments, a single-stranded guide nucleic acid comprises ribonucleic acid (gRNA).

In some embodiments, a single-stranded guide nucleic acid comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 5, 8, 17, 23, and/or 26. In some embodiments, a single-stranded guide nucleic acid comprises a sequence of Strand Ref.: 5, 8, 17, 23, and/or 26.

In some embodiments, a single-stranded guide nucleic acid comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 5. In some embodiments, a single-stranded guide nucleic acid comprises a sequence of Strand Ref.: 5. In some embodiments, a single-stranded guide nucleic acid comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 8. In some embodiments, a single-stranded guide nucleic acid comprises a sequence of Strand Ref.: 8. In some embodiments, a single-stranded guide nucleic acid comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 17. In some embodiments, a single-stranded guide nucleic acid comprises a sequence of Strand Ref.: 17. In some embodiments, a single-stranded guide nucleic acid comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 23. In some embodiments, a single-stranded guide nucleic acid comprises a sequence of Strand Ref.: 23. In some embodiments, a single-stranded guide nucleic acid comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 26. In some embodiments, a single-stranded guide nucleic acid comprises a sequence of Strand Ref.: 26.

In some embodiments, an ADAR recruiting molecule and/or an RNA targeting molecule comprises a double-stranded RNA duplex comprising an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 3 and an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 4 and a single-stranded guide nucleic acid comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 5. In some embodiments, an ADAR recruiting molecule and/or an RNA targeting molecule comprises a double-stranded RNA duplex comprising an RNA strand comprising the sequence of Strand Ref.: 3 and an RNA strand comprising the sequence of Strand Ref.: 4 and a single-stranded guide nucleic acid comprising the sequence of Strand Ref.: 5.

In some embodiments, an ADAR recruiting molecule and/or an RNA targeting molecule comprises a double-stranded RNA duplex comprising an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 6 and an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 7 and a single-stranded guide nucleic acid comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 8. In some embodiments, an ADAR recruiting molecule and/or an RNA targeting molecule comprises a double-stranded RNA duplex comprising an RNA strand comprising the sequence of Strand Ref.: 6 and an RNA strand comprising the sequence of Strand Ref.: 7 and a single-stranded guide nucleic acid comprising the sequence of Strand Ref.: 8. In some embodiments, an ADAR recruiting molecule and/or an RNA targeting molecule comprises a double-stranded RNA duplex comprising an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 15 and an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 16 and a single-stranded guide nucleic acid comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 17. In some embodiments, an ADAR recruiting molecule and/or an RNA targeting molecule comprises a double-stranded RNA duplex comprising an RNA strand comprising the sequence of Strand Ref.: 15 and an RNA strand comprising the sequence of Strand Ref.: 16 and a single-stranded guide nucleic acid comprising the sequence of Strand Ref.: 17. In some embodiments, an ADAR recruiting molecule and/or an RNA targeting molecule comprises a double-stranded RNA duplex comprising an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 18 and an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 19 and a single-stranded guide nucleic acid comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 17. In some embodiments, an ADAR recruiting molecule and/or an RNA targeting molecule comprises a double-stranded RNA duplex comprising an RNA strand comprising the sequence of Strand Ref.: 18 and an RNA strand comprising the sequence of Strand Ref.: 19 and a single-stranded guide nucleic acid comprising the sequence of Strand Ref.: 17.

In some embodiments, an ADAR recruiting molecule and/or an RNA targeting molecule comprises a double-stranded RNA duplex comprising an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 24 and an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 25 and a single-stranded guide nucleic acid comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 23. In some embodiments, an ADAR recruiting molecule and/or an RNA targeting molecule comprises a double-stranded RNA duplex comprising an RNA strand comprising the sequence of Strand Ref.: 24 and an RNA strand comprising the sequence of Strand Ref.: 25 and a single-stranded guide nucleic acid comprising the sequence of Strand Ref.: 23. In some embodiments, an ADAR recruiting molecule and/or an RNA targeting molecule comprises a double-stranded RNA duplex comprising an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 27 and an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 28 and a single-stranded guide nucleic acid comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 26. In some embodiments, an ADAR recruiting molecule and/or an RNA targeting molecule comprises a double-stranded RNA duplex comprising an RNA strand comprising the sequence of Strand Ref.: 27 and an RNA strand comprising the sequence of Strand Ref.: 28 and a single-stranded guide nucleic acid comprising the sequence of Strand Ref.: 26.

In some embodiments, an ADAR recruiting molecule and/or an RNA targeting molecule comprises a double-stranded RNA duplex comprising an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 3 and an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 13, wherein Strand Ref.: 13 comprises the second RNA strand of the double-stranded RNA duplex and the single-stranded guide nucleic acid. In some embodiments, an ADAR recruiting molecule and/or an RNA targeting molecule comprises a double-stranded RNA duplex comprising an RNA strand comprising the sequence of Strand Ref.: 3 and an RNA strand comprising the sequence of Strand Ref.: 7 and a single-stranded guide nucleic acid comprising the sequence of Strand Ref.: 13 wherein Strand Ref.: 13 comprises the second RNA strand of the double-stranded RNA duplex and the single-stranded guide nucleic acid.

In some embodiments, an ADAR recruiting molecule and/or an RNA targeting molecule comprises a double-stranded RNA duplex comprising an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 6 and an RNA strand with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to Strand Ref.: 13, wherein Strand Ref.: 14 comprises the second RNA strand of the double-stranded RNA duplex and the single-stranded guide nucleic acid. In some embodiments, an ADAR recruiting molecule and/or an RNA targeting molecule comprises a double-stranded RNA duplex comprising an RNA strand comprising the sequence of Strand Ref.: 6 and an RNA strand comprising the sequence of Strand Ref.: 7 and a single-stranded guide nucleic acid comprising the sequence of Strand Ref.: 13 wherein Strand Ref.: 14 comprises the second RNA strand of the double-stranded RNA duplex and the single-stranded guide nucleic acid. Any of the ADAR recruiting molecules and/or RNA targeting molecules as disclosed herein, may employ the use of the sequences disclosed herein, such that the modified sequences of the double-stranded RNA duplex of Strand Ref.: 3-4, 6-7, 24-25 may be arranged in any permutation or combination (e.g., substituted) with their unmodified counterparts as represented by Strand Ref.: 15-16, 18-19, 27-28 and the modified single-stranded guide nucleic acid sequences of Strand Ref.: 5 and 8, may be arranged in any permutation or combination (e.g., substituted) with their unmodified counterparts as represented by Strand Ref.: 17, and the modified single-stranded guide nucleic acid sequence of Strand Ref.: 23 may be arranged in any permutation or combination (e.g., substituted) with its unmodified counterpart as represented by Strand Ref.: 26. Further, the combined modified double-stranded RNA duplex sequences and single-stranded guide nucleic acid sequences of Strand Ref.: 13-14 may be arranged or substituted with their unmodified counterparts of Strand Ref.: 21-22. As can be shown in the Exemplary Sequences disclosed herein (Table 8), an RNA targeting molecule may comprise a sequence having at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to any one of Strand Ref.: 29-963. In some embodiments, an RNA targeting molecule comprises a sequence of any one of Strand Ref.: 29-963.

In some embodiments, a double-stranded RNA duplex comprises a sequence having at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to any odd Strand Ref. (e.g., sequence identifier) of any one of Strand Ref.: 29-752 (e.g., Strand Ref.: 29, 31, 33, 35, etc.). In some embodiments, a double-stranded RNA duplex comprises a sequence of any odd Strand Ref. (e.g., sequence identifier) of any one of Strand Ref.: 29-752 (e.g., Strand Ref.: 29, 31, 33, 35, etc.). In some embodiments, a second strand of a double-stranded RNA duplex comprises the portion of complementarity to any odd Strand Ref. (e.g., sequence identifier) of any one of Strand Ref.: 29-752 (e.g., Strand Ref.: 29, 31, 33, 35, etc.) from any even Strand Ref. (e.g., sequence identifier) of any one of Strand Ref.: 29-752 (e.g., Strand Ref.: 30, 32, 34, 36, etc.).

In some embodiments, a double-stranded RNA duplex comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to any one of the sequences as disclosed in Table 8 which are described as “Recruiting Domains” (e.g., any one of Strand Ref.: 754, 755, 757, 758, 760, 761, 763, 764, 766, 767, 769, 770, 772, 773, 775, 776, 778, 779, 781, 782, 784, 785, 787, 788, 790, 791, 793, 794, 796, 797, 799, 800, 802, 803, 805, 806, 808, 809, 811, 812, 814, 815, 817, or 818). In some embodiments, a double-stranded RNA duplex comprises a sequence of any one of the sequences as disclosed in Table 8 which are described as “Recruiting Domains” (e.g., any one of Strand Ref.: 754, 755, 757, 758, 760, 761, 763, 764, 766, 767, 769, 770, 772, 773, 775, 776, 778, 779, 781, 782, 784, 785, 787, 788, 790, 791, 793, 794, 796, 797, 799, 800, 802, 803, 805, 806, 808, 809, 811, 812, 814, 815, 817, or 818).

In some embodiments, a single-stranded guide nucleic acid comprises a portion of any even Strand Ref. (e.g., sequence identifier) of any one of Strand Ref.: 29-752 (e.g., Strand Ref.: 30, 32, 34, 36, etc.), which is not complementary to any odd Strand Ref. (e.g., sequence identifier) of any one of Strand Ref.: 29-752 (e.g., Strand Ref.: 29, 31, 33, 35, etc.). In some embodiments, a single-stranded guide nucleic acid comprises a sequence with at least 70% identity (e.g., 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity) to any one of the sequences as disclosed in Table 8 which are described as “Editing Domains” (e.g., any one of Strand Ref.: 753, 756, 759, 762, 765, 768, 771, 774, 777, 780, 783, 786, 789, 792, 795, 798, 801, 804, 807, 810, 813, or 816). In some embodiments, a single-stranded guide nucleic acid comprises a sequence of any one of the sequences as disclosed in Table 8 which are described as “Editing Domains” (e.g., any one of Strand Ref.: 753, 756, 759, 762, 765, 768, 771, 774, 777, 780, 783, 786, 789, 792, 795, 798, 801, 804, 807, 810, 813, or 816). In some embodiments, an RNA targeting molecule comprises any of the compounds as disclosed in Table 8. In some embodiments, an RNA targeting molecule comprises any of the compounds in Table 8 including a linker.

### Methods of Use

In some aspects, the disclosure relates to methods of using the molecules and/or compositions of the disclosure. As used herein, in some embodiments, the term “molecules,” as used to describe the subject matter of this disclosure, refers in some instances to an ADAR recruiting molecule and/or RNA targeting molecule, individually and collectively, in singular and in plural, and compositions thereof (including without limitation, e.g., the nucleic acid strands (e.g., oligonucleotides), duplexes, and molecules thereof). Accordingly, for example, when referring to the molecules and/or compositions of the disclosure, it is to be interpreted to include all such combinations and permutations of an ADAR recruiting molecule and/or an RNA targeting molecule of the disclosure in each, and all, embodiments. However, the term molecule may be used herein as a general term to refer to other types of molecules, and its use may vary depending on a particular context.

In some embodiments, the molecules and/or compositions of the disclosure are administered to effectuate deamination of a target adenosine. In some embodiments, the target adenosine is an adenosine on a nucleic acid comprised of RNA. In some embodiments, the RNA is selected from messenger RNA (mRNA), transfer RNA (tRNA), ribosomal RNA (rRNA), or micro RNA (miRNA). In some embodiments, the RNA is mRNA.

In some embodiments, the target adenosine is the result of a mutation (e.g., insertion, deletion, substitution, conversion) or genetic defect. In some embodiments, the target adenosine is not the result of a mutation (e.g., insertion, deletion, substitution, conversion) or genetic defect. In some embodiments, the target adenosine is located in a protein coding region of the nucleic acid. In some embodiments, the target adenosine is located in a non-protein coding region of the nucleic acid (e.g., intron, UTR, miRNA, pseudogene).

In some embodiments, the molecules and/or compositions of the disclosure are used to correct the result of a G to A mutation, for example, by deamination, which changes the A to an inosine, which mimics G during translation. In some embodiments, the deamination causes a change in the protein translated from an RNA. In some embodiments, the deamination does not cause a change in the protein translated from an RNA. In some embodiments, the deamination creates or changes (e.g., deletes, alters) a translational start site. In some embodiments, the deamination creates or changes (e.g., deletes, alters) a translational stop (e.g., termination) site. For example, in some embodiments, the deamination introduces a low- or high-usage stop codon to change the expression level of a protein. In some embodiments, the deamination causes an elongated translated protein (e.g., additional amino acid residues not present on the wild-type protein, may occur on the N-terminus, C-terminus, or internally). In some embodiments, the deamination causes a truncated translated protein (e.g., fewer amino acid residues than the wild-type protein, may occur on the N-terminus, C-terminus, or internally). In some embodiments, the deamination causes non-translation of the wild-type protein.

In some embodiments, the molecules and/or compositions of the disclosure are administered to a subject. The term “subject,” as used herein, refers to any organism in need of treatment or diagnosis using the subject matter (e.g., the molecules and/or compositions disclosed) herein. For example, without limitation, subjects may include mammals and non-mammals. As used herein, a “mammal,” refers to any animal constituting the class Mammalia (e.g., a human, mouse, rat, cat, dog, sheep, rabbit, horse, cow, goat, pig, guinea pig, hamster, chicken, turkey, or a non-human primate (e.g., Marmoset, Macaque)). In some embodiments, the mammal is a human. In some embodiments, the administration is to treat a subject. The terms “treatment,” “treat,” and “treating,” as may be used interchangeably herein, refer to partially or completely alleviating, ameliorating, relieving, delaying onset of, inhibiting progression of, reducing severity of, and/or reducing incidence of one or more symptoms or features of a particular indication, disease, disorder, condition, and/or symptom thereof. In some embodiments, the treatment refers to a clinical intervention. In some embodiments, treatment may be administered after one or more symptoms have developed and/or after a disease has been diagnosed. In other embodiments, treatment may be administered in the absence of symptoms (e.g., to prevent or delay onset of a symptom or inhibit onset or progression of a disease). For example, treatment may be administered to a susceptible individual (e.g., subject) prior to the onset of symptoms (e.g., in light of a history of symptoms and/or in light of genetic or other susceptibility factors). In some embodiments, the treatment is used and/or administered as a prophylaxis. Treatment may also be continued after symptoms have resolved, for example, to prevent or delay their recurrence.

In some embodiments, the molecules and/or compositions are administered to the subject to treat a disease or disorder. The disease or disorder may be any disease or disorder which may benefit from the treatment using the molecules and/or compositions described herein, or which may benefit from selective or directed deamination of a target adenosine or directed deamination of a multitude of target adenosines (e.g., of multiple copies of target adenosines, or of multiple adenosines at different locations on the same nucleic acid, or of multiple adenosines on different or multiple nucleic acids (at the same location or different locations)). In some embodiments, the disease or disorder is one which is related to, caused by, or affected by, the expression (e.g., transcription), or non-expression (e.g., lack of transcription) of at least one adenosine in an RNA. In some embodiments, the adenosine occurs at a target position within the RNA. For example, the disease or disorder may be related to a point mutation (e.g., insertion, deletion, substitution, conversion) of a nucleotide. In some embodiments, the disease or disorder is selected from Cystic fibrosis, Hurler Syndrome, alpha-1-antitrypsin (A1AT) deficiency, Parkinson's disease, Alzheimer's disease, albinism, Amyotrophic lateral sclerosis, Asthma, beta-thalassemia (β-thalassemia), Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B, and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, Sturge-Weber Syndrome, and cancer.

In some embodiments, the molecules and/or compositions are administered to the subject in an effective amount to treat the disease or disorder. The terms “effective amount,” “therapeutically effective amount,” and “pharmaceutically effective amount,” as may be used interchangeably herein, refer to an amount of a biologically active agent (e.g., the molecules and/or compositions of the instant disclosure) sufficient to elicit a desired biological response. For example, in some embodiments, an effective amount of an ADAR recruiting molecule and/or an RNA targeting molecule may refer to the amount sufficient to target a nucleic acid and target adenosine to effectuate deamination thereof. As will be appreciated by the skilled artisan, the effective amount of a molecule and/or composition as described herein may vary depending on various factors as, for example, on the desired biological response (e.g., on desired therapeutic effect, the number of targets to be deaminated, the complexity of the targeting, etc.), on the cell or tissue being targeted, and on the agent being used.

In some embodiments, the molecules and/or compositions of the disclosure can be used to target a transcript (e.g., mRNA) expressed from a gene that is associated with a particular disease or condition. In this context, it should be appreciated that a transcript expressed from a gene can refer to a transcript that is encoded by, expressed from, or derived from an expression product of, the gene. For example, in some embodiments, the transcript has a sequence corresponding to an uninterrupted sequence of the gene from which it is expressed. In some embodiments, an expression product of the gene undergoes additional processing steps (e.g., post-transcriptional modification) to produce the transcript.

In some embodiments, the molecules and/or compositions of the disclosure can be used to target a transcript (e.g., mRNA) expressed from a gene selected from Table B. Table B provides a list of example diseases (column A) and genes associated therewith (column B). For each gene listed in Table B, a corresponding sequence and location in the human genome is provided (columns C, D), along with an example transcript expressed from the gene (column E).

Referring to Table B, in some embodiments, the disclosure provides methods of treating at least one disease selected from column A by targeting (e.g., to increase the expression level of a protein (e.g., by changing a codon to a different codon that is used more frequently in the host but that encodes the same amino acid), to decrease the expression level of a protein (e.g., by changing a codon to a different codon that is used less frequently in the host but that encodes the same amino acid), to change one or more amino acids in a protein, and/or to edit a coding sequence as appropriate) a transcript expressed from a corresponding disease-associate gene in column B. In some embodiments, the transcript is the corresponding transcript in column E. In some embodiments, the transcript is different from the corresponding transcript in column E. In some embodiments, the molecules and/or compositions of the disclosure target a transcript selected from column E, or a different transcript expressed from the corresponding disease-associated gene in column B.

TABLE B
Non-Limiting Examples of Targets

| | B. Gene | C. NCBI | D. Reference | E. Reference |
|---|---|---|---|---|
| A. Disease | Name | Gene ID | Accession#, Genomic | Accession#, mRNA |
| Cystic fibrosis | CFTR | 1080 | NC_000007.14 | NM_000492.4 |
| | | | 117480025 . . . 117668665 | |
| Hurler Syndrome | IDUA | 3425 | NC_000004.12 | NM_000203.5 |
| (Mucopolysaccharidosis) | | | 986997 . . . 1004564 | |
| alpha- I-antitrypsin | SERPINA1 | 5265 | NC_000014.9 | NM_000295.5 |
| (AlAT) deficiency | | | 94376747 . . . 94390654 comp | |
| Parkinson's disease | GBA | 2629 | NC_000001.11 | NM_000157.4 |
| | | | 155234452 . . . 155244627 comp | |
| | LRRK2 | 120892 | NC_000012.12 | NM_198578.4 |
| | | | 40224890 . . . 40369285 | |
| | PRKN | 5071 | NC_000006.12 | NM_004562.3 |
| | | | 161347417 . . . 162727802 Comp | |
| | SNCA | 6622 | NC_000004.12 | NM_000345.4 |
| | | | 89724099 . . . 89838324 comp | |
| Alzheimer's disease | MAPT | 4137 | NC_000017.11 | NM_001123066.4 |
| | | | 45894538 . . . 46028334 | |
| | APOE | 348 | NC_000019.10 | NM_000041.4 |
| | | | 44905796 . . . 44909393 | |
| | APP | 351 | NC_000021.9 | NM_000484.4 |
| | | | 25880550 . . . 26171128 comp | |
| | PSEN1 | 5663 | NC_000014.9 | NM_000021.4 |
| | | | 73136436 . . . 73223691 | |
| | PSEN2 | 5664 | NC_000001.11 | NM_000447.3 |
| | | | 226870594 . . . 226903829 | |
| albinism | TYR | 7299 | NC_000011.10 | NM_000372.5 |
| | | | 89177565 . . . 89295759 | |
| | OCA2 | 4948 | NC_000015.10 | NM_000275.3 |
| | | | 27719008 . . . 28099342 comp | |
| | TYRP1 | 7306 | NC_000009.12 | NM_000550.3 |
| | | | 12693375 . . . 12710285 | |
| | SLC45A2 | 51151 | NC_000005.10 | NM_001012509.4 |
| | | | 33944623 . . . 33984693 comp | |
| | GPR143 | 4935 | NC_000023.11 | NM_000273.3 |
| | | | 9725346 . . . 9786260, comp | |
| | LYST | 1130 | NC_000001.11 | NM_000081.4 |
| | | | 235661031 . . . 235883713 comp | |
| Amyotrophic lateral | SOD1 | 6647 | NC_000021.9 | NM_000454.5 |
| sclerosis | | | 31659693 . . . 31668931 | |
| | FUS | 2521 | NC_000016.10 | NM_001170634.1 |
| | | | 31180110 . . . 31194871 | |
| | NEK1 | 4750 | NC_000004.12 | NM_001199397.3 |
| | | | 169392809 . . . 169612629 comp | |
| | C9ORF72 | 203228 | NC_000009.12 | NM_001256054.3 |
| | | | 27546546 . . . 27573866 comp | |
| | TDP43 | 23435 | NC_000001.11 | NM_007375.4 |
| | | | 11012654 . . . 11030528 | |
| | UBQLN2 | 29978 | NC_000023.11 | NM_013444.4 |
| | | | 56563627 . . . 56567868 | |
| | KIF5A | 3798 | NC_000012.12 | NM_001354705.2 |
| | | | 57550039 . . . 57586633 | |
| Asthma | ORMDL3 | 94103 | NC_000017.11 | NM_001320801.2 |
| | | | 39921041 . . . 39927601 comp | |
| | HLA-dQ | 3117 | NC_000006.12 | NM_002122.5 |
| | | | 32637406 . . . 32654846 | |
| | SMAD3 | 4088 | NC_000015.10 | NM_001145102.2 |
| | | | 67065602 . . . 67195195 | |
| | ADAM33 | 80332 | NC_000020.11 | NM_001282447.3 |
| | | | 3667973 . . . 3682108 comp | |
| beta-thalassemia (β- | HBB | 3043 | NC_000011.10 | NM_000518.5 |
| thalassemia) | | | 5225464 . . . 5227071 comp | |
| Cadasil syndrome | Notch3 | 4854 | NC_000019.10 | NM_000435.3 |
| | | | 15159038 . . . 15200995 comp | |
| Charcot-Marie- | PMP22 | 5376 | NC_000017.11 | NM_000304.4 |
| Tooth disease | | | 15229779 . . . 15265326 comp | |
| | MPZ | 4359 | NC_000001.11 | NM_000530.8 |
| | | | 161303594 . . . 161309969 comp | |
| Chronic Obstructive | TNF | 7124 | NC_000006.12 | NM_000594.4 |
| Pulmonary Disease | | | 31575565 . . . 31578336 | |
| (COPD) | TGFB1 | 7040 | NC_000019.10 | NM_000660.7 |
| | | | 41330323 . . . 41353922 comp | |
| | GSTP1 | 2950 | NC_000011.10 | NM_000852.4 |
| | | | 67583812 . . . 67586653 | |
| | GSTM1 | 2944 | NC_000001.11 | NM_000561.4 |
| | | | 109687817 . . . 109693745 | |
| | SOD3 | 6649 | NC_000004.12 | NM_003102.4 |
| | | | 24795478 . . . 24800845 | |
| | ASAH1 | 427 | NC_000008.11 | NM_001127505.3 |
| | | | 18055992 . . . 18084961 comp | |
| | IGHMBP2 | 3508 | NC_000011.10 | NM_002180.3 |
| | | | 68903855 . . . 68940601 | |
| | PLEKHG5 | 57449 | NC_000001.11 | NM_001042663.3 |
| | | | 6466092 . . . 6520092 comp | |
| Duchenne/Becker | DMD | 1756 | NC_000023.11 | NM_000109.4 |
| muscular dystrophy | | | 31119219 . . . 33339460 comp | |
| Dystrophic | COL7A1 | 1294 | NC_000003.12 | NM_000094.4 |
| Epidermolysis | | | 48564073 . . . 48595329 comp | |
| bullosa | | | | |
| Epidermylosis | KRT5 | 3852 | NC_000012.12 | NM_000424.4 |
| bullosa simplex | | | 52514575 . . . 52520394 comp | |
| | KRT14 | 3861 | NC_000017.11 | NM_000526.5 |
| | | | 41582279 . . . 41586895 comp | |
| Fabry disease | GLA | 2717 | NC_000023.11 | NM_000169.3 |
| | | | 101397803 . . . 101407925 comp | |
| Factor V Leiden | F5 | 2153 | NC_000001.11 | NM_000130.5 |
| associated disorders | | | 169511951 . . . 169586630 comp | |
| Familial | APC | 324 | NC_000005.10 | NM_000038.6 |
| Adenomatous | | | 112707498 . . . 112846239 | |
| Polyposis (FAP) | | | | |
| Galactosemia | GALT | 2592 | NC_000009.12 | NM_000155.4 |
| | | | 34646675 . . . 34651035 | |
| | GALK1 | 2584 | NC_000017.11 | NM_000154.2 |
| | | | 75751469 . . . 75765192 comp | |
| | GALE | 2582 | NC_000001.11 | NM_000403.4 |
| | | | 23795599 . . . 23800754 comp | |
| Gaucher's Disease | GBA | 2629 | NC_000001.11 | NM_000157.4 |
| | | | 155234452 . . . 155244627 comp | |
| Glucose-6- | G6PD | 2539 | NC_000023.11 | NM_000402.4 |
| phosphate | | | 154531390 . . . 154547569 comp | |
| dehydrogenase | | | | |
| deficiency | | | | |
| Haemophilia | F8 | 2157 | NC_000023.11 | NM_000132.4 |
| | | | 154835792 . . . 155022723 comp | |
| | F9 | 2158 | NC_000023.11 | NM_000133.4 |
| | | | 139530720 . . . 139563459 | |
| Hereditary | HFE | 3077 | NC_000006.12 | NM_000410.4 |
| Hematochromatosis | | | 26087347 . . . 26098343 | |
| Hunter Syndrome | IDS | 3423 | NC_000023.11 | NM_000202.8 |
| | | | 149476988 . . . 149505306 comp | |
| Huntington's disease | HTT | 3064 | NC_000004.12 | NM_001388492.1 |
| | | | 3074681 . . . 3243960 | |
| Inflammatory | NOD2 | 64127 | NC_000016.10 | NM_001293557.2 |
| Bowel Disease | | | 50693587 . . . 50733077 | |
| (IBD) | SMAD7 | 4092 | NC_000018.10 | NM_001190821.2 |
| | | | 48919853 . . . 48950965 comp | |
| | ATG16L | 55054 | NC_000002.12 | NM_001190266.2 |
| | | | 233251647 . . . 233295674 | |
| | IL23 | 51561 | NC_000012.12 | NM_016584.3 |
| | | | 56334159 . . . 56340410 | |
| | PTPN2 | 5771 | NC_000018.10 | NM_001207013.2 |
| | | | 12785478 . . . 12884351 comp | |
| Inherited | A4GALT | 53947 | NC_000022.11 | NM_001318038.3 |
| polyagglutination | | | 42692115 . . . 42721301 comp | |
| syndrome | | | | |
| Leber congenital | CEP290 | 80184 | NC_000012.12 | NM_025114.4 |
| amaurosis | | | 88049013 . . . 88142216 comp | |
| | CRB1 | 23418 | NC_000001.11 | NM_001193640.2 |
| | | | 197201504 . . . 197478455 | |
| | GUCY2D | 3000 | NC_000017.11 | NM_000180.4 |
| | | | 8002615 . . . 8020342 | |
| | RPE65 | 6121 | NC_000001.11 | NM_000329.3 |
| | | | 68428822 . . . 68450322 comp | |
| Lesch-Nyhan | HPRT1 | 3251 | NC_000023.11 | NM_000194.3 |
| syndrome | | | 134460165 . . . 134500668 | |
| Lynch syndrome | MLH1 | 4292 | NC_000003.12 | NM_000249.4 |
| | | | 36993487 . . . 37050846 | |
| | MSH2 | 4436 | NC_000002.12 | NM_000251.3 |
| | | | 47403067 . . . 47634501 | |
| | MSH6 | 2956 | NC_000002.12 | NM_000179.3 |
| | | | 47783145 . . . 47806954 | |
| | PMS2 | 5395 | NC_000007.14 | NM_000535.7 |
| | | | 5970925 . . . 6009106 comp | |
| | EPCAM | 4072 | NC_000002.12 | NM_002354.3 |
| | | | 47369311 . . . 47387020 | |
| Marfan syndrome | FBN1 | 2200 | NC_000015.10 | NM_000138.5 |
| | | | 48408313 . . . 48645709 comp | |
| Muscular Dystrophy | LMNA | 4000 | NC_000001.11 | NM_001257374.3 |
| | | | 156082573 . . . 156140081 | |
| | DUX4 | 100288687 | NC_000004.12 | NM_001293798.3 |
| | | | 190173774 . . . 190185911 | |
| | DYSF | 8291 | NC_000002.12 | NM_001130455.2 |
| | | | 71453154 . . . 71686763 | |
| | FKTN | 2218 | NC_000009.12 | NM_001079802.2 |
| | | | 105558117 . . . 105655950 | |
| Myotonic dystrophy | DMPK | 1760 | NC_000019.10 | NM_001081560.3 |
| types I | | | 45769709 . . . 45782490 comp | |
| Myotonic dystrophy | CNBP | 7555 | NC_000003.12 | NM_001127192.2 |
| types II | | | 129167827 . . . 129183896 comp | |
| Neurofibromatosis | NF1 | 4763 | NC_000017.11 | NM_000267.3 |
| | | | 31094927 . . . 31377677 | |
| | NF2 | 4771 | NC_000022.11 | NM_000268.4 |
| | | | 29603556 . . . 29698600 | |
| | SMARCB1 | 6598 | NC_000022.11 | NM_001007468.3 |
| | | | 23786966 . . . 23838009 | |
| | LZTR1 | 8216 | NC_000022.11 | NM_006767.4 |
| | | | 20982297 . . . 20999032 | |
| Niemann-Pick | SMPD1 | 6609 | NC_000011.10 | NM_000543.5 |
| disease type A, B | | | 6390474 . . . 6394998 | |
| and C | NPC1 | 4864 | NC_000018.10 | NM_000271.5 |
| | | | 23506184 . . . 23586506, comp | |
| | NPC2 | 10577 | NC_000014.9 | NM_001363688.1 |
| | | | 74479935 . . . 74493512, comp | |
| NY-esol related | CTAG1B | 1485 | NC_000023.11 | NM_001327.3 |
| cancer | | | 154617609 . . . 154619282 comp | |
| Peutz-Jeghers | STK11 | 6794 | NC_000019.10 | NM_000455.5 |
| Syndrome | | | 1205778 . . . 1228431 | |
| Phenylketonuria | PAH | 5053 | NC_000012.12 | NM_000277.3 |
| | | | 102836889 . . . 102958441 comp | |
| Pompe's disease | GAA | 2548 | NC_000017.11 | NM_000152.5 |
| | | | 80101535 . . . 80119881 | |
| Primary Ciliary | DNAI1 | 27019 | NC_000009.12 | NM_001281428.2 |
| Disease | | | 34458805 . . . 34520984 | |
| | DNAH5 | 1767 | NC_000005.10 | NM_001369.3 |
| | | | 13690328 . . . 14011829, comp | |
| Prothrombin | F2 | 2147 | NC_000011.10 | NM_000506.5 |
| mutation related | | | 46719213 . . . 46739506 | |
| disorders | | | | |
| Pulmonary | BMPR2 | 659 | NC_000002.12 | NM_001204.7 |
| Hypertension | | | 202376310 . . . 202567751 | |
| | ACVRL1 | 94 | NC_000012.12 | NM_000020.3 |
| | | | 51906913 . . . 51923361 | |
| | CAV1 | 857 | NC_000007.14 | NM_001172895.1 |
| | | | 116525009 . . . 116561185 | |
| | ENG | 2022 | NC_000009.12 | NM_000118.3 |
| | | | 127815012 . . . 127854773 comp | |
| | SMAD9 | 4093 | NC_000013.11 | NM_001127217.3 |
| | | | 36844831 . . . 36920721 comp | |
| Retinitis Pigmentosa | RHO | 6010 | NC_000003.12 | NM_000539.3 |
| | | | 129528639 . . . 129535344 | |
| Sandhoff Disease | HEXB | 3074 | NC_000005.10 | NM_000521.4 |
| | | | 74640023 . . . 74721288 | |
| X-linked or Severe | IL2RG | 3561 | NC_000023.11 | NM_000206.3 |
| Combined Immune | | | 71107404 . . . 71111577 comp | |
| Deficiency | | | | |
| Syndrome (SCID) | | | | |
| Sickle Cell Anemia | HBB | 3043 | NC_000011.10 | NM_000518.5 |
| | | | 5225464 . . . 5227071 comp | |
| Spinal Muscular | SMN1 | 6606 | NC_000005.10 | NM_000344.4 |
| Atrophy | | | 70924941 . . . 70953015 | |
| Stargardt's Disease | ABCA4 | 24 | NC_000001.11 | NM_000350.3 |
| | | | 93992834 . . . 94121148 comp | |
| Tay-Sachs Disease | HEXA | 3073 | NC_000015.10 | NM_000520.6 |
| | | | 72340924 . . . 72376014 comp | |
| Usher syndrome | USH2A | 7399 | NC_000001.11 | NM_007123.6 |
| | | | 215622891 . . . 216423448 comp | |
| | CLRN1 | 7401 | NC_000003.12 | NM_001195794.1 |
| | | | 150926163 . . . 150972999 comp | |
| Sturge-Weber | GNAQ | 2776 | NC_000009.12 | NM_002072.5 |
| Syndrome | | | 77716097 . . . 78031811 comp | |

### Administration, Compositions, and Kits

### Administration

To administer any of the molecules and/or compositions herein (e.g., nucleic acids, duplexes) or to practice any of the methods disclosed herein, an effective amount of the molecules and/or compositions as described herein can be administered to a subject (e.g., a human) via a suitable route (as discussed herein). In some embodiments, the subject may be in need thereof, suspected to be in need, or at risk of needing the molecules and/or compositions as described herein. In some embodiments, the molecules and/or compositions may be administered to treat a disease or disorder related to RNA editing, related to expression of an target adenosine, or related to non-expression of a target adenosine.

The administration of the molecules and/or compositions of the disclosure may be by any acceptable means and any of the molecules and/or compositions may be administered by any administration route known in the art. For example, in some embodiments, the molecules and/or compositions may be administered via conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some embodiments, the administration route is enteral or gastrointestinal (e.g., oral) and the formulation is formulated for enteral or gastrointestinal administration (e.g., oral). In some embodiments, the administration route is parenteral and the formulation is formulated for parenteral administration. In some embodiments, the administration route is via injection and the formulation is formulated for injection. In some embodiments, the administration route is sublingual and the formulation is formulated for sublingual administration. In some embodiments, the administration route is buccal and the formulation is formulated for buccal administration. In some embodiments, the administration route is nasal and the formulation is formulated for nasal administration. In some embodiments, the administration route is transdermal and the formulation is formulated for transdermal administration. In some embodiments, the administration route is subcutaneous and the formulation is formulated for subcutaneous administration. In some embodiments, the administration route is perivascular and the formulation is formulated for perivascular administration. In some embodiments, the administration route is topical and the formulation is formulated for topical administration. In some embodiments, the administration route is rectal (e.g., intrarectal) and the composition is formulated for rectal administration. In some embodiments, the administration route is intravenously (e.g., by venous or arterial puncture), and the formulation is formulated for intravenous (e.g., by venous or arterial puncture) administration.

### Compositions

In some embodiments, the molecules and/or compositions disclosed herein (e.g., nucleic acids, ADAR recruiting molecules, targeting molecules, etc.) may further comprise a pharmaceutically acceptable composition. In some embodiments, the molecules and/or compositions disclosed herein (e.g., nucleic acids, ADAR recruiting molecules, targeting molecules, etc.) can be formulated for administration to a subject as a pharmaceutically acceptable composition, which, as used herein, comprises the molecules and/or compositions disclosed herein (e.g., nucleic acids, ADAR recruiting molecules, targeting molecules, etc.) and another pharmaceutically acceptable carrier, diluent, or excipient. A carrier, diluent, or excipient that is “pharmaceutically acceptable” includes one that is sterile and pyrogen free. Suitable pharmaceutical carriers, diluents, and excipients are well known in the art. The carrier(s) must be “acceptable” in the sense of being compatible with the inhibitor and not deleterious to the recipients thereof (e.g., subject).

Formulations suitable for parenteral administration include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. Aqueous solutions may be suitably buffered (preferably to a pH of from about 3 to about 9). The preparation of suitable parenteral formulations under sterile conditions is readily accomplished by standard pharmaceutical techniques well-known to those skilled in the art.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the agents and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the agents, can be dissolved and administered in a pharmaceutical excipient such as Water-for-Injection, 0.9% saline, or 5% glucose solution.

Any of the molecules and/or compositions disclosed herein (e.g., nucleic acids, ADAR recruiting molecules, targeting molecules, etc.) may be administered by any administration route known in the art, such as parenteral administration, oral administration, buccal administration, sublingual administration (e.g., tablets, capsules, ovules, elixirs, solutions or suspensions, which may contain flavoring or coloring agents, for immediate-, delayed- or controlled-release applications) topical administration, or inhalation, in the form of a pharmaceutical formulation (e.g., comprising a composition) comprising the active ingredient, optionally in the form of a non-toxic organic, or inorganic, acid, or base, addition salt, in a pharmaceutically acceptable dosage form. Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds (e.g., miR-224 inhibitors) of the disclosure may be combined with various sweetening or flavoring agents, coloring matter or dyes, with emulsifying and/or suspending agents and with diluents such as water, ethanol, propylene glycol and glycerin, and combinations thereof. Additionally, lubricating agents such as magnesium stearate, stearic acid, glyceryl behenate and talc may be included.

The formulations may be presented in unit-dose or multi-dose containers, for example sealed ampoules or vials, and may be stored in a freeze-dried (lyophilized) condition requiring only the addition of the sterile liquid carrier immediately prior to use.

### Kits

In an aspect, the disclosure relates to kits for administering one or more molecules and/or compositions disclosed herein (e.g., nucleic acids, ADAR recruiting molecules, targeting molecules, etc.) to a subject for the treatment of disorder related to a mutation or expression of a target adenosine. The representative kits include one or more dosage units comprising an effective amount of one or more molecules and/or compositions disclosed herein (e.g., nucleic acids, ADAR recruiting molecules, targeting molecules, etc.) for administration to a subject, at a given frequency, and/or in a given manner (e.g., route of administration).

In some embodiments, the kits provide cells comprising any of the molecules and/or compositions disclosed herein (e.g., nucleic acids, ADAR recruiting molecules, targeting molecules, etc.).

Instructions for performing any of the methods disclosed here and administering the agent may also be included in the kits described herein.

The kits may be organized to indicate a single formulation containing an molecules and/or compositions disclosed herein (e.g., nucleic acids, ADAR recruiting molecules, targeting molecules, etc.) or combination of formulations, each containing molecules and/or compositions disclosed herein (e.g., nucleic acids, ADAR recruiting molecules, targeting molecules, etc.). The composition may be sub-divided to contain appropriate quantities of an molecules and/or compositions disclosed herein (e.g., nucleic acids, ADAR recruiting molecules, targeting molecules, etc.). The unit dosage can be packaged compositions such as packeted (i.e., contained in a packet) powders, vials, ampoules, prefilled syringes, tablets, caplets, capsules, or sachets containing liquids.

The agents described herein may be a single dose or for continuous or periodic discontinuous administration. For continuous administration, a kit may include an agent described herein in each dosage unit. When varying concentrations of an agent described herein, the components of the composition containing the agent described herein, or relative ratios of the agent described herein or other agents within a composition over time is desired, a kit may contain a sequence of dosage units.

The kit may contain packaging or a container with an agent described herein formulated for the desired delivery route. The kit may also contain dosing instructions, an insert regarding the agent described herein, instructions for monitoring circulating levels of the agent, or combinations thereof. Materials for using the agent may further be included and include, without limitation, reagents, well plates, containers, markers, or labels, and the like. Such kits may be packaged in a manner suitable for treatment of a desired indication (e.g., disorder).

Other suitable components to include in such kits will be readily apparent to one of skill in the art, taking into consideration the desired indication and the delivery route. The kits also may include, or be packaged with, instruments for assisting with the injection/administration of the agent to the subject. Such instruments include, without limitation, an inhalant, syringe, pipette, forceps, measuring spoon, eye dropper, or any such medically approved delivery means. Other instrumentation may include a device that permits reading or monitoring reactions in vitro.

The agent may be provided in dried, lyophilized, or liquid forms. When reagents or components are provided as a dried form, reconstitution generally is by the addition of a solvent. The solvent may be provided in another packaging means and may be selected by one skilled in the art.

A number of packages or kits are known to those skilled in the art for dispensing pharmaceutical agents. In certain embodiments, the package is a labeled blister package, dial dispenser package, or bottle.

### EXAMPLES

### Example 1: Recruiting Molecule can Impact ADAR Editing Efficiency

The following oligonucleotide experimental designs were tested:
Composition RL0079, comprising oligo RL0079 (Strand Ref.: 1)—negative control that did not show any editing;
Composition RH0001, comprising oligo RH0001 (Strand Ref.: 2)—positive control;
Composition RD0016, a duplex comprising oligo RS0003 (Strand Ref.: 3) and oligo RL0016d (Strand Ref.: 4) with guide oligo RL0016g (Strand Ref.: 5);
Composition RD0034, a duplex comprising oligo RS0008 (Strand Ref.: 6) and oligo RL0034d (Strand Ref.: 7) with a guide oligo RL0034g (Strand Ref.: 8); and
Composition RD0037, a duplex comprising oligo RS0009 (Strand Ref.: 6) and oligo RL0037 (Strand Ref.: 20).

Editing Procedures in HeLa Cells

HeLa, Hep-G2, and MCF-7 cells were cultured in Eagle's Minimal Essential Medium (EMEM) supplemented with 10% fetal bovine serum (FBS), 100 U/mL penicillin and 100 μg/mL streptomycin at 37° C. in an atmosphere of 5% CO₂. U-2 OS and SK-BR-3 cells were cultured in McCoy's 5A Medium supplemented with 10% fetal bovine serum (FBS), 100 U/mL penicillin and 100 μg/mL streptomycin at 37° C. in an atmosphere of 5% CO₂. NCI-H1395, NCI-H1623, and NCI-H1993 cells were cultured in RPMI-1640 Medium supplemented with 10% fetal bovine serum (FBS), 100 U/mL penicillin and 100 μg/mL streptomycin at 37° C. in an atmosphere of 5% CO₂. All cell lines were acquired from ATCC. All reagents for cell culture were acquired from ThermoFisher Scientific™

Cells were seeded in 24-well plates at 1×10⁵ HeLa cells/well in 500 μL of growth medium without antibiotics (EMEM+10% FBS) one day prior to the transfection. The next day, cells were transfected with 100 nM 3′ UTR GAPDH RNA oligonucleotide complexed with 3 μL Lipofectamaine RNAiMAX. After 24 hours (h), cells were harvested and RNA extracted using the RNeasy Micro Kit (QIAGEN). Reverse transcription was performed using SuperScript IV (Invitrogen) followed by PCR with Platinum II Taq polymerase (ThermoFisher). The resulting DNA was purified on QIAquick PCR Purification columns (QIAGEN) and analyzed by Sanger sequencing (Genewiz).

Results

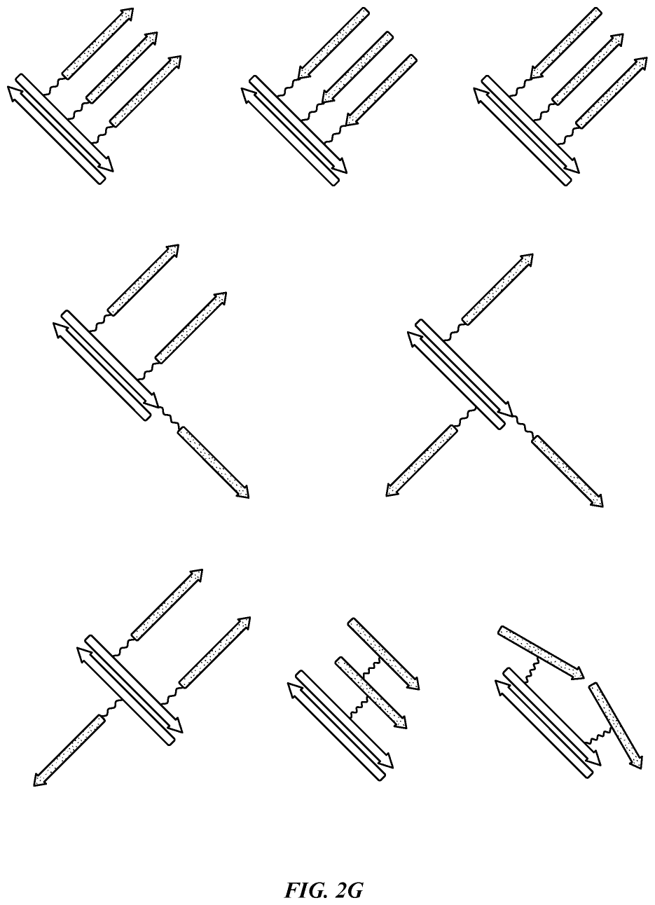

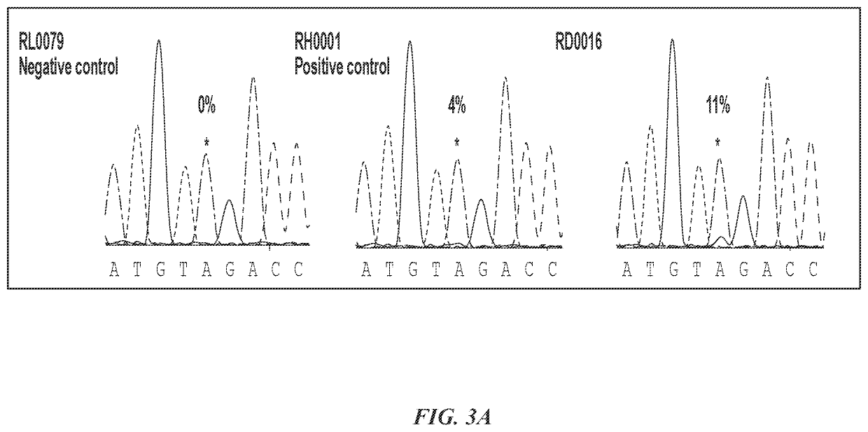

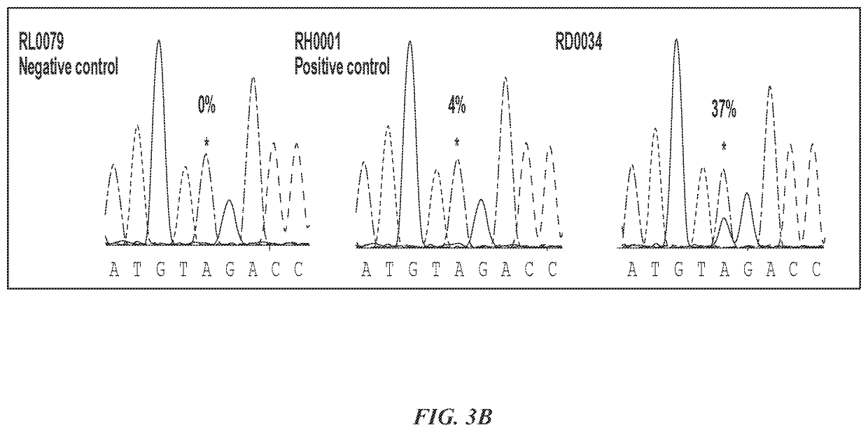

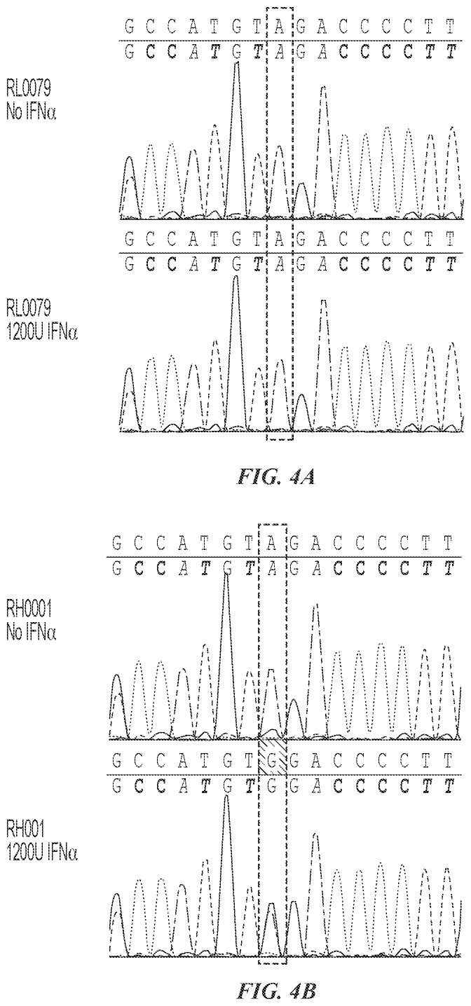

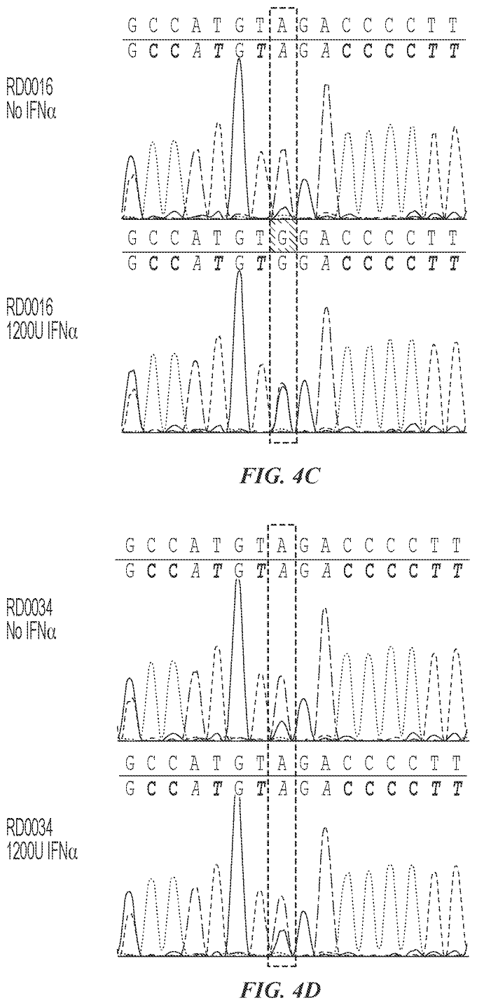

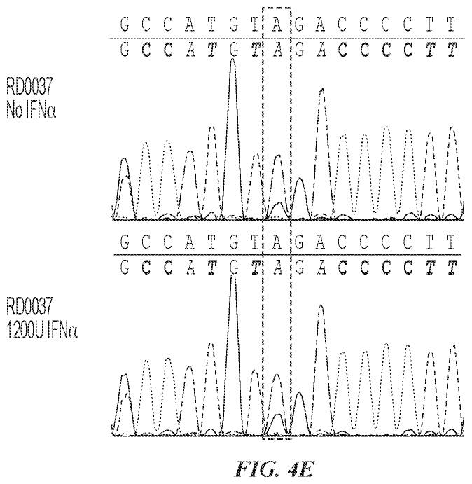

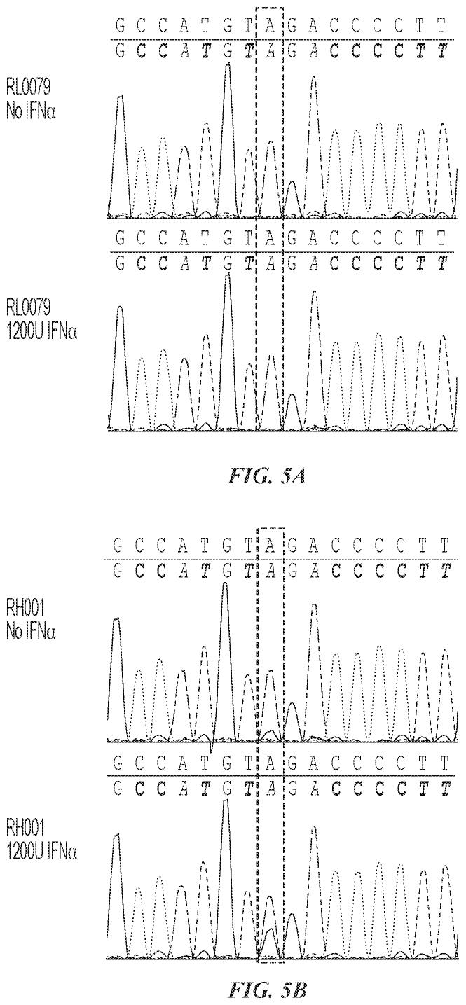

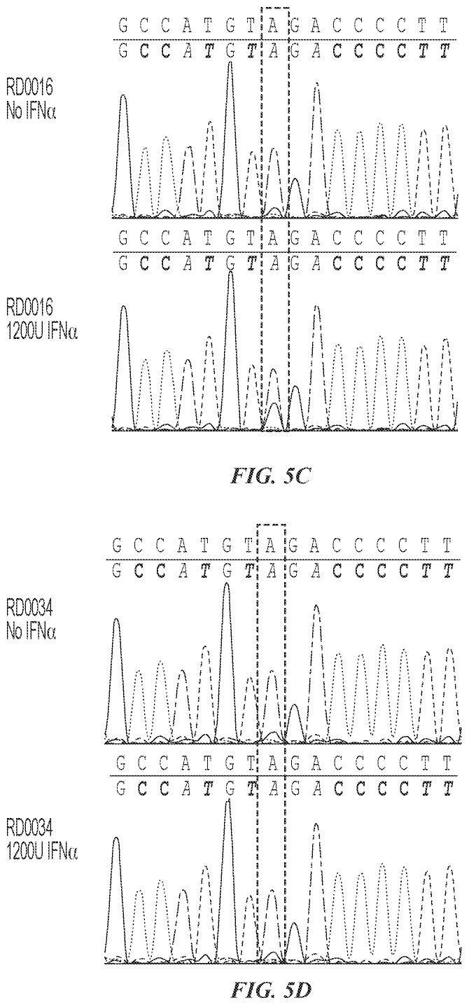

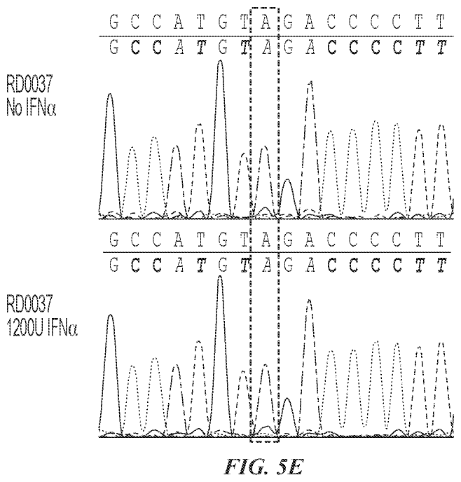

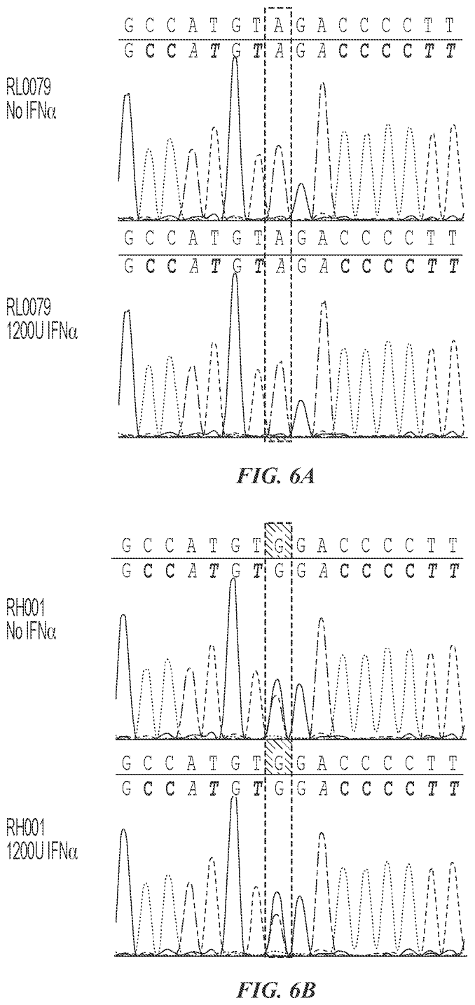

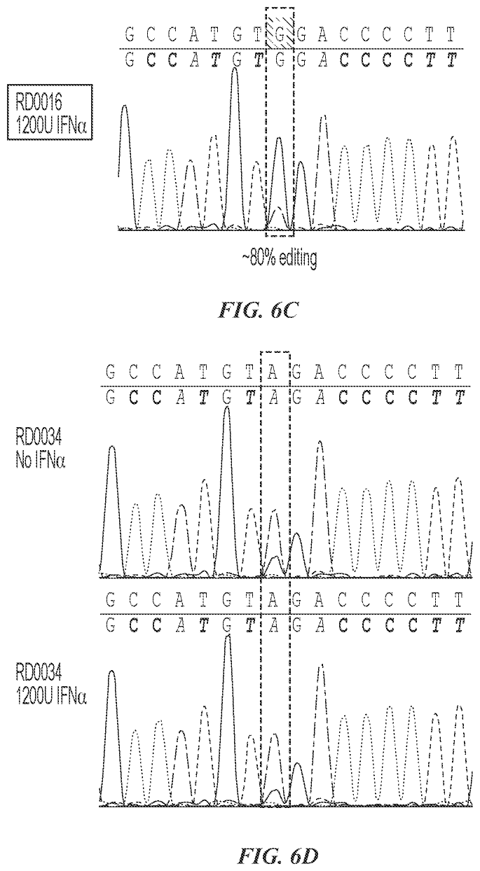

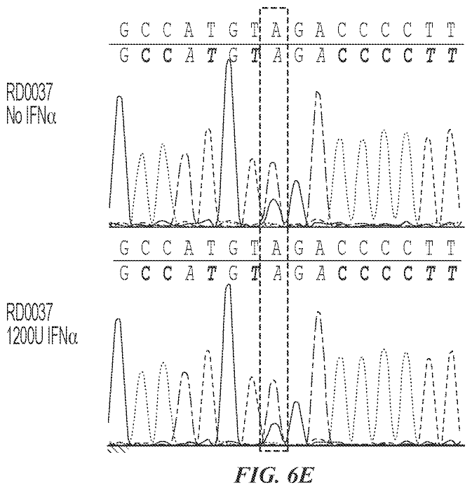

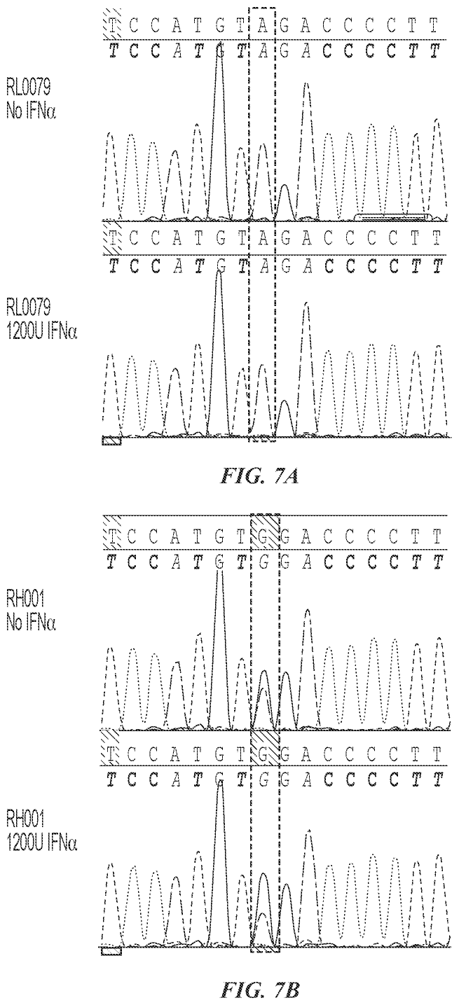

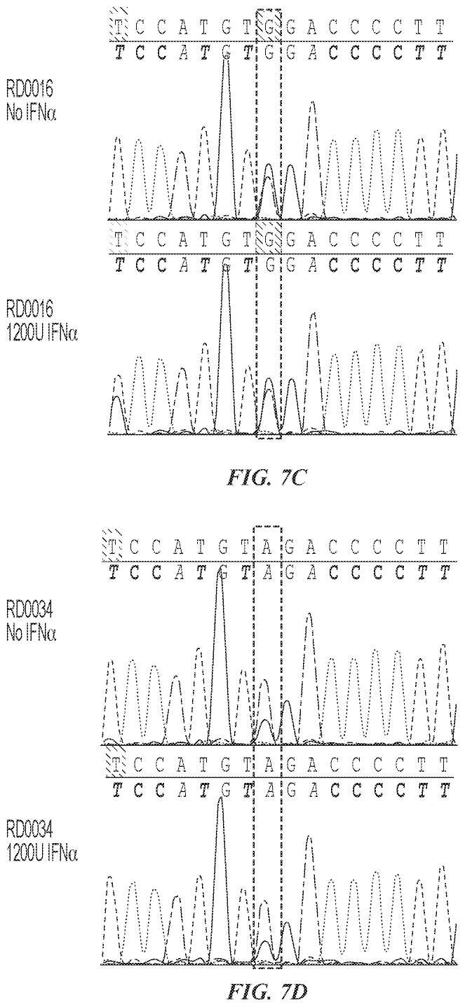

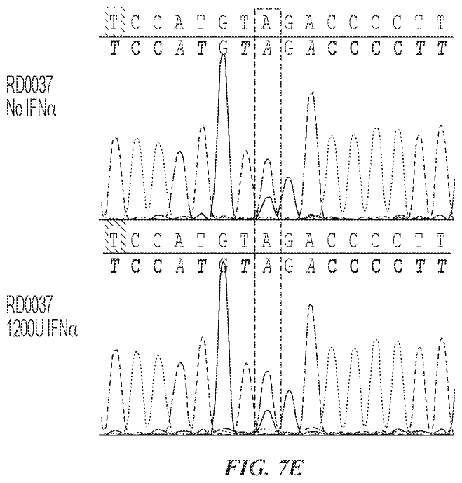

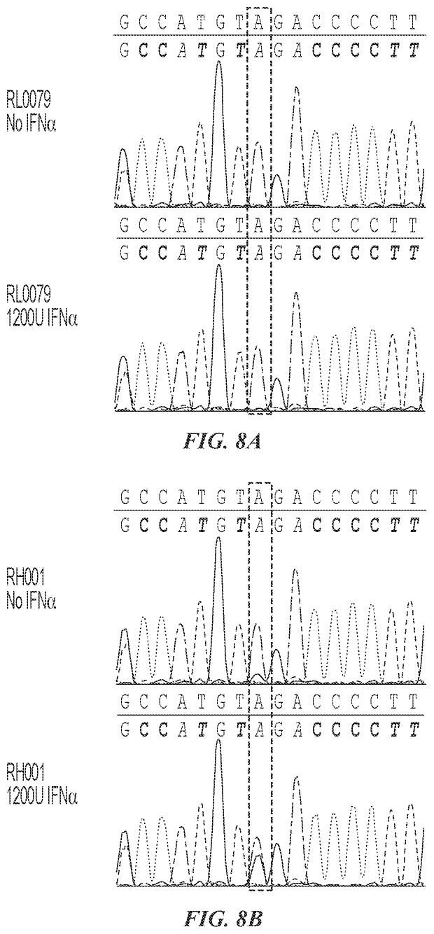

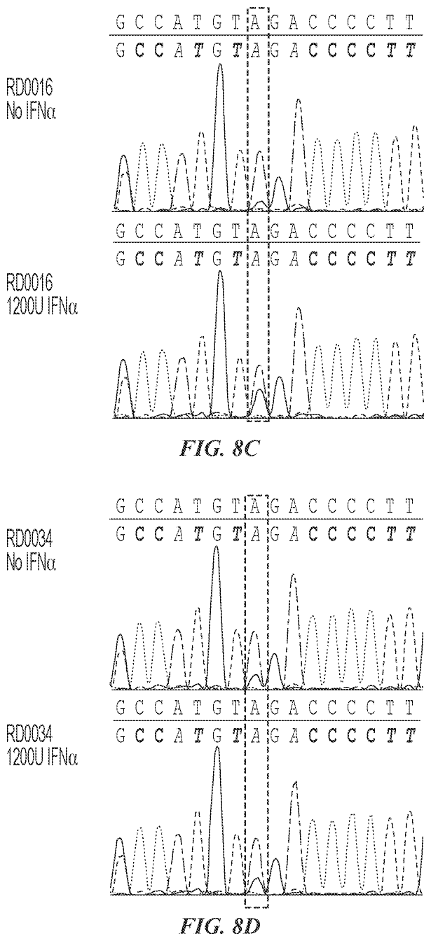

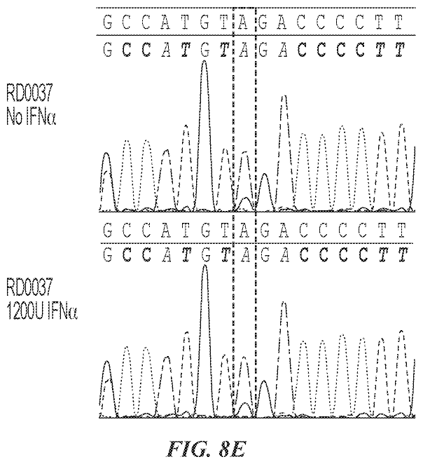

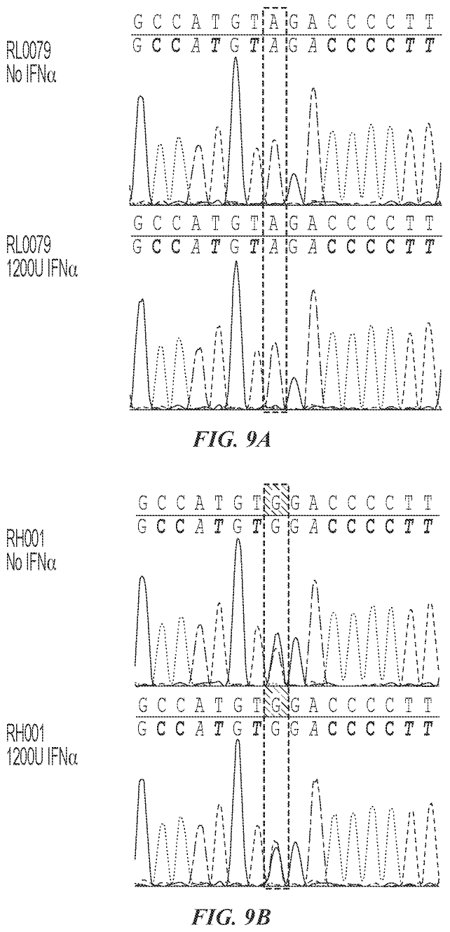

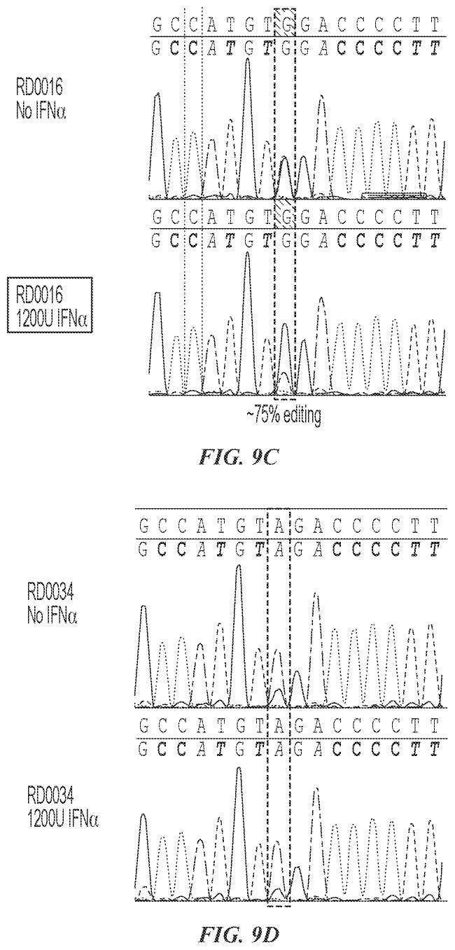

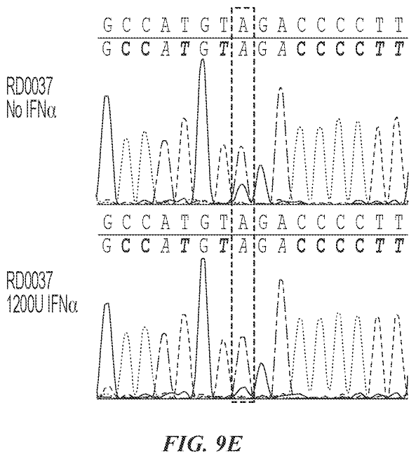

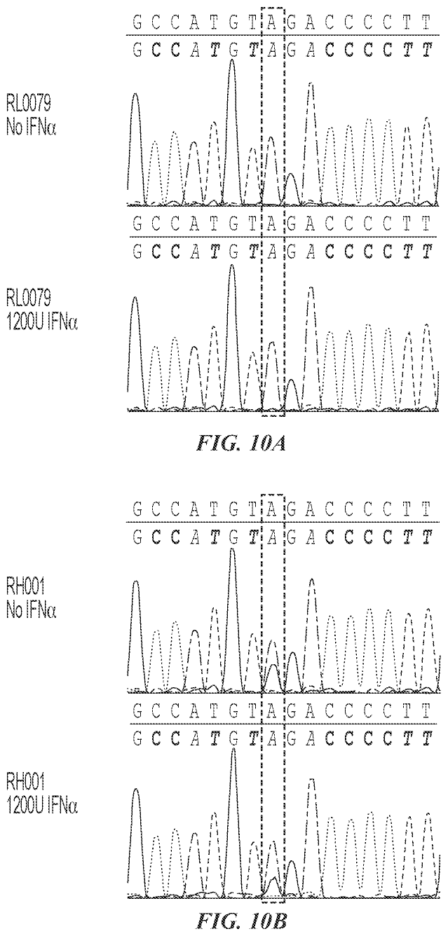

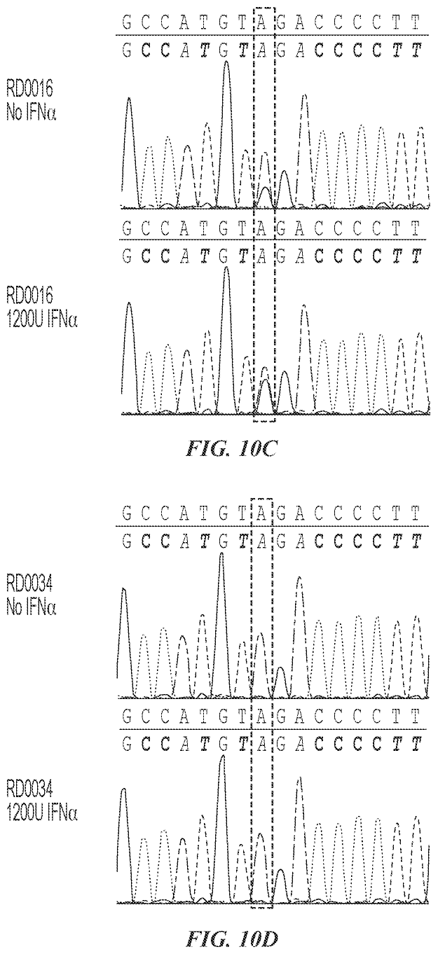

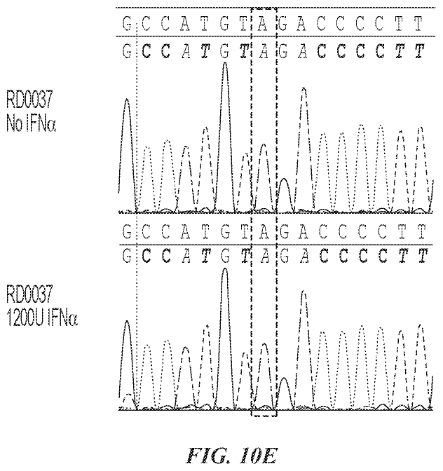

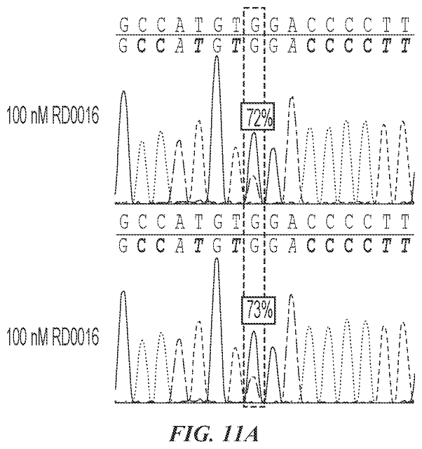

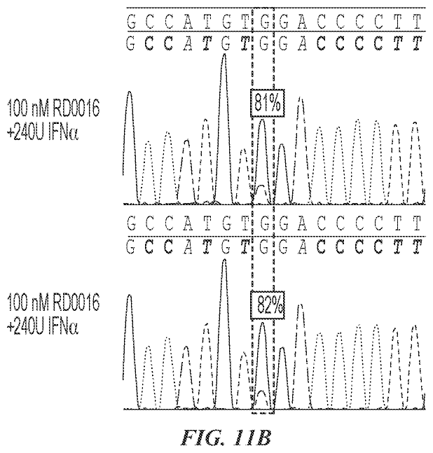

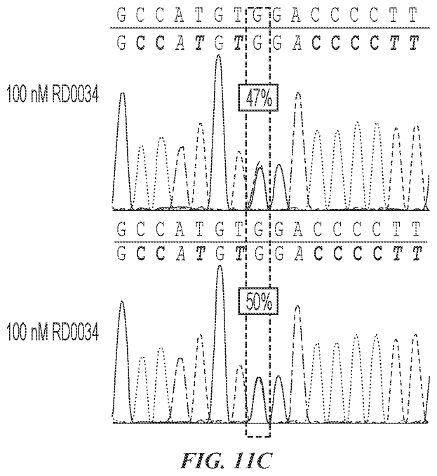

Results of the various oligo sequences are shown in FIGS. 3A-11C. In HeLa cells, RD0034 showed 37% editing and did not respond to INFα (FIGS. 3B and 4D), while RD0016 showed good response to INFα (FIGS. 3A and 4C). In the NCI-H1993 cell line, RD0016 showed 50%-60% editing efficiency without the use of INFα (FIG. 7C). In the NCI-H1395 cell line, RD0016 showed ˜80% editing with INFα (FIG. 6C). In the NCI-H1623 cell line, RD0016 showed ˜72%-73% editing without INFα (FIG. 11A). In the NCI-H1623 cell line, RD0016 showed ˜81%-82% editing with INFα (FIG. 11B). In the NCI-H1623 cell line, RD0034 showed ˜47%-50% editing without INFα (FIG. 11C).

### Example 2: Linkers in Recruiting Molecule and Targeting Molecules can Impact ADAR Editing Efficiency

Editing Procedures in HeLa Cells

SK-BR-3 cells were cultured in McCoy's 5A Medium supplemented with 10% fetal bovine serum (FBS), 100 U/mL penicillin and 100 μg/mL streptomycin at 37° C. in an atmosphere of 5% CO₂. Cell lines were acquired from ATCC. All reagents for cell culture were acquired from ThermoFisher Scientific™.

Cells were seeded in 24-well plates at 1×10⁵ HeLa cells/well in 500 μL of growth medium without antibiotics (EMEM+10% FBS) one day prior to the transfection. The next day, cells were transfected with 5 different concentrations (100 nM, 10 nM, 3.3 nM, 1.1 nM, and 0.37 nM) of 3′ UTR GAPDH RNA oligonucleotide complexed with 3 μL Lipofectamaine RNAiMAX. After 24 hours (h), cells were harvested and RNA extracted using the RNeasy Micro Kit (QIAGEN). Reverse transcription was performed using SuperScript IV (Invitrogen) followed by PCR with Platinum II Taq polymerase (ThermoFisher). The resulting DNA was purified on QIAquick PCR Purification columns (QIAGEN) and analyzed by Sanger sequencing (Genewiz).

Results

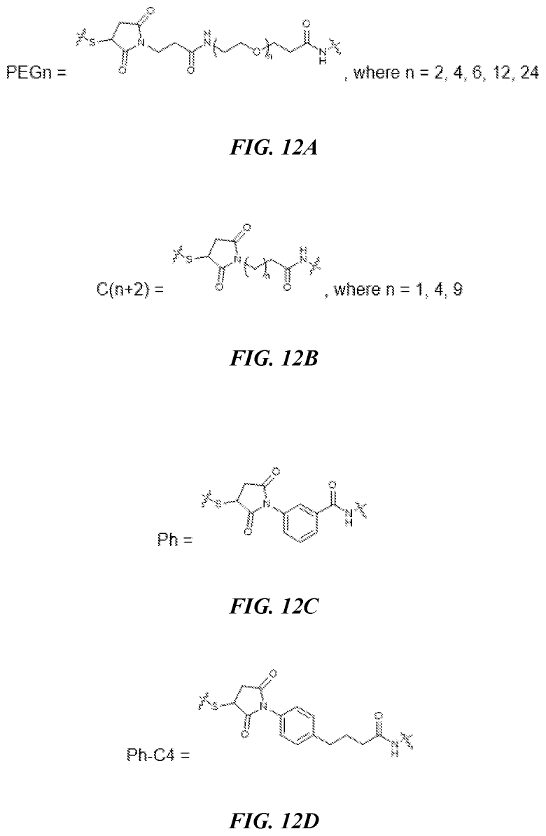

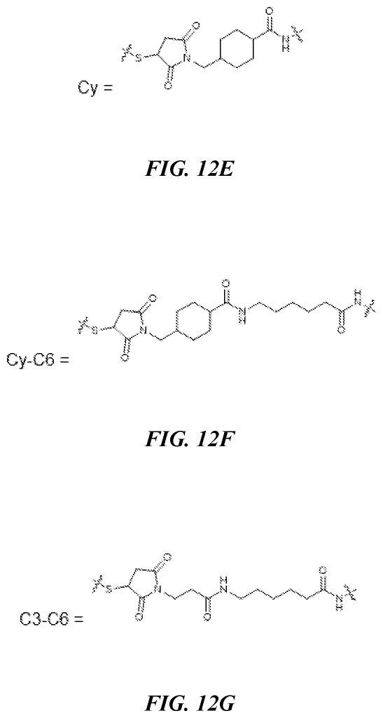

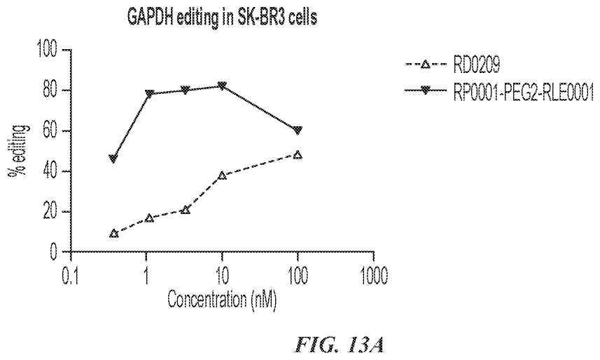

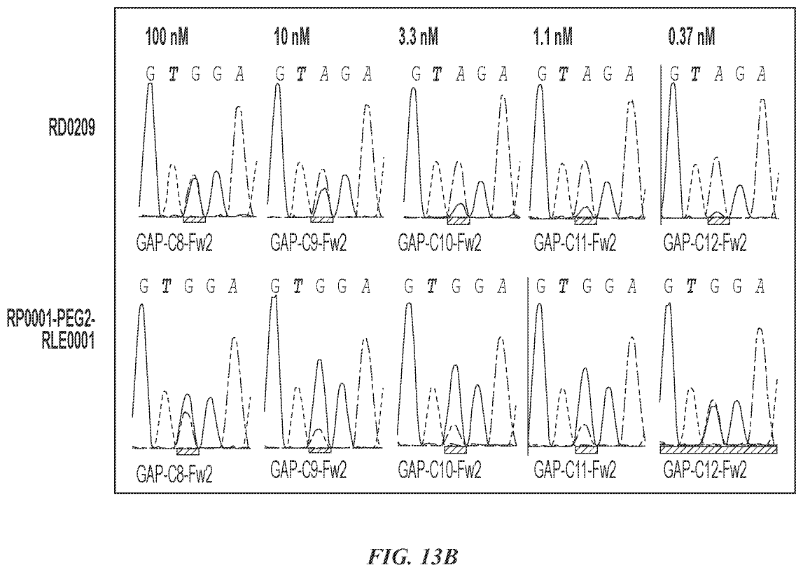

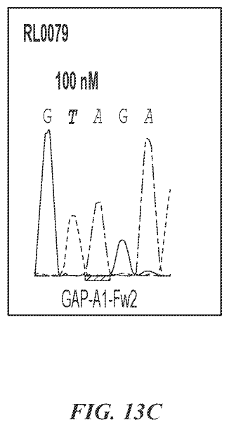

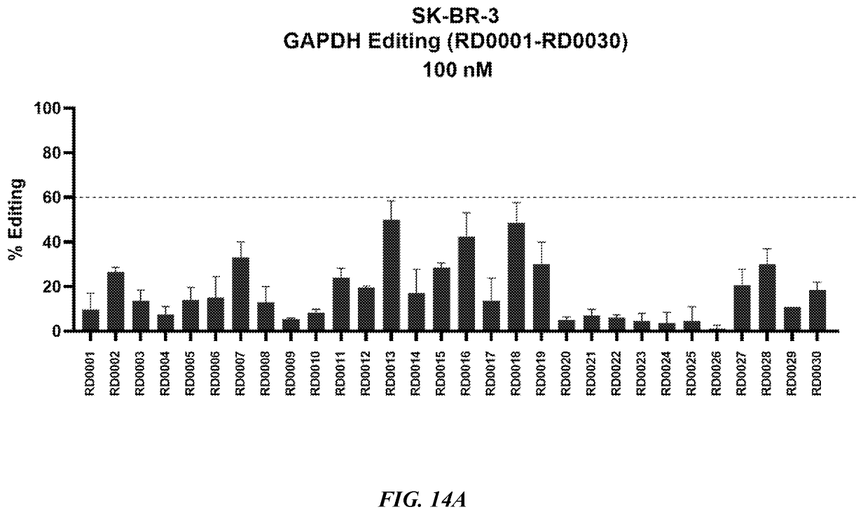

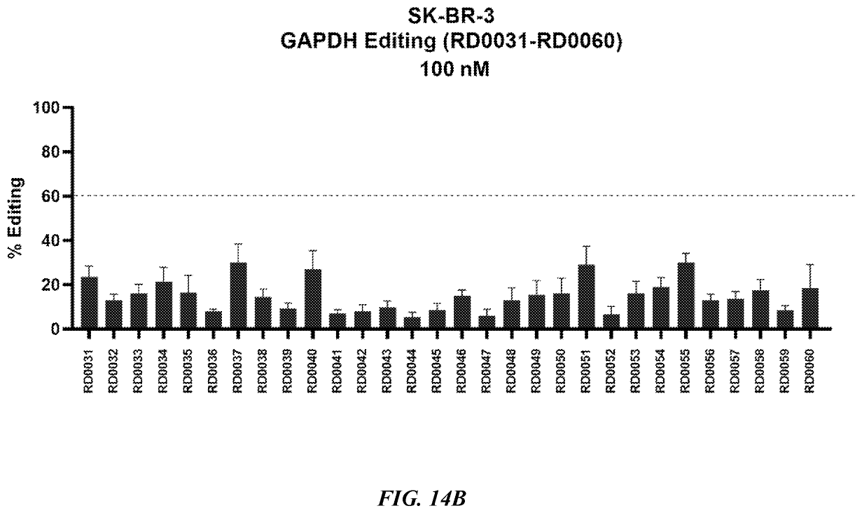

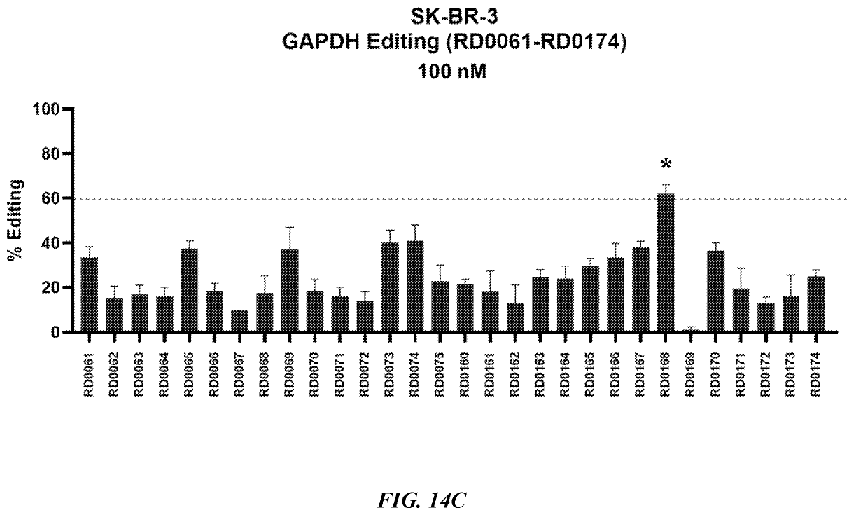

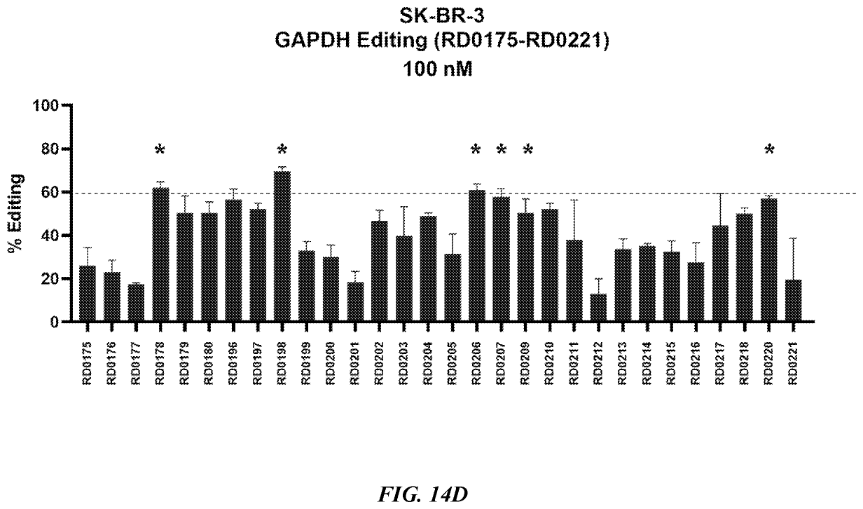

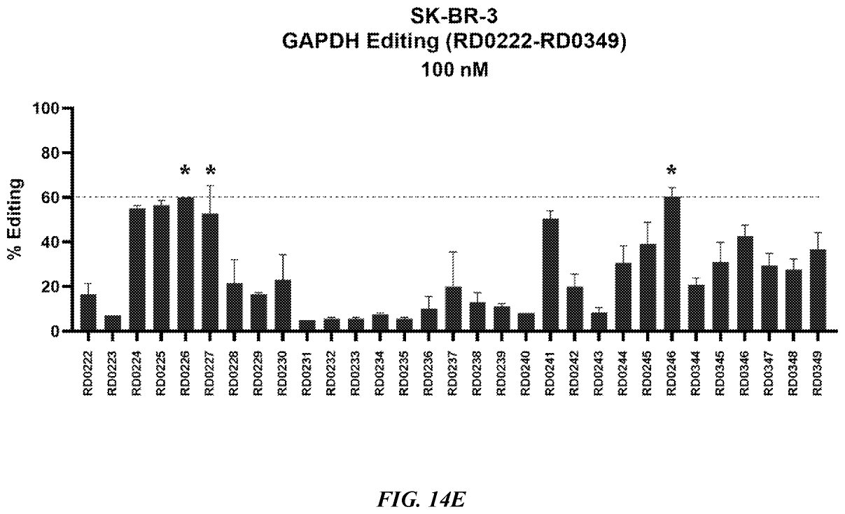

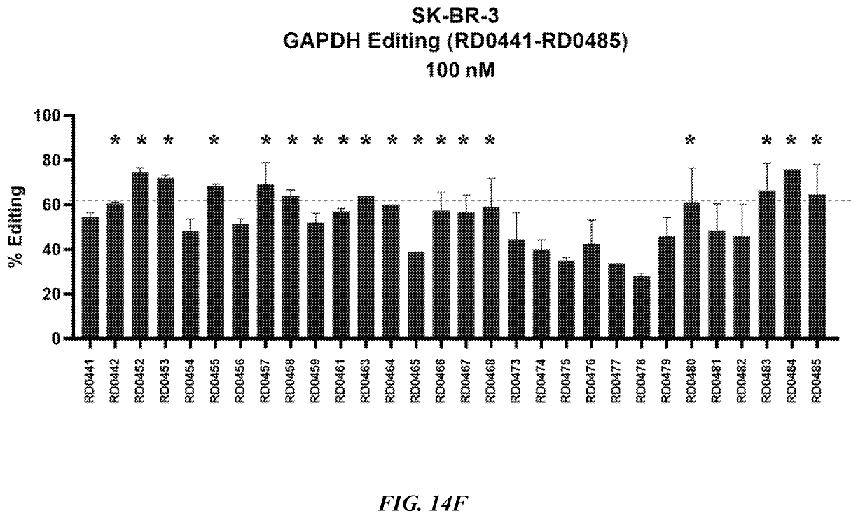

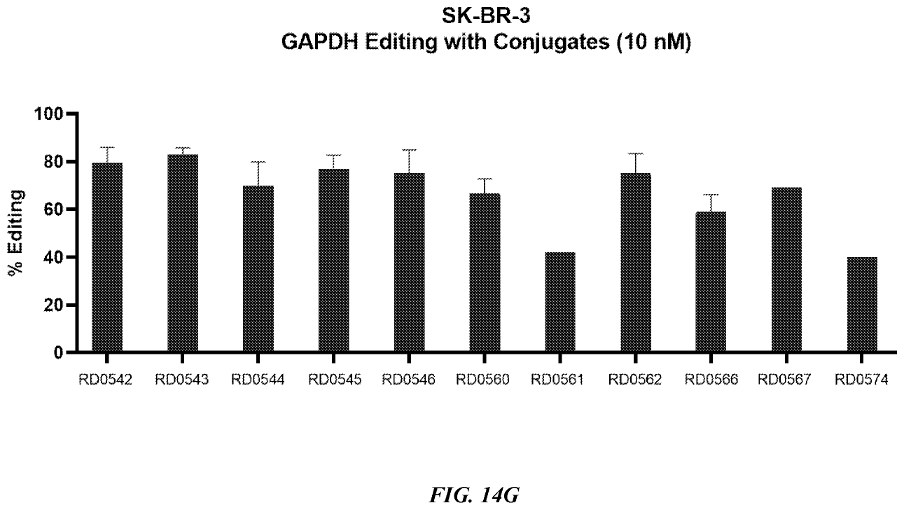

Results of the various oligo sequences are shown in FIGS. 13A-13C and FIGS. 14A-14G using various linkers, of which non-limiting examples are shown in FIGS. 12A-12G. Table 1 below lists the GAPDH editing efficiencies of the various compositions of oligo sequences as represented in FIGS. 14A-14F, for a 100 nM oligo transfection concentration.

TABLE 1
GAPDH editing efficiencies
of oligo compositions (100 nM)

| | Average | |
|---|---|---|
| | (%) | |
| | editing | |
| RD# | efficiency | StDev |
| RD0001 | 6 | 3.06 |
| RD0002 | 26 | 1.86 |
| RD0003 | 14 | 5.00 |
| RD0004 | 8 | 3.75 |
| RD0005 | 14 | 5.46 |
| RD0006 | 10 | 0.71 |
| RD0007 | 33 | 6.89 |
| RD0008 | 13 | 7.31 |
| RD0009 | 5 | 0.58 |
| RD0010 | 8 | 1.53 |
| RD0011 | 24 | 4.24 |
| RD0012 | 20 | 0.71 |
| RD0013 | 50 | 8.75 |
| RD0014 | 11 | 4.24 |
| RD0015 | 29 | 2.12 |
| RD0016 | 42 | 10.51 |
| RD0017 | 9 | 6.24 |
| RD0018 | 49 | 9.08 |
| RD0019 | 30 | 9.90 |
| RD0020 | 5 | 1.68 |
| RD0021 | 7 | 3.09 |
| RD0022 | 6 | 1.43 |
| RD0023 | 5 | 3.38 |
| RD0024 | 4 | 5.06 |
| RD0025 | 4 | 6.27 |
| RD0026 | 1 | 1.73 |
| RD0027 | 21 | 7.27 |
| RD0028 | 30 | 6.48 |
| RD0029 | 11 | 0.40 |
| RD0030 | 18 | 3.77 |
| RD0031 | 23 | 5.32 |
| RD0032 | 13 | 3.25 |
| RD0033 | 16 | 4.75 |
| RD0034 | 22 | 6.36 |
| RD0035 | 16 | 7.48 |
| RD0036 | 8 | 1.09 |
| RD0037 | 30 | 8.41 |
| RD0038 | 14 | 3.64 |
| RD0039 | 9 | 2.30 |
| RD0040 | 27 | 8.49 |
| RD0041 | 7 | 1.72 |
| RD0042 | 8 | 2.96 |
| RD0043 | 10 | 2.79 |
| RD0044 | 5 | 2.49 |
| RD0045 | 9 | 3.12 |
| RD0046 | 15 | 2.48 |
| RD0047 | 6 | 2.79 |
| RD0048 | 13 | 5.75 |
| RD0049 | 16 | 5.89 |
| RD0050 | 16 | 6.76 |
| RD0051 | 29 | 8.94 |
| RD0052 | 7 | 3.44 |
| RD0053 | 16 | 5.67 |
| RD0054 | 19 | 4.07 |
| RD0055 | 30 | 4.77 |
| RD0056 | 13 | 2.54 |
| RD0057 | 13 | 3.73 |
| RD0058 | 18 | 4.66 |
| RD0059 | 9 | 2.22 |
| RD0060 | 19 | 10.71 |
| RD0061 | 34 | 5.03 |
| RD0062 | 15 | 5.05 |
| RD0063 | 17 | 4.12 |
| RD0064 | 16 | 3.93 |
| RD0065 | 37 | 3.63 |
| RD0066 | 19 | 3.61 |
| RD0067 | 10 | 0.35 |
| RD0068 | 18 | 7.54 |
| RD0069 | 37 | 10.10 |
| RD0070 | 19 | 4.77 |
| RD0071 | 16 | 4.22 |
| RD0072 | 14 | 3.83 |
| RD0073 | 40 | 6.10 |
| RD0074 | 41 | 7.44 |
| RD0075 | 23 | 6.91 |
| RD0160 | 21 | 2.10 |
| RD0161 | 18 | 9.42 |
| RD0162 | 13 | 8.69 |
| RD0163 | 24 | 3.80 |
| RD0164 | 24 | 5.71 |
| RD0165 | 30 | 3.52 |
| RD0166 | 33 | 6.18 |
| RD0167 | 38 | 2.85 |
| RD0168 | 52 | 4.28 |
| RD0169 | 1 | 1.41 |
| RD0170 | 37 | 3.45 |
| RD0171 | 20 | 9.19 |
| RD0172 | 13 | 2.83 |
| RD0173 | 16 | 9.64 |
| RD0174 | 25 | 2.88 |
| RD0175 | 26 | 8.83 |
| RD0176 | 23 | 5.58 |
| RD0177 | 18 | 0.83 |
| RD0178 | 62 | 2.53 |
| RD0179 | 51 | 7.78 |
| RD0180 | 51 | 4.95 |
| RD0196 | 56 | 5.21 |
| RD0197 | 52 | 2.88 |
| RD0198 | 70 | 1.90 |
| RD0199 | 33 | 4.24 |
| RD0200 | 30 | 5.66 |
| RD0201 | 19 | 4.95 |
| RD0202 | 47 | 4.91 |
| RD0203 | 40 | 13.74 |
| RD0204 | 49 | 1.54 |
| RD0205 | 32 | 9.19 |
| RD0206 | 61 | 2.91 |
| RD0207 | 58 | 4.00 |
| RD0209 | 47 | 8.27 |
| RD0210 | 52 | 2.73 |
| RD0211 | 38 | 18.27 |
| RD0212 | 13 | 7.01 |
| RD0213 | 33 | 5.19 |
| RD0215 | 33 | 4.83 |
| RD0216 | 28 | 8.97 |
| RD0217 | 45 | 14.78 |
| RD0218 | 50 | 2.84 |
| RD0220 | 57 | 1.48 |
| RD0221 | 19 | 19.29 |
| RD0222 | 17 | 4.66 |
| RD0223 | 7 | 0.28 |
| RD0224 | 55 | 1.70 |
| RD0225 | 57 | 1.79 |
| RD0226 | 60 | 0.16 |
| RD0227 | 53 | 12.99 |
| RD0228 | 22 | 10.59 |
| RD0229 | 17 | 0.75 |
| RD0230 | 23 | 11.08 |
| RD0231 | 5 | N/A |
| RD0232 | 6 | 0.92 |
| RD0233 | 5 | 0.51 |
| RD0234 | 7 | 0.81 |
| RD0235 | 6 | 0.71 |
| RD0236 | 10 | 5.55 |
| RD0237 | 20 | 15.57 |
| RD0238 | 13 | 3.98 |
| RD0239 | 11 | 1.37 |
| RD0240 | 8 | 0.10 |
| RD0241 | 51 | 3.76 |
| RD0242 | 20 | 5.57 |
| RD0243 | 8 | 2.37 |
| RD0244 | 31 | 7.44 |
| RD0245 | 39 | 9.90 |
| RD0246 | 60 | 4.17 |
| RD0344 | 21 | 3.49 |
| RD0345 | 31 | 8.92 |
| RD0346 | 43 | 4.95 |
| RD0347 | 29 | 5.43 |
| RD0348 | 28 | 4.40 |
| RD0349 | 37 | 7.49 |
| RD0441 | 54 | 2.38 |
| RD0442 | 61 | 0.67 |
| RD0452 | 70 | 8.65 |
| RD0453 | 64 | 13.38 |
| RD0454 | 48 | 5.56 |
| RD0455 | 68 | 0.58 |
| RD0456 | 52 | 2.46 |
| RD0457 | 69 | 10.08 |
| RD0458 | 64 | 3.11 |
| RD0459 | 52 | 3.89 |
| RD0461 | 57 | 1.65 |
| RD0463 | 64 | N/A |
| RD0464 | 60 | N/A |
| RD0465 | 39 | N/A |
| RD0466 | 58 | 7.74 |
| RD0467 | 57 | 7.70 |
| RD0468 | 59 | 12.40 |
| RD0473 | 45 | 11.77 |
| RD0474 | 40 | 4.61 |
| RD0475 | 35 | 1.49 |
| RD0476 | 42 | 10.87 |
| RD0477 | 34 | N/A |
| RD0478 | 28 | 1.06 |
| RD0479 | 46 | 8.29 |
| RD0480 | 61 | 15.28 |
| RD0481 | 48 | 11.94 |
| RD0482 | 46 | 13.98 |
| RD0483 | 67 | 11.88 |
| RD0484 | 76 | N/A |
| RD0485 | 65 | 13.52 |

Table 2 below shows the GAPDH editing efficiencies of various compositions comprising conjugated oligos, with a 10 nM oligo transfection concentration.

TABLE 2
GAPDH editing efficiencies of oligo compositions (10 nM)

| | Average editing | |
|---|---|---|
| RD# | efficiency (%) | StDev |
| RD0542 | 73 | 7.92 |
| RD0543 | 83 | 2.83 |
| RD0544 | 70 | 9.90 |
| RD0545 | 71 | 10.60 |
| RD0546 | 75 | 7.00 |
| RD0560 | 61 | 6.54 |
| RD0561 | 42 | 0.50 |
| RD0562 | 73 | 5.25 |
| RD0566 | 59 | 7.07 |
| RD0567 | 71 | 1.91 |
| RD0574 | 53 | 7.77 |

### Example 3: A to I RNA Editing in Murine Primary Hepatocyte on Mouse Glyceraldehyde 3-Phosphate Dehydrogenase (GAPDH) mRNA

Mouse hepatocytes were transfected with 100 μM oligonucleotide complexed with 0.5 μL Lipofectamine RNAiMAx (Invitrogen) for 24 h. Total RNA was extracted from hepatocytes with RNAqueous Kit according to the manufacturer's protocol (Invitrogen). Complementary DNA was synthesized, and DNA amplified, using the SuperScript One-Step RT-PCR Kit (Invitrogen) and specific primers for human GAPDH. The resulting DNA was purified using MinElute 96UF plates (Qiagen) and analyzed by Sanger sequencing (Genewiz). Adenosine-to-inosine editing yields were quantified by measuring the height of the guanosine and adenosine peaks at the respective site and dividing the guanosine peak height by the sum of the guanosine and adenosine peak heights.

Results

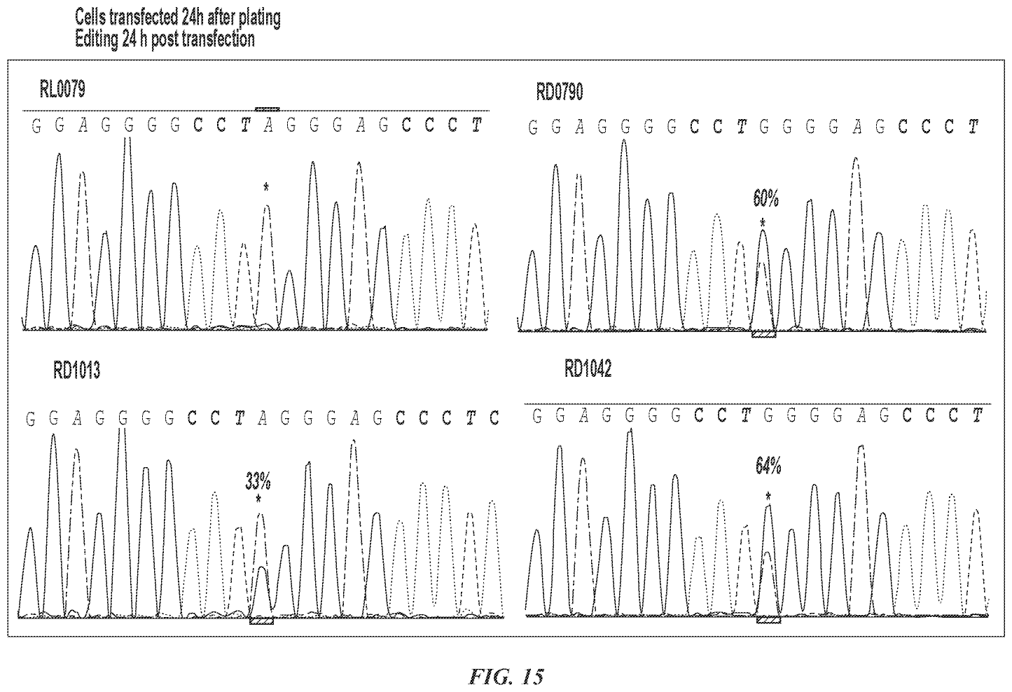

Results from sequencing are shown in FIG. 15 and summarized in Table 3A below.

TABLE 3A
GAPDH editing efficiencies in murine primary hepatocytes

| Compound ID | Editing Efficiency | Target Gene | Species |
|---|---|---|---|
| RL0079 | 0% | Negative control | Negative control |
| RD0790 | 60% | GAPDH | mouse |
| RD1013 | 33% | GAPDH | mouse |
| RD1042 | 64% | GAPDH | mouse |

Table 3B below details the sequences and structures of the different compounds used.

TABLE 3B
Compound information

| | | Linker | | |
|---|---|---|---|---|
| | | between | | |
| | | editing | | |
| | Editing domain on long | domain and | | |
| | strand sequence | recruiting | Sequence | |
| Conjugate ID | information 5′- 3′ | domain | (<Strand Ref.: ##>) | Description |
| RD0790 | (X1)*mG*mG*mC.mU.mC. | PEG2 | (X1)*mG*mG*mC.mU.mC. | Editing domain |
| | mC.rC*rC*rA.mG.mG. | | mC.rC*rC*rA.mG.mG.mC. | on long strand |
| | mC.mC.mC*mC*mU*m | | mC.mC*mC*mU*mC*mC | sequence |
| | C*mC (SEQ ID NO: 114) | | (<Strand Ref.: 819>)(SEQ | information 5′- |
| | | | ID NO: 114) | 3′ of |
| | | | | composition |
| | | | | RD0790 with |
| | | | | linker = PEG2 |
| RD1013 | (X1)*mG*mG*mC.mU.mC. | PEG2 | (X1)*mG*mG*mC.mU.mC. | Editing domain |
| | mC.rC*rC*rA.mG.mG. | | mC.rC*rC*rA.mG.mG.mC. | on long strand |
| | mC.mC.mC*mC*mU*mC* | | mC.mC*mC*mU*mC*mC | sequence |
| | mC (SEQ ID NO: 114) | | (<Strand Ref.: 820>)(SEQ | information 5′- |
| | | | ID NO: 114) | 3′ of |
| | | | | composition |
| | | | | RD1013 with |
| | | | | linker = PEG2 |
| RD1042 | (X1)*mG*mG*mC.mU.mC. | PEG6 | (X1)*mG*mG*mC.mU.mC. | Editing domain |
| | mC.rC*rC*rA.mG.mG. | | mC.rC*rC*rA.mG.mG.mC. | on long strand |
| | mC.mC.mC*mC*mU*mC* | | mC.mC*mC*mU*mC*mC | sequence |
| | mC (SEQ ID NO: 114) | | (<Strand Ref.: 821>)(SEQ | information 5′- |
| | | | ID NO: 114) | 3′ of |
| | | | | composition |
| | | | | RD1042 with |
| | | | | linker = PEG6 |

### Example 4: A to I RNA Editing in Primary Monkey Liver Fibroblasts on Monkey Glyceraldehyde 3-Phosphate Dehydrogenase (GAPDH) mRNA

MK-6019 cells (cynomolgus monkey primary liver fibroblasts) were cultured in Complete Fibroblast Growth medium obtained from Cell Biologics (Cat #M2267) at 37° C. in an atmosphere of 5% CO₂. The cells were acquired from Cell Biologics. Cells were seeded in 96-well plates at 2×10⁵ cells/well in 100 μL of growth medium without antibiotics one day prior to the transfection. The next day, cells were transfected with 10 nM of GAPDH RNA oligonucleotide complexed with 0.6 μL Lipofectamaine RNAiMAX. After 24 hours (h), cells were harvested and RNA extracted using the RNAqueous-96 Kit (ThermoFisher Cat #AM1920). Reverse transcription was performed using SuperScript IV (Invitrogen) followed by PCR with Platinum II Taq polymerase (ThermoFisher). The resulting DNA was purified on MinElute 96 UF PCR Purification Kit (QIAGEN Cat #28051) and analyzed by Sanger sequencing (Genewiz).

Results

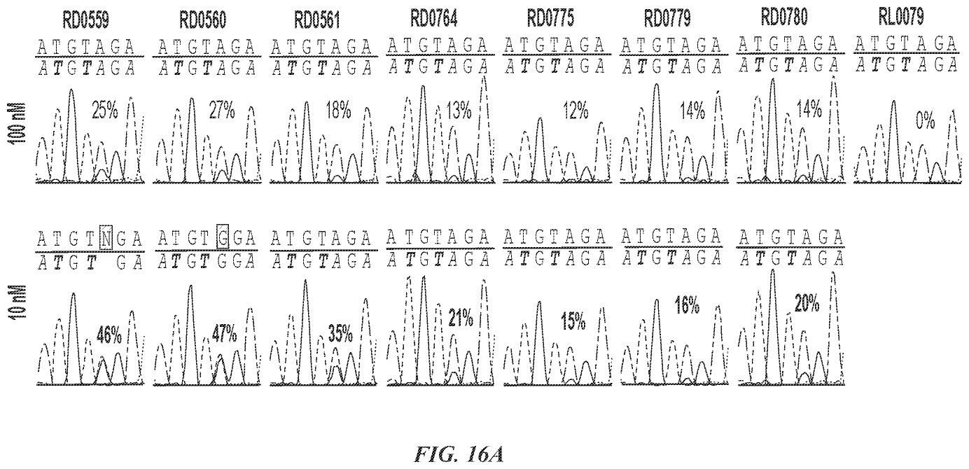

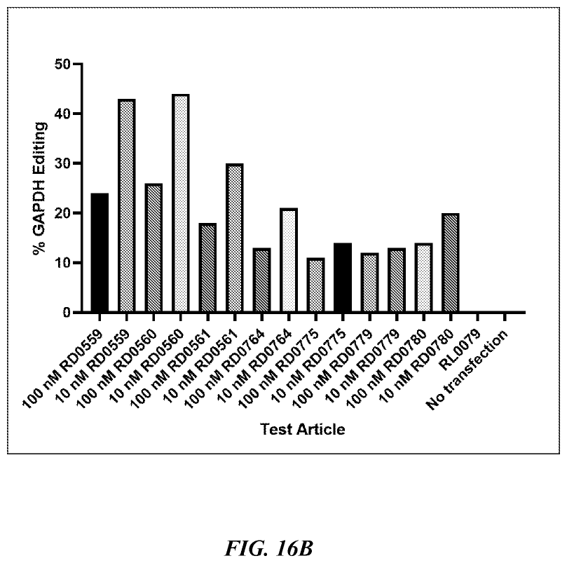

Results from sequencing are shown in FIG. 16A. The GAPDH editing efficiencies for the compounds are shown in FIG. 16B and summarized in Table 4A below.

TABLE 4A
GAPDH editing efficiencies in MK-6019 cells

| | Editing | Editing | | |
|---|---|---|---|---|
| Compound | Efficiency at | Efficiency | Target | |
| ID | 100 nM | at 10 nM | Gene | Species |
| RL0079 | 0% | | Negative | Negative |
| | | | control | control |
| RD0559 | 24% | 43% | GAPDH | Cyno monkey |
| RD0560 | 26% | 44% | GAPDH | Cyno monkey |
| RD0561 | 18% | 30% | GAPDH | Cyno monkey |
| RD0764 | 13% | 21% | GAPDH | Cyno monkey |
| RD0775 | 11% | 14% | GAPDH | Cyno monkey |
| RD0779 | 12% | 13% | GAPDH | Cyno monkey |
| RD0780 | 14% | 20% | GAPDH | Cyno monkey |

Table 4B below details the sequences and structures of the different compounds used.

TABLE 4B
Compound information

| | | Linker | | |
|---|---|---|---|---|
| | | between | | |
| | | editing | short strand | |
| | Editing domain on long | domain and | complementary to | Recruiting domain on |
| | strand sequence | recruiting | recruiting domain | long strand, sequence |
| Conjugate ID | information 5′- 3′ | domain | on Long strand 5′-3′ | information 5′-3′ |
| RD0559 | (X1)*mA*mG*mG.mG. | PEG2 | mG*mG*mU*fG*mU. | mA*fU*rG.fU.mU.rG. |
| | mG.mU.rC.rC.rA.mC.mA. | | mC.fG.fA.fG.fA.fA. | mU.mU.mC.mU.fC.rG. |
| | mU.mG.mG*mA*mA*mA* | | fG.fA.fG.fG.fA.fG. | fU.mC.fU.rC.fC.mU.mC. |
| | mC (SEQ ID NO: 113) | | fA.fA.fC.mA*fA*mU* | rG.rA.mC.rA*mC*mC* |
| | | | fA*mU (SEQ ID | (X2)(SEQ ID NO: |
| | | | NO: 3) | 4) |
| RD0560 | (X1)*mA*mG*mG.mG. | PEG2 | mG*mG*mU*fG*mU. | mA*fU*rG.fU.mU.rG. |
| | mG.mU.rC*rC*rA.mC.mA. | | mC.fG.fA.fG.fA.fA. | mU.mU.mC.mU.fC.rG. |
| | mU.mG.mG*mA*mA* | | fG.fA.fG.fG.fA.fG. | fU.mC.fU.rC.fC.mU. |
| | mA*mC (SEQ ID NO: | | fA.fA.fC.mA*fA*mU* | mC.rG.rA.mC.rA*mC* |
| | 113) | | fA*mU (SEQ ID | mC*(X2)(SEQ ID NO: |
| | | | NO: 3) | 4) |
| RD0561 | (X1)*mA*mG*mG.mG. | PEG2 | mG*mG*mU*fG*mU. | mA*fU*rG.fU.mU.rG. |
| | mG.mU.dC*dC*dA.mC. | | mC.fG.fA.fG.fA.fA. | mU.mU.mC.mU.fC.rG. |
| | mA.mU.mG.mG*mA*mA* | | fG.fA.fG.fG.fA.fG. | fU.mC.fU.rC.fC.mU. |
| | mA*mC (SEQ ID NO: | | fA.fA.fC.mA*fA*mU* | mC.rG.rA.mC.rA*mC* |
| | 113) | | fA*mU (SEQ ID | mC*(X2)(SEQ ID NO: |
| | | | NO: 3) | 4) |
| RD0764 | (X1)*mA*mG*mG.mG. | PEG2 | mG*mG*mU*fG*mU. | mU*fG*mU*mU*mC. |
| | mG.mU.rC*rC*rA.mC.mA. | | mC.fG.fA.fG.fA.fA. | mU.fC.fG.fU.mC.fU.fC. |
| | mU.mG.mG*mA*mA* | | fG.fA.fG.fG.fA.fG* | fC.mU.mC.fG.fA.mC* |
| | mA*mC (SEQ ID NO: | | fA*fA*fC*mA | fA*mC*mC*(X2) |
| | 113) | | (SEQ ID NO: 53) | (SEQ ID NO: 116) |
| RD0775 | (X1)*mA*mG*mG.mG. | PEG2 | mG*mG*mU*fG*mU. | mG*mC*mA*fU*fA.fU. |
| | mG.mU.rC*rC*rA.mC.mA. | | mC.fG.fA.fG.fA.fA. | mU.fG.mU.mU.mC. |
| | mU.mG.mG*mA*mA* | | fG.fA.fG.fG.fA.fG. | mU.fC.fG.fU.mC.fU.fC. |
| | mA*mC (SEQ ID NO: | | fA.fA.fC.mA*fA*mU* | fC.mU.mC.fG.fA.mC* |
| | 113) | | fA*mU (SEQ ID | fA*mC*mC*(X2) |
| | | | NO: 3) | (SEQ ID NO: 117) |
| RD0779 | (X1)*mA*mG*mG.mG. | PEG2 | mG*mG*mU*fG*mG. | mU*fG*mU*mU*mC. |
| | mG.mU.rC*rC*rA.mC.mA. | | mG.fU.fG.fG.fA.fA. | mU.fC.fG.fU.mC.fU. |
| | mU.mG.mG*mA*mA* | | fG.fA.fG.fG.fA.fG* | mC.fC.mC.mA.fC.fC.mC* |
| | mA*mC (SEQ ID NO: | | fA*fA*fC*mA | fA*mC*mC*(X2) |
| | 113) | | (SEQ ID NO: 84) | (SEQ ID NO: 115) |
| RD0780 | (X1)*mA*mG*mG.mG. | PEG2 | mG*mG*mU*fG*mG. | mU*mU*fG*mU*mU. |
| | mG.mU.rC*rC*rA.mC.mA. | | mG.fU.fG.fG.fA.fA. | mC.mU.fC.fG.fU.mC.fU. |
| | mU.mG.mG*mA*mA* | | fG.fA.fG.fG.fA.fG* | mC.fC.mC.mA.fC.fC. |
| | mA*mC (SEQ ID NO: | | fA*fA*fC*mA | mC*fA*mC*mC*(X2) |
| | 113) | | (SEQ ID NO: 84) | (SEQ ID NO: 118) |

### Example 5: A to I RNA Editing in Transgenic Mouse Hepatocytes on a Target mRNA Molecule that Codes for a Human Protein Mutational Variant

Primary Mouse Hepatocyte Isolation

Transgenic mice expressing a human protein mutational variant (e.g., a protein comprising a one-amino-acid point mutation compared to the wild-type protein) were obtained from colonies maintained at Saint Louis University. A two-step collagenase perfusion was performed to isolate primary hepatocytes from mouse livers. Briefly, a needle was inserted into the portal vein, and then 25 mL of pre-perfusion solution (HBSS) was injected by using a peristaltic pump at 6 mL/min. Then, the liver was perfused with the perfusion solution containing collagenase by using a peristaltic pump at 3 mL/min. After collagenase perfusion, digested tissue is further shredded with scissors and the cell suspension was centrifuged at 50×g for 3 min. The pellet was suspended in M199 medium and cells plated on collagen-coated 96 well plates (˜2×10⁴ cells in 200 μL medium).

Target RNA Editing

Mouse hepatocytes were transfected with 100 μM oligonucleotide complexed with 0.5 μL Lipofectamine RNAiMAx (Invitrogen) for 24 h. Total RNA was extracted from hepatocytes with RNAqueous Kit according to the manufacturer's protocol (Invitrogen). Complementary DNA was synthesized and DNA amplified using the SuperScript One-Step RT-PCR Kit (Invitrogen) and specific primers for the human protein. The resulting DNA was purified using MinElute 96UF plates (Qiagen) and analyzed by Sanger sequencing (Genewiz). Adenosine-to-inosine editing yields were quantified by measuring the height of the guanosine and adenosine peaks at the respective site and dividing the guanosine peak height by the sum of the guanosine and adenosine peak heights.

Results

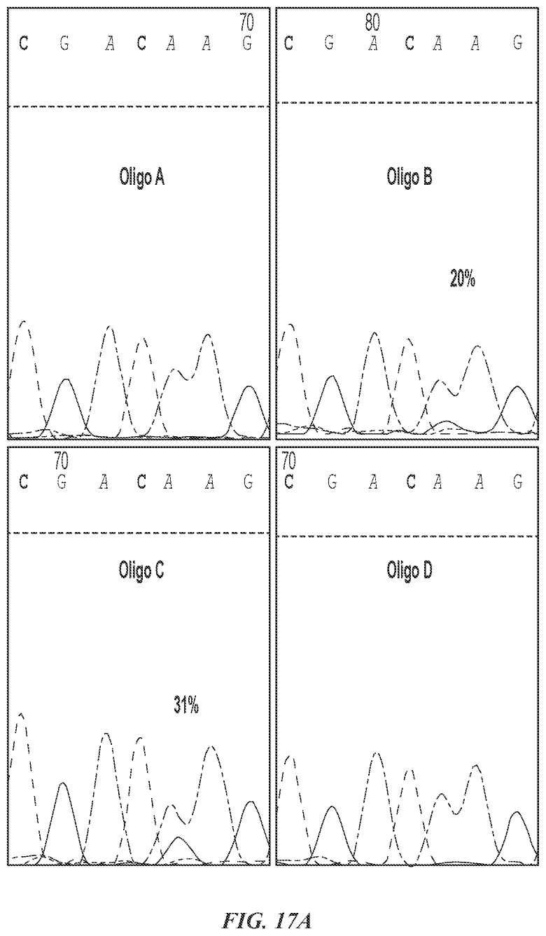

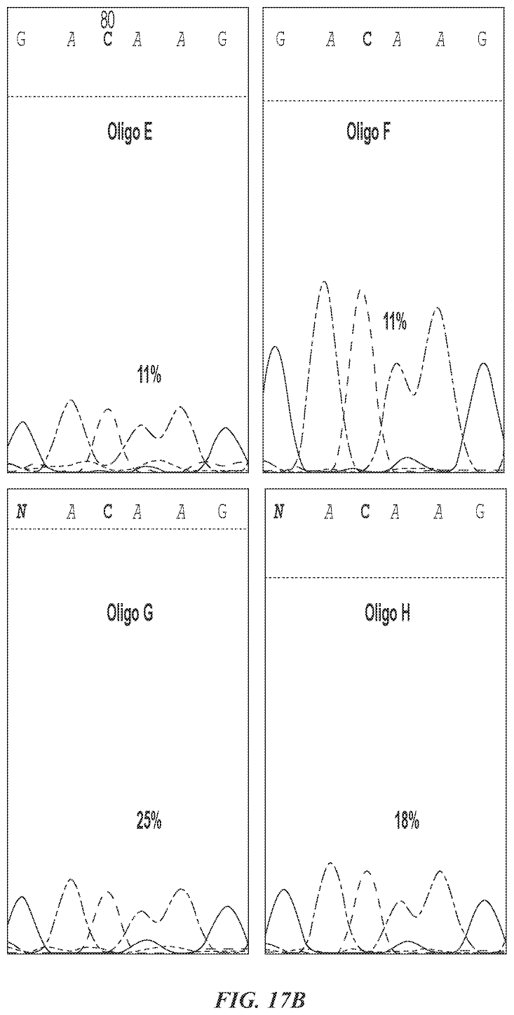

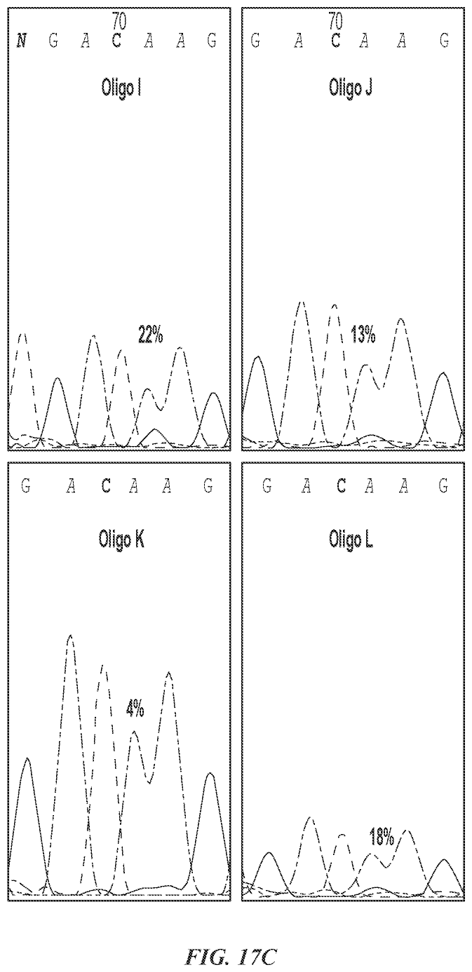

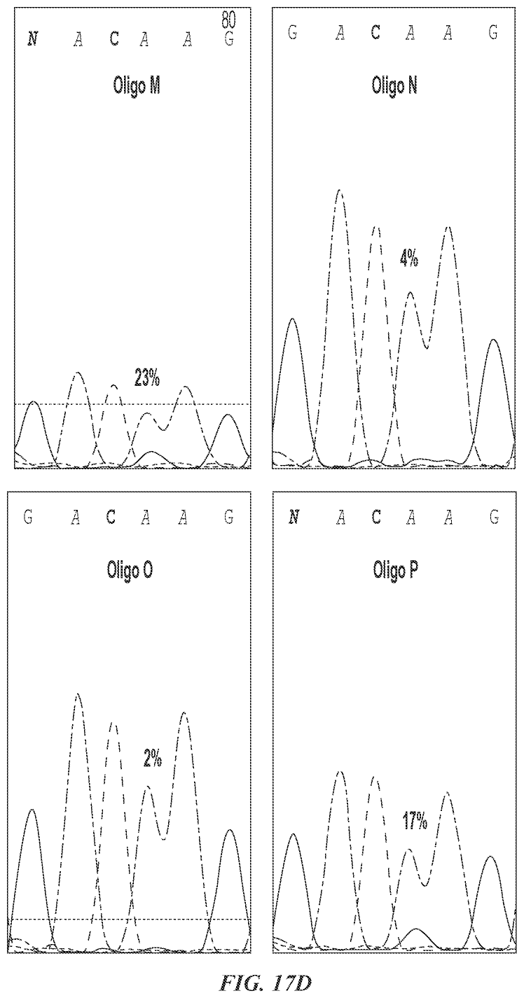

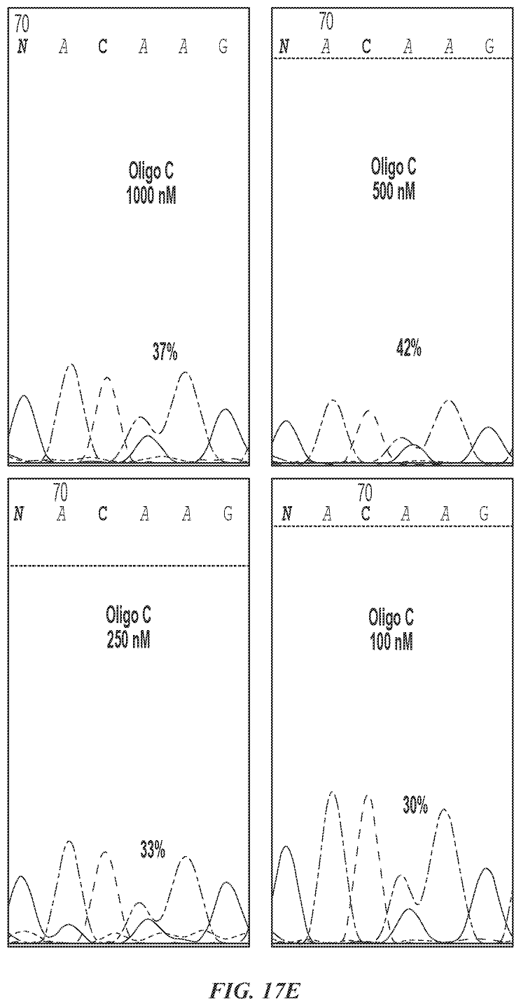

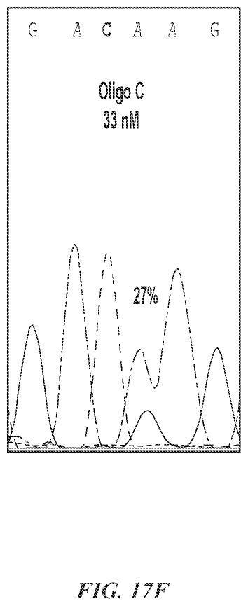

Results from sequencing are shown in FIGS. 17A-17F and summarized in Table 5 below.

TABLE 5
Compound information and Target RNA Editing Efficiencies

| | Linker | | | |
|---|---|---|---|---|
| | between | | | |
| | editing | short strand | | |
| | domain and | complementary to | Recruiting domain on | Editing |
| | recruiting | recruiting domain | long strand, sequence | Efficiency at |
| Conjugate ID | domain | on Long strand 5′-3′ | information 5′-3′ | 100 nM |
| Oligo O | PEG2 | mG*fG*mG*fU*mG. | mG*fC*mA*fU*mA.fU. | 2% |
| | | fG.fA.fA.mG.fA.fG. | mU.fG.mU.fU.mC.fU. | |
| | | fU.mA.fG.mA.fA. | mA.fG.mU.fC.mU. | |
| | | mC.fA.mA.fU*mA*fU* | mC.mC.fC.mA*fC*mC* | |
| | | mG*mC (SEQ ID | fC*(X2)(SEQ ID | |
| | | NO: 5) | NO: 6) | |
| Oligo D | PEG2 | mG*fG*mU*fG*mU. | mA*fU*mG*fU*mU.fG. | nd |
| | | fC.mG.fA.mG.fA. | mU.fU.mC.fU.mC.fG. | |
| | | mA.fG.mA.fG.mG.fA. | mU.fC.mU.mC.mC. | |
| | | mG.fA.mA.fC.mA* | fU.mC.fG.mA.fC*mA* | |
| | | fA*mU*fA*mU | fC*mC*(X2)(SEQ ID | |
| | | (SEQ ID NO: 3) | NO: 4) | |
| Oligo H | PEG2 | mG*mG*mU*fG* | mA*fU*fA.fU.mU.fG. | 18% |
| | | mU.mC.fG.fA.fG.fA. | mU.mU.mC.mU.fC.fG. | |
| | | fA.fG.fA.fG.fG.fA.fG. | fU.mC.fU.fC.fC.mU. | |
| | | fA.fA.fC.mA*fA*mU* | mC.fG.fA.mC.fA*mC* | |
| | | fA*mU (SEQ ID | mC*(X2)(SEQ ID NO: | |
| | | NO: 3) | 168) | |
| Oligo M | PEG2 | mG*mG*mU*fG* | mA*fU*fA.fU.mU.fG. | 23% |
| | | mU.mC.fG.fA.fG.fA. | mU.mU.mC.mU.fC.fG. | |
| | | fA.fG.fA.fG.fG.fA.fG. | fU.mC.fU.mC.fC.mU. | |
| | | fA.fA.fC.mA*fA*mU* | mC.fG.fA.mC.fA*mC* | |
| | | fA*mU (SEQ ID | mC*(X2)(SEQ ID NO: | |
| | | NO: 3) | 168) | |
| Oligo Q | PEG2 | mG*mG*mU*fG* | mA*fU*mA*fU*mU.fG. | nd |
| | | mU.mC.fG.fA.fG.fA. | mU.fU.mC.fU.mC.fG. | |
| | | fA.fG.fA.fG.fG.fA.fG. | mU.fC.mU.mC.mC. | |
| | | fA.fA.fC.mA*fA*mU* | fU.mC.fG.mA.fC*mA* | |
| | | fA*mU (SEQ ID | fC*mC*(X2)(SEQ ID | |
| | | NO: 3) | NO: 168) | |
| Oligo P | PEG2 | mG*mG*mU*fG* | mA*fU*fG.fU.mU.fG. | 17% |
| | | mU.mC.fG.fA.fG.fA. | mU.mU.mC.mU.fC.fG. | |
| | | fA.fG.fA.fG.fG.fA.fG. | fU.mC.fU.fC.fC.mU. | |
| | | fA.fA.fC.mA*fA*mU* | mC.fG.fA.mC.fA*mC* | |
| | | fA*mU (SEQ ID | mC*(X2)(SEQ ID NO: | |
| | | NO: 3) | 4) | |
| Oligo E | PEG2 | mG*fG*mU*fG*mU. | H1.mA*fU*mG*fU* | 11% |
| | | fC.mG.fA.mG.fA. | mU.fG.mU.fU.mC.fU. | |
| | | mA.fG.mA.fG.mG.fA. | mC.fG.mU.fC.mU.mC. | |
| | | mG.fA.mA.fC.mA | mC.fU.mC.fG.mA.fC* | |
| | | *fA*mU*fA*mU | mA*fC*mC*(X2) | |
| | | (SEQ ID NO: 3) | (SEQ ID NO: 4) | |
| Oligo I | PEG2 | mG*fG*mG*fU*mG. | H1.mG*fC*mA*fU*mA. | 22% |
| | | fG.fA.fA.mG.fA.fG. | fU.mU.fG.mU.fU.mC. | |
| | | fU.mA.fG.mA.fA. | fU.mA.fG.mU.fC.mU. | |
| | | mC.fA.mA.fU*mA*fU* | mC.mC.fC.mA*fC* | |
| | | mG*mC (SEQ ID | mC*fC*(X2)(SEQ ID | |
| | | NO: 5) | NO: 6) | |

### Example 6. Evaluation of RNA Editing Constructs in Human Fibroblasts

H-6019 cells (human primary liver fibroblasts) were cultured in Complete Fibroblast Growth medium obtained from Cell Biologics (Cat #M2267) at 37° C. in an atmosphere of 5% CO₂. The cells were acquired from Cell Biologics. Cells were seeded in 96-well plates at 2×10⁵ cells/well in 100 μL of growth medium without antibiotics one day prior to the transfection. The next day, cells were transfected with 10 nM of GAPDH RNA oligonucleotide complexed with 0.6 μL Lipofectamaine RNAiMAX. After 24 hours (h), cells were harvested and RNA extracted using the RNAqueous-96 Kit (ThermoFisher Cat #AM1920). Reverse transcription was performed using SuperScript IV (Invitrogen) followed by PCR with Platinum II Taq polymerase (ThermoFisher). The resulting DNA was purified on MinElute 96 UF PCR Purification Kit (QIAGEN Cat #28051) and analyzed by Sanger sequencing (Genewiz).

Results

The GAPDH editing efficiencies and compound information for the compounds are summarized below in Tables 6A and 6B, respectively.

TABLE 6A
GAPDH editing efficiencies in H-6019 cells

| | Editing Efficiency | Editing Efficiency | |
|---|---|---|---|
| Compound ID | 10 nM_1 | 10 nM_2 | Target Gene |
| RD0542 | 59% | 57% | GAPDH |
| RD0560 | 50% | 49% | GAPDH |
| RD0562 | 59% | 60% | GAPDH |
| RD0750 | 23% | 23% | GAPDH |
| RD0752 | 29% | 25% | GAPDH |
| RD0754 | 16% | 17% | GAPDH |
| RD0755 | 57% | 58% | GAPDH |
| RD0756 | 22% | 25% | GAPDH |
| RD0764 | 14% | 14% | GAPDH |
| RD0765 | 5% | 4% | GAPDH |
| RD0766 | 12% | 11% | GAPDH |
| RD0767 | 8% | 9% | GAPDH |
| RD0774 | 19% | 14% | GAPDH |
| RD0775 | 19% | 15% | GAPDH |
| RD0779 | 15% | 15% | GAPDH |
| RD0780 | 15% | 16% | GAPDH |
| RD0995 | 28% | 24% | GAPDH |
| RD0996 | 22% | 20% | GAPDH |
| RD0997 | 21% | 21% | GAPDH |
| RD0999 | 22% | 19% | GAPDH |
| RD1014 | *0% | *0% | GAPDH |
| RD1015 | *0% | 3% | GAPDH |

*Editing efficiency below detectable limit

TABLE 6B
Compound information

| | | Linker | | |
|---|---|---|---|---|
| | | between | | |
| | | editing | short strand | |
| | Editing domain on long | domain and | complementary to | Recruiting domain on |
| | strand sequence | recruiting | recruiting domain | long strand, sequence |
| Conjugate ID | information 5′- 3′ | domain | on Long strand 5′-3′ | information 5′-3′ |
| RD0542 | (X1)*mA*mG*mG.mG. | PEG2 | mG*mG*mU*fG* | mA*fU*rG.fU.mU.rG. |
| | mG.mU.rC.rC.rA.mC.mA. | | mU.mC.fG.fA.fG.fA. | mU.mU.mC.mU.fC.rG. |
| | mU.mG.mG*mC*mA* | | fA.fG.fA.fG.fG.fA.fG. | fU.mC.fU.rC.fC.mU. |
| | mA*mC (SEQ ID NO: | | fA.fA.fC.mA*fA*mU* | mC.rG.rA.mC.rA*mC* |
| | 1) | | fA*mU (SEQ ID | mC*(X2)(SEQ ID |
| | | | NO: 3) | NO: 4) |
| RD0560 | (X1)*mA*mG*mG.mG. | PEG2 | mG*mG*mU*fG* | mA*fU*rG.fU.mU.rG. |
| | mG.mU.rC*rC*rA.mC. | | mU.mC.fG.fA.fG.fA. | mU.mU.mC.mU.fC.rG. |
| | mA.mU.mG.mG*mA*mA* | | fA.fG.fA.fG.fG.fA.fG. | fU.mC.fU.rC.fC.mU. |
| | mA*mC (SEQ ID NO: | | fA.fA.fC.mA*fA*mU* | mC.rG.rA.mC.rA*mC* |
| | 113) | | fA*mU (SEQ ID | mC*(X2)(SEQ ID |
| | | | NO: 3) | NO: 4) |
| RD0562 | (X1)*mA*mG*mG.mG. | PEG2 | mG*mG*mU*fG* | mA*fU*rG.fU.mU.rG. |
| | mG.mU.rC*rC*rA.mC. | | mU.mC.fG.fA.fG.fA. | mU.mU.mC.mU.fC.rG. |
| | mA.mU.mG.mG*mC*mA* | | fA.fG.fA.fG.fG.fA.fG. | fU.mC.fU.rC.fC.mU. |
| | mA*mC (SEQ ID NO: | | fA.fA.fC.mA*fA*mU* | mC.rG.rA.mC.rA*mC* |
| | 1) | | fA*mU (SEQ ID | mC*(X2)(SEQ ID |
| | | | NO: 3) | NO: 4) |
| RD0750 | mA*mG*mG*mG*mG. | PEG2 | mG*mG*mU*fG* | mA*fU*rG.fU.mU.rG. |
| | mU.rC.rC.rA.mC.mA.mU. | | mU.mC.fG.fA.fG.fA. | mU.mU.mC.mU.fC.rG. |
| | mG.mG.mC*mA*mA* | | fA.fG.fA.fG.fG.fA.fG. | fU.mC.fU.rC.fC.mU. |
| | mC*(X1)(SEQ ID NO: | | fA.fA.fC.mA*fA*mU* | mC.rG.rA.mC.rA*mC* |
| | 1) | | fA*mU (SEQ ID | mC*(X2)(SEQ ID |
| | | | NO: 3 | NO: 4) |
| RD0752 | (X1)*mA*mG*mG.mG. | PEG2 | mG*mG*mU*fG* | mU*fG*mU*mU*mC. |
| | mG.mU.rC.rC.rA.mC.mA. | | mU.mC.fG.fA.fG.fA. | mU.fC.fG.fU.mC.fU.fC. |
| | mU.mG.mG*mC*mA* | | fA.fG.fA.fG.fG.fA.fG. | fC.mU.mC.fG.fA.mC* |
| | mA*mC (SEQ ID NO: | | *fA*fA*fC*mA | fA*mC*mC*(X2) |
| | 1) | | (SEQ ID NO: 53) | (SEQ ID NO: 116) |
| RD0754 | (X1)*mA*mG*mG.mG. | C11 | mG*mG*mU*fG* | mA*fU*rG.fU.mU.rG. |
| | mG.mU.rC.rC.rA.mC.mA. | | mU.mC.fG.fA.fG.fA. | mU.mU.mC.mU.fC.rG. |
| | mU.mG.mG*mC*mA* | | fA.fG.fA.fG.fG.fA.fG. | fU.mC.fU.rC.fC.mU. |
| | mA*mC (SEQ ID NO: | | fA.fA.fC.mA*fA*mU* | mC.rG.rA.mC.rA*mC* |
| | 1) | | fA*mU (SEQ ID | mC*(X2)(SEQ ID |
| | | | NO: 3) | NO: 4) |
| RD0755 | (X1)*mA*mG*mG.mG. | Ph | mG*mG*mU*fG* | mA*fU*rG.fU.mU.rG. |
| | mG.mU.rC.rC.rA.mC.mA. | | mU.mC.fG.fA.fG.fA. | mU.mU.mC.mU.fC.rG. |
| | mU.mG.mG*mC*mA* | | fA.fG.fA.fG.fG.fA.fG. | fU.mC.fU.rC.fC.mU. |
| | mA*mC (SEQ ID NO: | | fA.fA.fC.mA*fA*mU* | mC.rG.rA.mC.rA*mC* |
| | 1) | | fA*mU (SEQ ID | mC*(X2)(SEQ ID |
| | | | NO: 3) | NO: 4) |
| RD0756 | (X1)*mA*mG*mG.mG. | Cy | mG*mG*mU*fG* | mA*fU*rG.fU.mU.rG. |
| | mG.mU.rC.rC.rA.mC.mA. | | mU.mC.fG.fA.fG.fA. | mU.mU.mC.mU.fC.rG. |
| | mU.mG.mG*mC*mA* | | fA.fG.fA.fG.fG.fA.fG. | fU.mC.fU.rC.fC.mU. |
| | mA*mC (SEQ ID NO: | | fA.fA.fC.mA*fA*mU* | mC.rG.rA.mC.rA*mC* |
| | 1) | | fA*mU (SEQ ID | mC*(X2)(SEQ ID |
| | | | NO: 3) | NO: 4) |
| RD0764 | (X1)*mA*mG*mG.mG. | PEG2 | mG*mG*mU*fG* | mU*fG*mU*mU*mC. |
| | mG.mU.rC*rC*rA.mC. | | mU.mC.fG.fA.fG.fA. | mU.fC.fG.fU.mC.fU.fC. |
| | mA.mU.mG.mG*mA*mA* | | fA.fG.fA.fG.fG.fA.fG* | fC.mU.mC.fG.fA.mC* |
| | mA*mC (SEQ ID NO: | | fA*fA*fC*mA | fA*mC*mC*(X2) |
| | 113) | | (SEQ ID NO: 53) | (SEQ ID NO: 116) |
| RD0765 | (X1)*mA*mG*mG.mG. | PEG2 | mG*mG*mU*fG* | mU*fG*mU*mU*mC. |
| | mG.mU.rC*rC*rA.mC. | | mU.mC.fG.fA.fG.fA. | mU.fC.fG.fU.mC.fU.fC. |
| | mA.mU.mG.mG*mC*mA* | | fA.fG.fA.fG.fG.fA.fG* | fC.mU.mC.fG.fA.mC* |
| | mA*mC (SEQ ID NO: | | fA*fA*fC*mA | fA*mC*mC*(X2) |
| | 1) | | (SEQ ID NO: 53) | (SEQ ID NO: 116) |
| RD0766 | (X1)*mA*mG*mG.mG. | PEG2 | mG*mG*mU*fG* | mU*fG*mU*mU*mC. |
| | mG.mU.dC*dC*dA.mC. | | mU.mC.fG.fA.fG.fA. | mU.fC.fG.fU.mC.fU.fC. |
| | mA.mU.mG.mG*mC*mA* | | fA.fG.fA.fG.fG.fA.fG* | fC.mU.mC.fG.fA.mC* |
| | mA*mC (SEQ ID NO: | | fA*fA*fC*mA | fA*mC*mC*(X2) |
| | 1) | | (SEQ ID NO: 53) | (SEQ ID NO: 116) |
| RD0767 | (X1)*mA*mG*mG.mG. | PEG2 | mG*fG*mU*fG*mU. | mA*fU*mG*fU*mU.fG. |
| | mG.mU.rC*rC*rA.mC. | | fC.mG.fA.mG.fA. | mU.fU.mC.fU.mC.fG. |
| | mA.mU.mG.mG*mC*mA* | | mA.fG.mA.fG.mG.fA. | mU.fC.mU.mC.mC. |
| | mA*mC (SEQ ID NO: | | mG.fA.mA.fC.mA* | fU.mC.fG.mA.fC*mA* |
| | 1) | | fA*mU*fA*mU | fC*mC.(X2)(SEQ ID |
| | | | (SEQ ID NO: 3) | NO: 4) |
| RD0774 | (X1)*mA*mG*mG.mG. | PEG2 | mG*mG*mU*fG* | mG*mC*mA*fU*fA.fU. |
| | mG.mU.rC*rC*rA.mC. | | mU.mC.fG.fA.fG.fA. | mU.fG.mU.mU.mC. |
| | mA.mU.mG.mG*mC*mA* | | fA.fG.fA.fG.fG.fA.fG. | mU.fC.fG.fU.mC.fU.fC. |
| | mA*mC (SEQ ID NO: | | fA.fA.fC.mA*fA*mU* | fC.mU.mC.fG.fA.mC* |
| | 1) | | fA*mU (SEQ ID | fA*mC*mC*(X2) |
| | | | NO: 3) | (SEQ ID NO: 117) |
| RD0775 | (X1)*mA*mG*mG.mG. | PEG2 | mG*mG*mU*fG* | mG*mC*mA*fU*fA.fU. |
| | mG.mU.rC*rC*rA.mC. | | mU.mC.fG.fA.fG.fA. | mU.fG.mU.mU.mC. |
| | mA.mU.mG.mG*mA*mA* | | fA.fG.fA.fG.fG.fA.fG. | mU.fC.fG.fU.mC.fU.fC. |
| | mA*mC (SEQ ID NO: | | fA.fA.fC.mA*fA*mU* | fC.mU.mC.fG.fA.mC* |
| | 113) | | fA*mU (SEQ ID | fA*mC*mC*(X2) |
| | | | NO: 3) | (SEQ ID NO: 117) |
| RD0779 | (X1)*mA*mG*mG.mG. | PEG2 | mG*mG*mU*fG* | mU*fG*mU*mU*mC. |
| | mG.mU.rC*rC*rA.mC. | | mG.mG.fU.fG.fG.fA. | mU.fC.fG.fU.mC.fU. |
| | mA.mU.mG.mG*mA*mA* | | fA.fG.fA.fG.fG.fA.fG* | mC.fC.mC.mA.fC.fC. |
| | mA*mC (SEQ ID NO: | | fA*fA*fC*mA | mC*fA*mC*mC*(X2) |
| | 113) | | (SEQ ID NO: 84) | (SEQ ID NO: 115) |
| RD0780 | (X1)*mA*mG*mG.mG. | PEG2 | mG*mG*mU*fG* | mU*mU*fG*mU*mU. |
| | mG.mU.rC*rC*rA.mC. | | mG.mG.fU.fG.fG.fA. | mC.mU.fC.fG.fU.mC. |
| | mA.mU.mG.mG*mA*mA* | | fA.fG.fA.fG.fG.fA.fG* | fU.mC.fC.mC.mA.fC.fC. |
| | mA*mC (SEQ ID NO: | | fA*fA*fC*mA | mC*fA*mC*mC*(X2) |
| | 113) | | (SEQ ID NO: 84) | (SEQ ID NO: 118) |
| RD0995 | (X1)*mA*mG*mG.mG. | Ph | mG*mG*mU*fG* | mU*fG*mU*mU*mC. |
| | mG.mU.rC*rC*rA.mC. | | mG.mG.fU.fG.fG.fA. | mU.fC.fG.fU.mC.fU. |
| | mA.mU.mG.mG*mC*mA* | | fA.fG.fA.fG.fG.fA.fG* | mC.fC.mC.mA.fC.fC. |
| | mA*mC (SEQ ID NO: | | fA*fA*fC*mA | mC*fA*mC*mC*(X2) |
| | 1) | | (SEQ ID NO: 84) | (SEQ ID NO: 115) |
| RD0996 | (X1)*mA*mG*mG.mG. | C3-(X1) | mG*mG*mU*fG* | mU*fG*mU*mU*mC. |
| | mG.mU.rC*rC*rA.mC. | | mG.mG.fU.fG.fG.fA. | mU.fC.fG.fU.mC.fU. |
| | mA.mU.mG.mG*mC*mA* | | fA.fG.fA.fG.fG.fA.fG* | mC.fC.mC.mA.fC.fC. |
| | mA*mC (SEQ ID NO: | | fA*fA*fC*mA | mC*fA*mC*mC*(X2) |
| | 1) | | (SEQ ID NO: 84) | (SEQ ID NO: 115) |
| RD0997 | (X1)*mA*mG*mG.mG. | C3 | mG*mG*mU*fG* | mU*fG*mU*mU*mC. |
| | mG.mU.rC*rC*rA.mC. | | mG.mG.fU.fG.fG.fA. | mU.fC.fG.fU.mC.fU. |
| | mA.mU.mG.mG*mC*mA* | | fA.fG.fA.fG.fG.fA.fG* | mC.fC.mC.mA.fC.fC. |
| | mA*mC (SEQ ID NO: | | fA*fA*fC*mA | mC*fA*mC*mC*(X2) |
| | 1) | | (SEQ ID NO: 84) | (SEQ ID NO: 115) |
| RD0999 | (X1)*mA*mG*mG.mG. | PEG2 | mG*mG*mU*fG* | mU*fG*mU*mU*mC. |
| | mG.mU.rC*rC*rA.mC. | | mG.mG.fU.fG.fG.fA. | mU.fC.fG.fU.mC.fU. |
| | mA.mU.mG.mG*mC*mA* | | fA.fG.fA.fG.fG.fA.fG* | mC.fC.mC.mA.fC.fC. |
| | mA*mC (SEQ ID NO: | | fA*fA*fC*mA | mC*fA*mC*mC*(X2) |
| | 1) | | (SEQ ID NO: 84) | (SEQ ID NO: 115) |
| RD1014 | (X1)*mA*mG*mG.mG. | PEG2 | mG*fG*mG*fU*mG. | mG*fC*mA*fU*mA.fU. |
| | mG.mU.rC*rC*rA.mC. | | fG.fA.fA.mG.fA.fG. | mU.fG.mU.fU.mC.fU. |
| | mA.mU.mG.mG*mA*mA* | | fU.mA.fG.mA.fA. | mA.fG.mU.fC.mU. |
| | mA*mC (SEQ ID NO: | | mC.fA.mA.fU*mA*fU* | mC.mC.fC.mA*fC*mC* |
| | 113) | | mG*mC (SEQ ID | fC*(X2)(SEQ ID |
| | | | NO: 5) | NO: 6) |
| RD1015 | (X1)*mA*mG*mG.mG. | PEG2 | mG*fG*mG*fU*mG. | mG*fC*mA*fU*mA.fU. |
| | mG.mU.rC*rC*rA.mC. | | fG.fA.fA.mG.fA.fG. | mU.fG.mU.fU.mC.fU. |
| | mA.mU.mG.mG*mC*mA* | | fU.mA.fG.mA.fA. | mA.fG.mU.fC.mU. |
| | mA*mC (SEQ ID NO: | | mC.fA.mA.fU*mA*fU* | mC.mC.fC.mA*fC*mC* |
| | 1) | | mG*mC (SEQ ID | fC*(X2)(SEQ ID |
| | | | NO: 5) | NO: 6) |

### Example 7: Evaluation of RNA Editing Constructs with Overhang in Recruiting Domain

SK-BR-3 cells were cultured in Complete Fibroblast Growth medium obtained from Cell Biologics (Cat #M2267) at 37° C. in an atmosphere of 5% CO₂. Cell lines were acquired from ATCC (ATCC Cat #HTB-30). Cells were seeded in 96-well plates at 2×10⁵ cells/well in 100 μL of growth medium without antibiotics one day prior to the transfection. The next day, cells were transfected with 10 nM of GAPDH RNA oligonucleotide complexed with 0.6 μL Lipofectamaine RNAiMAX. After 24 hours (h), cells were harvested and RNA extracted using the RNAqueous-96 Kit (ThermoFisher Cat #AM1920). Reverse transcription was performed using SuperScript IV (Invitrogen) followed by PCR with Platinum II Taq polymerase (ThermoFisher). The resulting DNA was purified on MinElute 96 UF PCR Purification Kit (QIAGEN Cat #28051) and analyzed by Sanger sequencing (Genewiz).

Results

Tables 7A and 7B below list the GAPDH editing efficiencies and compound information, respectively, of the various compositions of oligo sequences.

TABLE 7A
GAPDH editing efficiencies of oligo compositions

| Compound ID | Editing Efficiency 10 nM | Target Gene |
|---|---|---|
| RD0781 | 29% | GAPDH |
| RD0999 | 40% | GAPDH |
| RD1017 | 37% | GAPDH |
| RD1018 | 41% | GAPDH |
| RD1019 | 46% | GAPDH |
| RD1021 | 29% | GAPDH |
| RD1022 | 34% | GAPDH |

TABLE 7B
Compound information

| | | Linker | | |
|---|---|---|---|---|
| | | between | | |
| | | editing | short strand | |
| | Editing domain on long | domain and | complementary to | Recruiting domain on |
| | strand sequence | recruiting | recruiting domain | long strand, sequence |
| Conjugate ID | information 5′- 3′ | domain | on Long strand 5′-3′ | information 5′-3′ |
| RD0781 | (X1)*mA*mG*mG.mG. | PEG2 | mG*fU*mG*fU*mG. | mC*mA*fC*mU*fG. |
| | mG.mU.rC*rC*rA. | | fG.fA.fA.mG. | mU.fU.mC.fU.mC. |
| | mC.mA.mU.mG.mG*mA* | | fA.fG.fG.mA. | fG.mU.fC.mU. |
| | mA*mA*mC (SEQ | | fG.mA.fA.mC*fA* | mC.mC.fC.mA*fC* |
| | ID NO: 113) | | mG*fU*mG | mC*fC*(X2) |
| | | | (SEQ ID NO: | (SEQ ID NO: |
| | | | 111) | 144) |
| RD0999 | (X1)*mA*mG*mG.mG. | PEG2 | mG*mG*mU*fG*mG. | mU*fG*mU*mU*mC. |
| | mG.mU.rC*rC*rA. | | mG.fU.fG.fG. | mU.fC.fG.fU.mC. |
| | mC.mA.mU.mG.mG*mC* | | fA.fA.fG.fA. | fU.mC.fC.mC. |
| | mA*mA*mC (SEQ | | fG.fG.fA.fG*fA* | mA.fC.fC.mC*fA* |
| | ID NO: 1) | | fA*fC*mA | mC*mC*(X2) |
| | | | (SEQ ID NO: | (SEQ ID NO: |
| | | | 84) | 115) |
| RD1017 | (X1)*mA*mG*mG.mG. | PEG2 | mG*mG*mU*fG*mG. | mU*fG*mU*mU*mC. |
| | mG.mU.rC*rC*rA. | | mG.fU.fG.fG. | mU.fC.fG.fU.mC. |
| | mC.mA.mU.mG.mG*mC* | | fA.fA.fG.fA. | fU.mC.fC.mC. |
| | mA*mA*mC (SEQ | | fG.fG.fA*fG*fA* | mA.fC.fC.mC*fA* |
| | ID NO: 1) | | fA*mC (SEQ | mC*mC*(X2) |
| | | | ID NO: 166) | (SEQ ID NO: |
| | | | | 115) |
| RD1018 | (X1)*mA*mG*mG.mG. | PEG2 | mG*mG*mU*fG*mG. | mU*mU*fG*mU*mU. |
| | mG.mU.rC*rC*rA. | | mG.fU.fG.fG. | mC.mU.fC.fG.fU. |
| | mC.mA.mU.mG.mG*mC* | | fA.fA.fG.fA. | mC.fU.mC.fC. |
| | mA*mA*mC (SEQ | | fG.fG.fA.fG*fA* | mC.mA.fC.fC.mC* |
| | ID NO: 1) | | fA*fC*mA | fA*mC*mC*(X2) |
| | | | (SEQ ID NO: | (SEQ ID NO: |
| | | | 84) | 118) |
| RD1019 | (X1)*mA*mG*mG.mG. | PEG2 | mG*mG*mU*fG*mG. | mG*mU*mU*mC*mU. |
| | mG.mU.rC*rC*rA. | | mG.fU.fG.fG. | fC.fG.fU.mC.fU. |
| | mC.mA.mU.mG.mG*mC* | | fA.fA.fG.fA. | mC.fC.mC.mA. |
| | mA*mA*mC (SEQ | | fG.fG.fA.fG*fA* | fC.fC.mC*fA*mC* |
| | ID NO: 1) | | fA*fC*mA | mC*(X2)(SEQ |
| | | | (SEQ ID NO: | ID NO: 167) |
| | | | 84) | |
| RD1021 | (X1)*mA*mG*mG.mG. | PEG6 | mG*fU*mG*fU*mG. | mC*mA*fC*mU*fG. |
| | mG.mU.rC*rC*rA. | | fG.fA.fA.mG. | mU.fU.mC.fU.mC. |
| | mC.mA.mU.mG.mG*mA* | | fA.fG.fG.mA. | fG.mU.fC.mU. |
| | mA*mA*mC (SEQ | | fG.mA.fA.mC*fA* | mC.mC.fC.mA*fC* |
| | ID NO: 113) | | mG*fU*mG | mC*fC*(X2) |
| | | | (SEQ ID NO: | (SEQ ID NO: |
| | | | 111) | 144) |
| RD1022 | (X1)*mA*mG*mG.mG. | PEG2 | mG*fU*mG*fU*mG. | mC*mA*fC*mU*fG. |
| | mG.mU.rC*rC*rA. | | fG.fA.fA.mG. | mU.fU.mC.fU.mC. |
| | mC.mA.mU.mG.mG*mC* | | fA.fG.fG.mA. | fG.mU.fC.mU. |
| | mA*mA*mC (SEQ | | fG.mA.fA.mC*fA* | mC.mC.fC.mA*fC* |
| | ID NO: 1) | | mG*fU*mG | mC*fC*(X2) |
| | | | (SEQ ID NO: | (SEQ ID NO: |
| | | | 111) | 144) |

### Example 8: Evaluation of RNA Editing Constructs in Wild Type Mice

After 4 days of acclimation, animals will be randomized by BW in 7 groups (n=3). Cage side observations will be daily. 20 mg/kg bolus injection of RD2242 through i.v. will be performed. The total volume as determined by BW should be injected in a period of 10 seconds. Three mice will be anesthetized with isoflurane and euthanized at 8, 24, 48, 72 and 120 hours post-dose. Livers will be collected and placed in RNA later. Liver samples will be homogenized and RNA will be extracted using the RNAqueous-96 Kit (ThermoFisher Cat #AM1920). Reverse transcription will be performed using SuperScript IV (Invitrogen) followed by PCR with Platinum II Taq polymerase (ThermoFisher). The resulting DNA will be purified on MinElute 96 UF PCR Purification Kit (QIAGEN Cat #28051) and analyzed by Sanger sequencing (Genewiz).

### Example 9: In Vivo Editing of Monkey mRNA in Cynomolgus Monkeys

Four non-naive cynomolgus monkeys will be dosed with two editing RNA oligonucleotides at 1-20 mg/kg (e.g., about 5 mg/kg). Blood from all animals will be collected from pre-bleeds 6 days prior to dosing, on the day of dosing and every week following oligonucleotide administration. Serum will be prepared and introduction of the point mutation will be analyzed by mass spectroscopy.

Liver biopsies (˜1-5 mg) will also be performed in all animals 6 days prior to dosing, 3 days and 15 days post dosing. Harvested liver tissue samples will be homogenized, and RNA extracted to check for RNA editing.

### Exemplary Sequences

This Table exhibits some exemplary sequences as disclosed by the instant Specification, but is not limiting. As the skilled artisan will readily appreciate, the sequences disclosed herein may recite terminal moieties (e.g., X₁, X₂), however, it shall be understood that such moieties may be removed. Accordingly, a sequence as identified herein, may be interpreted to optionally include a terminal moiety (e.g., X₁, X₂) where present or absent from the terminal end of an oligo sequence. Similarly, the skilled artisan will readily appreciate, the sequences disclosed herein may recite specific linkers (e.g., PEG2), however, it shall be understood that any appropriate linker disclosed herein may be used. Accordingly, a sequence as identified herein, may be interpreted to optionally include a specified linker (or another linker as disclosed herein).

TABLE 8
Exemplary Sequences

| | SEQ | |
|---|---|---|
| Sequence^{€} | ID | |
| (<Strand Ref.: ##>) | NO: | Description^{†} |
| [mA][2flG][mG][2flG][mG][2flU]CCA[2flC] | 1 | RL0079 oligo of composition |
| [mA][2flU][mG][2flG][mC][2flA]*[mA] | | RL0079 - Negative Control |
| *[2flc] | | (Single oligo negative control |
| (<Strand Ref.: 1>) | | without ADAR recruiting) |
| | | (NT) |
| GGUGUCGAGAAGAGGAGAACAAUAUGCUAAAUGUUGU | 2 | RH0001 oligo of composition |
| UCUCGUCUCCUCGACACC[mA][mG][mG][mG][mG] | | RH0001 - Positive Control |
| [mU]CCA[mC][mA][mU][mG][mG]*[mC]*[mA]* | | (Single oligo Hairpin)(NT) |
| [mA]*[mC] | | |
| (<Strand Ref.: 2>) | | |
| [mG]*[mG]*[mU] G[mU][mC]GAGAAGAGGAGAAC | 3 | RS0003duplex oligo of |
| [mA]A[mU]*A*[mU] | | composition RD0016 - RNA |
| (<Strand Ref.: 3>) | | Duplex (NT) |
| [mA]*[2flU]*G[2flU][mU]G[mU][mU][mC] | 4 | RL0016duplex oligo of |
| [mU][2flC]G[2flu][mC][2flu]C[2flc][mU] | | composition RD0016 - RNA |
| [mC]GA[mC]A[mC][mC] | | Duplex (NT) |
| (<Strand Ref.: 4>) | | |
| [mA][mG][mG][mG][mG][mU] CCA[mC][mA] | 1 | RL0016guide oligo of |
| [mU][mG][mG]*[mC]*[mA]*[mA]*[mC] | | composition RD0016 - RNA |
| (<Strand Ref.: 5>) | | Duplex (NT) |
| G*G*GUGGAAGAGUAGAACAAUAU*G*C | 5 | RS0008duplex oligo of |
| (<Strand Ref.: 6>) | | composition RD0034 - RNA |
| | | Duplex (NT) |
| G*C*AUAUUGUUCUAGUCUCCCACCC | 6 | RL0034duplex oligo of |
| (<Strand Ref.: 7>) | | composition RD0034 - RNA |
| | | Duplex (NT) |
| [mA][mG][mG][mG][mG][mU] CCA[mC][mA] | 1 | RL0034guide oligo of |
| [mU][mG][mG]*[mC]*[mA]*[mA]*[mC] | | composition RD0034 - RNA |
| (<Strand Ref.: 8>) | | Duplex (NT) |
| G*G*GUGGAACAGUACAACAAUAU*G*C | 7 | RS0009 oligo of composition |
| (<Strand Ref.: 9>) | | RD0037 - RNA Duplex (NT) |
| GUGGAAUAGUAUAACAAUAUgcuaaAUGUUGUUAUAG | 8 | GluR2 site (natural hairpin) |
| UGUCCCAC | | (NT) |
| (<Strand Ref.: 10>) | | |
| ATATATAGCCA | 9 | Definitional Example |
| (<Strand Ref.: 11>) | | Sequence |
| TGGCCATATAT | 10 | Definitional Example |
| (<Strand Ref.: 12>) | | Sequence |
| [mA]*[2flU]*G[2flU][mU]G[mU][mU][mC] | 11 | Connected RL0016 oligo |
| [mU][2flC]G[2flU][mC][2flu]C[2flC][mU] | | (duplex and guide connected) |
| [mC]GA[mC]A[mC][mC][mA][mG][mG][mG] | | |
| [mG][mU]CCA[mC][mA][mU][mG][mG]*[mC]* | | |
| [mA]*[mA]*[mC ] | | |
| (<Strand Ref.: 13>) | | |
| G*C*AUAUUGUUCUAGUCUCCCACCC[mA][mG][mG] | 12 | Connected RL0034 oligo |
| [mG][mG][mU]CCA[mC][mA][mU][mG][mG]* | | (duplex and guide connected) |
| [mC]*[mA]*[mA]*[mC] | | |
| (<Strand Ref.: 14>) | | |
| GGUGUCGAGAAGAGGAGAACAAUAU | 3 | RS0003 duplex oligo of |
| (<Strand Ref.: 15>) | | composition RD0016 - RNA |
| | | Duplex (NT) |
| AUGUUGUUCUCGUCUCCUCGACACC | 4 | RL0016 duplex oligo of |
| (<Strand Ref.: 16>) | | composition RD0016 - RNA |
| | | Duplex (NT) |
| AGGGGUCCACAUGGCAAC | 1 | RL0016 guide and RL0034g |
| (<Strand Ref.: 17>) | | oligo of composition RD0016 |
| | | - RNA Duplex (NT) |
| GGGUGGAAGAGUAGAACAAUAUGC | 5 | RS0008 duplex oligo of |
| (<Strand Ref.: 18>) | | composition RD0034 - RNA |
| | | Duplex (NT) |
| GCAUAUUGUUCUAGUCUCCCACCC | 6 | RL0034 duplex oligo of |
| (<Strand Ref.: 19>) | | composition RD0034 - RNA |
| | | Duplex (NT) |
| G*C*AUAUUGUUGUAGUGUCCCACCC[mA][mG][mG] | 13 | RL0037 oligo of composition |
| [mG][mG][mU] CCA[mC][mA][mU][mG][mG]* | | RD0037 - RNA Duplex (NT) |
| [mC]*[mA]*[mA]*[mC] | | |
| (<Strand Ref.: 20>) | | |
| AUGUUGUUCUCGUCUCCUCGACACCAGGGGUCCACAU | 11 | Connected RL0016 oligo |
| GGCAAC | | (duplex and guide connected) |
| (<Strand Ref.: 21>) | | |
| GCAUAUUGUUCUAGUCUCCCACCCAGGGGUCCACAUG | 12 | Connected RL0034 oligo |
| GCAAC | | (duplex and guide connected) |
| (<Strand Ref.: 22>) | | |
| (X₁)*(mA)*(mG)*(mG)(mG)(mG)(mU)(rc)(rC) | 1 | RLE0001 guide oligo of |
| (rA)(mC)(mA)(mU)(mG)(mG)*(mC)*(mA)* | | composition |
| (mA)*(mC) | | X₁ = FormUla (X₁) |
| (<Strand Ref.: 23>) | | |
| (mA)*(fU)*(rG)(fU)(mU)(rG)(mU)(mU)(mC) | 4 | RLR0001 duplex oligo of |
| (mU)(fC)(rG)(fU)(mC)(fU)(rc)(fC)(mU) | | composition RP0001 - RNA |
| (mC)(rG)(rA)(mC)(rA)*(mC)*(mC)*(X₂) | | Duplex (NT) |
| (<Strand Ref.: 24>) | | X₂ = FormUla(X2) |
| (mG)*(mG)*(mU)*(fG)*(mU)(mC)(fG)(fA) | 3 | RS0074duplex oligo of |
| (fG)(fA)(fA)(fG)(fA)(fG)(fG)(fA)(fG) | | composition RP0001 - RNA |
| (fA)(fA)(fC)(mA)*(fA)*(mU)*(fA)*(mU) | | Duplex (NT) |
| (<Strand Ref.: 25>) | | |
| X₁AGGGGUCCACAUGGCAAC | 1 | RLE0001 guide oligo of |
| (<Strand Ref.: 26>) | | composition |
| | | X₁ = FormUla (X₁) |
| AUGUUGUUCUCGUCUCCUCGACACCX₂ | 4 | RLR0001 duplex oligo of |
| (<Strand Ref.: 27>) | | composition RP0001 - RNA |
| | | Duplex (NT) |
| | | X₂ = FormUla(X2) |
| GGUGUCGAGAAGAGGAGAACAAUAU | 3 | RS0074duplex oligo of |
| (<Strand Ref.: 28>) | | composition RP0001 - RNA |
| | | Duplex (NT) |
| rG.rG.rU.rG.rU.rC.rG.rA.rG.rA.rA.rG.rA. | 3 | RS0001 strand of compound |
| rG.rG.rA.rG.rA.rA.rC.rA.rA.rU.rA.rU | | RD0001 (NT) |
| (<Strand Ref.: 29>) | | |
| rA.rU.rG.rU.rU.rG.rU.rU.rC.rU.rC.rG.rU. | 11 | RL0001 strand of compound |
| rC.rU.rC.rC.rU.rC.rG.rA.rC.rA.rC.rC. | | RD0001 (NT) |
| rA.rG.rG.rG.rG.rU.rC.rC.rA.rC.rA.rU. | | |
| rG.rG.rC.rA.rA.rC | | |
| (<Strand Ref.: 30>) | | |
| GGUGUCGAGAAGAGGAGAACAAUAU | 3 | RS0001-UM strand of |
| (<Strand Ref.: 31>) | | compound RD0001 (NT) |
| AUGUUGUUCUCGUCUCCUCGACACCAGGGGUCCACAU | 11 | RL0001-UM strand of |
| GGCAAC | | compound RD0001 (NT) |
| (<Strand Ref.: 32>) | | |
| rG.rG.rU.rG.rU.rC.rG.rA.rG.rA.rA.rG.rA. | 3 | RS0001 strand of compound |
| rG.rG.rA.rG.rA.rA.rC.rA.rA.rU.rA.rU | | RD0002 (NT) |
| (<Strand Ref.: 33>) | | |
| rA.rU.rG.rU.rU.rG.rU.rU.rC.rU.rC.rG.rU. | 11 | RL0002 strand of compound |
| rC.rU.rC.rC.rU.rC.rG.rA.rC.rA.rC.rC. | | RD0002 (NT) |
| .mA.mG.mG.mG.mG.mU.rC.rC.rA.mC.mA.mU. | | |
| mG.mG*mC*mA*mA*mC | | |
| (<Strand Ref.: 34>) | | |
| GGUGUCGAGAAGAGGAGAACAAUAU | 3 | RS0001-UM strand of |
| (<Strand Ref.: 35>) | | compound RD0002 (NT) |
| AUGUUGUUCUCGUCUCCUCGACACCAGGGGUCCACAU | 11 | RL0002-UM strand of |
| GGCAAC | | compound RD0002 (NT) |
| (<Strand Ref.: 36>) | | |
| rG.rG.rU.rG.rU.rC.rG.rA.rG.rA.rA.rG.rA. | 3 | RS0001 strand of compound |
| rG.rG.rA.rG.rA.rA.rC.rA.rA.rU.rA.rU | | RD0003 (NT) |
| (<Strand Ref.: 37>) | | |
| rA.rU.rG.rU.rU.rG.rU.rU.rC.rU.rC.rG.rU. | 11 | RL0003 strand of compound |
| rC.rU.rC.rC.rU.rC.rG.rA.rC.rA.rC.rC. | | RD0003 (NT) |
| rA.rG.rG.rG.rG.rU.rC.rC.rA.rC.rA.rU. | | |
| rG.rG.rC.rA*rA*rC | | |
| (<Strand Ref.: 38>) | | |
| GGUGUCGAGAAGAGGAGAACAAUAU | 3 | RS0001-UM strand of |
| (<Strand Ref.: 39>) | | compound RD0003 (NT) |
| AUGUUGUUCUCGUCUCCUCGACACCAGGGGUCCACAU | 11 | RL0003-UM strand of |
| GGCAAC | | compound RD0003 (NT) |
| (<Strand Ref.: 40>) | | |
| rG.rG.rU.rG.rU.rC.rG.rA.rG.rA.rA.rG.rA. | 3 | RS0001 strand of compound |
| rG.rG.rA.rG.rA.rA.rC.rA.rA.rU.rA.rU | | RD0004 (NT) |
| (<Strand Ref.: 41>) | | |
| rA.rU.rG.rU.rU.rG.rU.rU.rC.rU.rC.rG.rU. | 11 | RL0004 strand of compound |
| rC.rU.rC.rC.rU.rC.rG.rA.rC.rA.rC.rC. | | RD0004 (NT) |
| fA.fG.fG.fG.fG.fU.rC.rC.rA.fC.fA.fU. | | |
| fG.fG.fC.fA*fA*fC | | |
| (<Strand Ref.: 42>) | | |
| GGUGUCGAGAAGAGGAGAACAAUAU | 3 | RS0001-UM strand of |
| (<Strand Ref.: 43>) | | compound RD0004 (NT) |
| AUGUUGUUCUCGUCUCCUCGACACCAGGGGUCCACAU | 11 | RL0004-UM strand of |
| GGCAAC | | compound RD0004 (NT) |
| (<Strand Ref.: 44>) | | |
| rG.rG.rU.rG.rU.rC.rG.rA.rG.rA.rA.rG.rA. | 3 | RS0001 strand of compound |
| rG.rG.rA.rG.rA.rA.rC.rA.rA.rU.rA.rU | | RD0005 (NT) |
| (<Strand Ref.: 45>) | | |
| rA.rU.rG.rU.rU.rG.rU.rU.rC.rU.rC.rG.rU. | 11 | RL0005 strand of compound |
| rC.rU.rC.rC.rU.rC.rG.rA.rC.rA.rC.rC. | | RD0005 (NT) |
| mA.fG.mG.fG.mG.fU.rC.rC.rA.fC.mA.fU. | | |
| mG.fG.mC.fA*mA*fC | | |
| (<Strand Ref.: 46>) | | |
| GGUGUCGAGAAGAGGAGAACAAUAU | 3 | RS0001-UM strand of |
| (<Strand Ref.: 47>) | | compound RD0005 (NT) |
| AUGUUGUUCUCGUCUCCUCGACACCAGGGGUCCACAU | 11 | RL0005-UM strand of |
| GGCAAC | | compound RD0005 (NT) |
| (<Strand Ref.: 48>) | | |
| rG*rG*rU.rG.rU.rC.rG.rA.rG.rA.rA.rG.rA. | 3 | RS0002 strand of compound |
| rG.rG.rA.rG.rA.rA.rC.rA.rA.rU*rA*rU | | RD0006 (NT) |
| (<Strand Ref.: 49>) | | |
| rA*rU*rG.rU.rU.rG.rU.rU.rC.rU.rC.rG.rU. | 11 | RL0006 strand of compound |
| rC.rU.rC.rC.rU.rC.rG.rA.rC.rA.rC.rC. | | RD0006 (NT) |
| rA.rG.rG.rG.rG.rU.rC.rC.rA.rC.rA.rU. | | |
| rG.rG.rC.rA.rA.rC | | |
| (<Strand Ref.: 50>) | | |
| GGUGUCGAGAAGAGGAGAACAAUAU | 3 | RS0002-UM strand of |
| (<Strand Ref.: 51>) | | compound RD0006 (NT) |
| AUGUUGUUCUCGUCUCCUCGACACCAGGGGUCCACAU | 11 | RL0006-UM strand of |
| GGCAAC | | compound RD0006 (NT) |
| (<Strand Ref.: 52>) | | |
| rG*rG*rU.rG.rU.rC.rG.rA.rG.rA.rA.rG.rA. | 3 | RS0002 strand of compound |
| rG.rG.rA.rG.rA.rA.rC.rA.rA.rU*rA*rU | | RD0007 (NT) |
| (<Strand Ref.: 53>) | | |
| rA*rU*rG.rU.rU.rG.rU.rU.rC.rU.rC.rG.rU. | 11 | RL0007 strand of compound |
| rC.rU.rC.rC.rU.rC.rG.rA.rC.rA.rC.rC. | | RD0007 (NT) |
| mA.mG.mG.mG.mG.mU.rC.rC.rA.mC.mA.mU. | | |
| mG.mG*mC*mA*mA*mC | | |
| (<Strand Ref.: 54>) | | |
| GGUGUCGAGAAGAGGAGAACAAUAU | 3 | RS0002-UM strand of |
| (<Strand Ref.: 55>) | | compound RD0007 (NT) |
| AUGUUGUUCUCGUCUCCUCGACACCAGGGGUCCACAU | 11 | RL0007-UM strand of |
| GGCAAC | | compound RD0007 (NT) |
| (<Strand Ref.: 56>) | | |
| rG*rG*rU.rG.rU.rC.rG.rA.rG.rA.rA.rG.rA. | 3 | RS0002 strand of compound |
| rG.rG.rA.rG.rA.rA.rC.rA.rA.rU*rA*rU | | RD0008 (NT) |
| (<Strand Ref.: 57>) | | |
| rA*rU*rG.rU.rU.rG.rU.rU.rC.rU.rC.rG.rU. | 11 | RL0008 strand of compound |
| rC.rU.rC.rC.rU.rC.rG.rA.rC.rA.rC.rC. | | RD0008 (NT) |
| rA.rG.rG.rG.rG.rU.rC.rC.rA.rC.rA.rU. | | |
| rG.rG.rC.rA*rA*rC | | |
| (<Strand Ref.: 58>) | | |
| GGUGUCGAGAAGAGGAGAACAAUAU | 3 | RS0002-UM strand of |
| (<Strand Ref.: 59>) | | compound RD0008 (NT) |
| AUGUUGUUCUCGUCUCCUCGACACCAGGGGUCCACAU | 11 | RL0008-UM strand of |
| GGCAAC | | compound RD0008 (NT) |
| (<Strand Ref.: 60>) | | |
| rG*rG*rU.rG.rU.rC.rG.rA.rG.rA.rA.rG.rA. | 3 | RS0002 strand of compound |
| rG.rG.rA.rG.rA.rA.rC.rA.rA.rU*rA*rU | | RD0009 (NT) |
| (<Strand Ref.: 61>) | | |
| rA*rU*rG.rU.rU.rG.rU.rU.rC.rU.rC.rG.rU. | 11 | RL0009 strand of compound |
| rC.rU.rC.rC.rU.rC.rG.rA.rC.rA.rC.rC. | | RD0009 (NT) |
| fA.fG.fG.fG.fG.fU.rC.rC.rA.fC.fA.fU. | | |
| fG.fG.fC.fA*fA*fC | | |
| (<Strand Ref.: 62>) | | |
| GGUGUCGAGAAGAGGAGAACAAUAU | 3 | RS0002-UM strand of |
| (<Strand Ref.: 63>) | | compound RD0009 (NT) |
| AUGUUGUUCUCGUCUCCUCGACACCAGGGGUCCACAU | 11 | RL0009-UM strand of |
| GGCAAC | | compound RD0009 (NT) |
| (<Strand Ref.: 64>) | | |
| rG*rG*rU.rG.rU.rC.rG.rA.rG.rA.rA.rG.rA. | 3 | RS0002 strand of compound |
| rG.rG.rA.rG.rA.rA.rC.rA.rA.rU*rA*rU | | RD0010 (NT) |
| (<Strand Ref.: 65>) | | |
| rA*rU*rG.rU.rU.rG.rU.rU.rC.rU.rC.rG.rU. | 11 | RL0010 strand of compound |
| rC.rU.rC.rC.rU.rC.rG.rA.rC.rA.rC.rC. | | RD0010 (NT) |
| mA.fG.mG.fG.mG.fU.rC.rC.rA.fC.mA.fU. | | |
| mG.fG.mC.fA*mA*fC | | |
| (<Strand Ref.: 66>) | | |
| GGUGUCGAGAAGAGGAGAACAAUAU | 3 | RS0002-UM strand of |
| (<Strand Ref.: 67>) | | compound RD0010 (NT) |
| AUGUUGUUCUCGUCUCCUCGACACCAGGGGUCCACAU | 1 | RL0010-UM strand of |
| GGCAAC | | compound RD0010 (NT) |
| (<Strand Ref.: 68>) | | |
| mG*mG*mU.rG.mU.mC.rG.rA.rG.rA.rA.rG.rA. | 3 | RS0003 strand of compound |
| rG.rG.rA.rG.rA.rA.rC.mA.rA.mU*rA*mU | | RD0011 (NT) |
| (<Strand Ref.: 69>) | | |
| mA*mU*rG.mU.mU.rG.mU.mU.mC.mU.mC.rG.mU. | 11 | RL0011 strand of compound |
| mC.mU.mC.mC.mU.mC.rG.rA.mC.rA.mC.mC. | | RD0011 (NT) |
| mA.mG.mG.mG.mG.mU.rC.rC.rA.mC.mA.mU. | | |
| mG.mG*mC*mA*mA*mC | | |
| (<Strand Ref.: 70>) | | |
| GGUGUCGAGAAGAGGAGAACAAUAU | 3 | RS0003-UM strand of |
| (<Strand Ref.: 71>) | | compound RD0011 (NT) |
| AUGUUGUUCUCGUCUCCUCGACACCAGGGGUCCACAU | 11 | RL0011-UM strand of |
| GGCAAC | | compound RD0011 (NT) |
| (<Strand Ref.: 72>) | | |
| mG*mG*mU.rG.mU.mC.rG.rA.rG.rA.rA.rG.rA. | 3 | RS0003 strand of compound |
| rG.rG.rA.rG.rA.rA.rC.mA.rA.mU*rA*mU | | RD0012 (NT) |
| (<Strand Ref.: 73>) | | |
| mA*mU*rG.mU.mU.rG.mU.mU.mC.mU.mC.rG.mU. | 11 | RL0012 strand of compound |
| mC.mU.mC.mC.mU.mC.rG.rA.mC.rA.mC.mC. | | RD0012 (NT) |
| rA.rG.rG.rG.rG.rU.rC.rC.rA.rC.rA.rU. | | |
| rG.rG.rC.rA*rA*rC | | |
| (<Strand Ref.: 74>) | | |
| GGUGUCGAGAAGAGGAGAACAAUAU | 3 | RS0003-UM strand of |
| (<Strand Ref.: 75>) | | compound RD0012 (NT) |
| AUGUUGUUCUCGUCUCCUCGACACCAGGGGUCCACAU | 11 | RL0012-UM strand of |
| GGCAAC | | compound RD0012 (NT) |
| (<Strand Ref.: 76>) | | |
| mG*mG*mU.rG.mU.mC.rG.rA.rG.rA.rA.rG.rA. | 3 | RS0003 strand of compound |
| rG.rG.rA.rG.rA.rA.rC.mA.rA.mU*rA*mU | | RD0013 (NT) |
| (<Strand Ref.: 77>) | | |
| mA*mU*rG.mU.mU.rG.mU.mU.mC.mU.mC.rG.mU. | 11 | RL0013 strand of compound |
| mC.mU.mC.mC.mU.mC.rG.rA.mC.rA.mC.mC. | | RD0013 (NT) |
| fA.fG.fG.fG.fG.fU.rC.rC.rA.fC.fA.fU. | | |
| fG.fG.fC.fA*fA*fC | | |
| (<Strand Ref.: 78>) | | |
| GGUGUCGAGAAGAGGAGAACAAUAU | 3 | RS0003-UM strand of |
| (<Strand Ref.: 79>) | | compound RD0013 (NT) |
| AUGUUGUUCUCGUCUCCUCGACACCAGGGGUCCACAU | | RL0013-UM strand of |
| GGCAAC | | compound RD0013 (NT) |
| (<Strand Ref.: 80>) | | |
| mG*mG*mU.rG.mU.mC.rG.rA.rG.rA.rA.rG.rA. | 3 | RS0003 strand of compound |
| rG.rG.rA.rG.rA.rA.rC.mA.rA.mU*rA*mU | | RD0014 (NT) |
| (<Strand Ref.: 81>) | | |
| mA*mU*rG.mU.mU.rG.mU.mU.mC.mU.mC.rG.mU. | 11 | RL0014 strand of compound |
| mC.mU.mC.mC.mU.mC.rG.rA.mC.rA.mC.mC. | | RD0014 (NT) |
| mA.fG.mG.fG.mG.fU.rC.rC.rA.fC.mA.fU. | | |
| mG.fG.mC.fA*mA*fC | | |
| (<Strand Ref.: 82>) | | |
| GGUGUCGAGAAGAGGAGAACAAUAU | 3 | RS0003-UM strand of |
| (<Strand Ref.: 83>) | | compound RD0014 (NT) |
| AUGUUGUUCUCGUCUCCUCGACACCAGGGGUCCACAU | 11 | RL0014-UM strand of |
| GGCAAC | | compound RD0014 (NT) |
| (<Strand Ref.: 84>) | | |
| mG*mG*mU.rG.mU.mC.rG.rA.rG.rA.rA.rG.rA. | 3 | RS0003 strand of compound |
| rG.rG.rA.rG.rA.rA.rC.mA.rA.mU*rA*mU | | RD0015 (NT) |
| (<Strand Ref.: 85>) | | |
| mA*fU*rG.fU.mU.rG.mU.mU.mC.mU.fC.rG.fU. | 11 | RL0015 strand of compound |
| mC.fU.rC.fC.mU.mC.rG.rA.mC.rA.mC.mC. | | RD0015 (NT) |
| rA.rG.rG.rG.rG.rU.rC.rC.rA.rC.rA.rU. | | |
| rG.rG.rC.rA.rA.rC | | |
| (<Strand Ref.: 86>) | | |
| GGUGUCGAGAAGAGGAGAACAAUAU | 3 | RS0003-UM strand of |
| (<Strand Ref.: 87>) | | compound RD0015 (NT) |
| AUGUUGUUCUCGUCUCCUCGACACCAGGGGUCCACAU | 1 | RL0015-UM strand of |
| GGCAAC | | compound RD0015 (NT) |
| (<Strand Ref.: 88>) | | |
| mG*mG*mU.rG.mU.mC.rG.rA.rG.rA.rA.rG.rA. | 3 | RS0003 strand of compound |
| rG.rG.rA.rG.rA.rA.rC.mA.rA.mU*rA*mU | | RD0016 (NT) |
| (<Strand Ref.: 89>) | | |
| mA*fU*rG.fU.mU.rG.mU.mU.mC.mU.fC.rG.fU. | 1 | RL0016 strand of compound |
| mC.fU.rC.fC.mU.mC.rG.rA.mC.rA.mC.mC. | | RD0016 (NT) |
| mA.mG.mG.mG.mG.mU.rC.rC.rA.mC.mA.mU. | | |
| mG.mG*mC*mA*mA*mC | | |
| (<Strand Ref.: 90>) | | |
| GGUGUCGAGAAGAGGAGAACAAUAU | 3 | RS0003-UM strand of |
| (<Strand Ref.: 91>) | | compound RD0016 (NT) |
| AUGUUGUUCUCGUCUCCUCGACACCAGGGGUCCACAU | | RL0016-UM strand of |
| GGCAAC | | compound RD0016 (NT) |
| (<Strand Ref.: 92>) | | |
| mG*mG*mU.rG.mU.mC.rG.rA.rG.rA.rA.rG.rA. | 3 | RS0003 strand of compound |
| rG.rG.rA.rG.rA.rA.rC.mA.rA.mU*rA*mU | | RD0017 (NT) |
| (<Strand Ref.: 93>) | | |
| mA*fU*rG.fU.mU.rG.mU.mU.mC.mU.fC.rG.fU. | 11 | RL0017 strand of compound |
| mC.fU.rC.fC.mU.mC.rG.rA.mC.rA.mC.mC. | | RD0017 (NT) |
| rA.rG.rG.rG.rG.rU.rC.rC.rA.rC.rA.rU. | | |
| rG.rG.rC.rA*rA*rC | | |
| (<Strand Ref.: 94>) | | |
| GGUGUCGAGAAGAGGAGAACAAUAU | 3 | RS0003-UM strand of |
| (<Strand Ref.: 95>) | | compound RD0017 (NT) |
| AUGUUGUUCUCGUCUCCUCGACACCAGGGGUCCACAU | 11 | RL0017-UM strand of |
| GGCAAC | | compound RD0017 (NT) |
| (<Strand Ref.: 96>) | | |
| mG*mG*mU.rG.mU.mC.rG.rA.rG.rA.rA.rG.rA. | 3 | RS0003 strand of compound |
| rG.rG.rA.rG.rA.rA.rC.mA.rA.mU*rA*mU | | RD0018 (NT) |
| (<Strand Ref.: 97>) | | |
| mA*fU*rG.fU.mU.rG.mU.mU.mC.mU.fC.rG.fU. | 11 | RL0018 strand of compound |
| mC.fU.rC.fC.mU.mC.rG.rA.mC.rA.mC.mC. | | RD0018 (NT) |
| fA.fG.fG.fG.fG.fU.rC.rC.rA.fC.fA.fU. | | |
| fG.fG.fC.fA*fA*fC | | |
| (<Strand Ref.: 98>) | | |
| GGUGUCGAGAAGAGGAGAACAAUAU | 3 | RS0003-UM strand of |
| (<Strand Ref.: 99>) | | compound RD0018 (NT) |
| AUGUUGUUCUCGUCUCCUCGACACCAGGGGUCCACAU | 11 | RL0018-UM strand of |
| GGCAAC | | compound RD0018 (NT) |
| (<Strand Ref.: 100>) | | |
| mG*mG*mU.rG.mU.mC.rG.rA.rG.rA.rA.rG.rA. | 3 | RS0003 strand of compound |
| rG.rG.rA.rG.rA.rA.rC.mA.rA.mU*rA*mU | | RD0019 (NT) |
| (<Strand Ref.: 101>) | | |
| mA*fU*rG.fU.mU.rG.mU.mU.mC.mU.fC.rG.fU. | 11 | RL0019 strand of compound |
| mC.fU.rC.fC.mU.mC.rG.rA.mC.rA.mC.mC. | | RD0019 (NT) |
| mA.fG.mG.fG.mG.fU.rC.rC.rA.fC.mA.fU. | | |
| mG.fG.mC.fA*mA*fC | | |
| (<Strand Ref.: 102>) | | |
| GGUGUCGAGAAGAGGAGAACAAUAU | 3 | RS0003-UM strand of |
| (<Strand Ref.: 103>) | | compound RD0019 (NT) |
| AUGUUGUUCUCGUCUCCUCGACACCAGGGGUCCACAU | 11 | RL0019-UM strand of |
| GGCAAC | | compound RD0019 (NT) |
| (<Strand Ref.: 104>) | | |
| rG.rG.rU.rG.rA.rA.rG.rA.rG.rG.rA.rG.rA. | 14 | RS0004 strand of compound |
| rA.rC.rA.rA.rU.rA.rU | | RD0020 (NT) |
| (<Strand Ref.: 105>) | | |
| rA.rU.rG.rU.rU.rG.rU.rU.rC.rU.rC.rG.rU. | 15 | RL0020 strand of compound |
| rC.rU.rC.rC.rA.rC.rC.rA.rG.rG.rG.rG. | | RD0020 (NT) |
| rU.rC.rC.rA.rC.rA.rU.rG.rG.rC.rA.rA. | | |
| rc | | |
| (<Strand Ref.: 106>) | | |
| GGUGAAGAGGAGAACAAUAU | 14 | RS0004-UM strand of |
| (<Strand Ref.: 107>) | | compound RD0020 (NT) |
| AUGUUGUUCUCGUCUCCACCAGGGGUCCACAUGGCAA | 15 | RL0020-UM strand of |
| C | | compound RD0020 (NT) |
| (<Strand Ref.: 108>) | | |
| rG.rG.rU.rG.rA.rA.rG.rA.rG.rG.rA.rG.rA. | 14 | RS0004 strand of compound |
| rA.rC.rA.rA.rU.rA.rU | | RD0021 (NT) |
| (<Strand Ref.: 109>) | | |
| rA.rU.rG.rU.rU.rG.rU.rU.rC.rU.rC.rG.rU. | 15 | RL0021 strand of compound |
| rC.rU.rC.rC.rA.rC.rC.mA.mG.mG.mG.mG. | | RD0021 (NT) |
| mU.rC.rC.rA.mC.mA.mU.mG.mG*mC*mA*mA* | | |
| mC | | |
| (<Strand Ref.: 110>) | | |
| GGUGAAGAGGAGAACAAUAU | 14 | RS0004-UM strand of |
| (<Strand Ref.: 111>) | | compound RD0021 (NT) |
| AUGUUGUUCUCGUCUCCACCAGGGGUCCACAUGGCAA | 15 | RL0021-UM strand of |
| C | | compound RD0021 (NT) |
| (<Strand Ref.: 112>) | | |
| rG.rG.rU.rG.rA.rA.rG.rA.rG.rG.rA.rG.rA. | 14 | RS0004 strand of compound |
| rA.rC.rA.rA.rU.rA.rU | | RD0022 (NT) |
| (<Strand Ref.: 113>) | | |
| rA.rU.rG.rU.rU.rG.rU.rU.rC.rU.rC.rG.rU. | 15 | RL0022 strand of compound |
| rC.rU.rC.rC.rA.rC.rC.rA.rG.rG.rG.rG. | | RD0022 (NT) |
| rU.rC.rC.rA.rC.rA.rU.rG.rG.rC.rA*rA* | | |
| rc | | |
| (<Strand Ref.: 114>) | | |
| GGUGAAGAGGAGAACAAUAU | 14 | RS0004-UM strand of |
| (<Strand Ref.: 115>) | | compound RD0022 (NT) |
| AUGUUGUUCUCGUCUCCACCAGGGGUCCACAUGGCAA | 15 | RL0022-UM strand of |
| C | | compound RD0022 (NT) |
| (<Strand Ref.: 116>) | | |
| rG.rG.rU.rG.rA.rA.rG.rA.rG.rG.rA.rG.rA. | 14 | RS0004 strand of compound |
| rA.rC.rA.rA.rU.rA.rU | | RD0023 (NT) |
| (<Strand Ref.: 117>) | | |
| rA.rU.rG.rU.rU.rG.rU.rU.rC.rU.rC.rG.rU. | 15 | RL0023 strand of compound |
| rC.rU.rC.rC.rA.rC.rC.fA.fG.fG.fG.fG. | | RD0023 (NT) |
| fU.rC.rC.rA.fC.fA.fU.fG.fG.fC.fA*fA* | | |
| fC | | |
| (<Strand Ref.: 118>) | | |
| GGUGAAGAGGAGAACAAUAU | 14 | RS0004-UM strand of |
| (<Strand Ref.: 119>) | | compound RD0023 (NT) |
| AUGUUGUUCUCGUCUCCACCAGGGGUCCACAUGGCAA | 1. | RL0023-UM strand of |
| C | | compound RD0023 (NT) |
| (<Strand Ref.: 120>) | | |
| rG.rG.rU.rG.rA.rA.rG.rA.rG.rG.rA.rG.rA. | 14 | RS0004 strand of compound |
| rA.rC.rA.rA.rU.rA.rU | | RD0024 (NT) |
| (<Strand Ref.: 121>) | | |
| rA.rU.rG.rU.rU.rG.rU.rU.rC.rU.rC.rG.rU. | 15 | RL0024 strand of compound |
| rC.rU.rC.rC.rA.rC.rC.mA.fG.mG.fG.mG. | | RD0024 (NT) |
| fU.rC.rC.rA.fC.mA.fU.mG.fG.mC.fA*mA* | | |
| fC | | |
| (<Strand Ref.: 122>) | | |
| GGUGAAGAGGAGAACAAUAU | 14 | RS0004-UM strand of |
| (<Strand Ref.: 123>) | | compound RD0024 (NT) |
| AUGUUGUUCUCGUCUCCACCAGGGGUCCACAUGGCAA | 15 | RL0024-UM strand of |
| C | | compound RD0024 (NT) |
| (<Strand Ref.: 124>) | | |
| rG.rG.rU.rG.rA.rA.rU.rA.rG.rU.rA.rU.rA. | 16 | RS0005 strand of compound |
| rA.rC.rA.rA.rU.rA.rU | | RD0025 (NT) |
| (<Strand Ref.: 125>) | | |
| rA.rU.rG.rU.rU.rG.rU.rU.rA.rU.rA.rG.rU. | 17 | RL0025 strand of compound |
| rA.rU.rC.rC.rA.rC.rC.mA.mG.mG.mG.mG. | | RD0025 (NT) |
| mU.rC.rC.rA.mC.mA.mU.mG.mG*mC*mA*mA* | | |
| mC | | |
| (<Strand Ref.: 126>) | | |
| GGUGAAUAGUAUAACAAUAU | 16 | RS0005-UM strand of |
| (<Strand Ref.: 127>) | | compound RD0025 (NT) |
| AUGUUGUUAUAGUAUCCACCAGGGGUCCACAUGGCAA | 17 | RL0025-UM strand of |
| C | | compound RD0025 (NT) |
| (<Strand Ref.: 128>) | | |
| rG.rG.rU.rG.rA.rA.rU.rA.rG.rU.rA.rU.rA. | 16 | RS0005 strand of compound |
| rA.rC.rA.rA.rU.rA.rU | | RD0026 (NT) |
| (<Strand Ref.: 129>) | | |
| rA.rU.rG.rU.rU.rG.rU.rU.rA.rU.rA.rG.rU. | 17 | RL0026 strand of compound |
| rA.rU.rC.rC.rA.rC.rC.rA.rG.rG.rG.rG. | | RD0026 (NT) |
| rU.rC.rC.rA.rC.rA.rU.rG.rG.rC.rA*rA* | | |
| rc | | |
| (<Strand Ref.: 130>) | | |
| GGUGAAUAGUAUAACAAUAU | 16 | RS0005-UM strand of |
| (<Strand Ref.: 131>) | | compound RD0026 (NT) |
| AUGUUGUUAUAGUAUCCACCAGGGGUCCACAUGGCAA | 17 | RL0026-UM strand of |
| C | | compound RD0026 (NT) |
| (<Strand Ref.: 132>) | | |
| rG.rG.rG.rU.rG.rG.rA.rA.rU.rA.rG.rU.rA. | 18 | RS0006 strand of compound |
| rU.rA.rA.rC.rA.rA.rU.rA.rU.rG.rC | | RD0027 (NT) |
| (<Strand Ref.: 133>) | | |
| rG.rC.rA.rU.rA.rU.rU.rG.rU.rU.rA.rU.rA. | 19 | RL0027 strand of compound |
| rG.rU.rA.rU.rC.rC.rC.rA.rC.rC.rC.rA. | | RD0027 (NT) |
| rG.rG.rG.rG.rU.rC.rC.rA.rC.rA.rU.rG. | | |
| rG.rC.rA*rA*rC | | |
| (<Strand Ref.: 134>) | | |
| GGGUGGAAUAGUAUAACAAUAUGC | 18 | RS0006-UM strand of |
| (<Strand Ref.: 135>) | | compound RD0027 (NT) |
| GCAUAUUGUUAUAGUAUCCCACCCAGGGGUCCACAUG | 19 | RL0027-UM strand of |
| GCAAC | | compound RD0027 (NT) |
| (<Strand Ref.: 136>) | | |
| rG.rG.rG.rU.rG.rG.rA.rA.rU.rA.rG.rU.rA. | 18 | RS0006 strand of compound |
| rU.rA.rA.rC.rA.rA.rU.rA.rU.rG.rC | | RD0028 (NT) |
| (<Strand Ref.: 137>) | | |
| rG.rC.rA.rU.rA.rU.rU.rG.rU.rU.rA.rU.rA. | 19 | RL0028 strand of compound |
| rG.rU.rA.rU.rC.rC.rC.rA.rC.rC.rC.mA. | | RD0028 (NT) |
| mG.mG.mG.mG.mU.rC.rC.rA.mC.mA.mU.mG. | | |
| mG*mC*mA*mA*mC | | |
| (<Strand Ref.: 138>) | | |
| GGGUGGAAUAGUAUAACAAUAUGC | 18 | RS0006-UM strand of |
| (<Strand Ref.: 139>) | | compound RD0028 (NT) |
| GCAUAUUGUUAUAGUAUCCCACCCAGGGGUCCACAUG | 19 | RL0028-UM strand of |
| GCAAC | | compound RD0028 (NT) |
| (<Strand Ref.: 140>) | | |
| rG.rG.rG.rU.rG.rG.rA.rA.rU.rA.rG.rU.rA. | 18 | RS0006 strand of compound |
| rU.rA.rA.rC.rA.rA.rU.rA.rU.rG.rC | | RD0029 (NT) |
| (<Strand Ref.: 141>) | | |
| rG.rC.rA.rU.rA.rU.rU.rG.rU.rU.rA.rU.rA. | 19 | RL0029 strand of compound |
| rG.rU.rA.rU.rC.rC.rC.rA.rC.rC.rC.mA. | | RD0029 (NT) |
| fG.mG.fG.mG.fU.rC.rC.rA.fC.mA.fU.mG. | | |
| fG.mC.fA*mA*fC | | |
| (<Strand Ref.: 142>) | | |
| GGGUGGAAUAGUAUAACAAUAUGC | 18 | RS0006-UM strand of |
| (< Strand Ref.: 143>) | | compound RD0029 (NT) |
| GCAUAUUGUUAUAGUAUCCCACCCAGGGGUCCACAUG | 19 | RL0029-UM strand of |
| GCAAC | | compound RD0029 (NT) |
| (<Strand Ref.: 144>) | | |
| rG*rG*rG.rU.rG.rG.rA.rA.rU.rA.rG.rU.rA. | 18 | RS0007 strand of compound |
| rU.rA.rA.rC.rA.rA.rU.rA.rU*rG*rC | | RD0030 (NT) |
| (<Strand Ref.: 145>) | | |
| rG*rC*rA.rU.rA.rU.rU.rG.rU.rU.rA.rU.rA. | 19 | RL0030 strand of compound |
| rG.rU.rA.rU.rC.rC.rC.rA.rC.rC.rC.rA. | | RD0030 (NT) |
| rG.rG.rG.rG.rU.rC.rC.rA.rC.rA.rU.rG. | | |
| rG.rC.rA*rA*rC | | |
| (<Strand Ref.: 146>) | | |
| GGGUGGAAUAGUAUAACAAUAUGC | 1 | RS0007-UM strand of |
| (<Strand Ref.: 147>) | | compound RD0030 (NT) |
| GCAUAUUGUUAUAGUAUCCCACCCAGGGGUCCACAUG | 19 | RL0030-UM strand of |
| GCAAC | | compound RD0030 (NT) |
| (<Strand Ref.: 148>) | | |
| rG*rG*rG.rU.rG.rG.rA.rA.rU.rA.rG.rU.rA. | 18 | RS0007 strand of compound |
| rU.rA.rA.rC.rA.rA.rU.rA.rU*rG*rC | | RD0031 (NT) |
| (<Strand Ref.: 149>) | | |
| rG*rC*rA.rU.rA.rU.rU.rG.rU.rU.rA.rU.rA. | 19 | RL0031 strand of compound |
| rG.rU.rA.rU.rC.rC.rC.rA.rC.rC.rC.mA. | | RD0031 (NT) |
| mG.mG.mG.mG.mU.rC.rC.rA.mC.mA.mU.mG. | | |
| mG*mC*mA*mA*mC | | |
| (<Strand Ref.: 150>) | | |
| GGGUGGAAUAGUAUAACAAUAUGC | 18 | RS0007-UM strand of |
| (<Strand Ref.: 151>) | | compound RD0031 (NT) |
| GCAUAUUGUUAUAGUAUCCCACCCAGGGGUCCACAUG | 19 | RL0031-UM strand of |
| GCAAC | | compound RD0031 (NT) |
| (<Strand Ref.: 152>) | | |
| rG*rG*rG.rU.rG.rG.rA.rA.rU.rA.rG.rU.rA. | 18 | RS0007 strand of compound |
| rU.rA.rA.rC.rA.rA.rU.rA.rU*rG*rC | | RD0032 (NT) |
| (<Strand Ref.: 153>) | | |
| rG*rC*rA.rU.rA.rU.rU.rG.rU.rU.rA.rU.rA. | 19 | RL0032 strand of compound |
| rG.rU.rA.rU.rC.rC.rC.rA.rC.rC.rC.mA. | | RD0032 (NT) |
| fG.mG.fG.mG.fU.rC.rC.rA.fC.mA.fU.mG. | | |
| fG.mC.fA*mA*fC | | |
| (<Strand Ref.: 154>) | | |
| GGGUGGAAUAGUAUAACAAUAUGC | 18 | RS0007-UM strand of |
| (<Strand Ref.: 155>) | | compound RD0032 (NT) |
| GCAUAUUGUUAUAGUAUCCCACCCAGGGGUCCACAUG | 19 | RL0032-UM strand of |
| GCAAC | | compound RD0032 (NT) |
| (<Strand Ref.: 156>) | | |
| rG*rG*rG.rU.rG.rG.rA.rA.rG.rA.rG.rU.rA. | 5 | RS0008 strand of compound |
| rG.rA.rA.rC.rA.rA.rU.rA.rU*rG*rC | | RD0033 (NT) |
| (<Strand Ref.: 157>) | | |
| rG*rC*rA.rU.rA.rU.rU.rG.rU.rU.rC.rU.rA. | 12 | RL0033 strand of compound |
| rG.rU.rC.rU.rC.rC.rC.rA.rC.rC.rC.rA. | | RD0033 (NT) |
| rG.rG.rG.rG.rU.rC.rC.rA.rC.rA.rU.rG. | | |
| rG.rC.rA*rA*rC | | |
| (<Strand Ref.: 158>) | | |
| GGGUGGAAGAGUAGAACAAUAUGC | 5 | RS0008-UM strand of |
| (<Strand Ref.: 159>) | | compound RD0033 (NT) |
| GCAUAUUGUUCUAGUCUCCCACCCAGGGGUCCACAUG | 12 | RL0033-UM strand of |
| GCAAC | | compound RD0033 (NT) |
| (<Strand Ref.: 160>) | | |
| rG*rG*rG.rU.rG.rG.rA.rA.rG.rA.rG.rU.rA. | 5 | RS0008 strand of compound |
| rG.rA.rA.rC.rA.rA.rU.rA.rU*rG*rC | | RD0034 (NT) |
| (<Strand Ref.: 161>) | | |
| rG*rC*rA.rU.rA.rU.rU.rG.rU.rU.rC.rU.rA. | 12 | RL0034 strand of compound |
| rG.rU.rC.rU.rC.rC.rC.rA.rC.rC.rC.mA. | | RD0034 (NT) |
| mG.mG.mG.mG.mU.rC.rC.rA.mC.mA.mU.mG. | | |
| mG*mC*mA*mA*mC | | |
| (<Strand Ref.: 162>) | | |
| GGGUGGAAGAGUAGAACAAUAUGC | 5 | RS0008-UM strand of |
| (<Strand Ref.: 163>) | | compound RD0034 (NT) |
| GCAUAUUGUUCUAGUCUCCCACCCAGGGGUCCACAUG | 12 | RL0034-UM strand of |
| GCAAC | | compound RD0034 (NT) |
| (<Strand Ref.: 164>) | | |
| rG*rG*rG.rU.rG.rG.rA.rA.rG.rA.rG.rU.rA. | 5 | RS0008 strand of compound |
| rG.rA.rA.rC.rA.rA.rU.rA.rU*rG*rC | | RD0035 (NT) |
| (<Strand Ref.: 165>) | | |
| rG*rC*rA.rU.rA.rU.rU.rG.rU.rU.rC.rU.rA. | 12 | RL0035 strand of compound |
| rG.rU.rC.rU.rC.rC.rC.rA.rC.rC.rC.mA. | | RD0035 (NT) |
| fG.mG.fG.mG.fU.rC.rC.rA.fC.mA.fU.mG. | | |
| fG.mC.fA*mA*fC | | |
| (<Strand Ref.: 166>) | | |
| GGGUGGAAGAGUAGAACAAUAUGC | 5 | RS0008-UM strand of |
| (<Strand Ref.: 167>) | | compound RD0035 (NT) |
| GCAUAUUGUUCUAGUCUCCCACCCAGGGGUCCACAUG | 12 | RL0035-UM strand of |
| GCAAC | | compound RD0035 (NT) |
| (<Strand Ref.: 168>) | | |
| rG*rG*rG.rU.rG.rG.rA.rA.rC.rA.rG.rU.rA. | 7 | RS0009 strand of compound |
| rC.rA.rA.rC.rA.rA.rU.rA.rU*rG*rC | | RD0036 (NT) |
| (<Strand Ref.: 169>) | | |
| rG*rC*rA.rU.rA.rU.rU.rG.rU.rU.rG.rU.rA. | 13 | RL0036 strand of compound |
| rG.rU.rG.rU.rC.rC.rC.rA.rC.rC.rC.rA. | | RD0036 (NT) |
| rG.rG.rG.rG.rU.rC.rC.rA.rC.rA.rU.rG. | | |
| rG.rC.rA*rA*rC | | |
| (<Strand Ref.: 170>) | | |
| GGGUGGAACAGUACAACAAUAUGC | 7 | RS0009-UM strand of |
| (<Strand Ref.: 171>) | | compound RD0036 (NT) |
| GCAUAUUGUUGUAGUGUCCCACCCAGGGGUCCACAUG | 13 | RL0036-UM strand of |
| GCAAC | | compound RD0036 (NT) |
| (<Strand Ref.: 172>) | | |
| rG*rG*rG.rU.rG.rG.rA.rA.rC.rA.rG.rU.rA. | 7 | RS0009 strand of compound |
| rC.rA.rA.rC.rA.rA.rU.rA.rU*rG*rC | | RD0037 (NT) |
| (<Strand Ref.: 173>) | | |
| rG*rC*rA.rU.rA.rU.rU.rG.rU.rU.rG.rU.rA. | 13 | RL0037 strand of compound |
| rG.rU.rG.rU.rC.rC.rC.rA.rC.rC.rC.mA. | | RD0037 (NT) |
| mG.mG.mG.mG.mU.rC.rC.rA.mC.mA.mU.mG. | | |
| mG*mC*mA*mA*mC | | |
| (<Strand Ref.: 174>) | | |
| GGGUGGAACAGUACAACAAUAUGC | 7 | RS0009-UM strand of |
| (<Strand Ref.: 175>) | | compound RD0037 (NT) |
| GCAUAUUGUUGUAGUGUCCCACCCAGGGGUCCACAUG | 13 | RL0037-UM strand of |
| GCAAC | | compound RD0037 (NT) |
| (<Strand Ref.: 176>) | | |
| rG*rG*rG.rU.rG.rG.rA.rA.rC.rA.rG.rU.rA. | 7 | RS0009 strand of compound |
| rC.rA.rA.rC.rA.rA.rU.rA.rU*rG*rC | | RD0038 (NT) |
| (<Strand Ref.: 177>) | | |
| rG*rC*rA.rU.rA.rU.rU.rG.rU.rU.rG.rU.rA. | 13 | RL0038 strand of compound |
| rG.rU.rG.rU.rC.rC.rC.rA.rC.rC.rC.mA. | | RD0038 (NT) |
| fG.mG.fG.mG.fU.rC.rC.rA.fC.mA.fU.mG. | | |
| fG.mC.fA*mA*fC | | |
| (<Strand Ref.: 178>) | | |
| GGGUGGAACAGUACAACAAUAUGC | 7 | RS0009-UM strand of |
| (<Strand Ref.: 179>) | | compound RD0038 (NT) |
| GCAUAUUGUUGUAGUGUCCCACCCAGGGGUCCACAUG | 13 | RL0038-UM strand of |
| GCAAC | | compound RD0038 (NT) |
| (<Strand Ref.: 180>) | | |
| rG*rG*rG.rU.rG.rG.rA.rA.rU.rA.rG.rU.rA. | 20 | RS0010 strand of compound |
| rC.rA.rA.rC.rA.rA.rU.rA.rU*rG*rC | | RD0039 (NT) |
| (<Strand Ref.: 181>) | | |
| rG*rC*rA.rU.rA.rU.rU.rG.rU.rU.rG.rU.rA. | 21 | RL0039 strand of compound |
| rG.rU.rA.rU.rC.rC.rC.rA.rC.rC.rC.rA. | | RD0039 (NT) |
| rG.rG.rG.rG.rU.rC.rC.rA.rC.rA.rU.rG. | | |
| rG.rC.rA*rA*rC | | |
| (<Strand Ref.: 182>) | | |
| GGGUGGAAUAGUACAACAAUAUGC | 20 | RS0010-UM strand of |
| (<Strand Ref.: 183>) | | compound RD0039 (NT) |
| GCAUAUUGUUGUAGUAUCCCACCCAGGGGUCCACAUG | 21 | RL0039-UM strand of |
| GCAAC | | compound RD0039 (NT) |
| (<Strand Ref.: 184>) | | |
| rG*rG*rG.rU.rG.rG.rA.rA.rU.rA.rG.rU.rA. | 20 | RS0010 strand of compound |
| rC.rA.rA.rC.rA.rA.rU.rA.rU*rG*rC | | RD0040 (NT) |
| (<Strand Ref.: 185>) | | |
| rG*rC*rA.rU.rA.rU.rU.rG.rU.rU.rG.rU.rA. | 21 | RL0040 strand of compound |
| rG.rU.rA.rU.rC.rC.rC.rA.rC.rC.rC.mA. | | RD0040 (NT) |
| mG.mG.mG.mG.mU.rC.rC.rA.mC.mA.mU.mG. | | |
| mG*mC*mA*mA*mC | | |
| (<Strand Ref.: 186>) | | |
| GGGUGGAAUAGUACAACAAUAUGC | 20 | RS0010-UM strand of |
| (<Strand Ref.: 187>) | | compound RD0040 (NT) |
| GCAUAUUGUUGUAGUAUCCCACCCAGGGGUCCACAUG | 21 | RL0040-UM strand of |
| GCAAC | | compound RD0040 (NT) |
| (<Strand Ref.: 188>) | | |
| rG*rG*rG.rU.rG.rG.rA.rA.rU.rA.rG.rU.rA. | 20 | RS0010 strand of compound |
| rC.rA.rA.rC.rA.rA.rU.rA.rU*rG*rC | | RD0041 (NT) |
| (<Strand Ref.: 189>) | | |
| rG*rC*rA.rU.rA.rU.rU.rG.rU.rU.rG.rU.rA. | 21 | RL0041 strand of compound |
| rG.rU.rA.rU.rC.rC.rC.rA.rC.rC.rC.mA. | | RD0041 (NT) |
| fG.mG.fG.mG.fU.rC.rC.rA.fC.mA.fU.mG. | | |
| fG.mC.fA*mA*fC | | |
| (<Strand Ref.: 190>) | | |
| GGGUGGAAUAGUACAACAAUAUGC | | RS0010-UM strand of |
| (<Strand Ref.: 191>) | | compound RD0041 (NT) |
| GCAUAUUGUUGUAGUAUCCCACCCAGGGGUCCACAUG | 21 | RL0041-UM strand of |
| GCAAC | | compound RD0041 (NT) |
| (<Strand Ref.: 192>) | | |
| rG*rG*rG.rU.rG.rG.rA.rA.rU.rA.rG.rU.rA. | 22 | RS0011 strand of compound |
| rU.rA.rA.rC.rA.rA.rU*rA*rU | | RD0042 (NT) |
| (<Strand Ref.: 193>) | | |
| rA*rU*rA.rU.rU.rG.rU.rU.rA.rU.rA.rG.rU. | 23 | RL0042 strand of compound |
| rA.rU.rC.rC.rC.rA.rC.rC.rC.rA.rG.rG. | | RD0042 (NT) |
| rG.rG.rU.rC.rC.rA.rC.rA.rU.rG.rG.rC. | | |
| rA*rA*rC | | |
| (<Strand Ref.: 194>) | | |
| GGGUGGAAUAGUAUAACAAUAU | 22 | RS0011-UM strand of |
| (<Strand Ref.: 195>) | | compound RD0042 (NT) |
| AUAUUGUUAUAGUAUCCCACCCAGGGGUCCACAUGGC | 23 | RL0042-UM strand of |
| AAC | | compound RD0042 (NT) |
| (<Strand Ref.: 196>) | | |
| rG*rG*rG.rU.rG.rG.rA.rA.rU.rA.rG.rU.rA. | 22 | RS0011 strand of compound |
| rU.rA.rA.rC.rA.rA.rU*rA*rU | | RD0043 (NT) |
| (<Strand Ref.: 197>) | | |
| rA*rU*rA.rU.rU.rG.rU.rU.rA.rU.rA.rG.rU. | 23 | RL0043 strand of compound |
| rA.rU.rC.rC.rC.rA.rC.rC.rC.mA.mG.mG. | | RD0043 (NT) |
| mG.mG.mU.rC.rC.rA.mC.mA.mU.mG.mG*mC* | | |
| mA*mA*mC | | |
| (<Strand Ref.: 198>) | | |
| GGGUGGAAUAGUAUAACAAUAU | 22 | RS0011-UM strand of |
| (<Strand Ref.: 199>) | | compound RD0043 (NT) |
| AUAUUGUUAUAGUAUCCCACCCAGGGGUCCACAUGGC | 23 | RL0043-UM strand of |
| AAC | | compound RD0043 (NT) |
| (<Strand Ref.: 200>) | | |
| rG*rG*rG.rU.rG.rG.rA.rA.rU.rA.rG.rU.rA. | 22 | RS0011 strand of compound |
| rU.rA.rA.rC.rA.rA.rU*rA*rU | | RD0044 (NT) |
| (<Strand Ref.: 201>) | | |
| rA*rU*rA.rU.rU.rG.rU.rU.rA.rU.rA.rG.rU. | 23 | RL0044 strand of compound |
| rA.rU.rC.rC.rC.rA.rC.rC.rC.mA.fG.mG. | | RD0044 (NT) |
| fG.mG.fU.rC.rC.rA.fC.mA.fU.mG.fG.mC. | | |
| fA*mA*fC | | |
| (<Strand Ref.: 202>) | | |
| GGGUGGAAUAGUAUAACAAUAU | 22 | RS0011-UM strand of |
| (<Strand Ref.: 203>) | | compound RD0044 (NT) |
| AUAUUGUUAUAGUAUCCCACCCAGGGGUCCACAUGGC | 23 | RL0044-UM strand of |
| AAC | | compound RD0044 (NT) |
| (<Strand Ref.: 204>) | | |
| rG*rG*rG.rU.rG.rG.rA.rA.rU.rA.rG.rU.rA. | 24 | RS0012 strand of compound |
| rU.rA.rA.rC.rA.rA.rU*rG*rC | | RD0045 (NT) |
| (<Strand Ref.: 205>) | | |
| rG*rC*rA.rU.rU.rG.rU.rU.rA.rU.rA.rG.rU. | 25 | RL0045 strand of compound |
| rA.rU.rC.rC.rC.rA.rC.rC.rC.rA.rG.rG. | | RD0045 (NT) |
| rG.rG.rU.rC.rC.rA.rC.rA.rU.rG.rG.rC. | | |
| rA*rA*rC | | |
| (<Strand Ref.: 206>) | | |
| GGGUGGAAUAGUAUAACAAUGC | 24 | RS0012-UM strand of |
| (<Strand Ref.: 207>) | | compound RD0045 (NT) |
| GCAUUGUUAUAGUAUCCCACCCAGGGGUCCACAUGGC | 25 | RL0045-UM strand of |
| AAC | | compound RD0045 (NT) |
| (<Strand Ref.: 208>) | | |
| rG*rG*rG.rU.rG.rG.rA.rA.rU.rA.rG.rU.rA. | 24 | RS0012 strand of compound |
| rU.rA.rA.rC.rA.rA.rU*rG*rC | | RD0046 (NT) |
| (<Strand Ref.: 209>) | | |
| rG*rC*rA.rU.rU.rG.rU.rU.rA.rU.rA.rG.rU. | 25 | RL0046 strand of compound |
| rA.rU.rC.rC.rC.rA.rC.rC.rC.mA.mG.mG. | | RD0046 (NT) |
| mG.mG.mU.rC.rC.rA.mC.mA.mU.mG.mG*mC* | | |
| mA*mA*mC | | |
| (<Strand Ref.: 210>) | | |
| GGGUGGAAUAGUAUAACAAUGC | 24 | RS0012-UM strand of |
| (<Strand Ref.: 211>) | | compound RD0046 (NT) |
| GCAUUGUUAUAGUAUCCCACCCAGGGGUCCACAUGGC | 25 | RL0046-UM strand of |
| AAC | | compound RD0046 (NT) |
| (<Strand Ref.: 212>) | | |
| rG*rG*rG.rU.rG.rG.rA.rA.rU.rA.rG.rU.rA. | 24 | RS0012 strand of compound |
| rU.rA.rA.rC.rA.rA.rU*rG*rC | | RD0047 (NT) |
| (<Strand Ref.: 213>) | | |
| rG*rC*rA.rU.rU.rG.rU.rU.rA.rU.rA.rG.rU. | 25 | RL0047 strand of compound |
| rA.rU.rC.rC.rC.rA.rC.rC.rC.mA.fG.mG. | | RD0047 (NT) |
| fG.mG.fU.rC.rC.rA.fC.mA.fU.mG.fG.mC. | | |
| fA*mA*fC | | |
| (<Strand Ref.: 214>) | | |
| GGGUGGAAUAGUAUAACAAUGC | 24 | RS0012-UM strand of |
| (<Strand Ref.: 215>) | | compound RD0047 (NT) |
| GCAUUGUUAUAGUAUCCCACCCAGGGGUCCACAUGGC | 25 | RL0047-UM strand of |
| AAC | | compound RD0047 (NT) |
| (<Strand Ref.: 216>) | | |
| rG*rG*rG.rU.rG.rG.rA.rA.rU.rA.rG.rU.rA. | 24 | RS0012 strand of compound |
| rU.rA.rA.rC.rA.rA.rU*rG*rC | | RD0048 (NT) |
| (<Strand Ref.: 217>) | | |
| rG*rC*rA.rU.rU.rG.rU.rU.rA.rU.rA.rG.rU. | 25 | RL0048 strand of compound |
| rA.rU.rC.rC.rC.rA.rC.rC.rC.rA.rG.rG. | | RD0048 (NT) |
| rG.rG.rU.rC.rC.rA.rC.rA.rU.rG.rG.rC. | | |
| rA.rA.rC | | |
| (<Strand Ref.: 218>) | | |
| GGGUGGAAUAGUAUAACAAUGC | 24 | RS0012-UM strand of |
| (<Strand Ref.: 219>) | | compound RD0048 (NT) |
| GCAUUGUUAUAGUAUCCCACCCAGGGGUCCACAUGGC | 25 | RL0048-UM strand of |
| AAC | | compound RD0048 (NT) |
| (<Strand Ref.: 220>) | | |
| rG*rG*rG.rU.rG.rG.rA.rA.rU.rA.rG.rU.rA. | 26 | RS0013 strand of compound |
| rC.rA.rA.rC.rA.rA.rU*rG*rC | | RD0049 (NT) |
| (<Strand Ref.: 221>) | | |
| rG*rC*rA.rU.rU.rG.rU.rU.rG.rU.rA.rG.rU. | 27 | RL0049 strand of compound |
| rA.rU.rC.rC.rC.rA.rC.rC.rC.rA.rG.rG. | | RD0049 (NT) |
| rG.rG.rU.rC.rC.rA.rC.rA.rU.rG.rG.rC. | | |
| rA.rA.rC | | |
| (<Strand Ref.: 222>) | | |
| GGGUGGAAUAGUACAACAAUGC | 26 | RS0013-UM strand of |
| (<Strand Ref.: 223>) | | compound RD0049 (NT) |
| GCAUUGUUGUAGUAUCCCACCCAGGGGUCCACAUGGC | 27 | RL0049-UM strand of |
| AAC | | compound RD0049 (NT) |
| (<Strand Ref.: 224>) | | |
| rG*rG*rG.rU.rG.rG.rA.rA.rU.rA.rG.rU.rA. | 26 | RS0013 strand of compound |
| rC.rA.rA.rC.rA.rA.rU*rG*rC | | RD0050 (NT) |
| (<Strand Ref.: 225>) | | |
| rG*rC*rA.rU.rU.rG.rU.rU.rG.rU.rA.rG.rU. | 27 | RL0050 strand of compound |
| rA.rU.rC.rC.rC.rA.rC.rC.rC.rA.rG.rG. | | RD0050 (NT) |
| rG.rG.rU.rC.rC.rA.rC.rA.rU.rG.rG.rC. | | |
| rA*rA*rC | | |
| (<Strand Ref.: 226>) | | |
| GGGUGGAAUAGUACAACAAUGC | 26 | RS0013-UM strand of |
| (<Strand Ref.: 227>) | | compound RD0050 (NT) |
| GCAUUGUUGUAGUAUCCCACCCAGGGGUCCACAUGGC | 27 | RL0050-UM strand of |
| AAC | | compound RD0050 (NT) |
| (<Strand Ref.: 228>) | | |
| rG*rG*rG.rU.rG.rG.rA.rA.rU.rA.rG.rU.rA. | 26 | RS0013 strand of compound |
| rC.rA.rA.rC.rA.rA.rU*rG*rC | | RD0051 (NT) |
| (<Strand Ref.: 229>) | | |
| rG*rC*rA.rU.rU.rG.rU.rU.rG.rU.rA.rG.rU. | 27 | RL0051 strand of compound |
| rA.rU.rC.rC.rC.rA.rC.rC.rC.mA.mG.mG. | | RD0051 (NT) |
| mG.mG.mU.rC.rC.rA.mC.mA.mU.mG.mG*mC* | | |
| mA*mA*mC | | |
| (<Strand Ref.: 230>) | | |
| GGGUGGAAUAGUACAACAAUGC | 26 | RS0013-UM strand of |
| (<Strand Ref.: 231>) | | compound RD0051 (NT) |
| GCAUUGUUGUAGUAUCCCACCCAGGGGUCCACAUGGC | 27 | RL0051-UM strand of |
| AAC | | compound RD0051 (NT) |
| (<Strand Ref.: 232>) | | |
| rG*rG*rG.rU.rG.rG.rA.rA.rU.rA.rG.rU.rA. | 26 | RS0013 strand of compound |
| rC.rA.rA.rC.rA.rA.rU*rG*rC | | RD0052 (NT) |
| (<Strand Ref.: 233>) | | |
| rG*rC*rA.rU.rU.rG.rU.rU.rG.rU.rA.rG.rU. | 27 | RL0052 strand of compound |
| rA.rU.rC.rC.rC.rA.rC.rC.rC.mA.fG.mG. | | RD0052 (NT) |
| fG.mG.fU.rC.rC.rA.fC.mA.fU.mG.fG.mC. | | |
| fA*mA*fC | | |
| (<Strand Ref.: 234>) | | |
| GGGUGGAAUAGUACAACAAUGC | 26 | RS0013-UM strand of |
| (<Strand Ref.: 235>) | | compound RD0052 (NT) |
| GCAUUGUUGUAGUAUCCCACCCAGGGGUCCACAUGGC | 27 | RL0052-UM strand of |
| AAC | | compound RD0052 (NT) |
| (<Strand Ref.: 236>) | | |
| rG.rG.rG.rU.rG.rG.rA.rA.rU.rA.rG.rU.rA. | 26 | RS0014 strand of compound |
| rC.rA.rA.rC.rA.rA.rU.rG.rC | | RD0053 (NT) |
| (<Strand Ref.: 237>) | | |
| rG.rC.rA.rU.rU.rG.rU.rU.rG.rU.rA.rG.rU. | 27 | RL0053 strand of compound |
| rA.rU.rC.rC.rC.rA.rC.rC.rC.rA.rG.rG. | | RD0053 (NT) |
| rG.rG.rU.rC.rC.rA.rC.rA.rU.rG.rG.rC. | | |
| rA.rA.rC | | |
| (<Strand Ref.: 238>) | | |
| GGGUGGAAUAGUACAACAAUGC | 26 | RS0014-UM strand of |
| (<Strand Ref.: 239>) | | compound RD0053 (NT) |
| GCAUUGUUGUAGUAUCCCACCCAGGGGUCCACAUGGC | 27 | RL0053-UM strand of |
| AAC | | compound RD0053 (NT) |
| (<Strand Ref.: 240>) | | |
| rG.rG.rG.rU.rG.rG.rA.rA.rU.rA.rG.rU.rA. | 26 | RS0014 strand of compound |
| rC.rA.rA.rC.rA.rA.rU.rG.rC | | RD0054 (NT) |
| (<Strand Ref.: 241>) | | |
| rG.rC.rA.rU.rU.rG.rU.rU.rG.rU.rA.rG.rU. | 27 | RL0054 strand of compound |
| rA.rU.rC.rC.rC.rA.rC.rC.rC.rA.rG.rG. | | RD0054 (NT) |
| rG.rG.rU.rC.rC.rA.rC.rA.rU.rG.rG.rC. | | |
| rA*rA*rC | | |
| (<Strand Ref.: 242>) | | |
| GGGUGGAAUAGUACAACAAUGC | 26 | RS0014-UM strand of |
| (<Strand Ref.: 243>) | | compound RD0054 (NT) |
| GCAUUGUUGUAGUAUCCCACCCAGGGGUCCACAUGGC | 27 | RL0054-UM strand of |
| AAC | | compound RD0054 (NT) |
| (<Strand Ref.: 244>) | | |
| rG.rG.rG.rU.rG.rG.rA.rA.rU.rA.rG.rU.rA. | 26 | RS0014 strand of compound |
| rC.rA.rA.rC.rA.rA.rU.rG.rC | | RD0055 (NT) |
| (<Strand Ref.: 245>) | | |
| rG.rC.rA.rU.rU.rG.rU.rU.rG.rU.rA.rG.rU. | 27 | RL0055 strand of compound |
| rA.rU.rC.rC.rC.rA.rC.rC.rC.mA.mG.mG. | | RD0055 (NT) |
| mG.mG.mU.rC.rC.rA.mC.mA.mU.mG.mG*mC* | | |
| mA*mA*mC | | |
| (<Strand Ref.: 246>) | | |
| GGGUGGAAUAGUACAACAAUGC | 26 | RS0014-UM strand of |
| (<Strand Ref.: 247>) | | compound RD0055 (NT) |
| GCAUUGUUGUAGUAUCCCACCCAGGGGUCCACAUGGC | 27 | RL0055-UM strand of |
| AAC | | compound RD0055 (NT) |
| (<Strand Ref.: 248>) | | |
| rG.rG.rG.rU.rG.rG.rA.rA.rU.rA.rG.rU.rA. | 26 | RS0014 strand of compound |
| rC.rA.rA.rC.rA.rA.rU.rG.rC | | RD0056 (NT) |
| (<Strand Ref.: 249>) | | |
| rG.rC.rA.rU.rU.rG.rU.rU.rG.rU.rA.rG.rU. | 27 | RL0056 strand of compound |
| rA.rU.rC.rC.rC.rA.rC.rC.rC.mA.fG.mG. | | RD0056 (NT) |
| fG.mG.fU.rC.rC.rA.fC.mA.fU.mG.fG.mC. | | |
| fA*mA*fC | | |
| (<Strand Ref.: 250>) | | |
| GGGUGGAAUAGUACAACAAUGC | 26 | RS0014-UM strand of |
| (<Strand Ref.: 251>) | | compound RD0056 (NT) |
| GCAUUGUUGUAGUAUCCCACCCAGGGGUCCACAUGGC | 27 | RL0056-UM strand of |
| AAC | | compound RD0056 (NT) |
| (<Strand Ref.: 252>) | | |
| rG.rG.rG.rU.rG.rG.rA.rA.rU.rA.rG.rU.rA. | 22 | RS0015 strand of compound |
| rU.rA.rA.rC.rA.rA.rU.rA.rU | | RD0057 (NT) |
| (<Strand Ref.: 253>) | | |
| rA.rU.rA.rU.rU.rG.rU.rU.rA.rU.rA.rG.rU. | 23 | RL0057 strand of compound |
| rA.rU.rC.rC.rC.rA.rC.rC.rC.rA.rG.rG. | | RD0057 (NT) |
| rG.rG.rU.rC.rC.rA.rC.rA.rU.rG.rG.rC. | | |
| rA*rA*rC | | |
| (<Strand Ref.: 254>) | | |
| GGGUGGAAUAGUAUAACAAUAU | 22 | RS0015-UM strand of |
| (<Strand Ref.: 255>) | | compound RD0057 (NT) |
| AUAUUGUUAUAGUAUCCCACCCAGGGGUCCACAUGGC | 23 | RL0057-UM strand of |
| AAC | | compound RD0057 (NT) |
| (<Strand Ref.: 256>) | | |
| rG.rG.rG.rU.rG.rG.rA.rA.rU.rA.rG.rU.rA. | 22 | RS0015 strand of compound |
| rU.rA.rA.rC.rA.rA.rU.rA.rU | | RD0058 (NT) |
| (<Strand Ref.: 257>) | | |
| rA.rU.rA.rU.rU.rG.rU.rU.rA.rU.rA.rG.rU. | 23 | RL0058 strand of compound |
| rA.rU.rC.rC.rC.rA.rC.rC.rC.mA.mG.mG. | | RD0058 (NT) |
| mG.mG.mU.rC.rC.rA.mC.mA.mU.mG.mG*mC* | | |
| mA*mA*mC | | |
| (<Strand Ref.: 258>) | | |
| GGGUGGAAUAGUAUAACAAUAU | 22 | RS0015-UM strand of |
| (<Strand Ref.: 259>) | | compound RD0058 (NT) |
| AUAUUGUUAUAGUAUCCCACCCAGGGGUCCACAUGGC | 23 | RL0058-UM strand of |
| AAC | | compound RD0058 (NT) |
| (<Strand Ref.: 260>) | | |
| rG.rG.rG.rU.rG.rG.rA.rA.rU.rA.rG.rU.rA. | 22 | RS0015 strand of compound |
| rU.rA.rA.rC.rA.rA.rU.rA.rU | | RD0059 (NT) |
| (<Strand Ref.: 261>) | | |
| rA.rU.rA.rU.rU.rG.rU.rU.rA.rU.rA.rG.rU. | 23 | RL0059 strand of compound |
| rA.rU.rC.rC.rC.rA.rC.rC.rC.mA.fG.mG. | | RD0059 (NT) |
| fG.mG.fU.rC.rC.rA.fC.mA.fU.mG.fG.mC. | | |
| fA*mA*fC | | |
| (<Strand Ref.: 262>) | | |
| GGGUGGAAUAGUAUAACAAUAU | 22 | RS0015-UM strand of |
| (<Strand Ref.: 263>) | | compound RD0059 (NT) |
| AUAUUGUUAUAGUAUCCCACCCAGGGGUCCACAUGGC | 23 | RL0059-UM strand of |
| AAC | | compound RD0059 (NT) |
| (<Strand Ref.: 264>) | | |
| rG.rG.rU.rG.rG.rG.rU.rG.rG.rA.rA.rU.rA. | 28 | RS0016 strand of compound |
| rG.rU.rA.rU.rA.rA.rC.rA.rA.rU.rA.rU | | RD0060 (NT) |
| (<Strand Ref.: 265>) | | |
| rA.rU.rG.rU.rU.rG.rU.rU.rA.rU.rA.rG.rU. | 29 | RL0060 strand of compound |
| rA.rU.rC.rC.rC.rA.rC.rC.rC.rA.rC.rC. | | RD0060 (NT) |
| rA.rG.rG.rG.rG.rU.rC.rC.rA.rC.rA.rU. | | |
| rG.rG.rC.rA.rA.rC | | |
| (<Strand Ref.: 266>) | | |
| GGUGGGUGGAAUAGUAUAACAAUAU | 28 | RS0016-UM strand of |
| (<Strand Ref.: 267>) | | compound RD0060 (NT) |
| AUGUUGUUAUAGUAUCCCACCCACCAGGGGUCCACAU | 29 | RL0060-UM strand of |
| GGCAAC | | compound RD0060 (NT) |
| (<Strand Ref.: 268>) | | |
| rG.rG.rU.rG.rG.rG.rU.rG.rG.rA.rA.rU.rA. | 28 | RS0016 strand of compound |
| rG.rU.rA.rU.rA.rA.rC.rA.rA.rU.rA.rU | | RD0061 (NT) |
| (<Strand Ref.: 269>) | | |
| rA.rU.rG.rU.rU.rG.rU.rU.rA.rU.rA.rG.rU. | 29 | RL0061 strand of compound |
| rA.rU.rC.rC.rC.rA.rC.rC.rC.rA.rC.rC. | | RD0061 (NT) |
| mA.mG.mG.mG.mG.mU.rC.rC.rA.mC.mA.mU. | | |
| mG.mG*mC*mA*mA*mC | | |
| (<Strand Ref.: 270>) | | |
| GGUGGGUGGAAUAGUAUAACAAUAU | 28 | RS0016-UM strand of |
| (<Strand Ref.: 271>) | | compound RD0061 (NT) |
| AUGUUGUUAUAGUAUCCCACCCACCAGGGGUCCACAU | 29 | RL0061-UM strand of |
| GGCAAC | | compound RD0061 (NT) |
| (<Strand Ref.: 272>) | | |
| rG.rG.rU.rG.rG.rG.rU.rG.rG.rA.rA.rU.rA. | 28 | RS0016 strand of compound |
| rG.rU.rA.rU.rA.rA.rC.rA.rA.rU.rA.rU | | RD0062 (NT) |
| (<Strand Ref.: 273>) | | |
| rA.rU.rG.rU.rU.rG.rU.rU.rA.rU.rA.rG.rU. | 29 | RL0062 strand of compound |
| rA.rU.rC.rC.rC.rA.rC.rC.rC.rA.rC.rC. | | RD0062 (NT) |
| mA.fG.mG.fG.mG.fU.rC.rC.rA.fC.mA.fU. | | |
| mG.fG.mC.fA*mA*fC | | |
| (<Strand Ref.: 274>) | | |
| GGUGGGUGGAAUAGUAUAACAAUAU | 28 | RS0016-UM strand of |
| (<Strand Ref.: 275>) | | compound RD0062 (NT) |
| AUGUUGUUAUAGUAUCCCACCCACCAGGGGUCCACAU | 29 | RL0062-UM strand of |
| GGCAAC | | compound RD0062 (NT) |
| (<Strand Ref.: 276>) | | |
| rG.rG.rU.rG.rG.rG.rU.rG.rG.rA.rA.rU.rA. | 28 | RS0016 strand of compound |
| rG.rU.rA.rU.rA.rA.rC.rA.rA.rU.rA.rU | | RD0063 (NT) |
| (<Strand Ref.: 277>) | | |
| rA.rU.rG.rU.rU.rG.rU.rU.rA.rU.rA.rG.rU. | 29 | RL0063 strand of compound |
| rA.rU.rC.rC.rC.rA.rC.rC.rC.rA.rC.rC. | | RD0063 (NT) |
| rA.rG.rG.rG.rG.rU.rC.rC.rA.rC.rA.rU. | | |
| rG.rG.rC.rA*rA*rC | | |
| (<Strand Ref.: 278>) | | |
| GGUGGGUGGAAUAGUAUAACAAUAU | 28 | RS0016-UM strand of |
| (<Strand Ref.: 279>) | | compound RD0063 (NT) |
| AUGUUGUUAUAGUAUCCCACCCACCAGGGGUCCACAU | 29 | RL0063-UM strand of |
| GGCAAC | | compound RD0063 (NT) |
| (<Strand Ref.: 280>) | | |
| rG.rG.rU.rG.rG.rG.rU.rG.rG.rA.rA.rG.rA. | 30 | RS0017 strand of compound |
| rG.rU.rA.rU.rA.rA.rC.rA.rA.rU.rG.rC | | RD0064 (NT) |
| (<Strand Ref.: 281>) | | |
| rG.rC.rG.rU.rU.rG.rU.rU.rA.rU.rA.rG.rU. | 31 | RL0064 strand of compound |
| rC.rU.rC.rC.rC.rA.rC.rC.rC.rA.rC.rC. | | RD0064 (NT) |
| rA.rG.rG.rG.rG.rU.rC.rC.rA.rC.rA.rU. | | |
| rG.rG.rC.rA.rA.rC | | |
| (<Strand Ref.: 282>) | | |
| GGUGGGUGGAAGAGUAUAACAAUGC | 30 | RS0017-UM strand of |
| (<Strand Ref.: 283>) | | compound RD0064 (NT) |
| GCGUUGUUAUAGUCUCCCACCCACCAGGGGUCCACAU | 31 | RL0064-UM strand of |
| GGCAAC | | compound RD0064 (NT) |
| (<Strand Ref.: 284>) | | |
| rG.rG.rU.rG.rG.rG.rU.rG.rG.rA.rA.rG.rA. | 30 | RS0017 strand of compound |
| rG.rU.rA.rU.rA.rA.rC.rA.rA.rU.rG.rC | | RD0065 (NT) |
| (<Strand Ref.: 285>) | | |
| rG.rC.rG.rU.rU.rG.rU.rU.rA.rU.rA.rG.rU. | 31 | RL0065 strand of compound |
| rC.rU.rC.rC.rC.rA.rC.rC.rC.rA.rC.rC. | | RD0065 (NT) |
| mA.mG.mG.mG.mG.mU.rC.rC.rA.mC.mA.mU. | | |
| mG.mG*mC*mA*mA*mC | | |
| (<Strand Ref.: 286>) | | |
| GGUGGGUGGAAGAGUAUAACAAUGC | 30 | RS0017-UM strand of |
| (<Strand Ref.: 287>) | | compound RD0065 (NT) |
| GCGUUGUUAUAGUCUCCCACCCACCAGGGGUCCACAU | 31 | RL0065-UM strand of |
| GGCAAC | | compound RD0065 (NT) |
| (<Strand Ref.: 288>) | | |
| rG.rG.rU.rG.rG.rG.rU.rG.rG.rA.rA.rG.rA. | 30 | RS0017 strand of compound |
| rG.rU.rA.rU.rA.rA.rC.rA.rA.rU.rG.rC | | RD0066 (NT) |
| (<Strand Ref.: 289>) | | |
| rG.rC.rG.rU.rU.rG.rU.rU.rA.rU.rA.rG.rU. | 31 | RL0066 strand of compound |
| rC.rU.rC.rC.rC.rA.rC.rC.rC.rA.rC.rC. | | RD0066 (NT) |
| rA.rG.rG.rG.rG.rU.rC.rC.rA.rC.rA.rU. | | |
| rG.rG.rC.rA*rA*rC | | |
| (<Strand Ref.: 290>) | | |
| GGUGGGUGGAAGAGUAUAACAAUGC | 30 | RS0017-UM strand of |
| (<Strand Ref.: 291>) | | compound RD0066 (NT) |
| GCGUUGUUAUAGUCUCCCACCCACCAGGGGUCCACAU | 31 | RL0066-UM strand of |
| GGCAAC | | compound RD0066 (NT) |
| (<Strand Ref.: 292>) | | |
| rG.rG.rU.rG.rG.rG.rU.rG.rG.rA.rA.rG.rA. | 30 | RS0017 strand of compound |
| rG.rU.rA.rU.rA.rA.rC.rA.rA.rU.rG.rC | | RD0067 (NT) |
| (<Strand Ref.: 293>) | | |
| rG.rC.rG.rU.rU.rG.rU.rU.rA.rU.rA.rG.rU. | 31 | RL0067 strand of compound |
| rC.rU.rC.rC.rC.rA.rC.rC.rC.rA.rC.rC. | | RD0067 (NT) |
| mA.fG.mG.fG.mG.fU.rC.rC.rA.fC.mA.fU. | | |
| mG.fG.mC.fA*mA*fC | | |
| (<Strand Ref.: 294>) | | |
| GGUGGGUGGAAGAGUAUAACAAUGC | 30 | RS0017-UM strand of |
| (<Strand Ref.: 295>) | | compound RD0067 (NT) |
| GCGUUGUUAUAGUCUCCCACCCACCAGGGGUCCACAU | 31 | RL0067-UM strand of |
| GGCAAC | | compound RD0067 (NT) |
| (<Strand Ref.: 296>) | | |
| rG.rG.rU.rG.rG.rG.rU.rG.rG.rA.rA.rC.rA. | 32 | RS0018 strand of compound |
| rG.rU.rA.rU.rA.rA.rC.rA.rA.rU.rG.rC | | RD0068 (NT) |
| (<Strand Ref.: 297>) | | |
| rG.rC.rG.rU.rU.rG.rU.rU.rA.rU.rA.rG.rU. | 33 | RL0068 strand of compound |
| rG.rU.rC.rC.rC.rA.rC.rC.rC.rA.rC.rC. | | RD0068 (NT) |
| rA.rG.rG.rG.rG.rU.rC.rC.rA.rC.rA.rU. | | |
| rG.rG.rC.rA.rA.rC | | |
| (<Strand Ref.: 298>) | | |
| GGUGGGUGGAACAGUAUAACAAUGC | 32 | RS0018-UM strand of |
| (<Strand Ref.: 299>) | | compound RD0068 (NT) |
| GCGUUGUUAUAGUGUCCCACCCACCAGGGGUCCACAU | 33 | RL0068-UM strand of |
| GGCAAC | | compound RD0068 (NT) |
| (<Strand Ref.: 300>) | | |
| rG.rG.rU.rG.rG.rG.rU.rG.rG.rA.rA.rC.rA. | 32 | RS0018 strand of compound |
| rG.rU.rA.rU.rA.rA.rC.rA.rA.rU.rG.rC | | RD0069 (NT) |
| (<Strand Ref.: 301>) | | |
| rG.rC.rG.rU.rU.rG.rU.rU.rA.rU.rA.rG.rU. | 33 | RL0069 strand of compound |
| rG.rU.rC.rC.rC.rA.rC.rC.rC.rA.rC.rC. | | RD0069 (NT) |
| mA.mG.mG.mG.mG.mU.rC.rC.rA.mC.mA.mU. | | |
| mG.mG*mC*mA*mA*mC | | |
| (<Strand Ref.: 302>) | | |
| GGUGGGUGGAACAGUAUAACAAUGC | 32 | RS0018-UM strand of |
| (<Strand Ref.: 303>) | | compound RD0069 (NT) |
| GCGUUGUUAUAGUGUCCCACCCACCAGGGGUCCACAU | 33 | RL0069-UM strand of |
| GGCAAC | | compound RD0069 (NT) |
| (< Strand Ref.: 304>) | | |
| rG.rG.rU.rG.rG.rG.rU.rG.rG.rA.rA.rC.rA. | 32 | RS0018 strand of compound |
| rG.rU.rA.rU.rA.rA.rC.rA.rA.rU.rG.rC | | RD0070 (NT) |
| (<Strand Ref.: 305>) | | |
| rG.rC.rG.rU.rU.rG.rU.rU.rA.rU.rA.rG.rU. | 33 | RL0070 strand of compound |
| rG.rU.rC.rC.rC.rA.rC.rC.rC.rA.rC.rC. | | RD0070 (NT) |
| rA.rG.rG.rG.rG.rU.rC.rC.rA.rC.rA.rU. | | |
| rG.rG.rC.rA*rA*rC | | |
| (<Strand Ref.: 306>) | | |
| GGUGGGUGGAACAGUAUAACAAUGC | 32 | RS0018-UM strand of |
| (<Strand Ref.: 307>) | | compound RD0070 (NT) |
| GCGUUGUUAUAGUGUCCCACCCACCAGGGGUCCACAU | 33 | RL0070-UM strand of |
| GGCAAC | | compound RD0070 (NT) |
| (<Strand Ref.: 308>) | | |
| rG.rG.rU.rG.rG.rG.rU.rG.rG.rA.rA.rC.rA. | 32 | RS0018 strand of compound |
| rG.rU.rA.rU.rA.rA.rC.rA.rA.rU.rG.rC | | RD0071 (NT) |
| (<Strand Ref.: 309>) | | |
| rG.rC.rG.rU.rU.rG.rU.rU.rA.rU.rA.rG.rU. | 33 | RL0071 strand of compound |
| rG.rU.rC.rC.rC.rA.rC.rC.rC.rA.rC.rC. | | RD0071 (NT) |
| mA.fG.mG.fG.mG.fU.rC.rC.rA.fC.mA.fU. | | |
| mG.fG.mC.fA*mA*fC | | |
| (<Strand Ref.: 310>) | | |
| GGUGGGUGGAACAGUAUAACAAUGC | 32 | RS0018-UM strand of |
| (<Strand Ref.: 311>) | | compound RD0071 (NT) |
| GCGUUGUUAUAGUGUCCCACCCACCAGGGGUCCACAU | 33 | RL0071-UM strand of |
| GGCAAC | | compound RD0071 (NT) |
| (<Strand Ref.: 312>) | | |
| rG.rG.rU.rG.rG.rG.rU.rG.rG.rA.rA.rU.rA. | 34 | RS0019 strand of compound |
| rG.rU.rA.rU.rA.rA.rC.rA.rA.rU.rA.rU | | RD0072 (NT) |
| .rG.rC | | |
| (<Strand Ref.: 313>) | | |
| rG.rC.rA.rU.rG.rU.rU.rG.rU.rU.rA.rU.rA. | 35 | RL0072 strand of compound |
| rG.rU.rA.rU.rC.rC.rC.rA.rC.rC.rC.rA. | | RD0072 (NT) |
| rC.rC.rA.rG.rG.rG.rG.rU.rC.rC.rA.rC. | | |
| rA.rU.rG.rG.rC.rA.rA.rC | | |
| (<Strand Ref.: 314>) | | |
| GGUGGGUGGAAUAGUAUAACAAUAUGC | 34 | RS0019-UM strand of |
| (<Strand Ref.: 315>) | | compound RD0072 (NT) |
| GCAUGUUGUUAUAGUAUCCCACCCACCAGGGGUCCAC | 35 | RL0072-UM strand of |
| AUGGCAAC | | compound RD0072 (NT) |
| (<Strand Ref.: 316>) | | |
| rG.rG.rU.rG.rG.rG.rU.rG.rG.rA.rA.rU.rA. | 34 | RS0019 strand of compound |
| rG.rU.rA.rU.rA.rA.rC.rA.rA.rU.rA.rU | | RD0073 (NT) |
| .rG.rC | | |
| (<Strand Ref.: 317>) | | |
| rG.rC.rA.rU.rG.rU.rU.rG.rU.rU.rA.rU.rA. | 35 | RL0073 strand of compound |
| rG.rU.rA.rU.rC.rC.rC.rA.rC.rC.rC.rA. | | RD0073 (NT) |
| rC.rC.mA.mG.mG.mG.mG.mU.rC.rC.rA.mC. | | |
| mA.mU.mG.mG*mC*mA*mA*mC | | |
| (<Strand Ref.: 318>) | | |
| GGUGGGUGGAAUAGUAUAACAAUAUGC | 34 | RS0019-UM strand of |
| (<Strand Ref.: 319>) | | compound RD0073 (NT) |
| GCAUGUUGUUAUAGUAUCCCACCCACCAGGGGUCCAC | 35 | RL0073-UM strand of |
| AUGGCAAC | | compound RD0073 (NT) |
| (<Strand Ref.: 320>) | | |
| rG.rG.rU.rG.rG.rG.rU.rG.rG.rA.r.rU.rA. | 36 | RS0020 strand of compound |
| rG.rU.rA.rC.rA.rA.rC.rA.rA.rU.rA.rU. | | RD0074 (NT) |
| rG.rC | | |
| (<Strand Ref.: 321>) | | |
| rG.rC.rA.rU.rG.rU.rU.rG.rU.rU.rG.rU.rA. | 37 | RL0074 strand of compound |
| rG.rU.rA.rU.rC.rC.rC.rA.rC.rC.rC.rA. | | RD0074 (NT) |
| rC.rC.mA.mG.mG.mG.mG.mU.rC.rC.rA.mC. | | |
| mA.mU.mG.mG*mC*mA*mA*mC | | |
| (<Strand Ref.: 322>) | | |
| GGUGGGUGGAAUAGUACAACAAUAUGC | 36 | RS0020-UM strand of |
| (<Strand Ref.: 323>) | | compound RD0074 (NT) |
| GCAUGUUGUUGUAGUAUCCCACCCACCAGGGGUCCAC | 37 | RL0074-UM strand of |
| AUGGCAAC | | compound RD0074 (NT) |
| (<Strand Ref.: 324>) | | |
| rG.rG.rU.rG.rG.rG.rU.rG.rG.rA.rA.rU.rA. | 36 | RS0020 strand of compound |
| rG.rU.rA.rC.rA.rA.rC.rA.rA.rU.rA.rU. | | RD0075 (NT) |
| rG.rC | | |
| (<Strand Ref.: 325>) | | |
| rG.rC.rA.rU.rG.rU.rU.rG.rU.rU.rG.rU.rA. | 37 | RL0075 strand of compound |
| rG.rU.rA.rU.rC.rC.rC.rA.rC.rC.rC.rA. | | RD0075 (NT) |
| rC.rC.rA.rG.rG.rG.rG.rU.rC.rC.rA.rC. | | |
| rA.rU.rG.rG.rC.rA.rA.rC | | |
| (<Strand Ref.: 326>) | | |
| GGUGGGUGGAAUAGUACAACAAUAUGC | 36 | RS0020-UM strand of |
| (<Strand Ref.: 327>) | | compound RD0075 (NT) |
| GCAUGUUGUUGUAGUAUCCCACCCACCAGGGGUCCAC | 37 | RL0075-UM strand of |
| AUGGCAAC | | compound RD0075 (NT) |
| (<Strand Ref.: 328>) | | |
| rG*rG*rG.rU.rG.rG.rA.rA.rG.rA.rG.rU.rA. | 5 | RS0008 strand of compound |
| rG.rA.rA.rC.rA.rA.rU.rA.rU*rG*rC | | RD0160 (NT) |
| (<Strand Ref.: 329>) | | |
| rG*rC*rA.rU.rG.rU.rU.rG.rU.rU.rC.rU.rA. | 38 | RL0091 strand of compound |
| rG.rU.rC.rU.rC.rC.rC.rA.rC.rC.rC.mA. | | RD0160 (NT) |
| mG.mG.mG.mG.mU.rC.rC.rA.mC.mA.mU.mG. | | |
| mG*mC*mA*mA*mC | | |
| (<Strand Ref.: 330>) | | |
| GGGUGGAAGAGUAGAACAAUAUGC | 5 | RS0008-UM strand of |
| (<Strand Ref.: 331>) | | compound RD0160 (NT) |
| GCAUGUUGUUCUAGUCUCCCACCCAGGGGUCCACAUG | 38 | RL0091-UM strand of |
| GCAAC | | compound RD0160 (NT) |
| (<Strand Ref.: 332>) | | |
| rG*rG*rG*rU.rG.rG.rA.rA.rG.rA.rG.rU.rA. | 5 | RS0021 strand of compound |
| rG.rA.rA.rC.rA.rA.rU.rA*rU*rG*rC | | RD0161 (NT) |
| (<Strand Ref.: 333>) | | |
| rG*rC*rA*rU.rG.rU.rU.rG.rU.rU.rC.rU.rA. | 38 | RL0092 strand of compound |
| rG.rU.rC.rU.rC.rC.rC.rA.rC.rC.rC.mA. | | RD0161 (NT) |
| mG.mG.mG.mG.mU.rC.rC.rA.mC.mA.mU.mG. | | |
| mG*mC*mA*mA*mC | | |
| (<Strand Ref.: 334>) | | |
| GGGUGGAAGAGUAGAACAAUAUGC | 5 | RS0021-UM strand of |
| (<Strand Ref.: 335>) | | compound RD0161 (NT) |
| GCAUGUUGUUCUAGUCUCCCACCCAGGGGUCCACAUG | 38 | RL0092-UM strand of |
| GCAAC | | compound RD0161 (NT) |
| (<Strand Ref.: 336>) | | |
| rG*rG*rG*rU*rG.rG.rA.rA.rG.rA.rG.rU.rA. | 5 | RS0022 strand of compound |
| rG.rA.rA.rC.rA.rA.rU*rA*rU*rG*rC | | RD0162 (NT) |
| (<Strand Ref.: 337>) | | |
| rG*rC*rA*rU*rG.rU.rU.rG.rU.rU.rC.rU.rA. | 38 | RL0093 strand of compound |
| rG.rU.rC.rU.rC.rC.rC.rA.rC.rC.rC.mA. | | RD0162 (NT) |
| mG.mG.mG.mG.mU.rC.rC.rA.mC.mA.mU.mG. | | |
| mG*mC*mA*mA*mC | | |
| (<Strand Ref.: 338>) | | |
| GGGUGGAAGAGUAGAACAAUAUGC | 5 | RS0022-UM strand of |
| (<Strand Ref.: 339>) | | compound RD0162 (NT) |
| GCAUGUUGUUCUAGUCUCCCACCCAGGGGUCCACAUG | 38 | RL0093-UM strand of |
| GCAAC | | compound RD0162 (NT) |
| (<Strand Ref.: 340>) | | |
| rG*rG*rG*rU*rG.rG.rA.rA.rG.rA.rG.rU.rA. | 39 | RS0023 strand of compound |
| rG.rA.rA.rC.rA*rA*rU*rG*rC | | RD0163 (NT) |
| (<Strand Ref.: 341>) | | |
| rG*rC*rG*rU*rU.rG.rU.rU.rC.rU.rA.rG.rU. | 40 | RL0094 strand of compound |
| rC.rU.rC.rC.rC.rA.rC.rC.rC.mA.mG.mG. | | RD0163 (NT) |
| mG.mG.mU.rC.rC.rA.mC.mA.mU.mG.mG*mC* | | |
| mA*mA*mC | | |
| (<Strand Ref.: 342>) | | |
| GGGUGGAAGAGUAGAACAAUGC | 39 | RS0023-UM strand of |
| (<Strand Ref.: 343>) | | compound RD0163 (NT) |
| GCGUUGUUCUAGUCUCCCACCCAGGGGUCCACAUGGC | 40 | RL0094-UM strand of |
| AAC | | compound RD0163 (NT) |
| (<Strand Ref.: 344>) | | |
| mG*mG*mG*mU*mG.mG.mA.mA.mG.mA.mG.mU.mA. | 5 | RS0024 strand of compound |
| mG.mA.mA.mC.mA.mA.mU*mA*mU*mG*mC | | RD0164 (NT) |
| (<Strand Ref.: 345>) | | |
| mG*mC*mA*mU*mA.mU.mU.mG.mU.mU.mC.mU.mA. | 12 | RL0095 strand of compound |
| mG.mU.mC.mU.mC.mC.mC.mA.mC.mC.mC.mA. | | RD0164 (NT) |
| mG.mG.mG.mG.mU.rC.rC.rA.mC.mA.mU.mG. | | |
| mG*mC*mA*mA*mC | | |
| (<Strand Ref.: 346>) | | |
| GGGUGGAAGAGUAGAACAAUAUGC | 5 | RS0024-UM strand of |
| (<Strand Ref.: 347>) | | compound RD0164 (NT) |
| GCAUAUUGUUCUAGUCUCCCACCCAGGGGUCCACAUG | 12 | RL0095-UM strand of |
| GCAAC | | compound RD0164 (NT) |
| (<Strand Ref.: 348>) | | |
| mG*mG*mG*mU*mG.mG.fA.mA.mG.mA.fG.mU.mA. | 5 | RS0025 strand of compound |
| mG.mA.mA.mC.mA.mA.mU*mA*mU*mG*mC | | RD0165 (NT) |
| (<Strand Ref.: 349>) | | |
| mG*mC*mA*mU*mA.mU.mU.mG.mU.mU.mC.mU.mA. | 12 | RL0096 strand of compound |
| fG.mU.mC.mU.mC.mC.mC.mA.mC.mC.mC.mA. | | RD0165 (NT) |
| mG.mG.mG.mG.mU.rC.rC.rA.mC.mA.mU.mG. | | |
| mG*mC*mA*mA*mC | | |
| (<Strand Ref.: 350>) | | |
| GGGUGGAAGAGUAGAACAAUAUGC | 5 | RS0025-UM strand of |
| (<Strand Ref.: 351>) | | compound RD0165 (NT) |
| GCAUAUUGUUCUAGUCUCCCACCCAGGGGUCCACAUG | 12 | RL0096-UM strand of |
| GCAAC | | compound RD0165 (NT) |
| (<Strand Ref.: 352>) | | |
| mG*fG*mG*fU*mG.fG.fA.fA.mG.fA.fG.fU.mA. | 5 | RS0026 strand of compound |
| A.fG.mA.fA.mC.fA.mA.fU*mA*fU*mG*mC | | RD0166 (NT) |
| (<Strand Ref.: 353>) | | |
| mG*mC*mA*mU*mA.mU.mU.mG.mU.mU.mC.mU.mA. | 12 | RL0096 strand of compound |
| fG.mU.mC.mU.mC.mC.mC.mA.mC.mC.mC.mA. | | RD0166 (NT) |
| mG.mG.mG.mG.mU.rC.rC.rA.mC.mA.mU.mG. | | |
| mG*mC*mA*mA*mC | | |
| (<Strand Ref.: 354>) | | |
| GGGUGGAAGAGUAGAACAAUAUGC | 5 | RS0026-UM strand of |
| (<Strand Ref.: 355>) | | compound RD0166 (NT) |
| GCAUAUUGUUCUAGUCUCCCACCCAGGGGUCCACAUG | 12 | RL0096-UM strand of |
| GCAAC | | compound RD0166 (NT) |
| (<Strand Ref.: 356>) | | |
| mG*mG*mG*mU*mG.mG.fA.mA.mG.mA.fG.mU.mA. | 5 | RS0025 strand of compound |
| mG.mA.mA.mC.mA.mA.mU*mA*mU*mG*mC | | RD0167 (NT) |
| (<Strand Ref.: 357>) | | |
| mG*fC*mA*fU*mA.fU.mU.fG.mU.fU.mC.fU.mA. | 12 | RL0097 strand of compound |
| fG.mU.fC.mU.mC.mC.fC.mA.fC.mC.fC.mA. | | RD0167 (NT) |
| mG.mG.mG.mG.mU.rC.rC.rA.mC.mA.mU.mG. | | |
| mG*mC*mA*mA*mC | | |
| (<Strand Ref.: 358>) | | |
| GGGUGGAAGAGUAGAACAAUAUGC | 5 | RS0025-UM strand of |
| (<Strand Ref.: 359>) | | compound RD0167 (NT) |
| GCAUAUUGUUCUAGUCUCCCACCCAGGGGUCCACAUG | 12 | RL0097-UM strand of |
| GCAAC | | compound RD0167 (NT) |
| (<Strand Ref.: 360>) | | |
| mG*fG*mG*fU*mG.fG.fA.fA.mG.fA.fG.fU.mA. | 5 | RS0026 strand of compound |
| fG.mA.fA.mC.fA.mA.fU*mA*fU*mG*mC | | RD0168 (NT) |
| (<Strand Ref.: 361>) | | |
| mG*fC*mA*fU*mA.fU.mU.fG.mU.fU.mC.fU.mA. | 12 | RL0097 strand of compound |
| fG.mU.fC.mU.mC.mC.fC.mA.fC.mC.fC.mA. | | RD0168 (NT) |
| mG.mG.mG.mG.mU.rC.rC.rA.mC.mA.mU.mG. | | |
| mG*mC*mA*mA*mC | | |
| (<Strand Ref.: 362>) | | |
| GGGUGGAAGAGUAGAACAAUAUGC | 5 | RS0026-UM strand of |
| (<Strand Ref.: 363>) | | compound RD0168 (NT) |
| GCAUAUUGUUCUAGUCUCCCACCCAGGGGUCCACAUG | 12 | RL0097-UM strand of |
| GCAAC | | compound RD0168 (NT) |
| (<Strand Ref.: 364>) | | |
| mG*mG*fG*mU*fG.mG.fA.mA.fG.mA.fG.mU.fA. | 5 | RS0027 strand of compound |
| mG.fA.mA.fC.mA.fA.mU*fA*mU*fG*mC | | RD0169 (NT) |
| (<Strand Ref.: 365>) | | |
| mG*mC*mA*mU*mA.mU.mU.mG.mU.mU.mC.mU.mA. | 6 | RL0098 strand of compound |
| fG.mU.mC.mU.mC.mC.mC.mA.mC.mC.mC | | RD0169 (NT) |
| (<Strand Ref.: 366>) | | |
| GGGUGGAAGAGUAGAACAAUAUGC | 5 | RS0027-UM strand of |
| (<Strand Ref.: 367>) | | compound RD0169 (NT) |
| GCAUAUUGUUCUAGUCUCCCACCC | 6 | RL0098-UM strand of |
| (<Strand Ref.: 368>) | | compound RD0169 (NT) |
| mG*mG*mG*mU*mG.mG.fA.mA.mG.mA.fG.mU.mA. | 5 | RS0025 strand of compound |
| mG.mA.mA.mC.mA.mA.mU*mA*mU*mG*mC | | RD0170 (NT) |
| (<Strand Ref.: 369>) | | |
| mG*mC*fA*mU*fA.mU.fU.mG.fU.mU.fC.mU.fA. | 12 | RL0099 strand of compound |
| fG.fU.mC.fU.mC.fC.mC.fA.mC.fC.mC.mA. | | RD0170 (NT) |
| mG.mG.mG.mG.mU.rC.rC.rA.mC.mA.mU.mG. | | |
| mG*mC*mA*mA*mC | | |
| (<Strand Ref.: 370>) | | |
| GGGUGGAAGAGUAGAACAAUAUGC | 5 | RS0025-UM strand of |
| (<Strand Ref.: 371>) | | compound RD0170 (NT) |
| GCAUAUUGUUCUAGUCUCCCACCCAGGGGUCCACAUG | 12 | RL0099-UM strand of |
| GCAAC | | compound RD0170 (NT) |
| (<Strand Ref.: 372>) | | |
| mG*mG*fG*mU*fG.mG.fA.mA.fG.mA.fG.mU.fA. | 5 | RS0027 strand of compound |
| mG.fA.mA.fC.mA.fA.mU*fA*mU*fG*mC | | RD0171 (NT) |
| (<Strand Ref.: 373>) | | |
| mG*mC*fA*mU*fA.mU.fU.mG.fU.mU.fC.mU.fA. | 12 | RL0099 strand of compound |
| fG.fU.mC.fU.mC.fC.mC.fA.mC.fC.mC.mA. | | RD0171 (NT) |
| mG.mG.mG.mG.mU.rC.rC.rA.mC.mA.mU.mG. | | |
| mG*mC*mA*mA*mC | | |
| (<Strand Ref.: 374>) | | |
| GGGUGGAAGAGUAGAACAAUAUGC | 5 | RS0027-UM strand of |
| (<Strand Ref.: 375>) | | compound RD0171 (NT) |
| GCAUAUUGUUCUAGUCUCCCACCCAGGGGUCCACAUG | 12 | RL0099-UM strand of |
| GCAAC | | compound RD0171 (NT) |
| (<Strand Ref.: 376>) | | |
| rG*rG*rG*rU*rG.rU.rA.rA.rG.rA.rG.rU.rA. | 41 | RS0028 strand of compound |
| rG.rA.rA.rC.rA.rA.rU*rA*rU*rG*rC | | RD0172 (NT) |
| (<Strand Ref.: 377>) | | |
| rG*rC*rA*rU*rA.rU.rU.rG.rU.rU.rC.rU.rA. | 42 | RL0100 strand of compound |
| rG.rU.rC.rU.rC.rA.rC.rA.rC.rC.rC.mA. | | RD0172 (NT) |
| mG.mG.mG.mG.mU.rC.rC.rA.mC.mA.mU.mG. | | |
| mG*mC*mA*mA*mC | | |
| (<Strand Ref.: 378>) | | |
| GGGUGUAAGAGUAGAACAAUAUGC | 41 | RS0028-UM strand of |
| (<Strand Ref.: 379>) | | compound RD0172 (NT) |
| GCAUAUUGUUCUAGUCUCACACCCAGGGGUCCACAUG | 42 | RL0100-UM strand of |
| GCAAC | | compound RD0172 (NT) |
| (<Strand Ref.: 380>) | | |
| rG*rG*rG*rU*rG.rU.rA.rA.rG.rA.rG.rU.rA. | 41 | RS0028 strand of compound |
| rG.rA.rA.rC.rA.rA.rU*rA*rU*rG*rC | | RD0173 (NT) |
| (<Strand Ref.: 381>) | | |
| rG*rC*rA*rU*rG.rU.rU.rG.rU.rU.rC.rU.rA. | 43 | RL0101 strand of compound |
| rG.rU.rC.rU.rC.rA.rC.rA.rC.rC.rC.mA. | | RD0173 (NT) |
| mG.mG.mG.mG.mU.rC.rC.rA.mC.mA.mU.mG. | | |
| mG*mC*mA*mA*mC | | |
| (<Strand Ref.: 382>) | | |
| GGGUGUAAGAGUAGAACAAUAUGC | 41 | RS0028-UM strand of |
| (<Strand Ref.: 383>) | | compound RD0173 (NT) |
| GCAUGUUGUUCUAGUCUCACACCCAGGGGUCCACAUG | 43 | RL0101-UM strand of |
| GCAAC | | compound RD0173 (NT) |
| (<Strand Ref.: 384>) | | |
| rG*rG*rG*rU*rG.rG.rA.rA.rG.rA.rG.rU.rA. | 44 | RS0029 strand of compound |
| rC.rA.rA.rC.rA.rA.rU*rA*rU*rG*rC | | RD0174 (NT) |
| (<Strand Ref.: 385>) | | |
| rG*rC*rA*rU*rA.rU.rU.rG.rU.rU.rG.rU.rA. | 45 | RL0102 strand of compound |
| rG.rU.rC.rU.rC.rC.rC.rA.rC.rC.rC.mA. | | RD0174 (NT) |
| mG.mG.mG.mG.mU.rC.rC.rA.mC.mA.mU.mG. | | |
| mG*mC*mA*mA*mC | | |
| (<Strand Ref.: 386>) | | |
| GGGUGGAAGAGUACAACAAUAUGC | 44 | RS0029-UM strand of |
| (<Strand Ref.: 387>) | | compound RD0174 (NT) |
| GCAUAUUGUUGUAGUCUCCCACCCAGGGGUCCACAUG | 45 | RL0102-UM strand of |
| GCAAC | | compound RD0174 (NT) |
| (<Strand Ref.: 388>) | | |
| rG*rG*rG*rU*rG.rG.rA.rA.rG.rA.rG.rU.rA. | 44 | RS0029 strand of compound |
| rC.rA.rA.rC.rA.rA.rU*rA*rU*rG*rC | | RD0175 (NT) |
| (<Strand Ref.: 389>) | | |
| rG*rC*rA*rU*rG.rU.rU.rG.rU.rU.rG.rU.rA. | 46 | RL0103 strand of compound |
| rG.rU.rC.rU.rC.rC.rC.rA.rC.rC.rC.mA. | | RD0175 (NT) |
| mG.mG.mG.mG.mU.rC.rC.rA.mC.mA.mU.mG. | | |
| mG*mC*mA*mA*mC | | |
| (<Strand Ref.: 390>) | | |
| GGGUGGAAGAGUACAACAAUAUGC | 44 | RS0029-UM strand of |
| (<Strand Ref.: 391>) | | compound RD0175 (NT) |
| GCAUGUUGUUGUAGUCUCCCACCCAGGGGUCCACAUG | 46 | RL0103-UM strand of |
| GCAAC | | compound RD0175 (NT) |
| (<Strand Ref.: 392>) | | |
| rG*rG*rG*rU*rG.rG.rA.rA.rG.rA.rG.rU.rA. | 47 | RS0030 strand of compound |
| rU.rA.rA.rC.rA.rA.rU*rA*rU*rG*rC | | RD0176 (NT) |
| (<Strand Ref.: 393>) | | |
| rG*rC*rA*rU*rG.rU.rU.rG.rU.rU.rA.rU.rA. | 48 | RL0104 strand of compound |
| rG.rU.rC.rU.rC.rC.rC.rA.rC.rC.rC.mA. | | RD0176 (NT) |
| mG.mG.mG.mG.mU.rC.rC.rA.mC.mA.mU.mG. | | |
| mG*mC*mA*mA*mC | | |
| (<Strand Ref.: 394>) | | |
| GGGUGGAAGAGUAUAACAAUAUGC | 47 | RS0030-UM strand of |
| (<Strand Ref.: 395>) | | compound RD0176 (NT) |
| GCAUGUUGUUAUAGUCUCCCACCCAGGGGUCCACAUG | 48 | RL0104-UM strand of |
| GCAAC | | compound RD0176 (NT) |
| (<Strand Ref.: 396>) | | |
| rG*rG*rG*rU*rG.rG.rA.rA.rG.rA.rG.rU.rA. | 47 | RS0030 strand of compound |
| rU.rA.rA.rC.rA.rA.rU*rA*rU*rG*rC | | RD0177 (NT) |
| (<Strand Ref.: 397>) | | |
| rG*rC*rA*rU*rA.rU.rU.rG.rU.rU.rA.rU.rA. | 49 | RL0105 strand of compound |
| rG.rU.rC.rU.rC.rC.rC.rA.rC.rC.rC.mA. | | RD0177 (NT) |
| mG.mG.mG.mG.mU.rC.rC.rA.mC.mA.mU.mG. | | |
| mG*mC*mA*mA*mC | | |
| (<Strand Ref.: 398>) | | |
| GGGUGGAAGAGUAUAACAAUAUGC | 47 | RS0030-UM strand of |
| (<Strand Ref.: 399>) | | compound RD0177 (NT) |
| GCAUAUUGUUAUAGUCUCCCACCCAGGGGUCCACAUG | 49 | RL0105-UM strand of |
| GCAAC | | compound RD0177 (NT) |
| (<Strand Ref.: 400>) | | |
| mG*mG*mU*rG*mU.mC.rG.rA.rG.rA.rA.rG.rA. | 3 | RS0031 strand of compound |
| rG.rG.rA.rG.rA.rA.rC.mA*rA*mU*rA*mU | | RD0178 (NT) |
| (<Strand Ref.: 401>) | | |
| mA*fU*rG*fU*mU.rG.mU.mU.mC.mU.fC.rG.fU. | 11 | RL0106 strand of compound |
| mC.fU.rC.fC.mU.mC.rG.rA.mC.rA.mC.mC. | | RD0178 (NT) |
| mA.mG.mG.mG.mG.mU.rC.rC.rA.mC.mA.mU. | | |
| mG.mG*mC*mA*mA*mC | | |
| (<Strand Ref.: 402>) | | |
| GGUGUCGAGAAGAGGAGAACAAUAU | 3 | RS0031-UM strand of |
| (<Strand Ref.: 403>) | | compound RD0178 (NT) |
| AUGUUGUUCUCGUCUCCUCGACACCAGGGGUCCACAU | 11 | RL0106-UM strand of |
| GGCAAC | | compound RD0178 (NT) |
| (<Strand Ref.: 404>) | | |
| mG*mG*mU*rG*mU.mC.rG.rA.rG.rA.rA.rG.rA. | 50 | RS0032 strand of compound |
| rG.rG.rA.rG.rA.rA.rC.mA.rA.mU*rA*mU* | | RD0179 (NT) |
| rG*rC | | |
| (<Strand Ref.: 405>) | | |
| rG*rC*mA*fU*rG.fU.mU.rG.mU.mU.mC.mU.fC. | 51 | RL0107 strand of compound |
| rG.fU.mC.fU.rC.fC.mU.mC.rG.rA.mC.rA. | | RD0179 (NT) |
| mC.mC.mA.mG.mG.mG.mG.mU.rC.rC.rA.mC. | | |
| mA.mU.mG.mG*mC*mA*mA*mC | | |
| (<Strand Ref.: 406>) | | |
| GGUGUCGAGAAGAGGAGAACAAUAUGC | 50 | RS0032-UM strand of |
| (<Strand Ref.: 407>) | | compound RD0179 (NT) |
| GCAUGUUGUUCUCGUCUCCUCGACACCAGGGGUCCAC | 5 | RL0107-UM strand of |
| AUGGCAAC | | compound RD0179 (NT) |
| (<Strand Ref.: 408>) | | |
| mG*mG*mU*rG*mU.mC.rG.rA.rG.rA.rA.rG.rA. | 50 | RS0032 strand of compound |
| rG.rG.rA.rG.rA.rA.rC.mA.rA.mU*rA*mU* | | RD0180 (NT) |
| rG*rC | | |
| (<Strand Ref.: 409>) | | |
| rG*rC*mA*fU*rG.fU.mU.rG.mU.mU.mC.mU.fC. | 51 | RL0108 strand of compound |
| rG.fU.mC.fU.mC.fC.mU.mC.rG.rA.mC.rA. | | RD0180 (NT) |
| mC.mC.mA.mG.mG.mG.mG.mU.rC.rC.rA.mC. | | |
| mA.mU.mG.mG*mC*mA*mA*mC | | |
| (<Strand Ref.: 410>) | | |
| GGUGUCGAGAAGAGGAGAACAAUAUGC | 50 | RS0032-UM strand of |
| (<Strand Ref.: 411>) | | compound RD0180 (NT) |
| GCAUGUUGUUCUCGUCUCCUCGACACCAGGGGUCCAC | 51 | RL0108-UM strand of |
| AUGGCAAC | | compound RD0180 (NT) |
| (<Strand Ref.: 412>) | | |
| mG*mG*mU*rG*mU.mC.rG.rA.rG.rA.rA.rG.rA. | 3 | RS0031 strand of compound |
| rG.rG.rA.rG.rA.rA.rC.mA*rA*mU*rA*mU | | RD0196 (NT) |
| (<Strand Ref.: 413>) | | |
| mA*fU*rG*fU*mU.rG.mU.mU.mC.mU.fC.rG.fU. | 11 | RL0124 strand of compound |
| mC.fU.mC.fC.mU.mC.rG.rA.mC.rA.mC.mC. | | RD0196 (NT) |
| mA.mG.mG.mG.mG.mU.rC.rC.rA.mC.mA.mU. | | |
| mG.mG*mC*mA*mA*mC | | |
| (<Strand Ref.: 414>) | | |
| GGUGUCGAGAAGAGGAGAACAAUAU | 3 | RS0031-UM strand of |
| (<Strand Ref.: 415>) | | compound RD0196 (NT) |
| AUGUUGUUCUCGUCUCCUCGACACCAGGGGUCCACAU | 11 | RL0124-UM strand of |
| GGCAAC | | compound RD0196 (NT) |
| (<Strand Ref.: 416>) | | |
| mG*mG*mU*rG*mU.mC.rG.rA.rG.mA.rA.rG.rA. | 3 | RS0033 strand of compound |
| mG.rG.rA.rG.rA.rA.rC.mA*rA*mU*rA*mU | | RD0197 (NT) |
| (<Strand Ref.: 417>) | | |
| mA*fU*rG*fU*mU.rG.mU.mU.mC.mU.fC.mG.fU. | 11 | RL0125 strand of compound |
| mC.fU.mC.fC.mU.mC.rG.rA.mC.rA.mC.mC. | | RD0197 (NT) |
| mA.mG.mG.mG.mG.mU.rC.rC.rA.mC.mA.mU. | | |
| mG.mG*mC*mA*mA*mC | | |
| (<Strand Ref.: 418>) | | |
| GGUGUCGAGAAGAGGAGAACAAUAU | 3 | RS0033-UM strand of |
| (<Strand Ref.: 419>) | | compound RD0197 (NT) |
| AUGUUGUUCUCGUCUCCUCGACACCAGGGGUCCACAU | | RL0125-UM strand of |
| GGCAAC | | compound RD0197 (NT) |
| (<Strand Ref.: 420>) | | |
| mG*mG*mU*rG*mU.mC.rG.rA.rG.fA.rA.rG.rA. | 3 | RS0034 strand of compound |
| fG.rG.rA.rG.rA.rA.rC.mA*rA*mU*rA*mU | | RD0198 (NT) |
| (<Strand Ref.: 421>) | | |
| mA*fU*rG*fU*mU.rG.mU.mU.mC.mU.fC.fG.fU. | 1 | RL0126 strand of compound |
| mC.fU.mC.fC.mU.mC.rG.rA.mC.rA.mC.mC. | | RD0198 (NT) |
| mA.mG.mG.mG.mG.mU.rC.rC.rA.mC.mA.mU. | | |
| mG.mG*mC*mA*mA*mC | | |
| (<Strand Ref.: 422>) | | |
| GGUGUCGAGAAGAGGAGAACAAUAU | 3 | RS0034-UM strand of |
| (<Strand Ref.: 423>) | | compound RD0198 (NT) |
| AUGUUGUUCUCGUCUCCUCGACACCAGGGGUCCACAU | 11 | RL0126-UM strand of |
| GGCAAC | | compound RD0198 (NT) |
| (<Strand Ref.: 424>) | | |
| mG*mG*mU*mG*mU.mC.mG.mA.mG.fA.mA.mG.mA. | 3 | RS0035 strand of compound |
| fG.mG.mA.mG.mA.mA.mC.mA*mA*mU*mA*mU | | RD0199 (NT) |
| (<Strand Ref.: 425>) | | |
| mA*mU*mG*mU*mU.mG.mU.mU.mC.mU.fC.fG.fU. | 1 | RL0127 strand of compound |
| mC.fU.mC.fC.mU.mC.mG.mA.mC.mA.mC.mC. | | RD0199 (NT) |
| mA.mG.mG.mG.mG.mU.rC.rC.rA.mC.mA.mU. | | |
| mG.mG*mC*mA*mA*mC | | |
| (<Strand Ref.: 426>) | | |
| GGUGUCGAGAAGAGGAGAACAAUAU | 3 | RS0035-UM strand of |
| (<Strand Ref.: 427>) | | compound RD0199 (NT) |
| AUGUUGUUCUCGUCUCCUCGACACCAGGGGUCCACAU | 11 | RL0127-UM strand of |
| GGCAAC | | compound RD0199 (NT) |
| (<Strand Ref.: 428>) | | |
| mG*mG*mU*mG*mU.mC.mG.mA.mG.fA.mA.mG.mA. | 3 | RS0035 strand of compound |
| fG.mG.mA.mG.mA.mA.mC.mA*mA*mU*mA*mU | | RD0200 (NT) |
| (<Strand Ref.: 429>) | | |
| mA*mU*mG*mU*mU.mG.mU.mU.mC.mU.mC.fG.mU. | 1 | RL0128 strand of compound |
| mC.mU.mC.mC.mU.mC.mG.mA.mC.mA.mC.mC. | | RD0200 (NT) |
| mA.mG.mG.mG.mG.mU.rC.rC.rA.mC.mA.mU. | | |
| mG.mG*mC*mA*mA*mC | | |
| (<Strand Ref.: 430>) | | |
| GGUGUCGAGAAGAGGAGAACAAUAU | 3 | RS0035-UM strand of |
| (<Strand Ref.: 431>) | | compound RD0200 (NT) |
| AUGUUGUUCUCGUCUCCUCGACACCAGGGGUCCACAU | 11 | RL0128-UM strand of |
| GGCAAC | | compound RD0200 (NT) |
| (<Strand Ref.: 432>) | | |
| mG*mG*mU*mG*mU.mC.mG.mA.mG.mA.mA.mG.mA. | 3 | RS0036 strand of compound |
| mG.mG.mA.mG.mA.mA.mC.mA*mA*mU*mA*mU | | RD0201 (NT) |
| (<Strand Ref.: 433>) | | |
| mA*mUmG*mU*mU.mG.mU.mU.mC.mU.mC.mG.mU. | 11 | RL0129 strand of compound |
| mC.mU.mC.mC.mU.mC.mG.mA.mC.mA.mC.mC. | | RD0201 (NT) |
| mA.mG.mG.mG.mG.mU.rC.rC.rA.mC.mA.mU. | | |
| mG.mG*mC*mA*mA*mC | | |
| (<Strand Ref.: 434>) | | |
| GGUGUCGAGAAGAGGAGAACAAUAU | 3 | RS0036-UM strand of |
| (<Strand Ref.: 435>) | | compound RD0201 (NT) |
| AUGUUGUUCUCGUCUCCUCGACACCAGGGGUCCACAU | 11 | RL0129-UM strand of |
| GGCAAC | | compound RD0201 (NT) |
| (<Strand Ref.: 436>) | | |
| mG*fG*mU*fG*mU.fC.mG.fA.mG.fA.mA.fG.mA. | 3 | RS0037 strand of compound |
| fG.mG.fA.mG.fA.mA.fC.mA*fA*mU*fA*mU | | RD0202 (NT) |
| (<Strand Ref.: 437>) | | |
| mA*mU*mG*mU*mU.mG.mU.mU.mC.mU.mC.fG.mU. | 11 | RL0128 strand of compound |
| mC.mU.mC.mC.mU.mC.mG.mA.mC.mA.mC.mC. | | RD0202 (NT) |
| mA.mG.mG.mG.mG.mU.rC.rC.rA.mC.mA.mU. | | |
| mG.mG*mC*mA*mA*mC | | |
| (<Strand Ref.: 438>) | | |
| GGUGUCGAGAAGAGGAGAACAAUAU | 3 | RS0037-UM strand of |
| (<Strand Ref.: 439>) | | compound RD0202 (NT) |
| AUGUUGUUCUCGUCUCCUCGACACCAGGGGUCCACAU | 11 | RL0128-UM strand of |
| GGCAAC | | compound RD0202 (NT) |
| (<Strand Ref.: 440>) | | |
| mG*mG*fU*mG*fU.mC.fG.mA.fG.fA.fA.mG.fA. | 3 | RS0038 strand of compound |
| fG.fG.mA.fG.mA.fA.mC.fA*mA*fU*mA*mU | | RD0203 (NT) |
| (<Strand Ref.: 441>) | | |
| mA*mU*mG*mU*mU.mG.mU.mU.mC.mU.mC.fG.mU. | 11 | RL0128 strand of compound |
| mC.mU.mC.mC.mU.mC.mG.mA.mC.mA.mC.mC. | | RD0203 (NT) |
| mA.mG.mG.mG.mG.mU.rC.rC.rA.mC.mA.mU. | | |
| mG.mG*mC*mA*mA*mC | | |
| (<Strand Ref.: 442>) | | |
| GGUGUCGAGAAGAGGAGAACAAUAU | 3 | RS0038-UM strand of |
| (<Strand Ref.: 443>) | | compound RD0203 (NT) |
| AUGUUGUUCUCGUCUCCUCGACACCAGGGGUCCACAU | 11 | RL0128-UM strand of |
| GGCAAC | | compound RD0203 (NT) |
| (<Strand Ref.: 444>) | | |
| mG*mG*mU*mG*mU.mC.mG.mA.mG.fA.mA.mG.mA. | 3 | RS0035 strand of compound |
| fG.mG.mA.mG.mA.mA.mC.mA*mA*mU*mA*mU | | RD0204 (NT) |
| (<Strand Ref.: 445>) | | |
| mA*fU*mG*fU*mU.fG.mU.fU.mC.fU.mC.fG.mU. | 11 | RL0130 strand of compound |
| fC.mU.mC.mC.fU.mC.fG.mA.fC.mA.fC.mC. | | RD0204 (NT) |
| mA.mG.mG.mG.mG.mU.rC.rC.rA.mC.mA.mU. | | |
| mG.mG*mC*mA*mA*mC | | |
| (<Strand Ref.: 446>) | | |
| GGUGUCGAGAAGAGGAGAACAAUAU | 3 | RS0035-UM strand of |
| (<Strand Ref.: 447>) | | compound RD0204 (NT) |
| AUGUUGUUCUCGUCUCCUCGACACCAGGGGUCCACAU | 11 | RL0130-UM strand of |
| GGCAAC | | compound RD0204 (NT) |
| (<Strand Ref.: 448>) | | |
| mG*mG*mU*mG*mU.mC.mG.mA.mG.fA.mA.mG.mA. | 3 | RS0035 strand of compound |
| fG.mG.mA.mG.mA.mA.mC.mA*mA*mU*mA*mU | | RD0205 (NT) |
| (<Strand Ref.: 449>) | | |
| mA*mU*fG*mU*fU.mG.fU.mU.fC.mU.fC.fG.fU. | 11 | RL0131 strand of compound |
| mC.fU.mC.fC.mU.fC.mG.fA.mC.fA.mC.fC. | | RD0205 (NT) |
| mA.mG.mG.mG.mG.mU.rC.rC.rA.mC.mA.mU. | | |
| mG.mG*mC*mA*mA*mC | | |
| (<Strand Ref.: 450>) | | |
| GGUGUCGAGAAGAGGAGAACAAUAU | 3 | RS0035-UM strand of |
| (<Strand Ref.: 451>) | | compound RD0205 (NT) |
| AUGUUGUUCUCGUCUCCUCGACACCAGGGGUCCACAU | 11 | RL0131-UM strand of |
| GGCAAC | | compound RD0205 (NT) |
| (<Strand Ref.: 452>) | | |
| mG*fG*mU*fG*mU.fC.mG.fA.mG.fA.mA.fG.mA. | 3 | RS0037 strand of compound |
| fG.mG.fA.mG.fA.mA.fC.mA*fA*mU*fA*mU | | RD0206 (NT) |
| (<Strand Ref.: 453>) | | |
| mA*mU*fG*mU*fU.mG.fU.mU.fC.mU.fC.fG.fU. | 11 | RL0131 strand of compound |
| .mC.fU.mC.fC.mU.fC.mG.fA.mC.fA.mC.fC. | | RD0206 (NT) |
| mA.mG.mG.mG.mG.mU.rC.rC.rA.mC.mA.mU. | | |
| mG.mG*mC*mA*mA*mC | | |
| (<Strand Ref.: 454>) | | |
| GGUGUCGAGAAGAGGAGAACAAUAU | 3 | RS0037-UM strand of |
| (<Strand Ref.: 455>) | | compound RD0206 (NT) |
| AUGUUGUUCUCGUCUCCUCGACACCAGGGGUCCACAU | 11 | RL0131-UM strand of |
| GGCAAC | | compound RD0206 (NT) |
| (<Strand Ref.: 456>) | | |
| mG*mG*fU*mG*fU.mC.fG.mA.fG.fA.fA.mG.fA. | 3 | RS0038 strand of compound |
| fG.fG.mA.fG.mA.fA.mC.fA*mA*fU*mA*mU | | RD0207 (NT) |
| (<Strand Ref.: 457>) | | |
| mA*fU*mG*fU*mU.fG.mU.fU.mC.fU.mC.fG.mU. | 11 | RL0130 strand of compound |
| fC.mU.mC.mC.fU.mC.fG.mA.fC.mA.fC.mC. | | RD0207 (NT) |
| mA.mG.mG.mG.mG.mU.rC.rC.rA.mC.mA.mU. | | |
| mG.mG*mC*mA*mA*mC | | |
| (<Strand Ref.: 458>) | | |
| GGUGUCGAGAAGAGGAGAACAAUAU | 3 | RS0038-UM strand of |
| (<Strand Ref.: 459>) | | compound RD0207 (NT) |
| AUGUUGUUCUCGUCUCCUCGACACCAGGGGUCCACAU | 1 | RL0130-UM strand of |
| GGCAAC | | compound RD0207 (NT) |
| (<Strand Ref.: 460>) | | |
| mG*mG*mU.fG.mU.mC.fG.fA.fG.fA.fA.fG.fA. | 3 | RS0039 strand of compound |
| fG.fG.fA.fG.fA.fA.fC.mA.fA.mU*fA*mU | | RD0209 (NT) |
| (<Strand Ref.: 461>) | | |
| mA*fU*fG.fU.mU.fG.mU.mU.mC.mU.fC.fG.fU. | 11 | RL0133 strand of compound |
| mC.fU.fC.fC.mU.mC.fG.fA.mC.fA.mC.mC. | | RD0209 (NT) |
| mA.mG.mG.mG.mG.mU.rC.rC.rA.mC.mA.mU. | | |
| mG.mG*mC*mA*mA*mC | | |
| (<Strand Ref.: 462>) | | |
| GGUGUCGAGAAGAGGAGAACAAUAU | 3 | RS0039-UM strand of |
| (<Strand Ref.: 463>) | | compound RD0209 (NT) |
| AUGUUGUUCUCGUCUCCUCGACACCAGGGGUCCACAU | 11 | RL0133-UM strand of |
| GGCAAC | | compound RD0209 (NT) |
| (<Strand Ref.: 464>) | | |
| mG*mG*mU.fG.mU.mC.fG.fA.fG.fA.fA.fG.fA. | 3 | RS0039 strand of compound |
| fG.fG.fA.fG.fA.fA.fC.mA.fA.mU*fA*mU | | RD0210 (NT) |
| (<Strand Ref.: 465>) | | |
| mA*fU*fG.fU.mU.fG.mU.mU.mC.mU.fC.fG.fU. | 11 | RL0134 strand of compound |
| mC.fU.mC.fC.mU.mC.fG.fA.mC.fA.mC.mC. | | RD0210 (NT) |
| mA.mG.mG.mG.mG.mU.rC.rC.rA.mC.mA.mU. | | |
| mG.mG*mC*mA*mA*mC | | |
| (<Strand Ref.: 466>) | | |
| GGUGUCGAGAAGAGGAGAACAAUAU | 3 | RS0039-UM strand of |
| (<Strand Ref.: 467>) | | compound RD0210 (NT) |
| AUGUUGUUCUCGUCUCCUCGACACCAGGGGUCCACAU | 11 | RL0134-UM strand of |
| GGCAAC | | compound RD0210 (NT) |
| (<Strand Ref.: 468>) | | |
| mG*mG*mU*mG*mU.mC.mG.mA.mG.rA.mA.mG.mA. | 3 | RS0040 strand of compound |
| rG.mG.mA.mG.mA.mA.mC.mA*mA*mU*mA*mU | | RD0211 (NT) |
| (<Strand Ref.: 469>) | | |
| mA*mU*mG*mU*mU.mG.mU.mU.mC.mU.fC.rG.fU. | 11 | RL0135 strand of compound |
| mC.fU.rC.fC.mU.mC.mG.mA.mC.mA.mC.mC. | | RD0211 (NT) |
| mA.mG.mG.mG.mG.mU.rC.rC.rA.mC.mA.mU. | | |
| mG.mG*mC*mA*mA*mC | | |
| (<Strand Ref.: 470>) | | |
| GGUGUCGAGAAGAGGAGAACAAUAU | 3 | RS0040-UM strand of |
| (<Strand Ref.: 471>) | | compound RD0211 (NT) |
| AUGUUGUUCUCGUCUCCUCGACACCAGGGGUCCACAU | 11 | RL0135-UM strand of |
| GGCAAC | | compound RD0211 (NT) |
| (<Strand Ref.: 472>) | | |
| mG*mG*mU*mG*mU.mC.mG.mA.mG.rA.mA.mG.mA. | 3 | RS0040 strand of compound |
| rG.mG.mA.mG.mA.mA.mC.mA*mA*mU*mA*mU | | RD0212 (NT) |
| (<Strand Ref.: 473>) | | |
| mA*mU*mG*mU*mU.mG.mU.mU.mC.mU.mC.rG.mU. | 11 | RL0136 strand of compound |
| mC.mU.rC.mC.mU.mC.mG.mA.mC.mA.mC.mC. | | RD0212 (NT) |
| mA.mG.mG.mG.mG.mU.rC.rC.rA.mC.mA.mU. | | |
| mG.mG*mC*mA*mA*mC | | |
| (<Strand Ref.: 474>) | | |
| GGUGUCGAGAAGAGGAGAACAAUAU | 3 | RS0040-UM strand of |
| (<Strand Ref.: 475>) | | compound RD0212 (NT) |
| AUGUUGUUCUCGUCUCCUCGACACCAGGGGUCCACAU | 11 | RL0136-UM strand of |
| GGCAAC | | compound RD0212 (NT) |
| (<Strand Ref.: 476>) | | |
| mG*fG*mU*fG*mU.fC.mG.fA.mG.rA.mA.fG.mA. | 3 | RS0041 strand of compound |
| rG.mG.fA.mG.fA.mA.fC.mA*fA*mU*fA*mU | | RD0213 (NT) |
| (<Strand Ref.: 477>) | | |
| mA*mU*mG*mU*mU.mG.mU.mU.mC.mU.mC.rG.mU. | 11 | RL0136 strand of compound |
| mC.mU.rC.mC.mU.mC.mG.mA.mC.mA.mC.mC. | | RD0213 (NT) |
| mA.mG.mG.mG.mG.mU.rC.rC.rA.mC.mA.mU. | | |
| mG.mG*mC*mA*mA*mC | | |
| (<Strand Ref.: 478>) | | |
| GGUGUCGAGAAGAGGAGAACAAUAU | 3 | RS0041-UM strand of |
| (<Strand Ref.: 479>) | | compound RD0213 (NT) |
| AUGUUGUUCUCGUCUCCUCGACACCAGGGGUCCACAU | 11 | RL0136-UM strand of |
| GGCAAC | | compound RD0213 (NT) |
| (<Strand Ref.: 480>) | | |
| mG*mG*fU*mG*fU.mC.fG.mA.fG.r.fA.mG.fA. | 3 | RS0042 strand of compound |
| rG.fG.mA.fG.mA.fA.mC.fA*mA*fU*mA*mU | | RD0214 (NT) |
| (<Strand Ref.: 481>) | | |
| mA*mU*mG*mU*mU.mG.mU.mU.mC.mU.mC.rG.mU. | | RL0136 strand of compound |
| mC.mU.rC.mC.mU.mC.mG.mA.mC.mA.mC.mC. | | RD0214 (NT) |
| mA.mG.mG.mG.mG.mU.rC.rC.rA.mC.mA.mU. | | |
| mG.mG*mC*mA*mA*mC | | |
| (<Strand Ref.: 482>) | | |
| GGUGUCGAGAAGAGGAGAACAAUAU | 3 | RS0042-UM strand of |
| (<Strand Ref.: 483>) | | compound RD0214 (NT) |
| AUGUUGUUCUCGUCUCCUCGACACCAGGGGUCCACAU | .1 | RL0136-UM strand of |
| GGCAAC | | compound RD0214 (NT) |
| (<Strand Ref.: 484>) | | |
| mG*mG*mU*mG*mU.mC.mG.mA.mG.rA.mA.mG.mA. | 3 | RS0040 strand of compound |
| rG.mG.mA.mG.mA.mA.mC.mA*mA*mU*mA*mU | | RD0215 (NT) |
| (<Strand Ref.: 485>) | | |
| mA*fU*mG*fU*mU.fG.mU.fU.mC.fU.mC.rG.mU. | 11 | RL0137 strand of compound |
| fC.mU.rC.mC.fU.mC.fG.mA.fC.mA.fC.mC. | | RD0215 (NT) |
| mA.mG.mG.mG.mG.mU.rC.rC.rA.mC.mA.mU. | | |
| mG.mG*mC*mA*mA*mC | | |
| (<Strand Ref.: 486>) | | |
| GGUGUCGAGAAGAGGAGAACAAUAU | 3 | RS0040-UM strand of |
| (<Strand Ref.: 487>) | | compound RD0215 (NT) |
| AUGUUGUUCUCGUCUCCUCGACACCAGGGGUCCACAU | 11 | RL0137-UM strand of |
| GGCAAC | | compound RD0215 (NT) |
| (<Strand Ref.: 488>) | | |
| mG*mG*mU*mG*mU.mC.mG.mA.mG.rA.mA.mG.mA. | 3 | RS0040 strand of compound |
| rG.mG.mA.mG.mA.mA.mC.mA*mA*mU*mA*mU | | RD0216 (NT) |
| (<Strand Ref.: 489>) | | |
| mA*mU*fG*mU*fU.mG.fU.mU.fC.mU.fC.rG.fU. | 11 | RL0138 strand of compound |
| mC.fU.rC.fC.mU.fC.mG.fA.mC.fA.mC.fC. | | RD0216 (NT) |
| mA.mG.mG.mG.mG.mU.rC.rC.rA.mC.mA.mU. | | |
| mG.mG*mC*mA*mA*mC | | |
| (<Strand Ref.: 490>) | | |
| GGUGUCGAGAAGAGGAGAACAAUAU | 3 | RS0040-UM strand of |
| (<Strand Ref.: 491>) | | compound RD0216 (NT) |
| AUGUUGUUCUCGUCUCCUCGACACCAGGGGUCCACAU | 11 | RL0138-UM strand of |
| GGCAAC | | compound RD0216 (NT) |
| (<Strand Ref.: 492>) | | |
| mG*fG*mU*fG*mU.fC.mG.fA.mG.rA.mA.fG.mA. | 3 | RS0041 strand of compound |
| rG.mG.fA.mG.fA.mA.fC.mA*fA*mU*fA*mU | | RD0217 (NT) |
| (<Strand Ref.: 493>) | | |
| mA*mU*fG*mU*fU.mG.fU.mU.fC.mU.fC.rG.fU. | 11 | RL0138 strand of compound |
| mC.fU.rC.fC.mU.fC.mG.fA.mC.fA.mC.fC. | | RD0217 (NT) |
| mA.mG.mG.mG.mG.mU.rC.rC.rA.mC.mA.mU. | | |
| mG.mG*mC*mA*mA*mC | | |
| (<Strand Ref.: 494>) | | |
| GGUGUCGAGAAGAGGAGAACAAUAU | 3 | RS0041-UM strand of |
| (<Strand Ref.: 495>) | | compound RD0217 (NT) |
| AUGUUGUUCUCGUCUCCUCGACACCAGGGGUCCACAU | 11 | RL0138-UM strand of |
| GGCAAC | | compound RD0217 (NT) |
| (<Strand Ref.: 496>) | | |
| mG*mG*fU*mG*fU.mC.fG.mA.fG.rA.fA.mG.fA. | 3 | RS0042 strand of compound |
| rG.fG.mA.fG.mA.fA.mC.fA*mA*fU*mA*mU | | RD0218 (NT) |
| (<Strand Ref.: 497>) | | |
| mA*fU*mG*fU*mU.fG.mU.fU.mC.fU.mC.rG.mU. | 11 | RL0137 strand of compound |
| fC.mU.rC.mC.fU.mC.fG.mA.fC.mA.fC.mC. | | RD0218 (NT) |
| mA.mG.mG.mG.mG.mU.rC.rC.rA.mC.mA.mU. | | |
| mG.mG*mC*mA*mA*mC | | |
| (<Strand Ref.: 498>) | | |
| GGUGUCGAGAAGAGGAGAACAAUAU | 3 | RS0042-UM strand of |
| (<Strand Ref.: 499>) | | compound RD0218 (NT) |
| AUGUUGUUCUCGUCUCCUCGACACCAGGGGUCCACAU | 11 | RL0137-UM strand of |
| GGCAAC | | compound RD0218 (NT) |
| (<Strand Ref.: 500>) | | |
| mG*mG*mU.fG.mU.mC.fG.fA.fG.rA.fA.fG.fA. | 3 | RS0043 strand of compound |
| rG.fG.fA.fG.fA.fA.fC.mA.fA.mU*fA*mU | | RD0220 (NT) |
| (<Strand Ref.: 501>) | | |
| mA*fU*fG.fU.mU.fG.mU.mU.mC.mU.fC.rG.fU. | 11 | RL0140 strand of compound |
| mC.fU.rC.fC.mU.mC.fG.fA.mC.fA.mC.mC. | | RD0220 (NT) |
| mA.mG.mG.mG.mG.mU.rC.rC.rA.mC.mA.mU. | | |
| mG.mG*mC*mA*mA*mC | | |
| (<Strand Ref.: 502>) | | |
| GGUGUCGAGAAGAGGAGAACAAUAU | 3 | RS0043-UM strand of |
| (<Strand Ref.: 503>) | | compound RD0220 (NT) |
| AUGUUGUUCUCGUCUCCUCGACACCAGGGGUCCACAU | 11 | RL0140-UM strand of |
| GGCAAC | | compound RD0220 (NT) |
| (<Strand Ref.: 504>) | | |
| mG.mG.mU.rG.mU.mC.rG.rA.rG.rA.rA.rG.rA. | 3 | RS0044 strand of compound |
| rG.rG.rA.rG.rA.rA.rC.mA.rA.mU*rA*mU | | RD0221 (NT) |
| (<Strand Ref.: 505>) | | |
| mA*fU*rG.fU.mU.rG.mU.mU.mC.mU.fC.rG.fU. | 11 | RL0141 strand of compound |
| mC.fU.rC.fC.mU.mC.rG.rA.mC.rA.mC.mC. | | RD0221 (NT) |
| mA.mG.mG.mG.mG.mU.fC.fC.fA.mC.mA.mU. | | |
| mG.mG*mC*mA*mA*mC | | |
| (<Strand Ref.: 506>) | | |
| GGUGUCGAGAAGAGGAGAACAAUAU | 3 | RS0044-UM strand of |
| (<Strand Ref.: 507>) | | compound RD0221 (NT) |
| AUGUUGUUCUCGUCUCCUCGACACCAGGGGUCCACAU | 11 | RL0141-UM strand of |
| GGCAAC | | compound RD0221 (NT) |
| (<Strand Ref.: 508>) | | |
| mG.mG.mU.rG.mU.mC.rG.rA.rG.rA.rA.rG.rA. | 3 | RS0044 strand of compound |
| rG.rG.rA.rG.rA.rA.rC.mA.rA.mU*rA*mU | | RD0222 (NT) |
| (<Strand Ref.: 509>) | | |
| mA*fU*rG.fU.mU.rG.mU.mU.mC.mU.fC.rG.fU. | 11 | RL0142 strand of compound |
| mC.fU.rC.fC.mU.mC.rG.rA.mC.rA.mC.mC. | | RD0222 (NT) |
| mA.mG.mG.mG.mG.mU.fC.rC.fA.mC.mA.mU. | | |
| mG.mG*mC*mA*mA*mC | | |
| (<Strand Ref.: 510>) | | |
| GGUGUCGAGAAGAGGAGAACAAUAU | 3 | RS0044-UM strand of |
| (<Strand Ref.: 511>) | | compound RD0222 (NT) |
| AUGUUGUUCUCGUCUCCUCGACACCAGGGGUCCACAU | 11 | RL0142-UM strand of |
| GGCAAC | | compound RD0222 (NT) |
| (<Strand Ref.: 512>) | | |
| mG.mG.mU.rG.mU.mC.rG.rA.rG.rA.rA.rG.rA. | 3 | RS0044 strand of compound |
| rG.rG.rA.rG.rA.rA.rC.mA.rA.mU*rA*mU | | RD0223 (NT) |
| (<Strand Ref.: 513>) | | |
| mA*fU*rG.fU.mU.rG.mU.mU.mC.mU.fC.rG.fU. | 11 | RL0143 strand of compound |
| mC.fU.rC.fC.mU.mC.rG.rA.mC.rA.mC.mC. | | RD0223 (NT) |
| mA.mG.mG.mG.mG.mU.rC.fC.rA.mC.mA.mU. | | |
| mG.mG*mC*mA*mA*mC | | |
| (<Strand Ref.: 514>) | | |
| GGUGUCGAGAAGAGGAGAACAAUAU | 3 | RS0044-UM strand of |
| (<Strand Ref.: 515>) | | compound RD0223 (NT) |
| AUGUUGUUCUCGUCUCCUCGACACCAGGGGUCCACAU | 11 | RL0143-UM strand of |
| GGCAAC | | compound RD0223 (NT) |
| (<Strand Ref.: 516>) | | |
| mG*mG*mU*rG*mU.mC.rG.rA.rG.rA.rA.rG.rA. | 50 | RS0045 strand of compound |
| rG.rG.rA.rG.rA.rA.rC.mA.rA.mU*rA*mU* | | RD0224 (NT) |
| mG*mC | | |
| (<Strand Ref.: 517>) | | |
| mG*mC*mA*fU*rG.fU.mU.rG.mU.mU.mC.mU.fC. | 51 | RL0144 strand of compound |
| rG.fU.mC.fU.rC.fC.mU.mC.rG.rA.mC.rA. | | RD0224 (NT) |
| mC.mC.mA.mG.mG.mG.mG.mU.rC.rC.rA.mC. | | |
| mA.mU.mG.mG*mC*mA*mA*mC | | |
| (<Strand Ref.: 518>) | | |
| GGUGUCGAGAAGAGGAGAACAAUAUGC | 50 | RS0045-UM strand of |
| (<Strand Ref.: 519>) | | compound RD0224 (NT) |
| GCAUGUUGUUCUCGUCUCCUCGACACCAGGGGUCCAC | 51 | RL0144-UM strand of |
| AUGGCAAC | | compound RD0224 (NT) |
| (<Strand Ref.: 520>) | | |
| mG*mG*mU*rG*mU.mC.rG.rA.rG.rA.rA.rG.rA. | 50 | RS0045 strand of compound |
| rG.rG.rA.rG.rA.rA.rC.mA.rA.mU*rA*mU* | | RD0225 (NT) |
| mG*mC | | |
| (<Strand Ref.: 521>) | | |
| mG*mC*mA*fU*rA.fU.mU.rG.mU.mU.mC.mU.fC. | 52 | RL0145 strand of compound |
| rG.fU.mC.fU.rC.fC.mU.mC.rG.rA.mC.rA. | | RD0225 (NT) |
| mC.mC.mA.mG.mG.mG.mG.mU.rC.rC.rA.mC. | | |
| mA.mU.mG.mG*mC*mA*mA*mC | | |
| (<Strand Ref.: 522>) | | |
| GGUGUCGAGAAGAGGAGAACAAUAUGC | 50 | RS0045-UM strand of |
| (<Strand Ref.: 523>) | | compound RD0225 (NT) |
| GCAUAUUGUUCUCGUCUCCUCGACACCAGGGGUCCAC | 52 | RL0145-UM strand of |
| AUGGCAAC | | compound RD0225 (NT) |
| (<Strand Ref.: 524>) | | |
| mG*mG*mU*rG*mU.mC.rG.rA.rG.rA.rA.rG.rA. | 53 | RS0046 strand of compound |
| rG.rG.rA.rG*rA*rA*rC*mA | | RD0226 (NT) |
| (<Strand Ref.: 525>) | | |
| mU*rG*mU*mU*mC.mU.fC.rG.fU.mC.fU.rC.fC. | 54 | RL0146 strand of compound |
| mU.mC.rG.rA.mC.rA.mC.mC.mA.mG.mG.mG. | | RD0226 (NT) |
| mG.mU.rC.rC.rA.mC.mA.mU.mG.mG*mC*mA* | | |
| mA*mC | | |
| (<Strand Ref.: 526>) | | |
| GGUGUCGAGAAGAGGAGAACA | 53 | RS0046-UM strand of |
| (<Strand Ref.: 527>) | | compound RD0226 (NT) |
| UGUUCUCGUCUCCUCGACACCAGGGGUCCACAUGGCA | 54 | RL0146-UM strand of |
| AC | | compound RD0226 (NT) |
| (<Strand Ref.: 528>) | | |
| mG*mG*mU*rG*mU.mC.rG.rA.rG.rA.rA.rG.rA. | 55 | RS0047 strand of compound |
| rG.rG.rA.rG*rA*rA*rC*mG | | RD0227 (NT) |
| (<Strand Ref.: 529>) | | |
| mC*rG*mU*mU*mC.mU.fC.rG.fU.mC.fU.rC.fC. | 56 | RL0147 strand of compound |
| mU.mC.rG.rA.mC.rA.mC.mC.mA.mG.mG.mG. | | RD0227 (NT) |
| mG.mU.rC.rC.rA.mC.mA.mU.mG.mG*mC*mA* | | |
| mA*mC | | |
| (<Strand Ref.: 530>) | | |
| GGUGUCGAGAAGAGGAGAACG | 55 | RS0047-UM strand of |
| (<Strand Ref.: 531>) | | compound RD0227 (NT) |
| CGUUCUCGUCUCCUCGACACCAGGGGUCCACAUGGCA | 56 | RL0147-UM strand of |
| AC | | compound RD0227 (NT) |
| (<Strand Ref.: 532>) | | |
| rG*mG*mG*rU*rG.rG.rA.rA.rG.rA.rG.rG.rA. | 57 | RS0048 strand of compound |
| rG*rA*rA*rC*mG | | RD0228 (NT) |
| (<Strand Ref.: 533>) | | |
| mC*rG*mU*mU*mC.mU.fC.rG.fU.mC.fU.rC.fC. | 58 | RL0148 strand of compound |
| mC.mA.rC.rC.mC.mA.mG.mG.mG.mG.mU.rC. | | RD0228 (NT) |
| rC.rA.mC.mA.mU.mG.mG*mC*mA*mA*mC | | |
| (<Strand Ref.: 534>) | | |
| GGGUGGAAGAGGAGAACG | 57 | RS0048-UM strand of |
| (<Strand Ref.: 535>) | | compound RD0228 (NT) |
| CGUUCUCGUCUCCCACCCAGGGGUCCACAUGGCAAC | 58 | RL0148-UM strand of |
| (<Strand Ref.: 536>) | | compound RD0228 (NT) |
| rG*mG*mG*rU*rG.rG.rA.rA.rG.rA.rG.rU.rA. | 59 | RS0049 strand of compound |
| rG*rA*rC*rC*mG | | RD0229 (NT) |
| (<Strand Ref.: 537>) | | |
| mC*rG*mG*mU*mC.mU.fA.rG.fU.mC.fU.rC.fC. | 60 | RL0149 strand of compound |
| mC.mA.rC.rC.mC.mA.mG.mG.mG.mG.mU.rC. | | RD0229 (NT) |
| rC.rA.mC.mA.mU.mG.mG*mC*mA*mA*mC | | |
| (<Strand Ref.: 538>) | | |
| GGGUGGAAGAGUAGACCG | 59 | RS0049-UM strand of |
| (<Strand Ref.: 539>) | | compound RD0229 (NT) |
| CGGUCUAGUCUCCCACCCAGGGGUCCACAUGGCAAC | 60 | RL0149-UM strand of |
| (<Strand Ref.: 540>) | | compound RD0229 (NT) |
| rG*mG*mG*rU*rG.rG.rA.rA.rG.rA.rG.rG.rA. | 61 | RS0050 strand of compound |
| rG*rA*rC*rC*mA | | RD0230 (NT) |
| (<Strand Ref.: 541>) | | |
| mU*rG*mG*mU*mC.mU.fC.rG.fU.mC.fU.rC.fC. | 62 | RLO150 strand of compound |
| mC.mA.rC.rC.mC.mA.mG.mG.mG.mG.mU.rC. | | RD0230 (NT) |
| rC.rA.mC.mA.mU.mG.mG*mC*mA*mA*mC | | |
| (<Strand Ref.: 542>) | | |
| GGGUGGAAGAGGAGACCA | 61 | RS0050-UM strand of |
| (<Strand Ref.: 543>) | | compound RD0230 (NT) |
| UGGUCUCGUCUCCCACCCAGGGGUCCACAUGGCAAC | 62 | RL0150-UM strand of |
| (<Strand Ref.: 544>) | | compound RD0230 (NT) |
| mG*rU*rG*rG.rA.rA.rG.rA.rG.rG.rA.rG*rA* | 63 | RS0051 strand of compound |
| rA*rC*mA | | RD0231 (NT) |
| (<Strand Ref.: 545>) | | |
| mU*rG*mU*mU*mC.mU.fC.rG.fU.mC.fU.rC.fC. | 64 | RL0151 strand of compound |
| mC.mA.rC.mA.mG.mG.mG.mG.mU.rC.rC.rA. | | RD0231 (NT) |
| mC.mA.mU.mG.mG*mC*mA*mA*mC | | |
| (<Strand Ref.: 546>) | | |
| GUGGAAGAGGAGAACA | 63 | RS0051-UM strand of |
| (<Strand Ref.: 547>) | | compound RD0231 (NT) |
| UGUUCUCGUCUCCCACAGGGGUCCACAUGGCAAC | 64 | RL0151-UM strand of |
| (<Strand Ref.: 548>) | | compound RD0231 (NT) |
| mG*rU*rG*rG.rA.rA.rG.rA.rG.rU.rA.rG*rA* | 65 | RS0052 strand of compound |
| rC*rC*mA | | RD0232 (NT) |
| (<Strand Ref.: 549>) | | |
| mU*rG*mG*mU*mC.mU.fA.rG.fU.mC.fU.rC.fC. | 66 | RL0152 strand of compound |
| mC.mA.rC.mA.mG.mG.mG.mG.mU.rC.rC.rA. | | RD0232 (NT) |
| mC.mA.mU.mG.mG*mC*mA*mA*mC | | |
| (<Strand Ref.: 550>) | | |
| GUGGAAGAGUAGACCA | 65 | RS0052-UM strand of |
| (<Strand Ref.: 551>) | | compound RD0232 (NT) |
| UGGUCUAGUCUCCCACAGGGGUCCACAUGGCAAC | 66 | RL0152-UM strand of |
| (<Strand Ref.: 552>) | | compound RD0232 (NT) |
| mG*rU*rG*rG.rA.rA.rG.rA.rG.rG.rA.rG*rA* | 67 | RS0053 strand of compound |
| rA*rC*mG | | RD0233 (NT) |
| (<Strand Ref.: 553>) | | |
| mC*rG*mU*mU*mC.mU.fC.rG.fU.mC.fU.rC.fC. | 68 | RL0153 strand of compound |
| mC.mA.rC.mA.mG.mG.mG.mG.mU.rC.rC.rA. | | RD0233 (NT) |
| .mC.mA.mU.mG.mG*mC*mA*mA*mC | | |
| (<Strand Ref.: 554>) | | |
| GUGGAAGAGGAGAACG | 57 | RS0053-UM strand of |
| (<Strand Ref.: 555>) | | compound RD0233 (NT) |
| CGUUCUCGUCUCCCACAGGGGUCCACAUGGCAAC | 68 | RL0153-UM strand of |
| (<Strand Ref.: 556>) | | compound RD0233 (NT) |
| mG*rU*rG*rG.rA.rA.rG.rA.rG.rU.rA.rG*rA* | 69 | RS0054 strand of compound |
| rC*rC*mG | | RD0234 (NT) |
| (<Strand Ref.: 557>) | | |
| mC*rG*mG*mU*mC.mU.fA.rG.fU.mC.fU.rC.fC. | 70 | RL0154 strand of compound |
| mC.mA.rC.mA.mG.mG.mG.mG.mU.rC.rC.rA. | | RD0234 (NT) |
| mC.mA.mU.mG.mG*mC*mA*mA*mC | | |
| (<Strand Ref.: 558>) | | |
| GUGGAAGAGUAGACCG | 69 | RS0054-UM strand of |
| (<Strand Ref.: 559>) | | compound RD0234 (NT) |
| CGGUCUAGUCUCCCACAGGGGUCCACAUGGCAAC | 70 | RL0154-UM strand of |
| (<Strand Ref.: 560>) | | compound RD0234 (NT) |
| mG*rU*rG*rG.rA.rA.rG.rA.rG.rG.rA.rG.rA. | 71 | RS0055 strand of compound |
| rA*rC*mA*rA*mC | | RD0235 (NT) |
| (<Strand Ref.: 561>) | | |
| rG*fU*mU*rG*mU.mU.mC.mU.fC.rG.fU.mC.fU. | 72 | RL0155 strand of compound |
| rC.fC.mC.mA.rC.mA.mG.mG.mG.mG.mU.rC. | | RD0235 (NT) |
| rC.rA.mC.mA.mU.mG.mG*mC*mA*mA*mC | | |
| (<Strand Ref.: 562>) | | |
| GUGGAAGAGGAGAACAAC | 71 | RS0055-UM strand of |
| (<Strand Ref.: 563>) | | compound RD0235 (NT) |
| GUUGUUCUCGUCUCCCACAGGGGUCCACAUGGCAAC | 72 | RL0155-UM strand of |
| (<Strand Ref.: 564>) | | compound RD0235 (NT) |
| mG*rU*rG*rG.rA.rA.rG.rA.rG.rG.rA.rG.rA. | 73 | RS0056 strand of compound |
| rA*rC*mA*rG*mC | | RD0236 (NT) |
| (<Strand Ref.: 565>) | | |
| rG*fC*mU*rG*mU.mU.mC.mU.fC.rG.fU.mC.fU. | 74 | RL0156 strand of compound |
| rC.fC.mC.mA.rC.mA.mG.mG.mG.mG.mU.rC. | | RD0236 (NT) |
| rC.rA.mC.mA.mU.mG.mG*mC*mA*mA*mC | | |
| (<Strand Ref.: 566>) | | |
| GUGGAAGAGGAGAACAGC | 73 | RS0056-UM strand of |
| (<Strand Ref.: 567>) | | compound RD0236 (NT) |
| GCUGUUCUCGUCUCCCACAGGGGUCCACAUGGCAAC | 74 | RL0156-UM strand of |
| (<Strand Ref.: 568>) | | compound RD0236 (NT) |
| mG*rU*rG*rG.rA.rA.rG.rA.rG.rG.rA.rG.rA. | 75 | RS0057 strand of compound |
| rA.rC.mA*rA*mC*mG*mC | | RD0237 (NT) |
| (<Strand Ref.: 569>) | | |
| mG*fC*rG*fU*mU.rG.mU.mU.mC.mU.fC.rG.fU. | 76 | RL0157 strand of compound |
| mC.fU.rC.fC.mC.mA.rC.mA.mG.mG.mG.mG. | | RD0237 (NT) |
| mU.rC.rC.rA.mC.mA.mU.mG.mG*mC*mA*mA* | | |
| mC | | |
| (<Strand Ref.: 570>) | | |
| GUGGAAGAGGAGAACAACGC | 75 | RS0057-UM strand of |
| (<Strand Ref.: 571>) | | compound RD0237 (NT) |
| GCGUUGUUCUCGUCUCCCACAGGGGUCCACAUGGCAA | 76 | RL0157-UM strand of |
| C | | compound RD0237 (NT) |
| (<Strand Ref.: 572>) | | |
| mG*rU*rG*rG.rA.rA.rG.rA.rG.rG.rA.rG.rA. | 77 | RS0058 strand of compound |
| rA.rC.mA*rA*mU*mG*mC | | RD0238 (NT) |
| (<Strand Ref.: 573>) | | |
| mG*fC*rA*fU*mU.rG.mU.mU.mC.mU.fC.rG.fU. | 78 | RL0158 strand of compound |
| mC.fU.rC.fC.mC.mA.rC.mA.mG.mG.mG.mG. | | RD0238 (NT) |
| mU.rC.rC.rA.mC.mA.mU.mG.mG*mC*mA*mA* | | |
| mC | | |
| (<Strand Ref.: 574>) | | |
| GUGGAAGAGGAGAACAAUGC | 77 | RS0058-UM strand of |
| (<Strand Ref.: 575>) | | compound RD0238 (NT) |
| GCAUUGUUCUCGUCUCCCACAGGGGUCCACAUGGCAA | 78 | RL0158-UM strand of |
| C | | compound RD0238 (NT) |
| (<Strand Ref.: 576>) | | |
| mG*rU*rG*rG.rA.rA.rG.rA.rG.rU.rA.rG.rA* | 79 | RS0059 strand of compound |
| rC*rC*mA*mC | | RD0239 (NT) |
| (<Strand Ref.: 577>) | | |
| mG*mU*rG*mG*mU.mC.mU.fA.rG.fU.mC.fU.rC. | 80 | RL0159 strand of compound |
| fC.mC.mA.rC.mA.mG.mG.mG.mG.mU.rC.rC. | | RD0239 (NT) |
| rA.mC.mA.mU.mG.mG*mC*mA*mA*mC | | |
| (<Strand Ref.: 578>) | | |
| GUGGAAGAGUAGACCAC | 79 | RS0059-UM strand of |
| (<Strand Ref.: 579>) | | compound RD0239 (NT) |
| GUGGUCUAGUCUCCCACAGGGGUCCACAUGGCAAC | 80 | RL0159-UM strand of |
| (<Strand Ref.: 580>) | | compound RD0239 (NT) |
| mG*rU*rG*rG.rA.rA.rG.rA.rG.rG.rA.rG.rA* | 81 | RS0060 strand of compound |
| rA*rC*mG*mC | | RD0240 (NT) |
| (<Strand Ref.: 581>) | | |
| mG*mC*rG*mU*mU.mC.mU.fC.rG.fU.mC.fU.rC. | 82 | RL0160 strand of compound |
| fC.mC.mA.rC.mA.mG.mG.mG.mG.mU.rC.rC. | | RD0240 (NT) |
| rA.mC.mA.mU.mG.mG*mC*mA*mA*mC | | |
| (<Strand Ref.: 582>) | | |
| GUGGAAGAGGAGAACGC | 81 | RS0060-UM strand of |
| (<Strand Ref.: 583>) | | compound RD0240 (NT) |
| GCGUUCUCGUCUCCCACAGGGGUCCACAUGGCAAC | 82 | RL0160-UM strand of |
| (<Strand Ref.: 584>) | | compound RD0240 (NT) |
| mG*fG*mU*fG*mU.fC.mG.fA.mG.fA.mA.fG.mA. | 3 | RS0037 strand of compound |
| fG.mG.fA.mG.fA.mA.fC.mA*fA*mU*fA*mU | | RD0241 (NT) |
| (<Strand Ref.: 585>) | | |
| mA*fU*mG*fU*mU.fG.mU.fU.mC.fU.mC.fG.mU. | 1 | RL0130 strand of compound |
| fC.mU.mC.mC.fU.mC.fG.mA.fC.mA.fC.mC. | | RD0241 (NT) |
| mA.mG.mG.mG.mG.mU.rC.rC.rA.mC.mA.mU. | | |
| mG.mG*mC*mA*mA*mC | | |
| (<Strand Ref.: 586>) | | |
| GGUGUCGAGAAGAGGAGAACAAUAU | 3 | RS0037-UM strand of |
| (<Strand Ref.: 587>) | | compound RD0241 (NT) |
| AUGUUGUUCUCGUCUCCUCGACACCAGGGGUCCACAU | 11 | RL0130-UM strand of |
| GGCAAC | | compound RD0241 (NT) |
| (<Strand Ref.: 588>) | | |
| mG*mG*fU*mG*fU.mC.fG.mA.fG.fA.fA.mG.fA. | 3 | RS0038 strand of compound |
| fG.fG.mA.fG.mA.fA.mC.fA*mA*fU*mA*mU | | RD0242 (NT) |
| (<Strand Ref.: 589>) | | |
| mA*mU*fG*mU*fU.mG.fU.mU.fC.mU.fC.fG.fU. | 1 | RL0131 strand of compound |
| mC.fU.mC.fC.mU.fC.mG.fA.mC.fA.mC.fC. | | RD0242 (NT) |
| mA.mG.mG.mG.mG.mU.rC.rC.rA.mC.mA.mU. | | |
| mG.mG*mC*mA*mA*mC | | |
| (<Strand Ref.: 590>) | | |
| GGUGUCGAGAAGAGGAGAACAAUAU | 3 | RS0038-UM strand of |
| (<Strand Ref.: 591>) | | compound RD0242 (NT) |
| AUGUUGUUCUCGUCUCCUCGACACCAGGGGUCCACAU | 11 | RL0131-UM strand of |
| GGCAAC | | compound RD0242 (NT) |
| (<Strand Ref.: 592>) | | |
| mG*mG*mU.mG.mU.mC.mG.mA.mG.rA.mA.mG.mA. | 3 | RS0061 strand of compound |
| rG.mG.mA.mG.mA.mA.mC.mA.mA.mU*mA*mU | | RD0243 (NT) |
| (<Strand Ref.: 593>) | | |
| mA*mU*mG.mU.mU.mG.mU.mU.mC.mU.mC.rG.mU. | 1 | RL0161 strand of compound |
| mC.mU.rC.mC.mU.mC.mG.mA.mC.mA.mC.mC. | | RD0243 (NT) |
| mA.mG.mG.mG.mG.mU.rC.rC.rA.mC.mA.mU. | | |
| mG.mG.mC.mA.mA.mC | | |
| (<Strand Ref.: 594>) | | |
| GGUGUCGAGAAGAGGAGAACAAUAU | 3 | RS0061-UM strand of |
| (<Strand Ref.: 595>) | | compound RD0243 (NT) |
| AUGUUGUUCUCGUCUCCUCGACACCAGGGGUCCACAU | 11 | RL0161-UM strand of |
| GGCAAC | | compound RD0243 (NT) |
| (<Strand Ref.: 596>) | | |
| mG*fG*mU*fG*mU.fC.mG.fA.mG.rA.mA.fG.mA. | 3 | RS0041 strand of compound |
| rG.mG.fA.mG.fA.mA.fC.mA*fA*mU*fA*mU | | RD0244 (NT) |
| (<Strand Ref.: 597>) | | |
| mA*fU*mG*fU*mU.fG.mU.fU.mC.fU.mC.rG.mU. | 11 | RL0137 strand of compound |
| fC.mU.rC.mC.fU.mC.fG.mA.fC.mA.fC.mC. | | RD0244 (NT) |
| mA.mG.mG.mG.mG.mU.rC.rC.rA.mC.mA.mU. | | |
| mG.mG*mC*mA*mA*mC | | |
| (<Strand Ref.: 598>) | | |
| GGUGUCGAGAAGAGGAGAACAAUAU | 3 | RS0041-UM strand of |
| (<Strand Ref.: 599>) | | compound RD0244 (NT) |
| AUGUUGUUCUCGUCUCCUCGACACCAGGGGUCCACAU | 11 | RL0137-UM strand of |
| GGCAAC | | compound RD0244 (NT) |
| (<Strand Ref.: 600>) | | |
| mG*mG*fU*mG*fU.mC.fG.mA.fG.rA.fA.mG.fA. | 3 | RS0042 strand of compound |
| rG.fG.mA.fG.mA.fA.mC.fA*mA*fU*mA*mU | | RD0245 (NT) |
| (<Strand Ref.: 601>) | | |
| mA*mU*fG*mU*fU.mG.fU.mU.fC.mU.fC.rG.fU. | 1 | RL0138 strand of compound |
| mC.fU.rC.fC.mU.fC.mG.fA.mC.fA.mC.fC. | | RD0245 (NT) |
| mA.mG.mG.mG.mG.mU.rC.rC.rA.mC.mA.mU. | | |
| mG.mG*mC*mA*mA*mC | | |
| (<Strand Ref.: 602>) | | |
| GGUGUCGAGAAGAGGAGAACAAUAU | 3 | RS0042-UM strand of |
| (<Strand Ref.: 603>) | | compound RD0245 (NT) |
| AUGUUGUUCUCGUCUCCUCGACACCAGGGGUCCACAU | 11 | RL0138-UM strand of |
| GGCAAC | | compound RD0245 (NT) |
| (<Strand Ref.: 604>) | | |
| mG*mG*mU*fG*mU.mC.fG.fA.fG.rA.fA.fG.fA. | 3 | RS0062 strand of compound |
| rG.fG.fA.fG.fA.fA.fC.mA.fA.mU*fA*mU | | RD0246 (NT) |
| (<Strand Ref.: 605>) | | |
| mA*fU*fG*fU*mU.fG.mU.mU.mC.mU.fC.rG.fU. | 11 | RL0162 strand of compound |
| mC.fU.rC.fC.mU.mC.fG.fA.mC.fA.mC.mC. | | RD0246 (NT) |
| mA.mG.mG.mG.mG.mU.rC.rC.rA.mC.mA.mU. | | |
| mG.mG*mC*mA*mA*mC | | |
| (<Strand Ref.: 606>) | | |
| GGUGUCGAGAAGAGGAGAACAAUAU | 3 | RS0062-UM strand of |
| (<Strand Ref.: 607>) | | compound RD0246 (NT) |
| AUGUUGUUCUCGUCUCCUCGACACCAGGGGUCCACAU | 11 | RL0162-UM strand of |
| GGCAAC | | compound RD0246 (NT) |
| (<Strand Ref.: 608>) | | |
| mG*mG*mG*mU*mG.mG.rA.mA.mU.mA.rG.mU.mA. | 26 | RS0063 strand of compound |
| mC.mA.mA.mC.mA*mA*mU*mG*mC | | RD0344 (NT) |
| (<Strand Ref.: 609>) | | |
| mG*mC*mA*mU*mU.mG.mU.mU.mG.mU.mA.rG.mU. | 27 | RL0167 strand of compound |
| mA.mU.mC.mC.mC.mA.mC.mC.mC.mA.mG.mG. | | RD0344 (NT) |
| mG.mG.mU.rC.rC.rA.mC.mA.mU.mG.mG*mC* | | |
| mA*mA*mC | | |
| (<Strand Ref.: 610>) | | |
| GGGUGGAAUAGUACAACAAUGC | 26 | RS0063-UM strand of |
| (<Strand Ref.: 611>) | | compound RD0344 (NT) |
| GCAUUGUUGUAGUAUCCCACCCAGGGGUCCACAUGGC | 27 | RL0167-UM strand of |
| AAC | | compound RD0344 (NT) |
| (<Strand Ref.: 612>) | | |
| mG*mG*mG*mU*mG.mG.rA.mA.mU.mA.rG.mU.mA. | 22 | RS0064 strand of compound |
| mU.mA.mA.mC.mA*mA*mU*mA*mU | | RD0345 (NT) |
| (<Strand Ref.: 613>) | | |
| mA*mU*mA*mU*mU.mG.mU.mU.mA.mU.mA.rG.mU. | 23 | RL0168 strand of compound |
| mA.mU.mC.mC.mC.mA.mC.mC.mC.mA.mG.mG. | | RD0345 (NT) |
| mG.mG.mU.rC.rC.rA.mC.mA.mU.mG.mG*mC* | | |
| mA*mA*mC | | |
| (<Strand Ref.: 614>) | | |
| GGGUGGAAUAGUAUAACAAUAU | 22 | RS0064-UM strand of |
| (<Strand Ref.: 615>) | | compound RD0345 (NT) |
| AUAUUGUUAUAGUAUCCCACCCAGGGGUCCACAUGGC | 23 | RL0168-UM strand of |
| AAC | | compound RD0345 (NT) |
| (<Strand Ref.: 616>) | | |
| mG*mG*mU*mG*mG.mG.mU.mG.mG.rA.mA.mU.mA. | 28 | RS0065 strand of compound |
| rG.mU.mA.mU.mA.mA.mC.mA*mA*mU*mA*mU | | RD0346 (NT) |
| (<Strand Ref.: 617>) | | |
| mA*mU*mG*mU*mU.mG.mU.mU.mA.mU.mA.rG.mU. | 29 | RL0169 strand of compound |
| mA.mU.mC.mC.mC.mA.mC.mC.mC.mA.mC.mC. | | RD0346 (NT) |
| mA.mG.mG.mG.mG.mU.rC.rC.rA.mC.mA.mU. | | |
| mG.mG*mC*mA*mA*mC | | |
| (<Strand Ref.: 618>) | | |
| GGUGGGUGGAAUAGUAUAACAAUAU | 28 | RS0065-UM strand of |
| (<Strand Ref.: 619>) | | compound RD0346 (NT) |
| AUGUUGUUAUAGUAUCCCACCCACCAGGGGUCCACAU | 29 | RL0169-UM strand of |
| GGCAAC | | compound RD0346 (NT) |
| (<Strand Ref.: 620>) | | |
| mG*mG*mU*mG*mG.mG.mU.mG.mG.rA.mA.mG.mA. | 30 | RS0066 strand of compound |
| rG.mU.mA.mU.mA.mA.mC.mA*mA*mU*mG*mC | | RD0347 (NT) |
| (<Strand Ref.: 621>) | | |
| mG*mC*mG*mU*mU.mG.mU.mU.mA.mU.mA.rG.mU. | 31 | RL0170 strand of compound |
| mC.mU.mC.mC.mC.mA.mC.mC.mC.mA.mC.mC. | | RD0347 (NT) |
| mA.mG.mG.mG.mG.mU.rC.rC.rA.mC.mA.mU. | | |
| mG.mG*mC*mA*mA*mC | | |
| (<Strand Ref.: 622>) | | |
| GGUGGGUGGAAGAGUAUAACAAUGC | 30 | RS0066-UM strand of |
| (<Strand Ref.: 623>) | | compound RD0347 (NT) |
| GCGUUGUUAUAGUCUCCCACCCACCAGGGGUCCACAU | 31 | RL0170-UM strand of |
| GGCAAC | | compound RD0347 (NT) |
| (<Strand Ref.: 624>) | | |
| mG*mG*mU*mG*mG.mG.mU.mG.mG.rA.mA.mC.mA. | 32 | RS0067 strand of compound |
| rG.mU.mA.mU.mA.mA.mC.mA*mA*mU*mG*mC | | RD0348 (NT) |
| (<Strand Ref.: 625>) | | |
| mG*mC*mG*mU*mU.mG.mU.mU.mA.mU.mA.rG.mU. | 33 | RL0171 strand of compound |
| mG.mU.mC.mC.mC.mA.mC.mC.mC.mA.mC.mC. | | RD0348 (NT) |
| mA.mG.mG.mG.mG.mU.rC.rC.rA.mC.mA.mU. | | |
| mG.mG*mC*mA*mA*mC | | |
| (<Strand Ref.: 626>) | | |
| GGUGGGUGGAACAGUAUAACAAUGC | 32 | RS0067-UM strand of |
| (<Strand Ref.: 627>) | | compound RD0348 (NT) |
| GCGUUGUUAUAGUGUCCCACCCACCAGGGGUCCACAU | 33 | RL0171-UM strand of |
| GGCAAC | | compound RD0348 (NT) |
| (<Strand Ref.: 628>) | | |
| mG*mG*mU*mG*mG.mG.mU.mG.mG.rA.mA.mU.mA. | 34 | RS0068 strand of compound |
| rG.mU.mA.mU.mA.mA.mC.mA.mA.mU.mA*mU* | | RD0349 (NT) |
| mG*mC | | |
| (<Strand Ref.: 629>) | | |
| mG*mC*mA*mU*mG.mU.mU.mG.mU.mU.mA.mU.mA. | 35 | RL0172 strand of compound |
| rG.mU.mA.mU.mC.mC.mC.mA.mC.mC.mC.mA. | | RD0349 (NT) |
| mC.mC.mA.mG.mG.mG.mG.mU.rC.rC.rA.mC. | | |
| mA.mU.mG.mG*mC*mA*mA*mC | | |
| (<Strand Ref.: 630>) | | |
| GGUGGGUGGAAUAGUAUAACAAUAUGC | 34 | RS0068-UM strand of |
| (<Strand Ref.: 631>) | | compound RD0349 (NT) |
| GCAUGUUGUUAUAGUAUCCCACCCACCAGGGGUCCAC | 35 | RL0172-UM strand of |
| AUGGCAAC | | compound RD0349 (NT) |
| (<Strand Ref.: 632>) | | |
| mG*mG*fU*mG*fU.mC.fG.mA.fG.fA.fA.mG.fA. | 3 | RS0038 strand of compound |
| fG.fG.mA.fG.mA.fA.mC.fA*mA*fU*mA*mU | | RD0441 (NT) |
| (<Strand Ref.: 633>) | | |
| mA*fU*mG*fU*mU.fG.mU.fU.mC.fU.mC.fG.mU. | 83 | RL0204 strand of compound |
| fC.mU.mC.mC.fU.mC.fG.mA.fC.mA.fC.mC. | | RD0441 (NT) |
| mA.mG.mG.mG.mG.mU.rC.rC.rA.mC.mA.mU. | | |
| mG.mG*mA*mA*mA*mC | | |
| (<Strand Ref.: 634>) | | |
| GGUGUCGAGAAGAGGAGAACAAUAU | 3 | RS0038-UM strand of |
| (<Strand Ref.: 635>) | | compound RD0441 (NT) |
| AUGUUGUUCUCGUCUCCUCGACACCAGGGGUCCACAU | 83 | RL0204-UM strand of |
| GGAAAC | | compound RD0441 (NT) |
| (<Strand Ref.: 636>) | | |
| mG*mG*mU*fG*mU.mC.fG.fA.fG.rA.fA.fG.fA. | 3 | RS0062 strand of compound |
| rG.fG.fA.fG.fA.fA.fC.mA.fA.mU*fA*mU | | RD0442 (NT) |
| (<Strand Ref.: 637>) | | |
| mA*fU*fG*fU*mU.fG.mU.mU.mC.mU.fC.rG.fU. | 83 | RL0205 strand of compound |
| mC.fU.rC.fC.mU.mC.fG.fA.mC.fA.mC.mC. | | RD0442 (NT) |
| mA.mG.mG.mG.mG.mU.rC.rC.rA.mC.mA.mU. | | |
| mG.mG*mA*mA*mA*mC | | |
| (<Strand Ref.: 638>) | | |
| GGUGUCGAGAAGAGGAGAACAAUAU | 3 | RS0062-UM strand of |
| (<Strand Ref.: 639>) | | compound RD0442 (NT) |
| AUGUUGUUCUCGUCUCCUCGACACCAGGGGUCCACAU | 83 | RL0205-UM strand of |
| GGAAAC | | compound RD0442 (NT) |
| (<Strand Ref.: 640>) | | |
| mG*mG*mU*fG*mG.mG.fU.fG.fG.fA.fA.fG.fA. | 84 | RS0078 strand of compound |
| fG.fG.fA.fG*fA*fA*fC*mA | | RD0452 (NT) |
| (<Strand Ref.: 641>) | | |
| mU*fG*mU*mU*mC.mU.fC.fG.fU.mC.fU.mC.fC. | 85 | RL0211 strand of compound |
| mC.mA.fC.fC.mC.fA.mC.mC.mA.mG.mG.mG. | | RD0452 (NT) |
| mG.mU.rC.rC.rA.mC.mA.mU.mG.mG*mC*mA* | | |
| mA*mC | | |
| (<Strand Ref.: 642>) | | |
| GGUGGGUGGAAGAGGAGAACA | 84 | RS0078-UM strand of |
| (<Strand Ref.: 643>) | | compound RD0452 (NT) |
| UGUUCUCGUCUCCCACCCACCAGGGGUCCACAUGGCA | 85 | RL0211-UM strand of |
| AC | | compound RD0452 (NT) |
| (<Strand Ref.: 644>) | | |
| mG*mG*mU*fG*mG.mG.fU.fG.fG.fA.fA.fG.fA. | 86 | RS0079 strand of compound |
| fG.fG.fA.fG*fA*fA*fC*mG | | RD0453 (NT) |
| (<Strand Ref.: 645>) | | |
| mC*fG*mU*mU*mC.mU.fC.fG.fU.mC.fU.mC.fC. | 87 | RL0212 strand of compound |
| mC.mA.fC.fC.mC.fA.mC.mC.mA.mG.mG.mG. | | RD0453 (NT) |
| mG.mU.rC.rC.rA.mC.mA.mU.mG.mG*mC*mA* | | |
| mA*mC | | |
| (<Strand Ref.: 646>) | | |
| GGUGGGUGGAAGAGGAGAACG | 86 | RS0079-UM strand of |
| (<Strand Ref.: 647>) | | compound RD0453 (NT) |
| CGUUCUCGUCUCCCACCCACCAGGGGUCCACAUGGCA | 87 | RL0212-UM strand of |
| AC | | compound RD0453 (NT) |
| (<Strand Ref.: 648>) | | |
| mG*mG*mG*fU.fG.fG.fA.fA.fG.fA.fG.fU.fA. | 5 | RS0080 strand of compound |
| fG.fA.fA.fC.mA.fA.mU*fA*mU*fG*mC | | RD0454 (NT) |
| (<Strand Ref.: 649>) | | |
| mG*fC*mA.fU.fA.fU.mU.fG.mU.mU.mC.mU.fA. | 12 | RL0213 strand of compound |
| fG.fU.mC.fU.mC.fC.mC.mA.fC.fC.mC.mA. | | RD0454 (NT) |
| mG.mG.mG.mG.mU.rC.rC.rA.mC.mA.mU.mG. | | |
| mG*mC*mA*mA*mC | | |
| (<Strand Ref.: 650>) | | |
| GGGUGGAAGAGUAGAACAAUAUGC | 5 | RS0080-UM strand of |
| (<Strand Ref.: 651>) | | compound RD0454 (NT) |
| GCAUAUUGUUCUAGUCUCCCACCCAGGGGUCCACAUG | 12 | RL0213-UM strand of |
| GCAAC | | compound RD0454 (NT) |
| (<Strand Ref.: 652>) | | |
| mG*mG*mU*fG*mG.mG.fU.fG.fG.fA.fA.fC.fA. | 88 | RS0081 strand of compound |
| fG.fG.fA.fC.fA.fA.fC.mA.fA.mU*fA*mU* | | RD0455 (NT) |
| fG*mC | | |
| (<Strand Ref.: 653>) | | |
| mG*fC*mA*fU*fA.fU.mU.fG.mU.mU.mG.mU.fC. | 89 | RL0214 strand of compound |
| fG.fU.mG.fU.mC.fC.mC.mA.fC.fC.mC.fA. | | RD0455 (NT) |
| mC.mC.mA.mG.mG.mG.mG.mU.rC.rC.rA.mC. | | |
| mA.mU.mG.mG*mC*mA*mA*mC | | |
| (<Strand Ref.: 654>) | | |
| GGUGGGUGGAACAGGACAACAAUAUGC | 88 | RS0081-UM strand of |
| (<Strand Ref.: 655>) | | compound RD0455 (NT) |
| GCAUAUUGUUGUCGUGUCCCACCCACCAGGGGUCCAC | 89 | RL0214-UM strand of |
| AUGGCAAC | | compound RD0455 (NT) |
| (<Strand Ref.: 656>) | | |
| mG*mG*mU*fG*mG.mG.fU.fG.fG.fA.fA.fC.fA. | 90 | RS0082 strand of compound |
| fG.fG.fA.fG*fA*fA*fC*mA | | RD0456 (NT) |
| (<Strand Ref.: 657>) | | |
| mU*fG*mU*mU*mC.mU.fC.fG.fU.mG.fU.mC.fC. | 91 | RL0215 strand of compound |
| mC.mA.fC.fC.mC.fA.mC.mC.mA.mG.mG.mG. | | RD0456 (NT) |
| mG.mU.rC.rC.rA.mC.mA.mU.mG.mG*mC*mA* | | |
| mA*mC | | |
| (<Strand Ref.: 658>) | | |
| GGUGGGUGGAACAGGAGAACA | 90 | RS0082-UM strand of |
| (<Strand Ref.: 659>) | | compound RD0456 (NT) |
| UGUUCUCGUGUCCCACCCACCAGGGGUCCACAUGGCA | 91 | RL0215-UM strand of |
| AC | | compound RD0456 (NT) |
| (<Strand Ref.: 660>) | | |
| mG*mG*mU*fG*mG.mG.fU.fG.fG.fA.fA.fC.fA. | 92 | RS0083 strand of compound |
| fG.fG.fA.fG*fA*fA*fC*mG | | RD0457 (NT) |
| (<Strand Ref.: 661>) | | |
| mC*fG*mU*mU*mC.mU.fC.fG.fU.mG.fU.mC.fC. | 93 | RL0216 strand of compound |
| mC.mA.fC.fC.mC.fA.mC.mC.mA.mG.mG.mG. | | RD0457 (NT) |
| mG.mU.rC.rC.rA.mC.mA.mU.mG.mG*mC*mA* | | |
| mA*mC | | |
| (<Strand Ref.: 662>) | | |
| GGUGGGUGGAACAGGAGAACG | 92 | RS0083-UM strand of |
| (<Strand Ref.: 663>) | | compound RD0457 (NT) |
| CGUUCUCGUGUCCCACCCACCAGGGGUCCACAUGGCA | 93 | RL0216-UM strand of |
| AC | | compound RD0457 (NT) |
| (<Strand Ref.: 664>) | | |
| mG*mG*mU*fG*mU.mC.fG.fA.fG.fA.fA.fG.fA. | 3 | RS0074 strand of compound |
| fG.fG.fA.fG.fA.fA.fC.mA*fA*mU*fA*mU | | RD0458 (NT) |
| (<Strand Ref.: 665>) | | |
| mA*fU*fG*fU*mU.fG.mU.mU.mC.mU.fC.fG.fU. | 82 | RL0217 strand of compound |
| mC.fU.mC.fC.mU.mC.fG.fA.mC.fA.mC.mC. | | RD0458 (NT) |
| mA.mG.mG.mG.mG.mU.rC.rC.rA.mC.mA.mU. | | |
| mG.mG*mA*mA*mA*mC | | |
| (<Strand Ref.: 666>) | | |
| GGUGUCGAGAAGAGGAGAACAAUAU | 3 | RS0074-UM strand of |
| (<Strand Ref.: 667>) | | compound RD0458 (NT) |
| AUGUUGUUCUCGUCUCCUCGACACCAGGGGUCCACAU | 83 | RL0217-UM strand of |
| GGAAAC | | compound RD0458 (NT) |
| (<Strand Ref.: 668>) | | |
| mG*mG*mU*fG*mG.mG.fU.fG.fG.fA.fA.fG.fA. | 94 | RS0076 strand of compound |
| fG.fG.fA.fG.fA.fA.fC.mA.fA.mU*fA*mU* | | RD0459 (NT) |
| fG*mC | | |
| (<Strand Ref.: 669>) | | |
| mG*fC*mA*fU*fG.fU.mU.fG.mU.mU.mC.mU.fC. | 95 | RL0218 strand of compound |
| fG.fU.mC.fU.mC.fC.mC.mA.fC.fC.mC.fA. | | RD0459 (NT) |
| mC.mC.mA.mG.mG.mG.mG.mU.rC.rC.rA.mC. | | |
| mA.mU.mG.mG*mA*mA*mA*mC | | |
| (<Strand Ref.: 670>) | | |
| GGUGGGUGGAAGAGGAGAACAAUAUGC | 9. | RS0076-UM strand of |
| (<Strand Ref.: 671>) | | compound RD0459 (NT) |
| GCAUGUUGUUCUCGUCUCCCACCCACCAGGGGUCCAC | 95 | RL0218-UM strand of |
| AUGGAAAC | | compound RD0459 (NT) |
| (<Strand Ref.: 672>) | | |
| mG*mG*mU*fG*mG.mG.fU.fG.fG.fA.fA.fG.fA. | 94 | RS0076 strand of compound |
| fG.fG.fA.fG.fA.fA.fC.mA.fA.mU*fA*mU* | | RD0461 (NT) |
| fG*mC | | |
| (<Strand Ref.: 673>) | | |
| mG*fC*mA*fU*fA.fU.mU.fG.mU.mU.mC.mU.fC. | 96 | RL0220 strand of compound |
| fG.fU.mC.fU.mC.fC.mC.mA.fC.fC.mC.fA. | | RD0461 (NT) |
| mC.mC.mA.mG.mG.mG.mG.mU.rC.rC.rA.mC. | | |
| mA.mU.mG.mG*mA*mA*mA*mC | | |
| (<Strand Ref.: 674>) | | |
| GGUGGGUGGAAGAGGAGAACAAUAUGC | 94 | RS0076-UM strand of |
| (<Strand Ref.: 675>) | | compound RD0461 (NT) |
| GCAUAUUGUUCUCGUCUCCCACCCACCAGGGGUCCAC | 96 | RL0220-UM strand of |
| AUGGAAAC | | compound RD0461 (NT) |
| (<Strand Ref.: 676>) | | |
| mG*mG*mU*fG*mG.mG.fU.fG.fG.fA.fA.fG.fA. | 84 | RS0078 strand of compound |
| fG.fG.fA.fG*fA*fA*fC*mA | | RD0463 (NT) |
| (<Strand Ref.: 677>) | | |
| mU*fG*mU*mU*mC.mU.fC.fG.fU.mC.fU.mC.fC. | 97 | RL0222 strand of compound |
| mC.mA.fC.fC.mC.fA.mC.mC.mA.mG.mG.mG. | | RD0463 (NT) |
| mG.mU.rC.rC.rA.mC.mA.mU.mG.mG*mA*mA* | | |
| mA*mC | | |
| (<Strand Ref.: 678>) | | |
| GGUGGGUGGAAGAGGAGAACA | 84 | RS0078-UM strand of |
| (<Strand Ref.: 679>) | | compound RD0463 (NT) |
| UGUUCUCGUCUCCCACCCACCAGGGGUCCACAUGGAA | 97 | RL0222-UM strand of |
| AC | | compound RD0463 (NT) |
| (<Strand Ref.: 680>) | | |
| mG*mG*mU*fG*mG.mG.fU.fG.fG.fA.fA.fG.fA. | 86 | RS0079 strand of compound |
| fG.fG.fA.fG*fA*fA*fC*mG | | RD0464 (NT) |
| (<Strand Ref.: 681>) | | |
| mC*fG*mU*mU*mC.mU.fC.fG.fU.mC.fU.mC.fC. | 98 | RL0223 strand of compound |
| mC.mA.fC.fC.mC.fA.mC.mC.mA.mG.mG.mG. | | RD0464 (NT) |
| mG.mU.rC.rC.rA.mC.mA.mU.mG.mG*mA*mA* | | |
| mA*mC | | |
| (<Strand Ref.: 682>) | | |
| GGUGGGUGGAAGAGGAGAACG | 86 | RS0079-UM strand of |
| (<Strand Ref.: 683>) | | compound RD0464 (NT) |
| CGUUCUCGUCUCCCACCCACCAGGGGUCCACAUGGAA | 98 | RL0223-UM strand of |
| AC | | compound RD0464 (NT) |
| (<Strand Ref.: 684>) | | |
| mG*mG*mG*fU.fG.fG.fA.fA.fG.fA.fG.fU.fA. | 5 | RS0080 strand of compound |
| fG.fA.fA.fC.mA.fA.mU*fA*mU*fG*mC | | RD0465 (NT) |
| (<Strand Ref.: 685>) | | |
| mG*fC*mA.fU.fA.fU.mU.fG.mU.mU.mC.mU.fA. | 99 | RL0224 strand of compound |
| fG.fU.mC.fU.mC.fC.mC.mA.fC.fC.mC.mA. | | RD0465 (NT) |
| mG.mG.mG.mG.mU.rC.rC.rA.mC.mA.mU.mG. | | |
| mG*mA*mA*mA*mC | | |
| (<Strand Ref.: 686>) | | |
| GGGUGGAAGAGUAGAACAAUAUGC | 5 | RS0080-UM strand of |
| (<Strand Ref.: 687>) | | compound RD0465 (NT) |
| GCAUAUUGUUCUAGUCUCCCACCCAGGGGUCCACAUG | 99 | RL0224-UM strand of |
| GAAAC | | compound RD0465 (NT) |
| (<Strand Ref.: 688>) | | |
| mG*mG*mU*fG*mG.mG.fU.fG.fG.fA.fA.fC.fA. | 88 | RS0081 strand of compound |
| fG.fG.fA.fC.fA.fA.fC.mA.fA.mU*fA*mU* | | RD0466 (NT) |
| fG*mC | | |
| (<Strand Ref.: 689>) | | |
| mG*fC*mA*fU*fA.fU.mU.fG.mU.mU.mG.mU.fC. | 100 | RL0225 strand of compound |
| fG.fU.mG.fU.mC.fC.mC.mA.fC.fC.mC.fA. | | RD0466 (NT) |
| mC.mC.mA.mG.mG.mG.mG.mU.rC.rC.rA.mC. | | |
| mA.mU.mG.mG*mA*mA*mA*mC | | |
| (<Strand Ref.: 690>) | | |
| GGUGGGUGGAACAGGACAACAAUAUGC | 88 | RS0081-UM strand of |
| (<Strand Ref.: 691>) | | compound RD0466 (NT) |
| GCAUAUUGUUGUCGUGUCCCACCCACCAGGGGUCCAC | 100 | RL0225-UM strand of |
| AUGGAAAC | | compound RD0466 (NT) |
| (<Strand Ref.: 692>) | | |
| mG*mG*mU*fG*mG.mG.fU.fG.fG.fA.fA.fC.fA. | 90 | RS0082 strand of compound |
| fG.fG.fA.fG*fA*fA*fC*mA | | RD0467 (NT) |
| (<Strand Ref.: 693>) | | |
| mU*fG*mU*mU*mC.mU.fC.fG.fU.mG.fU.mC.fC. | 101 | RL0226 strand of compound |
| mC.mA.fC.fC.mC.fA.mC.mC.mA.mG.mG.mG. | | RD0467 (NT) |
| mG.mU.rC.rC.rA.mC.mA.mU.mG.mG*mA*mA* | | |
| mA*mC | | |
| (<Strand Ref.: 694>) | | |
| GGUGGGUGGAACAGGAGAACA | 90 | RS0082-UM strand of |
| (<Strand Ref.: 695>) | | compound RD0467 (NT) |
| UGUUCUCGUGUCCCACCCACCAGGGGUCCACAUGGAA | 101 | RL0226-UM strand of |
| AC | | compound RD0467 (NT) |
| (<Strand Ref.: 696>) | | |
| mG*mG*mU*fG*mG.mG.fU.fG.fG.fA.fA.fC.fA. | 92 | RS0083 strand of compound |
| fG.fG.fA.fG*fA*fA*fC*mG | | RD0468 (NT) |
| (<Strand Ref.: 697>) | | |
| mC*fG*mU*mU*mC.mU.fC.fG.fU.mG.fU.mC.fC. | 102 | RL0227 strand of compound |
| mC.mA.fC.fC.mC.fA.mC.mC.mA.mG.mG.mG. | | RD0468 (NT) |
| mG.mU.rC.rC.rA.mC.mA.mU.mG.mG*mA*mA* | | |
| mA*mC | | |
| (<Strand Ref.: 698>) | | |
| GGUGGGUGGAACAGGAGAACG | 92 | RS0083-UM strand of |
| (<Strand Ref.: 699>) | | compound RD0468 (NT) |
| CGUUCUCGUGUCCCACCCACCAGGGGUCCACAUGGAA | 102 | RL0227-UM strand of |
| AC | | compound RD0468 (NT) |
| (<Strand Ref.: 700>) | | |
| mG*fG*mG*fU*mG.fG.fA.fA.mG.fA.fG.fU.mA. | 5 | RS0026 strand of compound |
| fG.mA.fA.mC.fA.mA.fU*mA*fU*mG*mC | | RD0473 (NT) |
| (<Strand Ref.: 701>) | | |
| mG*fC*mA*fU*mA.fU.mU.fG.mU.fU.mC.fU.mA. | 99 | RL0228 strand of compound |
| fG.mU.fC.mU.mC.mC.fC.mA.fC.mC.fC.mA. | | RD0473 (NT) |
| mG.mG.mG.mG.mU.rC.rC.rA.mC.mA.mU.mG. | | |
| mG*mA*mA*mA*mC | | |
| (<Strand Ref.: 702>) | | |
| GGGUGGAAGAGUAGAACAAUAUGC | 5 | RS0026-UM strand of |
| (<Strand Ref.: 703>) | | compound RD0473 (NT) |
| GCAUAUUGUUCUAGUCUCCCACCCAGGGGUCCACAUG | 99 | RL0228-UM strand of |
| GAAAC | | compound RD0473 (NT) |
| (<Strand Ref.: 704>) | | |
| mG*fG*mU*fG*mU.fC.mG.fA.mG.fA.mA.fG.mA. | 3 | RS0037 strand of compound |
| fG.mG.fA.mG.fA.mA.fC.mA*fA*mU*fA*mU | | RD0474 (NT) |
| (<Strand Ref.: 705>) | | |
| mA*fU*mG*fU*mU.fG.mU.fU.mC.fU.mC.fG.mU. | 83 | RL0204 strand of compound |
| fC.mU.mC.mC.fU.mC.fG.mA.fC.mA.fC.mC. | | RD0474 (NT) |
| mA.mG.mG.mG.mG.mU.rC.rC.rA.mC.mA.mU. | | |
| mG.mG*mA*mA*mA*mC | | |
| (<Strand Ref.: 706>) | | |
| GGUGUCGAGAAGAGGAGAACAAUAU | 3 | RS0037-UM strand of |
| (<Strand Ref.: 707>) | | compound RD0474 (NT) |
| AUGUUGUUCUCGUCUCCUCGACACCAGGGGUCCACAU | 83 | RL0204-UM strand of |
| GGAAAC | | compound RD0474 (NT) |
| (<Strand Ref.: 708>) | | |
| mG*fG*mU*fG*mU.fC.mG.fA.mG.rA.mA.fG.mA. | 3 | RS0041 strand of compound |
| rG.mG.fA.mG.fA.mA.fC.mA*fA*mU*fA*mU | | RD0475 (NT) |
| (<Strand Ref.: 709>) | | |
| mA*fU*mG*fU*mU.fG.mU.fU.mC.fU.mC.rG.mU. | 83 | RL0229 strand of compound |
| fC.mU.rC.mC.fU.mC.fG.mA.fC.mA.fC.mC. | | RD0475 (NT) |
| mA.mG.mG.mG.mG.mU.rC.rC.rA.mC.mA.mU. | | |
| mG.mG*mA*mA*mA*mC | | |
| (<Strand Ref.: 710>) | | |
| GGUGUCGAGAAGAGGAGAACAAUAU | 3 | RS0041-UM strand of |
| (<Strand Ref.: 711>) | | compound RD0475 (NT) |
| AUGUUGUUCUCGUCUCCUCGACACCAGGGGUCCACAU | 83 | RL0229-UM strand of |
| GGAAAC | | compound RD0475 (NT) |
| (<Strand Ref.: 712>) | | |
| mG*fG*mG*fU*mG.fG.fA.fA.mG.fA.fG.fU.mA. | 5 | RS0026 strand of compound |
| fG.mA.fA.mC.fA.mA.fU*mA*fU*mG*mC | | RD0476 (NT) |
| (<Strand Ref.: 713>) | | |
| mG*fC*mA*fU*mA.fU.mU.fG.mU.fU.mC.fU.mA. | 99 | RL0230 strand of compound |
| fG.mU.fC.mU.mC.mC.fC.mA.fC.mC.fC.mA. | | RD0476 (NT) |
| mG.mG.mG.mG.mU.rC*rC*rA.mC.mA.mU.mG. | | |
| mG*mA*mA*mA*mC | | |
| (<Strand Ref.: 714>) | | |
| GGGUGGAAGAGUAGAACAAUAUGC | 5 | RS0026-UM strand of |
| (<Strand Ref.: 715>) | | compound RD0476 (NT) |
| GCAUAUUGUUCUAGUCUCCCACCCAGGGGUCCACAUG | 99 | RL0230-UM strand of |
| GAAAC | | compound RD0476 (NT) |
| (<Strand Ref.: 716>) | | |
| mG*fG*mG*fU*mG.fG.fA.fA.mG.fA.fG.fU.mA. | 5 | RS0026 strand of compound |
| fG.mA.fA.mC.fA.mA.fU*mA*U*mG*mC | | RD0477 (NT) |
| (<Strand Ref.: 717>) | | |
| mG*fC*mA*fU*mA.fU.mU.fG.mU.fU.mC.fU.mA. | 99 | RL0231 strand of compound |
| fG.mU.fC.mU.mC.mC.fC.mA.fC.mC.fC.mA. | | RD0477 (NT) |
| mG.mG.mG.mG.mU.dc*dC*dA.mC.mA.mU.mG. | | |
| mG*mA*mA*mA*mC | | |
| (<Strand Ref.: 718>) | | |
| GGGUGGAAGAGUAGAACAAUAUGC | 5 | RS0026-UM strand of |
| (<Strand Ref.: 719>) | | compound RD0477 (NT) |
| GCAUAUUGUUCUAGUCUCCCACCCAGGGGUdCdCdAC | 99 | RL0231-UM strand of |
| AUGGAAAC | | compound RD0477 (NT) |
| (<Strand Ref.: 720>) | | |
| mG*fG*mU*fG*mU.fC.mG.fA.mG.fA.mA.fG.mA. | 3 | RS0037 strand of compound |
| fG.mG.fA.mG.fA.mA.fC.mA*fA*mU*fA*mU | | RD0478 (NT) |
| (<Strand Ref.: 721>) | | |
| mA*fU*mG*fU*mU.fG.mU.fU.mC.fU.mC.fG.mU. | 83 | RL0232 strand of compound |
| fC.mU.mC.mC.fU.mC.fG.mA.fC.mA.fC.mC. | | RD0478 (NT) |
| mA.mG.mG.mG.mG.mU.dc*dC*dA.mC.mA.mU. | | |
| mG.mG*mA*mA*mA*mC | | |
| (<Strand Ref.: 722>) | | |
| GGUGUCGAGAAGAGGAGAACAAUAU | 3 | RS0037-UM strand of |
| (<Strand Ref.: 723>) | | compound RD0478 (NT) |
| AUGUUGUUCUCGUCUCCUCGACACCAGGGGUdCdCdA | 83 | RL0232-UM strand of |
| CAUGGAAAC | | compound RD0478 (NT) |
| (<Strand Ref.: 724>) | | |
| mG*fG*mG*fU*mG.fG.fA.fA.mG.fA.fG.fG.mA. | 103 | RS0084 strand of compound |
| fG.mA.fA.mC.fA.mA.fU*mA*fU*mG*mC | | RD0479 (NT) |
| (<Strand Ref.: 725>) | | |
| mG*fC*mA*fU*mA.fU.mU.fG.mU.fU.mC.fU.mC. | 104 | RL0233 strand of compound |
| fG.mU.fC.mU.mC.mC.fC.mA.fC.mC.fC.mA. | | RD0479 (NT) |
| mG.mG.mG.mG.mU.rC.rC.rA.mC.mA.mU.mG. | | |
| mG*mC*mA*mA*mC | | |
| (<Strand Ref.: 726>) | | |
| GGGUGGAAGAGGAGAACAAUAUGC | 103 | RS0084-UM strand of |
| (<Strand Ref.: 727>) | | compound RD0479 (NT) |
| GCAUAUUGUUCUCGUCUCCCACCCAGGGGUCCACAUG | 104 | RL0233-UM strand of |
| GCAAC | | compound RD0479 (NT) |
| (<Strand Ref.: 728>) | | |
| mG*fG*mG*fU*mG.fG.fA.fA.mG.fA.fG.fU.mA. | 5 | RS0026 strand of compound |
| fG.mA.fA.mC.fA.mA.fU*mA*fU*mG*mC | | RD0480 (NT) |
| (<Strand Ref.: 729>) | | |
| mG*fC*mG*fU*mA.fU.mU.fG.mU.fU.mC.fU.mA. | 105 | RL0234 strand of compound |
| fG.mU.fC.mU.mC.mC.fC.mA.fC.mC.fC.mA. | | RD0480 (NT) |
| mG.mG.mG.mG.mU.rC.rC.rA.mC.mA.mU.mG. | | |
| mG*mC*mA*mA*mC | | |
| (<Strand Ref.: 730>) | | |
| GGGUGGAAGAGUAGAACAAUAUGC | 5 | RS0026-UM strand of |
| (<Strand Ref.: 731>) | | compound RD0480 (NT) |
| GCGUAUUGUUCUAGUCUCCCACCCAGGGGUCCACAUG | 105 | RL0234-UM strand of |
| GCAAC | | compound RD0480 (NT) |
| (<Strand Ref.: 732>) | | |
| mG*fG*mG*fU*mG.fG.fA.fA.mG.fA.fG.fG.mA. | 103 | RS0084 strand of compound |
| fG.mA.fA.mC.fA.mA.fU*mA*fU*mG*mC | | RD0481 (NT) |
| (<Strand Ref.: 733>) | | |
| mG*fC*mG*fU*mA.fU.mU.fG.mU.fU.mC.fU.mC. | 106 | RL0235 strand of compound |
| fG.mU.fC.mU.mC.mC.fC.mA.fC.mC.fC.mA. | | RD0481 (NT) |
| mG.mG.mG.mG.mU.rC.rC.rA.mC.mA.mU.mG. | | |
| mG*mC*mA*mA*mC | | |
| (<Strand Ref.: 734>) | | |
| GGGUGGAAGAGGAGAACAAUAUGC | 103 | RS0084-UM strand of |
| (<Strand Ref.: 735>) | | compound RD0481 (NT) |
| GCGUAUUGUUCUCGUCUCCCACCCAGGGGUCCACAUG | 106 | RL0235-UM strand of |
| GCAAC | | compound RD0481 (NT) |
| (<Strand Ref.: 736>) | | |
| mG*fG*mG*fU*mG.fG.fA.fA.mG.fA.fG.fG.mA. | 107 | RS0085 strand of compound |
| fG.mA.fA.mC*fA*mA*fU*mA | | RD0482 (NT) |
| (<Strand Ref.: 737>) | | |
| mU*mA*fU*mU*fG.mU.fU.mC.fU.mC.fG.mU.fC. | 108 | RL0236 strand of compound |
| mU.mC.mC.fC.mA.fC.mC.fC.mA.mG.mG.mG. | | RD0482 (NT) |
| mG.mU.rC.rC.rA.mC.mA.mU.mG.mG*mC*mA* | | |
| mA*mC | | |
| (<Strand Ref.: 738>) | | |
| GGGUGGAAGAGGAGAACAAUA | 107 | RS0085-UM strand of |
| (<Strand Ref.: 739>) | | compound RD0482 (NT) |
| UAUUGUUCUCGUCUCCCACCCAGGGGUCCACAUGGCA | 108 | RL0236-UM strand of |
| AC | | compound RD0482 (NT) |
| (<Strand Ref.: 740>) | | |
| mG*fG*mG*fU*mG.fG.fA.fA.mG.fA.fG.fG.mA. | 109 | RS0086 strand of compound |
| fG.mA.fA.mC*fA*mG*fC*mG | | RD0483 (NT) |
| (<Strand Ref.: 741>) | | |
| mC*mG*fC*mU*fG.mU.fU.mC.fU.mC.fG.mU.fC. | 110 | RL0237 strand of compound |
| mU.mC.mC.fC.mA.fC.mC.fC.mA.mG.mG.mG. | | RD0483 (NT) |
| mG.mU.rC.rC.rA.mC.mA.mU.mG.mG*mC*mA* | | |
| mA*mC | | |
| (<Strand Ref.: 742>) | | |
| GGGUGGAAGAGGAGAACAGCG | 109 | RS0086-UM strand of |
| (<Strand Ref.: 743>) | | compound RD0483 (NT) |
| CGCUGUUCUCGUCUCCCACCCAGGGGUCCACAUGGCA | 110 | RL0237-UM strand of |
| AC | | compound RD0483 (NT) |
| (<Strand Ref.: 744>) | | |
| mG*fU*mG*fU*mG.fG.fA.fA.mG.fA.fG.fG.mA. | 111 | RS0087 strand of compound |
| fG.mA.fA.mC*fA*mG*fU*mG | | RD0484 (NT) |
| (<Strand Ref.: 745>) | | |
| mC*mA*fC*mU*fG.mU.fU.mC.fU.mC.fG.mU.fC. | 112 | RL0238 strand of compound |
| mU.mC.mC.fC.mA.fC.mC.fC.mA.mG.mG.mG. | | RD0484 (NT) |
| mG.mU.rC.rC.rA.mC.mA.mU.mG.mG*mC*mA* | | |
| mA*mC | | |
| (<Strand Ref.: 746>) | | |
| GUGUGGAAGAGGAGAACAGUG | 111 | RS0087-UM strand of |
| (<Strand Ref.: 747>) | | compound RD0484 (NT) |
| CACUGUUCUCGUCUCCCACCCAGGGGUCCACAUGGCA | 112 | RL0238-UM strand of |
| AC | | compound RD0484 (NT) |
| (<Strand Ref.: 748>) | | |
| mG*fU*mG*fU*mG.fG.fA.fA.mG.fA.fG.fG.mA. | 111 | RS0087 strand of compound |
| fG.mA.fA.mC*fA*mG*fU*mG | | RD0485 (NT) |
| (<Strand Ref.: 749>) | | |
| mC*mG*fC*mU*fG.mU.fU.mC.fU.mC.fG.mU.fC. | 110 | RL0237 strand of compound |
| mU.mC.mC.fC.mA.fC.mC.fC.mA.mG.mG.mG. | | RD0485 (NT) |
| mG.mU.rC.rC.rA.mC.mA.mU.mG.mG*mC*mA* | | |
| mA*mC | | |
| (<Strand Ref.: 750>) | | |
| GUGUGGAAGAGGAGAACAGUG | 111 | RS0087-UM strand of |
| (<Strand Ref.: 751>) | | compound RD0485 (NT) |
| CGCUGUUCUCGUCUCCCACCCAGGGGUCCACAUGGCA | 110 | RL0237-UM strand of |
| AC | | compound RD0485 (NT) |
| (<Strand Ref.: 752>) | | |
| (X1)*mA*mG*mG.mG.mG.mU.rC.rC.rA.mC.mA. | 1 | Editing domain on long strand |
| mU.mG.mG*mC*mA*mA*mC | | sequence information 5′- 3′ |
| (<Strand Ref.: 753>) | | strand of composition RD0542 |
| | | with linker = PEG2 |
| mG*mG*mU*fG*mU.mC.fG.fA.fG.fA.fA.fG.fA. | 3 | short strand complementary to |
| fG.fG.fA.fG.fA.fA.fC.mA*fA*mU*fA*mU | | recruiting domain on Long |
| (<Strand Ref.: 754>) | | strand 5′-3′ of composition |
| | | RD0542 with linker = PEG2 |
| mA*fU*rG.fU.mU.rG.mU.mU.mC.mU.fC.rG.fU. | 4 | Recruiting domain on long |
| mC.fu.rC.fC.mU.mC.rG.rA.mC.rA*mC*mC* | | strand, sequence information |
| (X2) | | 5′-3′ of composition RD0542 |
| (<Strand Ref.: 755>) | | with linker = PEG2 |
| (X1)AGGGGUCCACAUGGCAAC | 1 | Editing domain on long strand |
| (<Strand Ref.: 756>) | | sequence information 5′- 3′- |
| | | UM of composition RD0542 |
| | | with linker = PEG2 |
| GGUGUCGAGAAGAGGAGAACAAUAU | 3 | short strand complementary to |
| (<Strand Ref.: 757>) | | recruiting domain on Long |
| | | strand 5′-3′-UM linker = PEG2 |
| AUGUUGUUCUCGUCUCCUCGACACC(X2) | 4 | Recruiting domain on long |
| (<Strand Ref.: 758>) | | strand, sequence information |
| | | 5′-3′-UM linker = PEG2 |
| (X1)*mA*mG*mG.mG.mG.mU.rC.rC.rA.mC.mA. | 1 | Editing domain on long strand |
| mU.mG.mG*mC*mA*mA*mC | | sequence information 5′- 3′ |
| (<Strand Ref.: 759>) | | strand of composition RD0543 |
| | | with linker = PEG6 |
| mG*mG*mU*fG*mU.mC.fG.fA.fG.fA.fA.fG.fA. | 3 | short strand complementary to |
| fG.fG.fA.fG.fA.fA.fC.mA*fA*mU*fA*mU | | recruiting domain on Long |
| (<Strand Ref.: 760>) | | strand 5′-3′ of composition |
| | | RD0543 with linker = PEG6 |
| mA*fU*rG.fU.mU.rG.mU.mU.mC.mU.fC.rG.fU. | 4 | Recruiting domain on long |
| mC.fU.rC.fC.mU.mC.rG.rA.mC.rA*mC*mC* | | strand, sequence information |
| (X2) | | 5′-3′ of composition RD0543 |
| (<Strand Ref.: 761>) | | with linker = PEG6 |
| (X1)AGGGGUCCACAUGGCAAC | 1 | Editing domain on long strand |
| (<Strand Ref.: 762>) | | sequence information 5′- 3′- |
| | | UM of composition RD0543 |
| | | with linker = PEG6 |
| GGUGUCGAGAAGAGGAGAACAAUAU | 3 | short strand complementary to |
| (<Strand Ref.: 763>) | | recruiting domain on Long |
| | | strand 5′-3′-UM linker = PEG6 |
| AUGUUGUUCUCGUCUCCUCGACACC(X2) | 4 | Recruiting domain on long |
| (<Strand Ref.: 764>) | | strand, sequence information |
| | | 5′-3′-UM linker = PEG6 |
| (X1)*mA*mG*mG.mG.mG.mU.rC.rC.rA.mC.mA. | 1 | Editing domain on long strand |
| mU.mG.mG*mC*mA*mA*mC | | sequence information 5′- 3′ |
| (<Strand Ref.: 765>) | | strand of composition RD0544 |
| | | with linker = PEG12 |
| mG*mG*mU*fG*mU.mC.fG.fA.fG.fA.fA.fG.fA. | 3 | short strand complementary to |
| fG.fG.fA.fG.fA.fA.fC.mA*fA*mU*fA*mU | | recruiting domain on Long |
| (<Strand Ref.: 766>) | | strand 5′-3′ of composition |
| | | RD0544 with linker = PEG12 |
| mA*fU*rG.fU.mU.rG.mU.mU.mC.mU.fC.rG.fU. | 4 | Recruiting domain on long |
| mC.fU.rC.fC.mU.mC.rG.rA.mC.rA*mC*mC* | | strand, sequence information |
| (X2) | | 5′-3′ of composition RD0544 |
| (<Strand Ref.: 767>) | | with linker = PEG12 |
| (X1)AGGGGUCCACAUGGCAAC | 1 | Editing domain on long strand |
| (<Strand Ref.: 768>) | | sequence information 5′- 3′- |
| | | UM of composition RD0544 |
| | | with linker = PEG12 |
| GGUGUCGAGAAGAGGAGAACAAUAU | 3 | short strand complementary to |
| (<Strand Ref.: 769>) | | recruiting domain on Long |
| | | strand 5′-3′-UM linker = |
| | | PEG12 |
| AUGUUGUUCUCGUCUCCUCGACACC(X2) | 4 | Recruiting domain on long |
| (<Strand Ref.: 770>) | | strand, sequence information |
| | | 5′-3′-UM linker = PEG12 |
| (X1)*mA*mG*mG.mG.mG.mU.rC.rC.rA.mC.mA. | 1 | Editing domain on long strand |
| mU.mG.mG*mC*mA*mA*mC | | sequence information 5′- 3′ |
| (<Strand Ref.: 771>) | | strand of composition RD0545 |
| | | with linker = PEG24 |
| mG*mG*mU*fG*mU.mC.fG.fA.fG.fA.fA.fG.fA. | 3 | short strand complementary to |
| fG.fG.fA.fG.fA.fA.fC.mA*fA*mU*fA*mU | | recruiting domain on Long |
| (<Strand Ref.: 772>) | | strand 5′-3′ of composition |
| | | RD0545 with linker = PEG24 |
| mA*fU*rG.fU.mU.rG.mU.mU.mC.mU.fC.rG.fU. | 4 | Recruiting domain on long |
| mC.fU.rC.fC.mU.mC.rG.rA.mC.rA*mC*mC* | | strand, sequence information |
| (X2) | | 5′-3′ of composition RD0545 |
| (<Strand Ref.: 773>) | | with linker = PEG24 |
| (X1)AGGGGUCCACAUGGCAAC | 1 | Editing domain on long strand |
| (<Strand Ref.: 774>) | | sequence information 5′- 3′- |
| | | UM of composition RD0545 |
| | | with linker = PEG24 |
| GGUGUCGAGAAGAGGAGAACAAUAU | 3 | short strand complementary to |
| (<Strand Ref.: 775>) | | recruiting domain on Long |
| | | strand 5′-3′-UM linker = |
| | | PEG24 |
| AUGUUGUUCUCGUCUCCUCGACACC(X2) | 4 | Recruiting domain on long |
| (<Strand Ref.: 776>) | | strand, sequence information |
| | | 5′-3′-UM linker = PEG24 |
| (X1)*mA*mG*mG.mG.mG.mU.rC.rC.rA.mC.mA. | 1 | Editing domain on long strand |
| mU.mG.mG*mC*mA*mA*mC | | sequence information 5′- 3′ |
| (<Strand Ref.: 777>) | | strand of composition RD0546 |
| | | with linker = C3 |
| mG*mG*mU*fG*mU.mC.fG.fA.fG.fA.fA.fG.fA. | 3 | short strand complementary to |
| fG.fG.fA.fG.fA.fA.fC.mA*fA*mU*fA*mU | | recruiting domain on Long |
| (<Strand Ref.: 778>) | | strand 5′-3′ of composition |
| | | RD0546 with linker = C3 |
| mA*fU*rG.fU.mU.rG.mU.mU.mC.mU.fC.rG.fU. | 4 | Recruiting domain on long |
| mC.fU.rC.fC.mU.mC.rG.rA.mC.rA*mC*mC* | | strand, sequence information |
| (X2) | | 5′-3′ of composition RD0546 |
| (<Strand Ref.: 779>) | | with linker = C3 |
| (X1)AGGGGUCCACAUGGCAAC | 1 | Editing domain on long strand |
| (<Strand Ref.: 780>) | | sequence information 5′- 3′- |
| | | UM of composition RD0546 |
| | | with linker = C3 |
| GGUGUCGAGAAGAGGAGAACAAUAU | 3 | short strand complementary to |
| (<Strand Ref.: 781>) | | recruiting domain on Long |
| | | strand 5′-3′-UM linker = C3 |
| AUGUUGUUCUCGUCUCCUCGACACC(X2) | 4 | Recruiting domain on long |
| (<Strand Ref.: 782>) | | strand, sequence information |
| | | 5′-3′-UM linker = C3 |
| (X1)*mA*mG*mG.mG.mG.mU.rC*rC*rA.mC.mA. | 113 | Editing domain on long strand |
| mU.mG.mG*mA*mA*mA*mC | | sequence information 5′- 3′ |
| (<Strand Ref.: 783>) | | strand of composition RD0560 |
| | | with linker = PEG2 |
| mG*mG*mU*fG*mU.mC.fG.fA.fG.fA.fA.fG.fA. | 3 | short strand complementary to |
| fG.fG.fA.fG.fA.fA.fC.mA*fA*mU*fA*mU | | recruiting domain on Long |
| (<Strand Ref.: 784>) | | strand 5′-3′ of composition |
| | | RD0560 with linker = PEG2 |
| mA*fU*rG.fU.mU.rG.mU.mU.mC.mU.fC.rG.fU. | 4 | Recruiting domain on long |
| mC.fU.rC.fC.mU.mC.rG.rA.mC.rA*mC*mC* | | strand, sequence information |
| (X2) | | 5′-3′ of composition RD0560 |
| (<Strand Ref.: 785>) | | with linker = PEG2 |
| (X1)AGGGGUCCACAUGGAAAC | 113 | Editing domain on long strand |
| (<Strand Ref.: 786>) | | sequence information 5′- 3′- |
| | | UM of composition RD0560 |
| | | with linker = PEG2 |
| GGUGUCGAGAAGAGGAGAACAAUAU | 3 | short strand complementary to |
| (<Strand Ref.: 787>) | | recruiting domain on Long |
| | | strand 5′-3′-UM linker = PEG2 |
| AUGUUGUUCUCGUCUCCUCGACACC(X2) | 4 | Recruiting domain on long |
| (<Strand Ref.: 788>) | | strand, sequence information |
| | | 5′-3′-UM linker = PEG2 |
| (X1)*mA*mG*mG.mG.mG.mU.dC*dC*dA.mC.mA. | 113 | Editing domain on long strand |
| mU.mG.mG*mA*mA*mA*mC | | sequence information 5′- 3′ |
| (<Strand Ref.: 789>) | | strand of composition RD0561 |
| | | with linker = PEG2 |
| mG*mG*mU*fG*mU.mC.fG.fA.fG.fA.fA.fG.fA. | 3 | short strand complementary to |
| fG.fG.fA.fG.fA.fA.fC.mA*fA*mU*fA*mU | | recruiting domain on Long |
| (<Strand Ref.: 790>) | | strand 5′-3′ of composition |
| | | RD0561 with linker = PEG2 |
| mA*fU*rG.fU.mU.rG.mU.mU.mC.mU.fC.rG.fU. | 4 | Recruiting domain on long |
| mC.fU.rC.fC.mU.mC.rG.rA.mC.rA*mC*mC* | | strand, sequence information |
| (X2) | | 5′-3′ of composition RD0561 |
| (<Strand Ref.: 791>) | | with linker = PEG2 |
| (X1)AGGGGUdCdCdACAUGGAAAC | 113 | Editing domain on long strand |
| (<Strand Ref.: 792>) | | sequence information 5′- 3′- |
| | | UM of composition RD0561 |
| | | with linker = PEG2 |
| GGUGUCGAGAAGAGGAGAACAAUAU | 3 | short strand complementary to |
| (<Strand Ref.: 793>) | | recruiting domain on Long |
| | | strand 5′-3′-UM linker = PEG2 |
| AUGUUGUUCUCGUCUCCUCGACACC(X2) | 4 | Recruiting domain on long |
| (<Strand Ref.: 794>) | | strand, sequence information |
| | | 5′-3′-UM linker = PEG2 |
| (X1)*mA*mG*mG.mG.mG.mU.rC*rC*rA.mC.mA. | 1 | Editing domain on long strand |
| mU.mG.mG*mC*mA*mA*mC | | sequence information 5′- 3′ |
| (<Strand Ref.: 795>) | | strand of composition RD0562 |
| | | with linker = PEG2 |
| mG*mG*mU*fG*mU.mC.fG.fA.fG.fA.fA.fG.fA. | 3 | short strand complementary to |
| fG.fG.fA.fG.fA.fA.fC.mA*fA*mU*fA*mU | | recruiting domain on Long |
| (<Strand Ref.: 796>) | | strand 5′-3′ of composition |
| | | RD0562 with linker = PEG2 |
| mA*fU*rG.fU.mU.rG.mU.mU.mC.mU.fC.rG.fU. | 4 | Recruiting domain on long |
| mC.fU.rC.fC.mU.mC.rG.rA.mC.rA*mC*mC* | | strand, sequence information |
| (X2) | | 5′-3′ of composition RD0562 |
| (<Strand Ref.: 797>) | | with linker = PEG2 |
| (X1)AGGGGUCCACAUGGCAAC | 1 | Editing domain on long strand |
| (<Strand Ref.: 798>) | | sequence information 5′- 3′- |
| | | UM of composition RD0562 |
| | | with linker = PEG2 |
| GGUGUCGAGAAGAGGAGAACAAUAU | 3 | short strand complementary to |
| (<Strand Ref.: 799>) | | recruiting domain on Long |
| | | strand 5′-3′-UM linker = PEG2 |
| AUGUUGUUCUCGUCUCCUCGACACC(X2) | 4 | Recruiting domain on long |
| (<Strand Ref.: 800>) | | strand, sequence information |
| | | 5′-3′-UM linker = PEG2 |
| (X1)*mA*mG*mG.mG.mG.mU.rC.rC.rA.mC.mA. | 1 | Editing domain on long strand |
| mU.mG.mG*mC*mA*mA*mC | | sequence information 5′- 3′ |
| (<Strand Ref.: 801>) | | strand of composition RD0566 |
| | | with linker = Cy-C6 |
| mG*mG*mU*fG*mU.mC.fG.fA.fG.fA.fA.fG.fA. | 3 | short strand complementary to |
| fG.fG.fA.fG.fA.fA.fC.mA*fA*mU*fA*mU | | recruiting domain on Long |
| (<Strand Ref.: 802>) | | strand 5′-3′ of composition |
| | | RD0566 with linker = Cy-C6 |
| mA*fU*rG.fU.mU.rG.mU.mU.mC.mU.fC.rG.fU. | 4 | Recruiting domain on long |
| mC.fU.rC.fC.mU.mC.rG.rA.mC.rA*mC*mC* | | strand, sequence information |
| (X2) | | 5′-3′ of composition RD0566 |
| (<Strand Ref.: 803>) | | with linker = Cy-C6 |
| (X1)AGGGGUCCACAUGGCAAC | 1 | Editing domain on long strand |
| (<Strand Ref.: 804>) | | sequence information 5′- 3′- |
| | | UM of composition RD0566 |
| | | with linker = Cy-C6 |
| GGUGUCGAGAAGAGGAGAACAAUAU | 3 | short strand complementary to |
| (<Strand Ref.: 805>) | | recruiting domain on Long |
| | | strand 5′-3′-UM linker = Cy- |
| | | C6 |
| AUGUUGUUCUCGUCUCCUCGACACC(X2) | 4 | Recruiting domain on long |
| (<Strand Ref.: 806>) | | strand, sequence information |
| | | 5′-3′-UM linker = Cy-C6 |
| (X1)*mA*mG*mG.mG.mG.mU.rC.rC.rA.mC.mA. | 1 | Editing domain on long strand |
| mU.mG.mG*mC*mA*mA*mC | | sequence information 5′- 3′ |
| (<Strand Ref.: 807>) | | strand of composition RD0567 |
| | | with linker = C3-C6 |
| mG*mG*mU*fG*mU.mC.fG.fA.fG.fA.fA.fG.fA. | 3 | short strand complementary to |
| fG.fG.fA.fG.fA.fA.fC.mA*fA*mU*fA*mU | | recruiting domain on Long |
| (<Strand Ref.: 808>) | | strand 5′-3′ of composition |
| | | RD0567 with linker = C3-C6 |
| mA*fU*rG.fU.mU.rG.mU.mU.mC.mU.fC.rG.fU. | 4 | Recruiting domain on long |
| mC.fU.rC.fC.mU.mC.rG.rA.mC.rA*mC*mC* | | strand, sequence information |
| (X2) | | 5′-3′ of composition RD0567 |
| (<Strand Ref.: 809>) | | with linker = C3-C6 |
| (X1)AGGGGUCCACAUGGCAAC | 1 | Editing domain on long strand |
| (<Strand Ref.: 810>) | | sequence information 5′- 3′- |
| | | UM of composition RD0567 |
| | | with linker = C3-C6 |
| GGUGUCGAGAAGAGGAGAACAAUAU | 3 | short strand complementary to |
| (<Strand Ref.: 811>) | | recruiting domain on Long |
| | | strand 5′-3′-UM linker = C3- |
| | | C6 |
| AUGUUGUUCUCGUCUCCUCGACACC(X2) | 4 | Recruiting domain on long |
| (<Strand Ref.: 812>) | | strand, sequence information |
| | | 5′-3′-UM linker = C3-C6 |
| (X1)*mA*mG*mG.mG.mG.mU.rC.rC.rA.mC.mA. | 1 | Editing domain on long strand |
| mU.mG.mG*mC*mA*mA*mC | | sequence information 5′- 3′ |
| (<Strand Ref.: 813>) | | strand of composition RD0574 |
| | | with linker = PEG2 |
| mG*fG*mG*fU*mG.fG.fA.fA.mG.fA.fG.fU.mA. | 5 | short strand complementary to |
| fG.mA.fA.mC.fA.mA.fU*mA*fU*mG*mC | | recruiting domain on Long |
| (<Strand Ref.: 814>) | | strand 5′-3′ of composition |
| | | RD0574 with linker = PEG2 |
| mG*fC*mA*fU*mA.fU.mU.fG.mU.fU.mC.fU.mA. | 6 | Recruiting domain on long |
| fG.mU.fC.mU.mC.mC.fC.mA*fC*mC*fC*(X2) | | strand, sequence information |
| (<Strand Ref.: 815>) | | 5′-3′ of composition RD0574 |
| | | with linker = PEG2 |
| (X1)AGGGGUCCACAUGGCAAC | 1 | Editing domain on long strand |
| (<Strand Ref.: 816>) | | sequence information 5′- 3′- |
| | | UM of composition RD0574 |
| | | with linker = PEG2 |
| GGGUGGAAGAGUAGAACAAUAUGC | 5 | short strand complementary to |
| (<Strand Ref.: 817>) | | recruiting domain on Long |
| | | strand 5′-3′-UM linker = PEG2 |
| GCAUAUUGUUCUAGUCUCCCACCC(X2) | 6 | Recruiting domain on long |
| (<Strand Ref.: 818>) | | strand, sequence information |
| | | 5′-3′-UM linker = PEG2 |
| (X1)*mG*mG*mC.mU.mC.mC.rC*rC*rA.mG.mG.mC.mC. | 114 | Editing domain on long strand |
| mC*mC*mU*mC*mC | | sequence information 5′- 3′ of |
| (<Strand Ref.: 819>) | | composition RD0790 with |
| | | linker = PEG2 |
| (X1)*mG*mG*mC.mU.mC.mC.rC*rC*rA.mG.mG.mC.mC. | 114 | Editing domain on long strand |
| mC*mC*mU*mC*mC | | sequence information 5′- 3′ of |
| (<Strand Ref.: 820>) | | composition RD1013 with |
| | | linker = PEG2 |
| (X1)*mG*mG*mC.mU.mC.mC.rC*rC*rA.mG.mG.mC.mC. | 114 | Editing domain on long strand |
| mC*mC*mU*mC*mC | | sequence information 5′ - 3′ of |
| (<Strand Ref.: 821>) | | composition RD1042 with |
| | | linker = PEG6 |
| (X1)*mA*mG*mG.mG.mG.mU.rC.rC.rA.mC.mA.mU.mG. | 113 | Editing domain on long strand |
| mG*mA*mA*mA*mC | | sequence information 5′ - 3′ of |
| (<Strand Ref.: 822>) | | composition RD0559 with |
| | | linker = PEG2 |
| (X1)*mA*mG*mG.mG.mG.mU.rC*rC*rA.mC.mA.mU.mG. | 113 | Editing domain on long strand |
| mG*mA*mA*mA*mC | | sequence information 5′- 3′ of |
| (<Strand Ref.: 823>) | | composition RD0560 with |
| | | linker = PEG2 |
| (X1)*mA*mG*mG.mG.mG.mU.dC*dC*dA.mC.mA.mU.mG. | 113 | Editing domain on long strand |
| mG*mA*mA*mA*mC | | sequence information 5′- 3′ of |
| (<Strand Ref.: 824>) | | composition RD0561 with |
| | | linker = PEG2 |
| (X1)*mA*mG*mG.mG.mG.mU.rC*rC*rA.mC.mA.mU.mG. | 113 | Editing domain on long strand |
| mG*mA*mA*mA*mC | | sequence information 5′ - 3′ of |
| (<Strand Ref.: 825>) | | composition RD0764 with |
| | | linker = PEG2 |
| (X1)*mA*mG*mG.mG.mG.mU.rC*rC*rA.mC.mA.mU.mG. | 113 | Editing domain on long strand |
| mG*mA*mA*mA*mC | | sequence information 5′ - 3′ of |
| (<Strand Ref.: 826>) | | composition RD0775 with |
| | | linker = PEG2 |
| (X1)*mA*mG*mG.mG.mG.mU.rC*rC*rA.mC.mA.mU.mG. | 113 | Editing domain on long strand |
| mG*mA*mA*mA*mC | | sequence information 5′ - 3′ of |
| (<Strand Ref.: 827>) | | composition RD0779 with |
| | | linker = PEG2 |
| (X1)*mA*mG*mG.mG.mG.mU.rC*rC*rA.mC.mA.mU.mG. | 113 | Editing domain on long strand |
| mG*mA*mA*mA*mC | | sequence information 5′ - 3′ of |
| (<Strand Ref.: 828>) | | composition RD0780 with |
| | | linker = PEG2 |
| mG*mG*mU*fG*mG.mG.fU.fG.fG.fA.fA.fG.fA.fG.fG. | 84 | short strand complementary to |
| fA.fG*fA*fA*fC*mA | | recruiting domain on Long |
| (<Strand Ref.: 829>) | | strand 5′-3′ of composition |
| | | RD0790 with linker = PEG2 |
| mG*mG*mU*fG*mU.mC.fG.fA.fG.fA.fA.fG.fA.fG.fG. | 3 | short strand complementary to |
| fA.fG.fA.fA.fC.mA*fA*mU*fA*mU | | recruiting domain on Long |
| (<Strand Ref.: 830>) | | strand 5′-3′ of composition |
| | | RD1013 with linker = PEG2 |
| mG*mG*mU*fG*mG.mG.fU.fG.fG.fA.fA.fG.fA.fG.fG. | 84 | short strand complementary to |
| fA.fG*fA*fA*fC*mA | | recruiting domain on Long |
| (<Strand Ref.: 831>) | | strand 5′ -3′ of composition |
| | | RD1042 with linker = PEG6 |
| mG*mG*mU*fG*mU.mC.fG.fA.fG.fA.fA.fG.fA.fG.fG. | 3 | short strand complementary to |
| fA.fG.fA.fA.fC.mA*fA*mU*fA*mU | | recruiting domain on Long |
| (<Strand Ref.: 832>) | | strand 5′-3′ of composition |
| | | RD0559 with linker = PEG2 |
| mG*mG*mU*fG*mU.mC.fG.fA.fG.fA.fA.fG.fA.fG.fG. | 3 | short strand complementary to |
| fA.fG.fA.fA.fC.mA*fA*mU*fA*mU | | recruiting domain on Long |
| (<Strand Ref.: 833>) | | strand 5′-3′ of composition |
| | | RD0560 with linker = PEG2 |
| mG*mG*mU*fG*mU.mC.fG.fA.fG.fA.fA.fG.fA.fG.fG. | 3 | short strand complementary to |
| fA.fG.fA.fA.fC.mA*fA*mU*fA*mU | | recruiting domain on Long |
| (<Strand Ref.: 834>) | | strand 5′-3′ of composition |
| | | RD0561 with linker = PEG2 |
| mG*mG*mU*fG*mU.mC.fG.fA.fG.fA.fA.fG.fA.fG.fG. | 53 | short strand complementary to |
| fA.fG*fA*fA*fC*mA | | recruiting domain on Long |
| (<Strand Ref.: 835>) | | strand 5′-3′ of composition |
| | | RD0764 with linker = PEG2 |
| mG*mG*mU*fG*mU.mC.fG.fA.fG.fA.fA.fG.fA.fG.fG. | 3 | short strand complementary to |
| fA.fG.fA.fA.fC.mA*fA*mU*fA*mU | | recruiting domain on Long |
| (<Strand Ref.: 836>) | | strand 5′-3′ of composition |
| | | RD0775 with linker = PEG2 |
| mG*mG*mU*fG*mG.mG.fU.fG.fG.fA.fA.fG.fA.fG.fG. | 84 | short strand complementary to |
| fA.fG*fA*fA*fC*mA | | recruiting domain on Long |
| (<Strand Ref.: 837>) | | strand 5′-3′ of composition |
| | | RD0779 with linker = PEG2 |
| mG*mG*mU*fG*mG.mG.fU.fG.fG.fA.fA.fG.fA.fG.fG. | 84 | short strand complementary to |
| fA.fG*fA*fA*fC*mA | | recruiting domain on Long |
| (<Strand Ref.: 838>) | | strand 5′-3′ of composition |
| | | RD0780 with linker = PEG2 |
| mU*fG*mU*mU*mC.mU.fC.fG.fU.mC.fU.mC.fC.mC.mA. | 115 | Recruiting domain on long |
| fC.fC.mC*fA*mC*mC*(X2) | | strand, sequence information |
| (<Strand Ref.: 839>) | | 5′-3′ of composition RD0790 |
| | | with linker = PEG2 |
| mA*fU*rG.fU.mU.rG.mU.mU.mC.mU.fC.rG.fU.mC.fU. | 4 | Recruiting domain on long |
| rC.fC.mU.mC.rG.rA.mC.rA*mC*mC*(X2) | | strand, sequence information |
| (<Strand Ref.: 840>) | | 5′-3′ of composition RD1013 |
| | | with linker = PEG2 |
| mU*fG*mU*mU*mC.mU.fC.fG.fU.mC.fU.mC.fC.mC.mA. | 115 | Recruiting domain on long |
| fC.fC.mC*fA*mC*mC*(X2) | | strand, sequence information |
| (<Strand Ref.: 841>) | | 5′-3′ of composition RD1042 |
| | | with linker = PEG6 |
| mA*fU*rG.fU.mU.rG.mU.mU.mC.mU.fC.rG.fU.mC.fU. | 4 | Recruiting domain on long |
| rC.fC.mU.mC.rG.rA.mC.rA*mC*mC*(X2) | | strand, sequence information |
| (<Strand Ref.: 842>) | | 5′-3′ of composition RD0559 |
| | | with linker = PEG2 |
| mA*fU*rG.fU.mU.rG.mU.mU.mC.mU.fC.rG.fU.mC.fU. | 4 | Recruiting domain on long |
| rC.fC.mU.mC.rG.rA.mC.rA*mC*mC*(X2) | | strand, sequence information |
| (<Strand Ref.: 843>) | | 5′-3′ of composition RD0560 |
| | | with linker = PEG2 |
| mA*fU*rG.fU.mU.rG.mU.mU.mC.mU.fC.rG.fU.mC.fU. | 4 | Recruiting domain on long |
| rC.fC.mU.mC.rG.rA.mC.rA*mC*mC*(X2) | | strand, sequence information |
| (<Strand Ref.: 844>) | | 5′-3′ of composition RD0561 |
| | | with linker = PEG2 |
| mU*fG*mU*mU*mC.mU.fC.fG.fU.mC.fU.fC.fC.mU.mC. | 116 | Recruiting domain on long |
| fG.fA.mC*fA*mC*mC*(X2) | | strand, sequence information |
| (<Strand Ref.: 845>) | | 5′-3′ of composition RD0764 |
| | | with linker = PEG2 |
| mG*mC*mA*fU*fA.fU.mU.fG.mU.mU.mC.mU.fC.fG.fU. | 117 | Recruiting domain on long |
| mC.fU.fC.fC.mU.mC.fG.fA.mC*fA*mC*mC*(X2) | | strand, sequence information |
| (<Strand Ref.: 846>) | | 5′-3′ of composition RD0775 |
| | | with linker = PEG2 |
| mU*fG*mU*mU*mC.mU.fC.fG.fU.mC.fU.mC.fC.mC.mA. | 115 | Recruiting domain on long |
| fC.fC.mC*fA*mC*mC*(X2) | | strand, sequence information |
| (<Strand Ref.: 847>) | | 5′-3′ of composition RD0779 |
| | | with linker = PEG2 |
| mU*mU*fG*mU*mU.mC.mU.fC.fG.fU.mC.fU.mC.fC.mC. | 118 | Recruiting domain on long |
| mA.fC.fC.mC*fA*mC*mC*(X2) | | strand, sequence information |
| (<Strand Ref.: 848>) | | 5′-3′ of composition RD0780 |
| | | with linker = PEG2 |
| (X1)GGCUCCCCAGGCCCCUCC | 114 | Editing domain on long strand |
| (<Strand Ref.: 849>) | | sequence information 5′- 3′ of |
| | | composition RD0790 with |
| | | linker = PEG2 - UM Sequence |
| (X1)GGCUCCCCAGGCCCCUCC | 114 | Editing domain on long strand |
| (<Strand Ref.: 850>) | | sequence information 5′ - 3′ of |
| | | composition RD1013 with |
| | | linker = PEG2 - UM Sequence |
| (X1)GGCUCCCCAGGCCCCUCC | 114 | Editing domain on long strand |
| (<Strand Ref.: 851>) | | sequence information 5′ - 3′ of |
| | | composition RD1042 with |
| | | linker = PEG6 - UM Sequence |
| (X1)AGGGGUCCACAUGGAAAC | 113 | Editing domain on long strand |
| (<Strand Ref.: 852>) | | sequence information 5′- 3′ of |
| | | composition RD0559 with |
| | | linker = PEG2 - UM Sequence |
| (X1)AGGGGUCCACAUGGAAAC | 113 | Editing domain on long strand |
| (<Strand Ref.: 853>) | | sequence information 5′- 3′ of |
| | | composition RD0560 with |
| | | linker = PEG2 - UM Sequence |
| (X1)AGGGGUdCdCdACAUGGAAAC | 113 | Editing domain on long strand |
| (<Strand Ref.: 854>) | | sequence information 5′ - 3′ of |
| | | composition RD0561 with |
| | | linker = PEG2 - UM Sequence |
| (X1)AGGGGUCCACAUGGAAAC | 113 | Editing domain on long strand |
| (<Strand Ref.: 855>) | | sequence information 5′- 3′ of |
| | | composition RD0764 with |
| | | linker = PEG2 - UM Sequence |
| (X1)AGGGGUCCACAUGGAAAC | 113 | Editing domain on long strand |
| (<Strand Ref.: 856>) | | sequence information 5′- 3′ of |
| | | composition RD0775 with |
| | | linker = PEG2 - UM Sequence |
| (X1)AGGGGUCCACAUGGAAAC | 113 | Editing domain on long strand |
| (<Strand Ref.: 857>) | | sequence information 5′ - 3′ of |
| | | composition RD0779 with |
| | | linker = PEG2 - UM Sequence |
| (X1)AGGGGUCCACAUGGAAAC | 113 | Editing domain on long strand |
| (<Strand Ref.: 858>) | | sequence information 5′ - 3′ of |
| | | composition RD0780 with |
| | | linker = PEG2 - UM Sequence |
| GGUGGGUGGAAGAGGAGAACA | 84 | short strand complementary to |
| (<Strand Ref.: 859>) | | recruiting domain on Long |
| | | strand 5′-3′ of composition |
| | | RD0790 with linker = PEG2 - |
| | | UM Sequence |
| GGUGUCGAGAAGAGGAGAACAAUAU | 3 | short strand complementary to |
| (<Strand Ref.: 860>) | | recruiting domain on Long |
| | | strand 5′ -3′ of composition |
| | | RD1013 with linker = PEG2 - |
| | | UM Sequence |
| GGUGGGUGGAAGAGGAGAACA | 84 | short strand complementary to |
| (<Strand Ref.: 861>) | | recruiting domain on Long |
| | | strand 5′-3′ of composition |
| | | RD1042 with linker = PEG6 - |
| | | UM Sequence |
| GGUGUCGAGAAGAGGAGAACAAUAU | 3 | short strand complementary to |
| (<Strand Ref.: 862>) | | recruiting domain on Long |
| | | strand 5′-3′ of composition |
| | | RD0559 with linker = PEG2 - |
| | | UM Sequence |
| GGUGUCGAGAAGAGGAGAACAAUAU | 3 | short strand complementary to |
| (<Strand Ref.: 863>) | | recruiting domain on Long |
| | | strand 5′-3′ of composition |
| | | RD0560 with linker = PEG2 - |
| | | UM Sequence |
| GGUGUCGAGAAGAGGAGAACAAUAU | 3 | short strand complementary to |
| (<Strand Ref.: 864>) | | recruiting domain on Long |
| | | strand 5′-3′ of composition |
| | | RD0561 with linker = PEG2 - |
| | | UM Sequence |
| GGUGUCGAGAAGAGGAGAACA | 53 | short strand complementary to |
| (<Strand Ref.: 865>) | | recruiting domain on Long |
| | | strand 5′-3′ of composition |
| | | RD0764 with linker = PEG2 - |
| | | UM Sequence |
| GGUGUCGAGAAGAGGAGAACAAUAU | 3 | short strand complementary to |
| (<Strand Ref.: 866>) | | recruiting domain on Long |
| | | strand 5′-3′ of composition |
| | | RD0775 with linker = PEG2 - |
| | | UM Sequence |
| GGUGGGUGGAAGAGGAGAACA | 8 | short strand complementary to |
| (<Strand Ref.: 867>) | | recruiting domain on Long |
| | | strand 5′-3′ of composition |
| | | RD0779 with linker = PEG2 - |
| | | UM Sequence |
| GGUGGGUGGAAGAGGAGAACA | 8. | short strand complementary to |
| (<Strand Ref.: 868>) | | recruiting domain on Long |
| | | strand 5′-3′ of composition |
| | | RD0780 with linker = PEG2 - |
| | | UM Sequence |
| UGUUCUCGUCUCCCACCCACC(X2) | 115 | Recruiting domain on long |
| (<Strand Ref.: 869>) | | strand, sequence information |
| | | 5′-3′ of composition RD0790 |
| | | with linker = PEG2 - UM |
| | | Sequence |
| AUGUUGUUCUCGUCUCCUCGACACC(X2) | 4 | Recruiting domain on long |
| (<Strand Ref.: 870>) | | strand, sequence information |
| | | 5′-3′ of composition RD1013 |
| | | with linker = PEG2 - UM |
| | | Sequence |
| UGUUCUCGUCUCCCACCCACC(X2) | 115 | Recruiting domain on long |
| (<Strand Ref.: 871>) | | strand, sequence information |
| | | 5′-3′ of composition RD1042 |
| | | with linker = PEG6 - UM |
| | | Sequence |
| AUGUUGUUCUCGUCUCCUCGACACC(X2) | 4 | Recruiting domain on long |
| (<Strand Ref.: 872>) | | strand, sequence information |
| | | 5′-3′ of composition RD0559 |
| | | with linker = PEG2 - UM |
| | | Sequence |
| AUGUUGUUCUCGUCUCCUCGACACC(X2) | 4 | Recruiting domain on long |
| (<Strand Ref.: 873>) | | strand, sequence information |
| | | 5′-3′ of composition RD0560 |
| | | with linker = PEG2 - UM |
| | | Sequence |
| AUGUUGUUCUCGUCUCCUCGACACC(X2) | 4 | Recruiting domain on long |
| (<Strand Ref.: 874>) | | strand, sequence information |
| | | 5′-3′ of composition RD0561 |
| | | with linker = PEG2 - UM |
| | | Sequence |
| UGUUCUCGUCUCCUCGACACC(X2) | 116 | Recruiting domain on long |
| (<Strand Ref.: 875>) | | strand, sequence information |
| | | 5′-3′ of composition RD0764 |
| | | with linker = PEG2 - UM |
| | | Sequence |
| GCAUAUUGUUCUCGUCUCCUCGACACC(X2) | 117 | Recruiting domain on long |
| (<Strand Ref.: 876>) | | strand, sequence information |
| | | 5′-3′ of composition RD0775 |
| | | with linker = PEG2 - UM |
| | | Sequence |
| UGUUCUCGUCUCCCACCCACC(X2) | 115 | Recruiting domain on long |
| (<Strand Ref.: 877>) | | strand, sequence information |
| | | 5′-3′ of composition RD0779 |
| | | with linker = PEG2 - UM |
| | | Sequence |
| UUGUUCUCGUCUCCCACCCACC(X2) | 118 | Recruiting domain on long |
| (<Strand Ref.: 878>) | | strand, sequence information |
| | | 5′-3′ of composition RD0780 |
| | | with linker = PEG2 - UM |
| | | Sequence |
| GGUGGGUGGAAGAGGAGAAC | 166 | Short strand of RD1017 duplex |
| (<Strand Ref.: 879>) | | |
| UGUUCUCGUCUCCCACCCACC | 115 | Long strand of RD1017 duplex |
| (<Strand Ref.: 880>) | | |
| GGUGGGUGGAAGAGGAGAACA | 84 | Short strand of RD1018 duplex |
| (<Strand Ref.: 881>) | | |
| UUGUUCUCGUCUCCCACCCACC | 118 | Long strand of RD1018 duplex |
| (<Strand Ref.: 882>) | | |
| GGUGGGUGGAAGAGGAGAACA | 84 | Long strand of RD1019 duplex |
| (<Strand Ref.: 883>) | | |
| GUUCUCGUCUCCCACCCACC | 167 | Short strand of RD1019 duplex |
| (<Strand Ref.: 884>) | | |
| mG*fG*mG*fU*mG.fG.fA.fA.mG.fA.fG.fU.mA.fG.mA.fA. | 5 | short strand complementary to |
| .mC.fA.mA.fU*mA*fU*mG*mC | | recruiting domain on Long |
| (<Strand Ref.: 885>) | | strand 5′-3′ of Oligo O with |
| | | linker = PEG2 |
| mG*fC*mA*fU*mA.fU.mU.fG.mU.fU.mC.fU.mA.fG.mU.fC. | 6 | Recruiting domain on long |
| mU.mC.mC.fC.mA*fC*mC*fC*(X2) | | strand, sequence information |
| (<Strand Ref.: 886>) | | 5′-3′ of Oligo O with linker = |
| | | PEG2 |
| mG*fG*mU*fG*mU.fC.mG.fA.mG.fA.mA.fG.mA.fG.mG.fA. | 3 | short strand complementary to |
| mG.fA.mA.fC.mA*fA*mU*fA*mU | | recruiting domain on Long |
| (<Strand Ref.: 887>) | | strand 5′-3′ of Oligo D with |
| | | linker = PEG2 |
| mA*fU*mG*fU*mU.fG.mU.fU.mC.fU.mC.fG.mU.fC.mU.mC. | 4 | Recruiting domain on long |
| mC.fU.mC.fG.mA.fC*mA*fC*mC*(X2) | | strand, sequence information |
| (<Strand Ref.: 888>) | | 5′-3′ of Oligo D with linker = |
| | | PEG2 |
| mG*mG*mU*fG*mU.mC.fG.fA.fG.fA.fA.fG.fA.fG.fG.fA. | 3 | short strand complementary to |
| fG.fA.fA.fC.mA*fA*mU*fA*mU | | recruiting domain on Long |
| (<Strand Ref.: 889>) | | strand 5′-3′ of Oligo H with |
| | | linker = PEG2 |
| mA*fU*fA.fU.mU.fG.mU.mU.mC.mU.fC.fG.fU.mC.fU.fC. | 168 | Recruiting domain on long |
| fC.mU.mC.fG.fA.mC.fA*mC*mC*(X2) | | strand, sequence information |
| (<Strand Ref.: 890>) | | 5′-3′ of Oligo H with linker = |
| | | PEG2 |
| mG*mG*mU*fG*mU.mC.fG.fA.fG.fA.fA.fG.fA.fG.fG.fA. | 3 | short strand complementary to |
| fG.fA.fA.fC.mA*fA*mU*fA*mU | | recruiting domain on Long |
| (<Strand Ref.: 891>) | | strand 5′-3′ of Oligo M with |
| | | linker = PEG2 |
| mA*fU*fA.fU.mU.fG.mU.mU.mC.mU.fC.fG.fU.mC.fU.mC. | 168 | Recruiting domain on long |
| fC.mU.mC.fG.fA.mC.fA*mC*mC*(X2) | | strand, sequence information |
| (<Strand Ref.: 892>) | | 5′-3′ of Oligo M with linker = |
| | | PEG2 |
| mG*mG*mU*fG*mU.mC.fG.fA.fG.fA.fA.fG.fA.fG.fG.fA. | 3 | short strand complementary to |
| fG.fA.fA.fC.mA*fA*mU*fA*mU | | recruiting domain on Long |
| (<Strand Ref.: 893>) | | strand 5′-3′ of Oligo Q with |
| | | linker = PEG2 |
| mA*fU*mA*fU*mU.fG.mU.fU.mC.fU.mC.fG.mU.fC.mU.mC. | 168 | Recruiting domain on long |
| mC.fU.mC.fG.mA.fC*mA*fC*mC*(X2) | | strand, sequence information |
| (<Strand Ref.: 894>) | | 5′-3′ of Oligo Q with linker = |
| | | PEG2 |
| mG*mG*mU*fG*mU.mC.fG.fA.fG.fA.fA.fG.fA.fG.fG.fA. | 3 | short strand complementary to |
| fG.fA.fA.fC.mA*fA*mU*fA*mU | | recruiting domain on Long |
| (<Strand Ref.: 895>) | | strand 5′-3′ of Oligo P with |
| | | linker = PEG2 |
| mA*fU*fG.fU.mU.fG.mU.mU.mC.mU.fC.fG.fU.mC.fU.fC. | 4 | Recruiting domain on long |
| fC.mU.mC.fG.fA.mC.fA*mC*mC*(X2) | | strand, sequence information |
| (<Strand Ref.: 896>) | | 5′-3′ of Oligo P with linker = |
| | | PEG2 |
| mG*fG*mU*fG*mU.fC.mG.fA.mG.fA.mA.fG.mA.fG.mG.fA. | 3 | short strand complementary to |
| mG.fA.mA.fC.mA*fA*mU*fA*mU | | recruiting domain on Long |
| (<Strand Ref.: 897>) | | strand 5′-3′ of Oligo E with |
| | | linker = PEG2 |
| H1.mA*fU*mG*fU*mU.fG.mU.fU.mC.fU.mC.fG.mU.fC.mU. | 4 | Recruiting domain on long |
| mC.mC.fU.mC.fG.mA.fC*mA*fC*mC*(X2) | | strand, sequence information |
| (<Strand Ref.: 898>) | | 5′-3′ of Oligo E with linker = |
| | | PEG2 |
| mG*fG*mG*fU*mG.fG.fA.fA.mG.fA.fG.fU.mA.fG.mA.fA. | 5 | short strand complementary to |
| mC.fA.mA.fU*mA*fU*mG*mC | | recruiting domain on Long |
| (<Strand Ref.: 899>) | | strand 5′-3′ of Oligo I with |
| | | linker = PEG2 |
| H1.mG*fC*mA*fU*mA.fU.mU.fG.mU.fU.mC.fU.mA.fG.mU. | 6 | Recruiting domain on long |
| fC.mU.mC.mC.fC.mA*fC*mC*fC*(X2) | | strand, sequence information |
| (<Strand Ref.: 900>) | | 5′-3′ of Oligo I with linker = |
| | | PEG2 |
| mA*mG*mG*mG*mG.mU.rC.rC.rA.mC.mA.mU.mG.mG.mC*mA. | 1 | Editing domain on long strand |
| *mA*mC*(X1) | | sequence information 5′ - 3′ of |
| (<Strand Ref.: 901>) | | RD0750 |
| mG*mG*mU*fG*mU.mC.fG.fA.fG.fA.fA.fG.fA.fG.fG.fA. | 3 | short strand complementary to |
| fG.fA.fA.fC.mA*fA*mU*fA*mU | | recruiting domain on Long |
| (<Strand Ref.: 902>) | | strand 5′-3′ of RD0750 |
| mA*fU*rG.fU.mU.rG.mU.mU.mC.mU.fC.rG.fU.mC.fU.rC. | 4 | Recruiting domain on long |
| fC.mU.mC.rG.rA.mC.rA*mC*mC*(X2) | | strand, sequence information |
| (<Strand Ref.: 903>) | | 5′-3′ of RD0750 |
| (X1)*mA*mG*mG.mG.mG.mU.rC.rC.rA.mC.mA.mU.mG.mG* | 1 | Editing domain on long strand |
| mC*mA*mA*mC | | sequence information 5′ - 3′ of |
| (<Strand Ref.: 904>) | | RD0752 |
| mG*mG*mU*fG*mU.mC.fG.fA.fG.fA.fA.fG.fA.fG.fG.fA. | 53 | short strand complementary to |
| fG*fA*fA*fC*mA | | recruiting domain on Long |
| (<Strand Ref.: 905>) | | strand 5′-3′ of RD0752 |
| mU*fG*mU*mU*mC.mU.fC.fG.fU.mC.fU.fC.fC.mU.mC.fG. | 116 | Recruiting domain on long |
| fA.mC*fA*mC*mC*(X2) | | strand, sequence information |
| (<Strand Ref.: 906>) | | 5′-3′ of RD0752 |
| (X1)*mA*mG*mG.mG.mG.mU.rC.rC.rA.mC.mA.mU.mG.mG* | 1 | Editing domain on long strand |
| mC*mA*mA*mC | | sequence information 5′ - 3′ of |
| (<Strand Ref.: 907>) | | RD0754 |
| mG*mG*mU*fG*mU.mC.fG.fA.fG.fA.fA.fG.fA.fG.fG.fA. | 3 | short strand complementary to |
| fG.fA.fA.fC.mA*fA*mU*fA*mU | | recruiting domain on Long |
| (<Strand Ref.: 908>) | | strand 5′-3′ of RD0754 |
| mA*fU*rG.fU.mU.rG.mU.mU.mC.mU.fC.rG.fU.mC.fU.rC. | 4 | Recruiting domain on long |
| fC.mU.mC.rG.rA.mC.rA*mC*mC*(X2) | | strand, sequence information |
| (<Strand Ref.: 909>) | | 5′-3′ of RD0754 |
| (X1)*mA*mG*mG.mG.mG.mU.rC.rC.rA.mC.mA.mU.mG.mG* | 1 | Editing domain on long strand |
| mC*mA*mA*mC | | sequence information 5′- 3′ of |
| (<Strand Ref.: 910>) | | RD0755 |
| mG*mG*mU*fG*mU.mC.fG.fA.fG.fA.fA.fG.fA.fG.fG.fA. | 3 | short strand complementary to |
| fG.fA.fA.fC.mA*fA*mU*fA*mU | | recruiting domain on Long |
| (<Strand Ref.: 911>) | | strand 5′-3′ of RD0755 |
| mA*fU*rG.fU.mU.rG.mU.mU.mC.mU.fC.rG.fU.mC.fU.rC. | 4 | Recruiting domain on long |
| fC.mU.mC.rG.rA.mC.rA*mC*mC*(X2) | | strand, sequence information |
| (<Strand Ref.: 912>) | | 5′-3′ of RD0755 |
| (X1)*mA*mG*mG.mG.mG.mU.rC.rC.rA.mC.mA.mU.mG.mG* | 1 | Editing domain on long strand |
| mC*mA*mA*mC | | sequence information 5′ - 3′ of |
| (<Strand Ref.: 913>) | | RD0756 |
| mG*mG*mU*fG*mU.mC.fG.fA.fG.fA.fA.fG.fA.fG.fG.fA. | 3 | short strand complementary to |
| fG.fA.fA.fC.mA*fA*mU*fA*mU | | recruiting domain on Long |
| (<Strand Ref.: 914>) | | strand 5′-3′ of RD0756 |
| mA*fU*rG.fU.mU.rG.mU.mU.mC.mU.fC.rG.fU.mC.fU.rC. | 4 | Recruiting domain on long |
| fC.mU.mC.rG.rA.mC.rA*mC*mC*(X2) | | strand, sequence information |
| (<Strand Ref.: 915>) | | 5′-3′ of RD0756 |
| (X1)*mA*mG*mG.mG.mG.mU.rC*rC*rA.mC.mA.mU.mG.mG* | 1 | Editing domain on long strand |
| mC*mA*mA*mC | | sequence information 5′ - 3′ of |
| (<Strand Ref.: 916>) | | RD0765 |
| mG*mG*mU*fG*mU.mC.fG.fA.fG.fA.fA.fG.fA.fG.fG.fA. | 53 | short strand complementary to |
| fG*fA*fA*fC*mA | | recruiting domain on Long |
| (<Strand Ref.: 917>) | | strand 5′-3′ of RD0765 |
| mU*fG*mU*mU*mC.mU.fC.fG.fU.mC.fU.fC.fC.mU.mC.fG. | 116 | Recruiting domain on long |
| fA.mC*fA*mC*mC*(X2) | | strand, sequence information |
| (<Strand Ref.: 918>) | | 5′-3′ of RD0765 |
| (X1)*mA*mG*mG.mG.mG.mU.dC*dC*dA.mC.mA.mU.mG.mG* | 1 | Editing domain on long strand |
| mC*mA*mA*mC | | sequence information 5′ - 3′ of |
| (<Strand Ref.: 919>) | | RD0766 |
| mG*mG*mU*fG*mU.mC.fG.fA.fG.fA.fA.fG.fA.fG.fG.fA. | 53 | short strand complementary to |
| fG*fA*fA*fC*mA | | recruiting domain on Long |
| (<Strand Ref.: 920>) | | strand 5′-3′ of RD0766 |
| mU*fG*mU*mU*mC.mU.fC.fG.fU.mC.fU.fC.fC.mU.mC.fG. | 116 | Recruiting domain on long |
| fA.mC*fA*mC*mC*(X2) | | strand, sequence information |
| (<Strand Ref.: 921>) | | 5′-3′ of RD0766 |
| (X1)*mA*mG*mG.mG.mG.mU.rC*rC*rA.mC.mA.mU.mG.mG* | 1 | Editing domain on long strand |
| mC*mA*mA*mC | | sequence information 5′ - 3′ of |
| (<Strand Ref.: 922>) | | RD0767 |
| mG*fG*mU*fG*mU.fC.mG.fA.mG.fA.mA.fG.mA.fG.mG.fA. | 3 | short strand complementary to |
| mG.fA.mA.fC.mA*fA*mU*fA*mU | | recruiting domain on Long |
| (<Strand Ref.: 923>) | | strand 5′-3′ of RD0767 |
| mA*fU*mG*fU*mU.fG.mU.fU.mC.fU.mC.fG.mU.fC.mU.mC. | 4 | Recruiting domain on long |
| mC.fU.mC.fG.mA.fC*mA*fC*mC.(X2) | | strand, sequence information |
| (<Strand Ref.: 924>) | | 5′-3′ of RD0767 |
| (X1)*mA*mG*mG.mG.mG.mU.rC*rC*rA.mC.mA.mU.mG.mG* | 1 | Editing domain on long strand |
| mC*mA*mA*mC | | sequence information 5′- 3′ of |
| (<Strand Ref.: 925>) | | RD0774 |
| mG*mG*mU*fG*mU.mC.fG.fA.fG.fA.fA.fG.fA.fG.fG.fA. | 3 | short strand complementary to |
| fG.fA.fA.fC.mA*fA*mU*fA*mU | | recruiting domain on Long |
| (<Strand Ref.: 926>) | | strand 5′-3′ of RD0774 |
| mG*mC*mA*fU*fA.fU.mU.fG.mU.mU.mC.mU.fC.fG.fU.mC. | 117 | Recruiting domain on long |
| fU.fC.fC.mU.mC.fG.fA.mC*fA*mC*mC*(X2) | | strand, sequence information |
| (<Strand Ref.: 927>) | | 5′-3′ of RD0774 |
| (X1)*mA*mG*mG.mG.mG.mU.rC*rC*rA.mC.mA.mU.mG.mG* | 1 | Editing domain on long strand |
| mC*mA*mA*mC | | sequence information 5′- 3′ of |
| (<Strand Ref.: 928>) | | RD0995 |
| mG*mG*mU*fG*mG.mG.fU.fG.fG.fA.fA.fG.fA.fG.fG.fA. | 84 | short strand complementary to |
| fG*fA*fA*fC*mA | | recruiting domain on Long |
| (<Strand Ref.: 929>) | | strand 5′-3′ of RD0995 |
| mU*fG*mU*mU*mC.mU.fC.fG.fU.mC.fU.mC.fC.mC.mA.fC. | 115 | Recruiting domain on long |
| fC.mC*fA*mC*mC*(X2) | | strand, sequence information |
| (<Strand Ref.: 930>) | | 5′-3′ of RD0995 |
| (X1)*mA*mG*mG.mG.mG.mU.rC*rC*rA.mC.mA.mU.mG.mG* | 1 | Editing domain on long strand |
| mC*mA*mA*mC | | sequence information 5′ - 3′ of |
| (<Strand Ref.: 931>) | | RD0996 |
| mG*mG*mU*fG*mG.mG.fU.fG.fG.fA.fA.fG.fA.fG.fG.fA. | 84 | short strand complementary to |
| fG*fA*fA*fC*mA | | recruiting domain on Long |
| (<Strand Ref.: 932>) | | strand 5′-3′ of RD0996 |
| mU*fG*mU*mU*mC.mU.fC.fG.fU.mC.fU.mC.fC.mC.mA.fC. | 115 | Recruiting domain on long |
| fC.mC*fA*mC*mC*(X2) | | strand, sequence information |
| (<Strand Ref.: 933>) | | 5′-3′ of RD0996 |
| (X1)*mA*mG*mG.mG.mG.mU.rC*rC*rA.mC.mA.mU.mG.mG* | 1 | Editing domain on long strand |
| mC*mA*mA*mC | | sequence information 5′- 3′ of |
| (<Strand Ref.: 934>) | | RD0997 |
| mG*mG*mU*fG*mG.mG.fU.fG.fG.fA.fA.fG.fA.fG.fG.fA. | 84 | short strand complementary to |
| fG*fA*fA*fC*mA | | recruiting domain on Long |
| (<Strand Ref.: 935>) | | strand 5′-3′ of RD0997 |
| mU*fG*mU*mU*mC.mU.fC.fG.fU.mC.fU.mC.fC.mC.mA.fC. | 115 | Recruiting domain on long |
| fC.mC*fA*mC*mC*(X2) | | strand, sequence information |
| (<Strand Ref.: 936>) | | 5′-3′ of RD0997 |
| (X1)*mA*mG*mG.mG.mG.mU.rC*rC*rA.mC.mA.mU.mG.mG* | 1 | Editing domain on long strand |
| mC*mA*mA*mC | | sequence information 5′- 3′ of |
| (<Strand Ref.: 937>) | | RD0999 |
| mG*mG*mU*fG*mG.mG.fU.fG.fG.fA.fA.fG.fA.fG.fG.fA. | 84 | short strand complementary to |
| fG*fA*fA*fC*mA | | recruiting domain on Long |
| (<Strand Ref.: 938>) | | strand 5′-3′ of RD0999 |
| MU*fG*mU*mU*mC.mU.fC.fG.fU.mC.fU.mC.fC.mC.mA.fC. | 115 | Recruiting domain on long |
| fC.mC*fA*mC*mC*(X2) | | strand, sequence information |
| (<Strand Ref.: 939>) | | 5′-3′ of RD0999 |
| (X1)*mA*mG*mG.mG.mG.mU.rC*rC*rA.mC.mA.mU.mG.mG* | 113 | Editing domain on long strand |
| mA*mA*mA*mC | | sequence information 5′- 3′ of |
| (<Strand Ref.: 940>) | | RD1014 |
| mG*fG*mG*fU*mG.fG.fA.fA.mG.fA.fG.fU.mA.fG.mA.fA. | 5 | short strand complementary to |
| mC.fA.mA.fU*mA*fU*mG*mC | | recruiting domain on Long |
| (<Strand Ref.: 941>) | | strand 5′-3′ of RD1014 |
| mG*fC*mA*fU*mA.fU.mU.fG.mU.fU.mC.fU.mA.fG.mU.fC. | 6 | Recruiting domain on long |
| mU.mC.mC.fC.mA*fC*mC*fC*(X2) | | strand, sequence information |
| (<Strand Ref.: 942>) | | 5′-3′ of RD1014 |
| (X1)*mA*mG*mG.mG.mG.mU.rC*rC*rA.mC.mA.mU.mG.mG* | 1 | Editing domain on long strand |
| mC*mA*mA*mC | | sequence information 5′- 3′ of |
| (<Strand Ref.: 943>) | | RD1015 |
| mG*fG*mG*fU*mG.fG.fA.fA.mG.fA.fG.fU.mA.fG.mA.fA. | 5 | short strand complementary to |
| mC.fA.mA.fU*mA*fU*mG*mC | | recruiting domain on Long |
| (<Strand Ref.: 944>) | | strand 5′-3′ of RD1015 |
| mG*fC*mA*fU*mA.fU.mU.fG.mU.fU.mC.fU.mA.fG.mU.fC. | 6 | Recruiting domain on long |
| mU.mC.mC.fC.mA*fC*mC*fC*(X2) | | strand, sequence information |
| (<Strand Ref.: 945>) | | 5′-3′ of RD1015 |
| (X1)*mA*mG*mG.mG.mG.mU.rC*rC*rA.mC.mA.mU.mG.mG* | 113 | Editing domain on long strand |
| mA*mA*mA*mC | | sequence information 5′- 3′ of |
| (<Strand Ref.: 946>) | | RD0781 |
| mG*fU*mG*fU*mG.fG.fA.fA.mG.fA.fG.fG.mA.fG.mA.fA. | 111 | short strand complementary to |
| mC*fA*mG*fU*mG | | recruiting domain on Long |
| (<Strand Ref.: 947>) | | strand 5′-3′ of RD0781 |
| mC*mA*fC*mU*fG.mU.fU.mC.fU.mC.fG.mU.fC.mU.mC.mC. | 144 | Recruiting domain on long |
| fC.mA*fC*mC*fC*(X2) | | strand, sequence information |
| (<Strand Ref.: 948>) | | 5′-3′ of RD0781 |
| (X1)*mA*mG*mG.mG.mG.mU.rC*rC*rA.mC.mA.mU.mG.mG* | 1 | Editing domain on long strand |
| mC*mA*mA*mC | | sequence information 5′- 3′ of |
| (<Strand Ref.: 949>) | | RD1017 |
| mG*mG*mU*fG*mG.mG.fU.fG.fG.fA.fA.fG.fA.fG.fG.fA* | 166 | short strand complementary to |
| fG*fA*fA*mC | | recruiting domain on Long |
| (<Strand Ref.: 950>) | | strand 5′-3′ of RD1017 |
| mU*fG*mU*mU*mC.mU.fC.fG.fU.mC.fU.mC.fC.mC.mA.fC. | 115 | Recruiting domain on long |
| fC.mC*fA*mC*mC*(X2) | | strand, sequence information |
| (<Strand Ref.: 951>) | | 5′-3′ of RD1017 |
| (X1)*mA*mG*mG.mG.mG.mU.rC*rC*rA.mC.mA.mU.mG.mG* | 1 | Editing domain on long strand |
| mC*mA*mA*mC | | sequence information 5′ - 3′ of |
| (<Strand Ref.: 952>) | | RD1018 |
| mG*mG*mU*fG*mG.mG.fU.fG.fG.fA.fA.fG.fA.fG.fG.fA. | 84 | short strand complementary to |
| fG*fA*fA*fC*mA | | recruiting domain on Long |
| (<Strand Ref.: 953>) | | strand 5′-3′ of RD1018 |
| mU*mU*fG*mU*mU.mC.mU.fC.fG.fU.mC.fU.mC.fC.mC.mA. | 118 | Recruiting domain on long |
| fC.fC.mC*fA*mC*mC*(X2) | | strand, sequence information |
| (<Strand Ref.: 954>) | | 5′-3′ of RD1018 |
| (X1)*mA*mG*mG.mG.mG.mU.rC*rC*rA.mC.mA.mU.mG.mG* | 1 | Editing domain on long strand |
| mC*mA*mA*mC | | sequence information 5′- 3′ of |
| (<Strand Ref.: 955>) | | RD1019 |
| mG*mG*mU*fG*mG.mG.fU.fG.fG.fA.fA.fG.fA.fG.fG.fA. | 84 | short strand complementary to |
| fG*fA*fA*fC*mA | | recruiting domain on Long |
| (<Strand Ref.: 956>) | | strand 5′ -3′ of RD1019 |
| mG*mU*mU*mC*mU.fC.fG.fU.mC.fU.mC.fC.mC.mA.fC.fC. | 167 | Recruiting domain on long |
| mC*fA*mC*mC*(X2) | | strand, sequence information |
| (<Strand Ref.: 957>) | | 5′-3′ of RD1019 |
| (X1)*mA*mG*mG.mG.mG.mU.rC*rC*rA.mC.mA.mU.mG.mG* | 113 | Editing domain on long strand |
| mA*mA*mA*mC | | sequence information 5′- 3′ of |
| (<Strand Ref.: 958>) | | RD1021 |
| mG*fU*mG*fU*mG.fG.fA.fA.mG.fA.fG.fG.mA.fG.mA.fA. | 111 | short strand complementary to |
| mC*fA*mG*fU*mG | | recruiting domain on Long |
| (<Strand Ref.: 959>) | | strand 5′-3′ of RD1021 |
| mC*mA*fC*mU*fG.mU.fU.mC.fU.mC.fG.mU.fC.mU.mC.mC. | 144 | Recruiting domain on long |
| fC.mA*fC*mC*fC*(X2) | | strand, sequence information |
| (<Strand Ref.: 960>) | | 5′-3′ of RD1021 |
| (X1)*mA*mG*mG.mG.mG.mU.rC*rC*rA.mC.mA.mU.mG.mG* | 1 | Editing domain on long strand |
| mC*mA*mA*mC | | sequence information 5′- 3′ of |
| (<Strand Ref.: 961>) | | RD1022 |
| mG*fU*mG*fU*mG.fG.fA.fA.mG.fA.fG.fG.mA.fG.mA.fA. | 111 | short strand complementary to |
| mC*fA*mG*fU*mG | | recruiting domain on Long |
| (<Strand Ref.: 962>) | | strand 5′-3′ of RD1022 |
| mC*mA*fC*mU*fG.mU.fU.mC.fU.mC.fG.mU.fC.mU.mC.mC. | 144 | Recruiting domain on long |
| fC.mA*fC*mC*fC*(X2) | | strand, sequence information |
| (<Strand Ref.: 963>) | | 5′-3′ of RD1022 |

^{€}Unless otherwise specified, nucleic acid sequences are described 5′ to 3′.
Notes for RL0079, RH0001, RD0016, RD0034, and RD0037: capitalized letter indicate unmodified RNA, * stands for phosphorothioate linkages,2′-O-Methyl RNA shown as: [mA], [mG], [mC], [mU]; 2′-fluoro RNA shown as [2flA], [2flU], [2flG], [2flC].
For Strand Ref.: 29-752 - unmodified RNA: rA, rU, rC, rG; ‘.’ stands for phosphoate linkage; ‘*’ stands for Phosphorothioate linkages; 2′-O-Methyl RNA: mA, mG, mC, and mU; 2′-fluoro RNA: fA, fU, fG, and fC; DNA, dA, dC, dG, and dU.
For Strand Ref.: 753-818; long strands (e.g., editing domain linked (e.g., bonded, joined etc.) to a strand of the double-stranded RNA duplex) are made of 3 (where a linker is present,2 where absent) covalently linked components, as from 5′ end to 3′ end - [Recruiting domain]-[linker]-[Editing domain], and the short strands is complementary to recruiting domain and form duplex; unmodified RNA: rA, rU, rC, and rG; ‘.’ stands for phosphoate linkage; ‘*’ stands for Phosphorothioate linkages 2′-O-Methyl RNA: mA, mG, mC, and mU.
^{†}‘NT’ denotes a nucleic acid sequence.
‘UM’ denotes unmodified nucleic acids.
A recruiting domain refers to the nucleic acid of the double-stranded RNA duplex, an editing domain refers to a single-stranded guide nucleic acid.

### Other Embodiments

Embodiment 1. An adenosine deaminase acting on ribonucleic acid (RNA) (ADAR) recruiting molecule comprising a double-stranded RNA duplex, wherein the double-stranded RNA duplex comprises two strands of RNA of an equal number of nucleotides, wherein: (a) the 5′ nucleotide of each RNA strand is complementary to the 3′ nucleotide of the other RNA strand; (b) at least one RNA strand of the double-stranded RNA duplex comprises at least one nucleoside modification and/or at least one backbone modification; and (c) the double-stranded RNA duplex comprises at least one base pair mismatch, wherein the mismatch is not positioned at either terminal nucleotide base pair of the double-stranded RNA duplex.

Embodiment 2. The ADAR recruiting molecule of embodiment 1, further comprising a single-stranded guide nucleic acid.

Embodiment 3. The ADAR recruiting molecule of any one of embodiment 1 or embodiment 2, wherein a double-stranded RNA duplex comprises at least one nucleoside modification and at least one backbone modification.

Embodiment 4. The ADAR recruiting molecule of any one of embodiment 1 or embodiments 2-3, wherein the double-stranded RNA duplex comprises at least one nucleoside modification comprises a 2′-aminoethyl, a 2′-deoxy-2′-fluoro-β-d-arabinonucleic acid, a 2′-O-methyl, a 2′-O-methoxyethyl (2′O-MOE), or a 2′-fluoro modification.

Embodiment 5. The ADAR recruiting molecule of any one of embodiment 1 or embodiments 2-4, wherein the at least one backbone modification of the double-stranded RNA duplex comprises a phosphorothioate modification.

Embodiment 6. The ADAR recruiting molecule of any one of embodiment 1 or embodiments 2-5, wherein the at least one backbone modification of the double-stranded RNA duplex is positioned within 1-5 nucleotides of the terminal nucleotide of the RNA strand on which it is located.

Embodiment 7. The ADAR recruiting molecule of any one of embodiment 1 or embodiments 2-6, wherein the at least one backbone modification of the double-stranded RNA duplex is positioned within 1-3 nucleotides of the terminal nucleotide of the RNA strand on which it is located.

Embodiment 8. The ADAR recruiting molecule of any one of embodiment 1 or embodiments 2-7, wherein the at least one backbone modification of the double-stranded RNA duplex is positioned within 1 nucleotide of the terminal nucleotide of the RNA strand on which it is located.

Embodiment 9. The ADAR recruiting molecule of any one of embodiment 1 or embodiments 2-8, wherein the double-stranded RNA duplex comprises more than one nucleoside modification.

Embodiment 10. The ADAR recruiting molecule of any one of embodiment 1 or embodiments 2-9, wherein the double-stranded RNA duplex comprises more than two nucleoside modifications.

Embodiment 11. The ADAR recruiting molecule of any one of embodiment 1 or embodiments 2-10, wherein more than 25% of the nucleosides in the double-stranded RNA duplex comprise a nucleoside modification.

Embodiment 12. The ADAR recruiting molecule of any one of embodiment 1 or embodiments 2-11, wherein more than 50% of the nucleosides in the double-stranded RNA duplex comprise a nucleoside modification.

Embodiment 13. The ADAR recruiting molecule of any one of embodiment 1 or embodiments 2-12, wherein more than 75% of the nucleosides in the double-stranded RNA duplex comprise a nucleoside modification.

Embodiment 14. The ADAR recruiting molecule of any one of embodiment 1 or embodiments 2-13, wherein the double-stranded RNA duplex comprises more than one backbone modification.

Embodiment 15. The ADAR recruiting molecule of any one of embodiment 1 or embodiments 2-14, wherein the double-stranded RNA duplex comprises more than two backbone modifications.

Embodiment 16. The ADAR recruiting molecule of any one of embodiment 1 or embodiments 2-15, wherein the double-stranded RNA duplex comprises more than three backbone modifications.

Embodiment 17. The ADAR recruiting molecule of any one of embodiment 1 or embodiments 2-16, wherein more than 25% of the internucleoside linkages of the double-stranded RNA duplex comprise a modification.

Embodiment 18. The ADAR recruiting molecule of any one of embodiment 1 or embodiments 2-17, wherein more than 50% of the internucleoside linkages of the double-stranded RNA duplex comprise a modification.

Embodiment 19. The ADAR recruiting molecule of any one of embodiment 1 or embodiments 2-18, wherein more than 75% of the internucleoside linkages of the double-stranded RNA duplex comprise a modification.

Embodiment 20. The ADAR recruiting molecule of any one of embodiment 1 or embodiments 2-19, further comprising nucleotides attached to the 3′ end or 5′ end of at least one of the RNA strands of the double-stranded RNA duplex creating a 3′ and/or 5′ end overhang.

Embodiment 21. The ADAR recruiting molecule of any one of embodiment 1 or embodiments 2-20, further comprising an additional moiety.

Embodiment 22. The ADAR recruiting molecule of any one of embodiment 1 or embodiments 2-21, further comprising a linker.

Embodiment 23. The ADAR recruiting molecule of any one of embodiment 2 or embodiments 3-22, wherein the single-stranded guide nucleic acid is guide Ribonucleic Acid (gRNA).

Embodiment 24. The ADAR recruiting molecule of any one of embodiment 2 or embodiments 3-23, wherein the single-stranded guide nucleic acid comprises at least one nucleoside modification.

Embodiment 25. The ADAR recruiting molecule of any one of embodiment 2 or embodiments 3-23, wherein the single-stranded guide nucleic acid comprises at least one backbone modification.

Embodiment 26. The ADAR recruiting molecule of any one of embodiment 2 or embodiments 3-25, wherein the single-stranded guide nucleic acid comprises at least one nucleoside modification and at least one backbone modification.

Embodiment 27. The ADAR recruiting molecule of any one of embodiment 2 or embodiments 3-26, wherein the single-stranded guide nucleic acid comprises at least two nucleoside modifications.

Embodiment 28. The ADAR recruiting molecule of any one of embodiment 2 or embodiments 3-27, wherein the single-stranded guide nucleic acid comprises at least three nucleoside modifications.

Embodiment 29. The ADAR recruiting molecule of any one of embodiment 2 or embodiments 3-28, wherein more than 25% of the nucleosides of the single-stranded guide nucleic acid comprise a nucleoside modification.

Embodiment 30. The ADAR recruiting molecule of any one of embodiment 2 or embodiments 3-29, wherein more than 50% of the nucleosides in the single-stranded guide nucleic acid comprise a nucleoside modification.

Embodiment 31. The ADAR recruiting molecule of any one of embodiment 2 or embodiments 3-30, wherein more than 75% of the nucleosides in the single-stranded guide nucleic acid comprise a nucleoside modification.

Embodiment 32. The ADAR recruiting molecule of any one of embodiment 2 or embodiments 3-31, wherein the single-stranded guide nucleic acid comprises at least one backbone modification.

Embodiment 33. The ADAR recruiting molecule of any one of embodiment 2 or embodiments 3-32, wherein the single-stranded guide nucleic acid comprises at least two backbone modifications.

Embodiment 34. The ADAR recruiting molecule of any one of embodiment 2 or embodiments 3-33, wherein the single-stranded guide nucleic acid comprises at least three backbone modifications.

Embodiment 35. The ADAR recruiting molecule of any one of embodiment 2 or embodiments 3-34, wherein more than 25% of the internucleoside linkages in the single-stranded guide nucleic acid comprise a phosphate modification.

Embodiment 36. The ADAR recruiting molecule of any one of embodiment 2 or embodiments 3-35, wherein more than 50% of the internucleoside linkages in the single-stranded guide nucleic acid comprise a phosphate modification.

Embodiment 37. The ADAR recruiting molecule of any one of embodiment 2 or embodiments 3-36, wherein more than 75% of the internucleoside linkages in the single-stranded guide nucleic acid comprise a phosphate modification.

Embodiment 38. The ADAR recruiting molecule of any one of embodiment 2 or embodiments 3-37, wherein the single-stranded guide nucleic acid comprises sufficient complementarity to hybridize with a target sequence.

Embodiment 39. The ADAR recruiting molecule of any one of embodiment 2 or embodiments 3-38, wherein the single-stranded guide nucleic acid comprises three consecutive non-modified nucleotides.

Embodiment 40. The ADAR recruiting molecule of any one of embodiment 2 or embodiments 3-39, wherein at least one of the three consecutive non-modified nucleotides pairs with a nucleotide adjacent to a target adenosine in the target sequence.

Embodiment 41. The ADAR recruiting molecule of any one of embodiment 39 or embodiment 40, wherein the middle nucleotide of the three consecutive non-modified nucleotides is opposite the target adenosine.

Embodiment 42. 42. The ADAR recruiting molecule of any one of embodiment 2 or embodiments 3-41, wherein a nucleotide opposite a target adenosine comprises: (a) cytosine (C); (b) a natural or modified nucleotide which does not base pair with adenosine (A); and/or (c) a natural or modified nucleotide which base pairs with guanine (G) or inosine (I).

Embodiment 43. The ADAR recruiting molecule of any one of embodiment 1 or embodiments 2-42, wherein each RNA strand of the double-stranded RNA duplex is at least 5 nucleotides in length.

Embodiment 44. The ADAR recruiting molecule of any one of embodiment 1 or embodiments 2-43, wherein each RNA strand of the double-stranded RNA duplex is fewer than or equal to 100 nucleotides in length.

Embodiment 45. The ADAR recruiting molecule of any one of embodiment 1 or embodiments 2-44, wherein each RNA strand of the double-stranded RNA duplex is about 5 to about 80 nucleotides in length.

Embodiment 46. The ADAR recruiting molecule of any one of embodiment 1 or embodiments 2-45, wherein each RNA strand of the double-stranded RNA duplex is about 5 to about 60 nucleotides in length.

Embodiment 47. The ADAR recruiting molecule of any one of embodiment 1 or embodiments 2-46, wherein each RNA strand of the double-stranded RNA duplex is about 5 to about 40 nucleotides in length.

Embodiment 48. The ADAR recruiting molecule of any one of embodiment 1 or embodiments 2-47, wherein each RNA strand of the double-stranded RNA duplex is about 5 to about 30 nucleotides in length.

Embodiment 49. The ADAR recruiting molecule of any one of embodiment 1 or embodiments 2-48, wherein each RNA strand of the double-stranded RNA duplex is about 5 to about 20 nucleotides in length.

Embodiment 50. The ADAR recruiting molecule of any one of embodiment 1 or embodiments 2-49, wherein each RNA strand of the double-stranded RNA duplex is about 5 to about 10 nucleotides in length.

Embodiment 51. The ADAR recruiting molecule of any one of embodiment 2 or embodiments 3-50, wherein the single-stranded guide nucleic acid is at least 5 nucleotides in length.

Embodiment 52. The ADAR recruiting molecule of any one of embodiment 2 or embodiments 3-51, wherein the single-stranded guide nucleic acid is fewer than or equal to 100 nucleotides in length.

Embodiment 53. The ADAR recruiting molecule of any one of embodiment 2 or embodiments 3-52, wherein the single-stranded guide nucleic acid is about 5 to about 80 nucleotides in length.

Embodiment 54. The ADAR recruiting molecule of any one of embodiment 2 or embodiments 3-53, wherein the single-stranded guide nucleic acid is about 5 to about 60 nucleotides in length.

Embodiment 55. The ADAR recruiting molecule of any one of embodiment 2 or embodiments 3-54, wherein the single-stranded guide nucleic acid is about 5 to about 40 nucleotides in length.

Embodiment 56. The ADAR recruiting molecule of any one of embodiment 2 or embodiments 3-55, wherein the single-stranded guide nucleic acid is about 5 to about 30 nucleotides in length.

Embodiment 57. The ADAR recruiting molecule of any one of embodiment 2 or embodiments 3-56, wherein the single-stranded guide nucleic acid is about 5 to about 20 nucleotides in length.

Embodiment 58. The ADAR recruiting molecule of any one of embodiment 2 or embodiments 3-57, wherein the single-stranded guide nucleic acid is about 5 to about 10 nucleotides in length.

Embodiment 59. The ADAR recruiting molecule of any one of embodiment 2 or embodiments 3-58, wherein the single-stranded guide nucleic acid comprises at least 50% complementarity with a target sequence.

Embodiment 60. The ADAR recruiting molecule of any one of embodiment 2 or embodiments 3-59, wherein the single-stranded guide nucleic acid comprises at least 70% complementarity with a target sequence.

Embodiment 61. The ADAR recruiting molecule of any one of embodiment 2 or embodiments 3-60, wherein the single-stranded guide nucleic acid comprises at least 80% complementarity with a target sequence.

Embodiment 62. The ADAR recruiting molecule of any one of embodiment 2 or embodiments 3-61, wherein the single-stranded guide nucleic acid comprises at least 90% complementarity with a target sequence.

Embodiment 63. The ADAR recruiting molecule of any one of embodiment 2 or embodiments 3-62, wherein the single-stranded guide nucleic acid comprises at least 95% complementarity with a target sequence.

Embodiment 64. An RNA targeting molecule comprising: (a) a double-stranded RNA duplex, wherein the double-stranded RNA duplex comprises two strands of RNA of an equal number of nucleotides, wherein the 5′ nucleotide of each RNA strand is complementary to the 3′ nucleotide of the other RNA strand, wherein the double-stranded RNA duplex comprises at least one base pair mismatch, wherein the mismatch is not positioned at either terminal nucleotide base pair of the double-stranded RNA duplex; and (b) a single-stranded guide nucleic acid.

Embodiment 65. The RNA targeting molecule of embodiment 64, wherein the double-stranded RNA duplex comprises at least one nucleoside modification, and/or at least one backbone modification.

Embodiment 66. The RNA targeting molecule of any one of embodiment 64 or embodiments 65, wherein the double-stranded RNA duplex comprises at least one nucleoside modification and at least one backbone modification.

Embodiment 67. The RNA targeting molecule of any one of embodiment 64 or embodiments 65-66, wherein the at least one nucleoside modification comprises a 2′-aminoethyl, a 2′-deoxy-2′-fluoro-β-d-arabinonucleic acid, a 2′-O-methyl, a 2′-O-methoxyethyl (2′O-MOE), or a 2′-fluoro modification.

Embodiment 68. The RNA targeting molecule of any one of embodiment 64 or embodiments 65-67, wherein the at least one backbone modification comprises a phosphorothioate modification.

Embodiment 69. The RNA targeting molecule of any one of embodiment 64 or embodiments 65-68, wherein the at least one backbone modification of the double-stranded RNA duplex is positioned within 1-5 nucleotides of the terminal nucleotide of the RNA strand on which it is located.

Embodiment 70. The RNA targeting molecule of any one of embodiment 64 or embodiments 65-69, wherein the at least one backbone modification of the double-stranded RNA duplex is positioned within 1-3 nucleotides of the terminal nucleotide of the RNA strand on which it is located.

Embodiment 71. The RNA targeting molecule of any one of embodiment 64 or embodiments 65-70, wherein the at least one backbone modification of the double-stranded RNA duplex is positioned within 1 nucleotide of the terminal nucleotide of the RNA strand on which it is located.

Embodiment 72. The RNA targeting molecule of any one of embodiment 64 or embodiments 65-71, wherein the double-stranded RNA duplex comprises more than one nucleoside modification.

Embodiment 73. The RNA targeting molecule of any one of embodiment 64 or embodiments 65-72, wherein the double-stranded RNA duplex comprises more than two nucleoside modifications.

Embodiment 74. The RNA targeting molecule of any one of embodiment 64 or embodiments 65-73, wherein more than 25% of the nucleosides in the double-stranded RNA duplex comprise a nucleoside modification.

Embodiment 75. The RNA targeting molecule of any one of embodiment 64 or embodiments 65-74, wherein more than 50% of the nucleosides in the double-stranded RNA duplex comprise a nucleoside modification.

Embodiment 76. The RNA targeting molecule of any one of embodiment 64 or embodiments 65-75, wherein more than 75% of the nucleosides in the double-stranded RNA duplex comprise a nucleoside modification.

Embodiment 77. The RNA targeting molecule of any one of embodiment 64 or embodiments 65-76, wherein the double-stranded RNA duplex comprises more than one backbone modification.

Embodiment 78. The RNA targeting molecule of any one of embodiment 64 or embodiments 65-77, wherein the double-stranded RNA duplex comprises more than two backbone modification.

Embodiment 79. The RNA targeting molecule of any one of embodiment 64 or embodiments 65-78, wherein the double-stranded RNA duplex comprises more than three backbone modification.

Embodiment 80. The RNA targeting molecule of any one of embodiment 64 or embodiments 65-79, wherein more than 25% of the internucleoside linkages of the double-stranded RNA duplex comprise a modification.

Embodiment 81. The RNA targeting molecule of any one of embodiment 64 or embodiments 65-80, wherein more than 50% of the internucleoside linkages of the double-stranded RNA duplex comprise a modification.

Embodiment 82. The RNA targeting molecule of any one of embodiment 64 or embodiments 65-81, wherein more than 75% of the internucleoside linkages of the double-stranded RNA duplex comprise a modification.

Embodiment 83. The RNA targeting molecule of any one of embodiment 64 or embodiments 65-82, further comprising nucleotides attached to the 3′ end or 5′ end of at least one of the RNA strands of the double-stranded RNA duplex creating a 3′ and/or 5′ end overhang.

Embodiment 84. The RNA targeting molecule of any one of embodiment 64 or embodiments 65-83, further comprising an additional moiety.

Embodiment 85. The RNA targeting molecule of any one of embodiment 64 or embodiments 65-84, further comprising a linker.

Embodiment 86. The RNA targeting molecule of any one of embodiments 64 or embodiments 65-85, wherein the single-stranded guide nucleic acid is guide ribonucleic acid (gRNA).

Embodiment 87. The RNA targeting molecule of any one of embodiments 64 or embodiments 65-86, wherein the single-stranded guide nucleic acid comprises at least one nucleoside modification.

Embodiment 88. The RNA targeting molecule of any one of embodiments 64 or embodiments 65-87, wherein the single-stranded guide nucleic acid comprises at least one backbone modification.

Embodiment 89. The RNA targeting molecule of any one of embodiment 64 or embodiments 65-88, wherein the single-stranded guide nucleic acid comprises at least one nucleoside modification and at least one backbone modification.

Embodiment 90. The RNA targeting molecule of any one of embodiment 64 or embodiments 65-89, wherein the single-stranded guide nucleic acid comprises at least two nucleoside modifications.

Embodiment 91. The RNA targeting molecule of any one of embodiment 64 or embodiments 65-90, wherein the single-stranded guide nucleic acid comprises at least three nucleoside modifications.

Embodiment 92. The RNA targeting molecule of any one of embodiment 64 or embodiments 65-91, more than 25% of the nucleosides of the single-stranded guide nucleic acid comprise a nucleoside modification.

Embodiment 93. The RNA targeting molecule of any one of embodiment 64 or embodiments 65-92, wherein more than 50% of the nucleosides in the single-stranded guide nucleic acid comprise a nucleoside modification.

Embodiment 94. The RNA targeting molecule of any one of embodiment 64 or embodiments 65-93, wherein more than 75% of the nucleosides in the single-stranded guide nucleic acid comprise a nucleoside modification.

Embodiment 95. The RNA targeting molecule of any one of embodiment 64 or embodiments 65-94, wherein the single-stranded guide nucleic acid comprises at least one backbone modification.

Embodiment 96. The RNA targeting molecule of any one of embodiment 64 or embodiments 65-95, wherein the single-stranded guide nucleic acid comprises at least two backbone modifications.

Embodiment 97. The RNA targeting molecule of any one of embodiment 64 or embodiments 65-96, wherein the single-stranded guide nucleic acid comprises at least three backbone modifications.

Embodiment 98. The RNA targeting molecule of any one of embodiment 64 or embodiments 65-97, wherein more than 25% of the internucleoside linkages in the single-stranded guide nucleic acid comprise a phosphate modification.

Embodiment 99. The RNA targeting molecule of any one of embodiment 64 or embodiments 65-98, wherein more than 50% of the internucleoside linkages in the single-stranded guide nucleic acid comprise a phosphate modification.

Embodiment 100. The RNA targeting molecule of any one of embodiment 64 or embodiments 65-99, wherein more than 75% of the internucleoside linkages in the single-stranded guide nucleic acid comprise a phosphate modification.

Embodiment 101. The RNA targeting molecule of any one of embodiment 64 or embodiments 65-100, wherein the single-stranded guide nucleic acid comprises sufficient complementarity to hybridize with a target sequence.

Embodiment 102. The RNA targeting molecule of any one of embodiment 64 or embodiments 65-101, wherein the single-stranded guide nucleic acid comprises three consecutive non-modified nucleotides.

Embodiment 103. The RNA targeting molecule of embodiment 102, wherein at least one of the three consecutive non-modified nucleotides of the single-stranded guide nucleic acid is complementary to a nucleotide adjacent to a target adenosine in the target sequence.

Embodiment 104. The RNA targeting molecule of any one of embodiment 102 or embodiment 103, wherein the middle nucleotide of the three consecutive non-modified nucleotide is opposite the target adenosine.

Embodiment 105. The RNA targeting molecule of any one of embodiment 64 or embodiment 65-104, wherein a nucleotide opposite a target adenosine comprises: (a) cytosine (C); (b) a natural or modified nucleotide which does not base pair with adenosine (A); and/or (c) a natural or modified nucleotide which base pairs with guanine (G) or inosine (I).

Embodiment 106. The RNA targeting molecule of any one of embodiment 64 or embodiments 65-105, wherein each RNA strand of the double-stranded RNA duplex is at least 5 nucleotides in length.

Embodiment 107. The RNA targeting molecule of any one of embodiment 64 or embodiments 65-106, wherein each RNA strand of the double-stranded RNA duplex is fewer than or equal to 100 nucleotides in length.

Embodiment 108. The RNA targeting molecule of any one of embodiment 64 or embodiments 65-107, wherein each RNA strand of the double-stranded RNA duplex is about 5 to about 80 nucleotides in length.

Embodiment 109. The RNA targeting molecule of any one of embodiment 64 or embodiments 65-108, wherein each RNA strand of the double-stranded RNA duplex is about 5 to about 60 nucleotides in length.

Embodiment 110. The RNA targeting molecule of any one of embodiment 64 or embodiments 65-109, wherein each RNA strand of the double-stranded RNA duplex is about 5 to about 40 nucleotides in length.

Embodiment 111. The RNA targeting molecule of any one of embodiment 64 or embodiments 65-110, wherein each RNA strand of the double-stranded RNA duplex is about 5 to about 30 nucleotides in length.

Embodiment 112. The RNA targeting molecule of any one of embodiment 64 or embodiments 65-111, wherein each RNA strand of the double-stranded RNA duplex is about 5 to about 20 nucleotides in length.

Embodiment 113. The RNA targeting molecule of any one of embodiment 64 or embodiments 65-112, wherein each RNA strand of the double-stranded RNA duplex is about 5 to about 10 nucleotides in length.

Embodiment 114. The RNA targeting molecule of any one of embodiment 64 or embodiments 65-113, wherein the single-stranded guide nucleic acid is at least 5 nucleotides in length.

Embodiment 115. The RNA targeting molecule of any one of embodiment 64 or embodiments 65-114, wherein the single-stranded guide nucleic acid is fewer than or equal to 100 nucleotides in length.

Embodiment 116. The RNA targeting molecule of any one of embodiment 64 or embodiments 65-115, wherein the single-stranded guide nucleic acid is about 5 to about 80 nucleotides in length.

Embodiment 117. The RNA targeting molecule of any one of embodiment 64 or embodiments 65-116, wherein the single-stranded guide nucleic acid is about 5 to about 60 nucleotides in length.

Embodiment 118. The RNA targeting molecule of any one of embodiment 64 or embodiments 65-117, wherein the single-stranded guide nucleic acid is about 5 to about 40 nucleotides in length.

Embodiment 119. The RNA targeting molecule of any one of embodiment 64 or embodiments 65-118, wherein the single-stranded guide nucleic acid is about 5 to about 30 nucleotides in length.

Embodiment 120. The RNA targeting molecule of any one of embodiment 64 or embodiments 65-119 wherein the single-stranded guide nucleic acid is about 5 to about 20 nucleotides in length.

Embodiment 121. The RNA targeting molecule of any one of embodiment 64 or embodiments 65-120, wherein the single-stranded guide nucleic acid is about 5 to about 10 nucleotides in length.

Embodiment 122. The RNA targeting molecule of any one of embodiment 64 or embodiments 65-121, wherein the single-stranded guide nucleic acid comprises at least 50% complementarity with a target sequence.

Embodiment 123. The RNA targeting molecule of any one of embodiment 64 or embodiments 65-122, wherein the single-stranded guide nucleic acid comprises at least 70% complementarity with a target sequence.

Embodiment 124. The RNA targeting molecule of any one of embodiment 64 or embodiments 65-123, wherein the single-stranded guide nucleic acid comprises at least 80% complementarity with a target sequence.

Embodiment 125. The RNA targeting molecule of any one of embodiment 64 or embodiments 65-124, wherein the single-stranded guide nucleic acid comprises at least 90% complementarity with a target sequence.

Embodiment 126. The RNA targeting molecule of any one of embodiment 64 or embodiments 65-125, wherein the single-stranded guide nucleic acid comprises at least 95% complementarity with a target sequence.

Embodiment 127. A method of deaminating a target nucleic acid in a subject, comprising, administering an effective amount of the ADAR recruiting molecule of any one of embodiments 2-63, and/or the RNA targeting molecule of any one of embodiments 64-126, wherein the ADAR recruiting molecule and/or the RNA targeting molecule comprises a single-stranded guide nucleic acid comprising a sequence which is sufficiently complementary to a target sequence to hybridize with the target sequence.

Embodiment 128. The method of embodiment 127, wherein the target sequence comprises a target adenosine.

Embodiment 129. A method of treating a subject, comprising administering the ADAR recruiting molecule of any one of embodiments 2-63, and/or the RNA targeting molecule of any one of embodiments 64-126, wherein the ADAR recruiting molecule and/or the RNA targeting molecule comprises a single-stranded guide nucleic acid comprising a sequence which is sufficiently complementary to a target sequence to hybridize with the target sequence.

Embodiment 130. The method of embodiment 129, wherein the target sequence comprises a target adenosine.

Embodiment 131. The method of embodiment 130, wherein the target adenosine is related to a disease or disorder, wherein the deamination of the target adenosine treats the disease or disorder.

Embodiment 132. The method of embodiment 131, wherein the disease or disorder is selected from: Cystic fibrosis, Hurler Syndrome, Parkinson's disease, Alzheimer's disease, albinism, Amyotrophic lateral sclerosis, Asthma, beta-thalassemia (β-thalassemia), Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B, and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, Sturge-Weber Syndrome, and cancer.

Embodiment 133. An RNA targeting molecule comprising: (a) a double-stranded RNA duplex comprising two RNA strands; (b) a single-stranded guide nucleic acid; and (c) a linker; wherein the double-stranded RNA duplex is connected to the single-stranded guide nucleic acid via the linker.

Embodiment 134. The RNA targeting molecule of embodiment 133, wherein the linker is connected to the 5′ terminal nucleotide of one of the two strands of the RNA duplex.

Embodiment 135. The RNA targeting molecule of embodiment 133, wherein the linker is connected to the 3′ terminal nucleotide of one of the two strands of the RNA duplex.

Embodiment 136. The RNA targeting molecule of embodiment 133, wherein the linker is connected to a nucleotide located between the 5′ terminal nucleotide and the 3′ terminal nucleotide of one of the two strands of the RNA duplex.

Embodiment 137. The RNA targeting molecule of embodiment 133, wherein the linker is connected to the 5′ nucleotide of the guide nucleic acid.

Embodiment 138. The RNA targeting molecule of embodiment 133, wherein the linker is connected to the 3′ nucleotide of the guide nucleic acid.

Embodiment 139. The RNA targeting molecule of embodiment 133, wherein the linker is connected to a nucleotide located between the 5′ terminal nucleotide and the 3′ terminal nucleotide the guide nucleic acid.

Embodiment 140. The RNA targeting molecule of embodiment 133, wherein the linker is connected to a sugar of the 5′ terminal nucleotide, or a 3′ hydroxyl or sugar of the 3′ terminal nucleotide of one strand of the RNA duplex.

Embodiment 141. The RNA targeting molecule of embodiment 133, wherein the linker is connected to a sugar of the 5′ terminal nucleotide, or a 3′ hydroxyl or sugar of the 3′ terminal nucleotide of the guide nucleic acid.

Embodiment 142. The RNA targeting molecule of embodiment 133, wherein the linker is connected to an internucleoside linkage of one RNA strand of the RNA duplex.

Embodiment 143. The RNA targeting molecule of embodiment 133, wherein the linker is connected to an internucleoside linkage of the guide nucleic acid.

Embodiment 144. The RNA targeting molecule of embodiment 133, wherein the linker is connected to a nucleoside sugar of one RNA strand of the RNA duplex.

Embodiment 145. The RNA targeting molecule of embodiment 133, wherein the linker is connected to a nucleoside sugar of the guide nucleic acid.

Embodiment 146. The RNA targeting molecule of embodiment 133, wherein the linker connects the 3′ end of the guide nucleic acid to the 5′ end of one RNA strand of the RNA duplex.

Embodiment 147. The RNA targeting molecule of embodiment 133, wherein the linker connects the 5′ end of the guide nucleic acid to the 3′ end of one RNA strand of the RNA duplex.

Embodiment 148. The RNA targeting molecule of embodiment 133, wherein the linker connects the 3′ end of the guide nucleic acid to the 3′ end of one RNA strand of the RNA duplex.

Embodiment 149. The RNA targeting molecule of embodiment 133, wherein the linker connects the 5′ end of the guide nucleic acid to the 5′ end of one RNA strand of the RNA duplex.

Embodiment 150. The RNA targeting molecule of embodiment 133, wherein the linker connects the 3′ end of the guide nucleic acid to a nucleotide located between the 5′ terminal nucleotide and the 3′ terminal nucleotide of one RNA strand of the RNA duplex.

Embodiment 151. The RNA targeting molecule of embodiment 150, wherein the linker is connected to an internucleoside linkage or a nucleoside sugar of one RNA strand of the RNA duplex.

Embodiment 152. The RNA targeting molecule of embodiment 133, wherein the linker connects the 5′ end of the guide nucleic acid to a nucleotide located between the 5′ terminal nucleotide and the 3′ terminal nucleotide of one RNA strand of the RNA duplex.

Embodiment 153. The RNA targeting molecule of embodiment 152, wherein the linker is connected to an internucleoside linkage or a nucleoside sugar of one RNA strand of the RNA duplex.

Embodiment 154. The RNA targeting molecule of embodiment 133, wherein the linker connects the 3′ end of one RNA strand of the RNA duplex to a nucleotide located between the 5′ terminal nucleotide and the 3′ terminal nucleotide of the guide nucleic acid.

Embodiment 155. The RNA targeting molecule of embodiment 154, wherein the linker is connected to an internucleoside linkage or a nucleoside sugar of the guide nucleic acid.

Embodiment 156. The RNA targeting molecule of embodiment 133, wherein the linker connects the 5′ end of one RNA strand of the RNA duplex to a nucleotide located between the 5′ terminal nucleotide and the 3′ terminal nucleotide of the guide nucleic acid.

Embodiment 157. The RNA targeting molecule of embodiment 156, wherein the linker is connected to an internucleoside linkage or a nucleoside sugar of the guide nucleic acid.

Embodiment 158. The RNA targeting molecule of embodiment 133, or any other prior embodiment, wherein the linker is an unbranched linker.

Embodiment 159. The RNA targeting molecule of embodiment 133, or any other prior embodiment, wherein the linker is a branched linker.

Embodiment 160. The RNA targeting molecule of embodiment 133, or any other prior embodiment, wherein the linker is a non-covalent linker comprising a first binding partner covalently attached to one strand of the double-stranded RNA duplex, and a second binding partner covalently attached to the single-stranded guide nucleic acid, wherein the first and second binding partners form a non-covalent complex connecting the double-stranded RNA duplex to the single-stranded guide nucleic acid.

Embodiment 161. The RNA targeting molecule of embodiment 160, wherein the first binding partner is a receptor the second binding partner is a ligand specific for the receptor.

Embodiment 162. The RNA targeting molecule of embodiment 160, wherein the second binding partner is a receptor and the first binding partner is a ligand specific for the receptor.

Embodiment 163. The RNA targeting molecule of embodiment 160, wherein the first binding partner is biotin and the second binding partner is streptavidin.

Embodiment 164. The RNA targeting molecule of embodiment 160, wherein the first binding partner is streptavidin and the second binding partner is biotin.

Embodiment 165. The RNA targeting molecule of embodiment 133, or any other prior embodiment, wherein the linker is a covalent linker.

Embodiment 166. The RNA targeting molecule of embodiment 133, or any other prior embodiment, wherein the linker is greater than or equal to 4 atoms in length.

Embodiment 167. The RNA targeting molecule of embodiment 133, or any other prior embodiment, wherein the linker is fewer than or equal to 180 atoms in length.

Embodiment 168. The RNA targeting molecule of embodiment 165, wherein the linker comprises an alkyl, alkenyl, alkynyl, substituted alkyl, substituted alkenyl, substituted alkynyl, repeated ethylene glycol group, ether, thioether, urea, carbonate, amine, amide, maleimide-thioether, disulfide, phosphodiester, sulfonamide linkage, a product of a click reaction, a triazole from an azide-alkyne cycloaddition, carbamate, a cleavable linker such as, a redox cleavable linker such as a reductively cleavable linker, a disulfide group, an acid cleavable linker, a hydrazone group, an ester group, an acetal group, or a ketal group, an esterase cleavable linker, an ester group, a phosphatase cleavable linker, a phosphate group, or a peptidase cleavable linker, a peptide bond, a bio-cleavable linker, DNA, RNA, disulfide, amide, functionalized monosaccharides, or oligosaccharides of galactosamine.

Embodiment 169. The RNA targeting molecule of embodiment 165, wherein the linker comprises a moiety derived from a click chemistry reaction.

Embodiment 170. The RNA targeting molecule of embodiment 169, wherein the moiety derived from a click chemistry reaction is a triazole, diazole, diazine, sulfide bond, maleimide ring, succinimide ring, ester, or amide.

Embodiment 171. The RNA targeting molecule of embodiment 165, wherein the linker comprises one or more amino acids.

Embodiment 172. The RNA targeting molecule of embodiment 165, wherein the linker comprises an organic molecule, group, polymer, or chemical domain.

Embodiment 173. The RNA targeting molecule of embodiment 172, wherein the chemical domain comprises an amide, urea, carbamate, carbonate, ester, acetal, ketal, phosphoramidite, hydrazone, imine, oxime, disulfide, silyl, hydrazine, hydrazone, thiol, imidazole, carbon-carbon bond, carbon-heteroatom bond, or azo domain.

Embodiment 174. The RNA targeting molecule of embodiment 165, wherein the linker is polymeric.

Embodiment 175. The RNA targeting molecule of embodiment 174, wherein the polymeric linker comprises polyethylene, polyethylene glycol, polyamide, polyester, or polyether.

Embodiment 176. The RNA targeting molecule of embodiment 133, or any other prior embodiment, wherein the linker comprises any one of Formula (I)-Formula (VII).

Embodiment 177. The RNA targeting molecule of embodiment 133, or any other prior embodiment, wherein the double-stranded RNA duplex comprises: (a) an RNA strand comprising a sequence with at least 70% identity to Strand Ref.: 24 or 27; and (b) an RNA strand comprising a sequence with at least 70% identity to Strand Ref.: 25 or 28; and wherein the single-stranded guide nucleic acid comprises a sequence with at least 70% identity to Strand Ref.: 23 or 26.

Embodiment 178. The RNA targeting molecule of embodiment 133, or any other prior embodiment, wherein the double-stranded RNA duplex comprises: (a) an RNA strand comprising a sequence according to Strand Ref.: 24 or 27; and (b) an RNA strand comprising a sequence according to Strand Ref.: 25 or 28; and wherein, the at least one single-stranded guide nucleic acid comprises a sequence according to Strand Ref.: 23 or 26.

Embodiment 179. The RNA targeting molecule of any one of embodiments 133-176, wherein the double-stranded RNA duplex comprises: (a) an RNA strand comprising a sequence with at least 70% identity to Strand Ref.: 353 or 355; and (b) an RNA strand comprising a sequence with at least 70% identity to Strand Ref.: 815 or 818.

Embodiment 180. The RNA targeting molecule of any one of embodiments 133-176, wherein the double-stranded RNA duplex comprises: (a) an RNA strand comprising a sequence according to Strand Ref.: 353 or 355; and (b) an RNA strand comprising a sequence according to Strand Ref.: 815 or 818.

Embodiment 181. The RNA targeting molecule of any one of embodiments 133-176, wherein the double-stranded RNA duplex comprises: (a) an RNA strand comprising a sequence with at least 70% identity to Strand Ref.: 641 or 643; and (b) an RNA strand comprising a sequence with at least 70% identity to Strand Ref.: 841 or 869.

Embodiment 182. The RNA targeting molecule of any one of embodiments 133-176, wherein the double-stranded RNA duplex comprises: (a) an RNA strand comprising a sequence according to Strand Ref.: 641 or 643; and (b) an RNA strand comprising a sequence according to Strand Ref.: 841 or 869.

Embodiment 183. The RNA targeting molecule of embodiment 133, or any other prior embodiment, comprising two or more double-stranded RNA duplexes.

Embodiment 184. The RNA targeting molecule of embodiment 133, or any other prior embodiments, comprising two or more single-stranded guide nucleic acids.

Embodiment 185. The RNA targeting molecule of embodiment 133, or any other prior embodiment, comprising 2-10 double-stranded RNA duplexes.

Embodiment 186. The RNA targeting molecule of embodiment 133, or any other prior embodiments, comprising 2-10 single-stranded guide nucleic acids.

Embodiment 187. The RNA targeting molecule of embodiment 133, or any other prior embodiment, comprising 2-5 double-stranded RNA duplexes.

Embodiment 188. The RNA targeting molecule of embodiment 133, or any other prior embodiments, comprising 2-5 single-stranded guide nucleic acids.

Embodiment 189. The RNA targeting molecule of any one of embodiments 133-188, wherein one strand of the double-stranded RNA duplex is not covalently connected to the other strand of the RNA duplex.

Embodiment 190. The RNA targeting molecule of any one of embodiments 133-189, wherein the double-stranded RNA duplex does not comprise a hairpin connecting one strand of the RNA duplex to the other strand of the RNA duplex.

Embodiment 191. The RNA targeting molecule of any one of embodiments 133-190, wherein the double-stranded RNA duplex comprises two RNA strands having an equal number of nucleotides.

Embodiment 192. The RNA targeting molecule of any one of embodiments 133-191, wherein the double-stranded RNA duplex comprises two RNA strands having a different number of nucleotides.

Embodiment 193. The RNA targeting molecule of any one of embodiments 133-192, wherein the linker does not comprise a nucleotide or nucleoside.

Embodiment 194. The RNA targeting molecule of any one of embodiments 133-193, wherein the linker is a non-nucleic acid linker.

Embodiment 195. An RNA targeting molecule comprising: (a) a first double-stranded RNA duplex comprising two RNA strands; (b) a second double-stranded RNA duplex comprising two RNA strands; (b) a single-stranded guide nucleic acid; and (c) a linker; wherein the first double-stranded RNA duplex is connected to the second double-stranded RNA duplex via the linker.

Embodiment 196. An RNA targeting molecule comprising: (a) a double-stranded RNA duplex comprising two RNA strands; (b) a first single-stranded guide nucleic acid; (c) a second single-stranded guide nucleic acid; and (c) a linker; wherein the first single-stranded guide nucleic acid is connected to the second single-stranded guide nucleic acid via the linker.

Embodiment 197. The RNA targeting molecule of embodiment 133, or any other prior embodiment, wherein the double-stranded RNA duplex comprises at least one mismatch.

Embodiment 198. The RNA targeting molecule of embodiment 133, or any other prior embodiment, wherein the single-stranded guide nucleic acid comprises at least two mismatches relative to a target sequence.

In addition to the embodiments expressly described herein, it is to be understood that all of the features disclosed in this disclosure may be combined in any combination (e.g., permutation, combination). Each element disclosed in the disclosure may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, and can make various changes and modifications of the invention to adapt it to various usages and conditions. Thus, other embodiments are also within the claims.

General Techniques

The practice of the subject matter of the disclosure will employ, unless otherwise indicated, conventional techniques of molecular biology (including recombinant techniques), microbiology, cell biology, biochemistry and immunology, which are within the skill of the art. Such techniques are explained fully in the literature, such as, but without limiting, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### EQUIVALENTS AND SCOPE

It is to be understood that this disclosure is not limited to any or all of the particular embodiments described expressly herein, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. As will be readily apparent to the skilled artisan, and should be understood from the terms used herein, where words or terms are defined herein, their applicability should not be limited to the embodiments immediately preceding or following the definition and should be used where context permits throughout the disclosure.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents (i.e., any lexicographical definition in the publications and patents cited that is not also expressly repeated in the disclosure should not be treated as such and should not be read as defining any terms appearing in the accompanying claims). If there is a conflict between any of the incorporated references and this disclosure, this disclosure shall control. In addition, any particular embodiment of this disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

In the claims articles such as “a,” “an,” and “the” may mean one or more than one unless indicated to the contrary or otherwise evident from the context. Wherever used herein, a pronoun in a gender (e.g., masculine, feminine, neuter, other, etc.) the pronoun shall be construed as gender neutral (e.g., construed to refer to all genders equally) regardless of the implied gender unless the context clearly indicates or requires otherwise. Wherever used herein, words used in the singular include the plural, and words used in the plural includes the singular, unless the context clearly indicates or requires otherwise. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists (e.g., in Markush group format), each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included in such ranges unless otherwise specified. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the disclosure, as defined in the following claims.

What is claimed is:

1. An adenosine deaminase acting on ribonucleic acid (RNA) (ADAR) recruiting molecule comprising a double-stranded RNA duplex, wherein the double-stranded RNA duplex comprises two strands of RNA of an equal number of nucleotides, which two RNA strands are not connected to one another by means of a hairpin, wherein:
(a) the 5′ nucleotide of each RNA strand is complementary to the 3′ nucleotide of the other RNA strand;
(b) at least one RNA strand of the double-stranded RNA duplex comprises at least one nucleoside modification and/or at least one backbone modification; and
(c) the double-stranded RNA duplex comprises at least one base pair mismatch, wherein the mismatch is not positioned at either terminal nucleotide base pair of the double-stranded RNA duplex.

2. The ADAR recruiting molecule of claim 1, further comprising a single-stranded guide nucleic acid.

3. The ADAR recruiting molecule of claim 1 or claim 2, wherein the double-stranded RNA duplex comprises at least one nucleoside modification and at least one backbone modification.

4. The ADAR recruiting molecule of any one of claims 1-3, wherein the at least one nucleoside modification of the double-stranded RNA duplex comprises a 2′-aminoethyl, a 2′-deoxy-2′-fluoro-β-d-arabinonucleic acid, a 2′-O-methyl, a 2′-O-methoxyethyl (2′O-MOE), or a 2′-fluoro modification.

5. The ADAR recruiting molecule of any one of claims 1-4, wherein the at least one backbone modification of the double-stranded RNA duplex comprises a phosphorothioate modification.

6. The ADAR recruiting molecule of any one of claims 1-5, wherein the at least one backbone modification of the double-stranded RNA duplex is positioned within 1-5 nucleotides of the terminal nucleotide of the RNA strand on which it is located.

7. The ADAR recruiting molecule of any one of claims 1-6, wherein the at least one backbone modification of the double-stranded RNA duplex is positioned within 1-3 nucleotides of the terminal nucleotide of the RNA strand on which it is located.

8. The ADAR recruiting molecule of any one of claims 1-7, wherein the at least one backbone modification of the double-stranded RNA duplex is positioned within 1 nucleotide of the terminal nucleotide of the RNA strand on which it is located.

9. The ADAR recruiting molecule of any one of claims 1-8, wherein the double-stranded RNA duplex comprises more than one nucleoside modification.

10. The ADAR recruiting molecule of any one of claims 1-9, wherein the double-stranded RNA duplex comprises more than two nucleoside modifications.

11. The ADAR recruiting molecule of any one of claims 1-10, wherein more than 25% of the nucleosides in the double-stranded RNA duplex comprise a nucleoside modification.

12. The ADAR recruiting molecule of any one of claims 1-11, wherein more than 50% of the nucleosides in the double-stranded RNA duplex comprise a nucleoside modification.

13. The ADAR recruiting molecule of any one of claims 1-12, wherein more than 75% of the nucleosides in the double-stranded RNA duplex comprise a nucleoside modification.

14. The ADAR recruiting molecule of any one of claims 1-13, wherein the double-stranded RNA duplex comprises more than one backbone modification.

15. The ADAR recruiting molecule of any one of claims 1-14, wherein the double-stranded RNA duplex comprises more than two backbone modifications.

16. The ADAR recruiting molecule of any one of claims 1-15, wherein the double-stranded RNA duplex comprises more than three backbone modifications.

17. The ADAR recruiting molecule of any one of claims 1-16, wherein more than 25% of the internucleoside linkages of the double-stranded RNA duplex comprise a modification.

18. The ADAR recruiting molecule of any one of claims 1-17, wherein more than 50% of the internucleoside linkages of the double-stranded RNA duplex comprise a modification.

19. The ADAR recruiting molecule of any one of claims 1-18, wherein more than 75% of the internucleoside linkages of the double-stranded RNA duplex comprise a modification.

20. The ADAR recruiting molecule of any one of claims 1-19, further comprising nucleotides attached to the 3′ end or 5′ end of at least one of the RNA strands of the double-stranded RNA duplex creating a 3′ or 5′ end overhang.

21. The ADAR recruiting molecule of any one of claims 1-20, further comprising an additional moiety.

22. The ADAR recruiting molecule of any one of claims 1-21, further comprising a linker.

23. The ADAR recruiting molecule of any one of claims 2-22, wherein the single-stranded guide nucleic acid is guide Ribonucleic Acid (gRNA).

24. The ADAR recruiting molecule of any one of claims 2-23, wherein the single-stranded guide nucleic acid comprises at least one nucleoside modification.

25. The ADAR recruiting molecule of any one of claims 2-23, wherein the single-stranded guide nucleic acid comprises at least one backbone modification.

26. The ADAR recruiting molecule of any one of claims 2-25, wherein the single-stranded guide nucleic acid comprises at least one nucleoside modification and at least one backbone modification.

27. The ADAR recruiting molecule of any one of claims 2-26, wherein the single-stranded guide nucleic acid comprises at least two nucleoside modifications.

28. The ADAR recruiting molecule of any one of claims 2-27, wherein the single-stranded guide nucleic acid comprises at least three nucleoside modifications.

29. The ADAR recruiting molecule of any one of claims 2-28, wherein more than 25% of the nucleosides of the single-stranded guide nucleic acid comprise a nucleoside modification.

30. The ADAR recruiting molecule of any one of claims 2-29, wherein more than 50% of the nucleosides in the single-stranded guide nucleic acid comprise a nucleoside modification.

31. The ADAR recruiting molecule of any one of claims 2-30, wherein more than 75% of the nucleosides in the single-stranded guide nucleic acid comprise a nucleoside modification.

32. The ADAR recruiting molecule of any one of claims 2-31, wherein the single-stranded guide nucleic acid comprises at least one backbone modification.

33. The ADAR recruiting molecule of any one of claims 2-32, wherein the single-stranded guide nucleic acid comprises at least two backbone modifications.

34. The ADAR recruiting molecule of any one of claims 2-33, wherein the single-stranded guide nucleic acid comprises at least three backbone modifications.

35. The ADAR recruiting molecule of any one of claims 2-34, wherein more than 25% of the internucleoside linkages in the single-stranded guide nucleic acid comprise a phosphate modification.

36. The ADAR recruiting molecule of any one of claims 2-35, wherein more than 50% of the internucleoside linkages in the single-stranded guide nucleic acid comprise a phosphate modification.

37. The ADAR recruiting molecule of any one of claims 2-36, wherein more than 75% of the internucleoside linkages in the single-stranded guide nucleic acid comprise a phosphate modification.

38. The ADAR recruiting molecule of any one of claims 2-37, wherein the single-stranded guide nucleic acid comprises sufficient complementarity to hybridize with a target sequence.

39. The ADAR recruiting molecule of any one of claims 2-38, wherein the single-stranded guide nucleic acid comprises three consecutive non-modified nucleotides.

40. The ADAR recruiting molecule of claim 39, wherein at least one of the three consecutive non-modified nucleotides pairs with a nucleotide adjacent to a target adenosine in the target sequence.

41. The ADAR recruiting molecule of claim 40, wherein the middle nucleotide of the three consecutive non-modified nucleotides is opposite the target adenosine.

42. The ADAR recruiting molecule of any one of claims 2-41, wherein a nucleotide opposite a target adenosine comprises:
(a) cytosine (C);
(b) a natural or modified nucleotide which does not base pair with adenosine (A); and/or
(c) a natural or modified nucleotide which base pairs with guanine (G) or inosine (I).

43. The ADAR recruiting molecule of any one of claims 1-42, wherein each RNA strand of the double-stranded RNA duplex is at least 10 nucleotides in length.

44. The ADAR recruiting molecule of any one of claims 1-43, wherein each RNA strand of the double-stranded RNA duplex is fewer than or equal to 100 nucleotides in length.

45. The ADAR recruiting molecule of any one of claims 1-44, wherein each RNA strand of the double-stranded RNA duplex is about 5 to about 80 nucleotides in length.

46. The ADAR recruiting molecule of any one of claims 1-45, wherein each RNA strand of the double-stranded RNA duplex is about 5 to about 60 nucleotides in length.

47. The ADAR recruiting molecule of any one of claims 1-46, wherein each RNA strand of the double-stranded RNA duplex is about 5 to about 40 nucleotides in length.

48. The ADAR recruiting molecule of any one of claims 1-47, wherein each RNA strand of the double-stranded RNA duplex is about 5 to about 30 nucleotides in length.

49. The ADAR recruiting molecule of any one of claims 1-48, wherein each RNA strand of the double-stranded RNA duplex is about 10 to about 27 nucleotides in length.

50. The ADAR recruiting molecule of any one of claims 1-49, wherein each RNA strand of the double-stranded RNA duplex is about 15 to about 26 nucleotides in length.

51. The ADAR recruiting molecule of any one of claims 2-50, wherein the single-stranded guide nucleic acid is at least 5 nucleotides in length.

52. The ADAR recruiting molecule of any one of claims 2-51, wherein the single-stranded guide nucleic acid is fewer than or equal to 100 nucleotides in length.

53. The ADAR recruiting molecule of any one of claims 2-52, wherein the single-stranded guide nucleic acid is about 5 to about 80 nucleotides in length.

54. The ADAR recruiting molecule of any one of claims 2-53, wherein the single-stranded guide nucleic acid is about 5 to about 60 nucleotides in length.

55. The ADAR recruiting molecule of any one of claims 2-54, wherein the single-stranded guide nucleic acid is about 5 to about 40 nucleotides in length.

56. The ADAR recruiting molecule of any one of claims 2-55, wherein the single-stranded guide nucleic acid is about 5 to about 30 nucleotides in length.

57. The ADAR recruiting molecule of any one of claims 2-56, wherein the single-stranded guide nucleic acid is about 10 to about 27 nucleotides in length.

58. The ADAR recruiting molecule of any one of claims 2-57, wherein the single-stranded guide nucleic acid is about 15 to about 26 nucleotides in length.

59. The ADAR recruiting molecule of any one of claims 2-58, wherein the single-stranded guide nucleic acid comprises at least 25% complementarity with a target sequence.

60. The ADAR recruiting molecule of any one of claims 2-59, wherein the single-stranded guide nucleic acid comprises at least 50% complementarity with a target sequence.

61. The ADAR recruiting molecule of any one of claims 2-60, wherein the single-stranded guide nucleic acid comprises at least 75% complementarity with a target sequence.

62. The ADAR recruiting molecule of any one of claims 2-61, wherein the single-stranded guide nucleic acid comprises at least 90% complementarity with a target sequence.

63. The ADAR recruiting molecule of any one of claims 2-62, wherein the single-stranded guide nucleic acid comprises at least 95% complementarity with a target sequence.

64. An RNA targeting molecule comprising:
(a) a double-stranded RNA duplex, wherein the double-stranded RNA duplex comprises two strands of RNA of an equal number of nucleotides, which two RNA strands are not connected to one another by means of a hairpin, wherein the 5′ nucleotide of each RNA strand is complementary to the 3′ nucleotide of the other RNA strand, wherein the double-stranded RNA duplex comprises at least one base pair mismatch, wherein the mismatch is not positioned at either terminal nucleotide base pair of the double-stranded RNA duplex; and
(b) a single-stranded guide nucleic acid.

65. The RNA targeting molecule of claim 64, wherein the double-stranded RNA duplex comprises at least one nucleoside modification and/or at least one backbone modification.

66. The RNA targeting molecule of claim 64 or claim 65, wherein the double-stranded RNA duplex comprises at least one nucleoside modification and at least one backbone modification.

67. The RNA targeting molecule of any one of claims 64-66, wherein the at least one nucleoside modification comprises a 2′-aminoethyl, a 2′-deoxy-2′-fluoro-β-d-arabinonucleic acid, a 2′-O-methyl, a 2′-O-methoxyethyl (2′O-MOE), or a 2′-fluoro modification.

68. The RNA targeting molecule of any one of claims 64-67, wherein the at least one backbone modification comprises a phosphorothioate modification.

69. The RNA targeting molecule of any one of claims 64-68, wherein the at least one backbone modification of the double-stranded RNA duplex is positioned within 1-5 nucleotides of the terminal nucleotide of the RNA strand on which it is located.

70. The RNA targeting molecule of any one of claims 64-69, wherein the at least one backbone modification of the double-stranded RNA duplex is positioned within 1-3 nucleotides of the terminal nucleotide of the RNA strand on which it is located.

71. The RNA targeting molecule of any one of claims 64-70, wherein the at least one backbone modification of the double-stranded RNA duplex is positioned within 1 nucleotide of the terminal nucleotide of the RNA strand on which it is located.

72. The RNA targeting molecule of any one of claims 64-71, wherein the double-stranded RNA duplex comprises more than one nucleoside modification.

73. The RNA targeting molecule of any one of claims 64-72, wherein the double-stranded RNA duplex comprises more than two nucleoside modifications.

74. The RNA targeting molecule of any one of claims 64-73, wherein more than 25% of the nucleosides in the double-stranded RNA duplex comprise a nucleoside modification.

75. The RNA targeting molecule of any one of claims 64-74, wherein more than 50% of the nucleosides in the double-stranded RNA duplex comprise a nucleoside modification.

76. The RNA targeting molecule of any one of claims 64-75, wherein more than 75% of the nucleosides in the double-stranded RNA duplex comprise a nucleoside modification.

77. The RNA targeting molecule of any one of claims 64-76, wherein the double-stranded RNA duplex comprises more than one backbone modification.

78. The RNA targeting molecule of any one of claims 64-77, wherein the double-stranded RNA duplex comprises more than two backbone modifications.

79. The RNA targeting molecule of any one of claims 64-78, wherein the double-stranded RNA duplex comprises more than three backbone modifications.

80. The RNA targeting molecule of any one of claims 64-79, wherein more than 25% of the internucleoside linkages of the double-stranded RNA duplex comprise a modification.

81. The RNA targeting molecule of any one of claims 64-80, wherein more than 50% of the internucleoside linkages of the double-stranded RNA duplex comprise a modification.

82. The RNA targeting molecule of any one of claims 64-81, wherein more than 75% of the internucleoside linkages of the double-stranded RNA duplex comprise a modification.

83. The RNA targeting molecule of any one of claims 64-82, further comprising nucleotides attached to the 3′ end or 5′ end of at least one of the RNA strands of the double-stranded RNA duplex creating a 3′ and/or 5′ end overhang.

84. The RNA targeting molecule of any one of claims 64-83, further comprising an additional moiety.

85. The RNA targeting molecule of any one of claims 64-84, further comprising a linker.

86. The RNA targeting molecule of any one of claims 64-85, wherein the single-stranded guide nucleic acid is guide ribonucleic acid (gRNA).

87. The RNA targeting molecule of any one of claims 64-86, wherein the single-stranded guide nucleic acid comprises at least one nucleoside modification.

88. The RNA targeting molecule of any one of claims 64-87, wherein the single-stranded guide nucleic acid comprises at least one backbone modification.

89. The RNA targeting molecule of any one of claims 64-88, wherein the single-stranded guide nucleic acid comprises at least one nucleoside modification and at least one backbone modification.

90. The RNA targeting molecule of any one of claims 64-89, wherein the single-stranded guide nucleic acid comprises at least two nucleoside modifications.

91. The RNA targeting molecule of any one of claims 64-90, wherein the single-stranded guide nucleic acid comprises at least three nucleoside modifications.

92. The RNA targeting molecule of any one of claims 64-91, more than 25% of the nucleosides of the single-stranded guide nucleic acid comprise a nucleoside modification.

93. The RNA targeting molecule of any one of claims 64-92, wherein more than 50% of the nucleosides in the single-stranded guide nucleic acid comprise a nucleoside modification.

94. The RNA targeting molecule of any one of claims 64-93, wherein more than 75% of the nucleosides in the single-stranded guide nucleic acid comprise a nucleoside modification.

95. The RNA targeting molecule of any one of claims 64-94, wherein the single-stranded guide nucleic acid comprises at least one backbone modification.

96. The RNA targeting molecule of any one of claims 64-95, wherein the single-stranded guide nucleic acid comprises at least two backbone modifications.

97. The RNA targeting molecule of any one of claims 64-96, wherein the single-stranded guide nucleic acid comprises at least three backbone modifications.

98. The RNA targeting molecule of any one of claims 64-97, wherein more than 25% of the internucleoside linkages in the single-stranded guide nucleic acid comprise a phosphate modification.

99. The RNA targeting molecule of any one of claims 64-98, wherein more than 50% of the internucleoside linkages in the single-stranded guide nucleic acid comprise a phosphate modification.

100. The RNA targeting molecule of any one of claims 64-99, wherein more than 75% of the internucleoside linkages in the single-stranded guide nucleic acid comprise a phosphate modification.

101. The RNA targeting molecule of any one of claims 64-100, wherein the single-stranded guide nucleic acid comprises sufficient complementarity to hybridize with a target sequence.

102. The RNA targeting molecule of any one of claims 64-101, wherein the single-stranded guide nucleic acid comprises three consecutive non-modified nucleotides.

103. The RNA targeting molecule of claim 102, wherein at least one of the three consecutive non-modified nucleotides of the single-stranded guide nucleic acid is complementary to a nucleotide adjacent to a target adenosine in the target sequence.

104. The RNA targeting molecule of claim 102 or claim 103, wherein the middle nucleotide of the three consecutive non-modified nucleotides is opposite the target adenosine.

105. The RNA targeting molecule of any one of claims 64-104, wherein a nucleotide opposite a target adenosine comprises:
(a) cytosine (C);
(b) a natural or modified nucleotide which does not base pair with adenosine (A); and/or
(c) a natural or modified nucleotide which base pairs with guanine (G) or inosine (I).

106. The RNA targeting molecule of any one of claims 64-105, wherein each RNA strand of the double-stranded RNA duplex is at least 5 nucleotides in length.

107. The RNA targeting molecule of any one of claims 64-106, wherein each RNA strand of the double-stranded RNA duplex is fewer than or equal to 100 nucleotides in length.

108. The RNA targeting molecule of any one of claims 64-107, wherein each RNA strand of the double-stranded RNA duplex is about 5 to about 80 nucleotides in length.

109. The RNA targeting molecule of any one of claims 64-108, wherein each RNA strand of the double-stranded RNA duplex is about 5 to about 60 nucleotides in length.

110. The RNA targeting molecule of any one of claims 64-109, wherein each RNA strand of the double-stranded RNA duplex is about 5 to about 40 nucleotides in length.

111. The RNA targeting molecule of any one of claims 64-110, wherein each RNA strand of the double-stranded RNA duplex is about 5 to about 30 nucleotides in length.

112. The RNA targeting molecule of any one of claims 64-111, wherein each RNA strand of the double-stranded RNA duplex is about 10 to about 27 nucleotides in length.

113. The RNA targeting molecule of any one of claims 64-112, wherein each RNA strand of the double-stranded RNA duplex is about 15 to about 26 nucleotides in length.

114. The RNA targeting molecule of any one of claims 64-113, wherein the single-stranded guide nucleic acid is at least 5 nucleotides in length.

115. The RNA targeting molecule of any one of claims 64-114, wherein the single-stranded guide nucleic acid is fewer than or equal to 100 nucleotides in length.

116. The RNA targeting molecule of any one of claims 64-115, wherein the single-stranded guide nucleic acid is about 5 to about 80 nucleotides in length.

117. The RNA targeting molecule of any one of claims 64-116, wherein the single-stranded guide nucleic acid is about 5 to about 60 nucleotides in length.

118. The RNA targeting molecule of any one of claims 64-117, wherein the single-stranded guide nucleic acid is about 5 to about 40 nucleotides in length.

119. The RNA targeting molecule of any one of claims 64-118, wherein the single-stranded guide nucleic acid is about 5 to about 30 nucleotides in length.

120. The RNA targeting molecule of any one of claims 64-119 wherein the single-stranded guide nucleic acid is about 10 to about 27 nucleotides in length.

121. The RNA targeting molecule of any one of claims 64-120, wherein the single-stranded guide nucleic acid is about 15 to about 26 nucleotides in length.

122. The RNA targeting molecule of any one of claims 64-121, wherein the single-stranded guide nucleic acid comprises at least 25% complementarity with a target sequence.

123. The RNA targeting molecule of any one of claims 64-122, wherein the single-stranded guide nucleic acid comprises at least 50% complementarity with a target sequence.

124. The RNA targeting molecule of any one of claims 64-123, wherein the single-stranded guide nucleic acid comprises at least 75% complementarity with a target sequence.

125. The RNA targeting molecule of any one of claims 64-124, wherein the single-stranded guide nucleic acid comprises at least 90% complementarity with a target sequence.

126. The RNA targeting molecule of any one of claims 64-125, wherein the single-stranded guide nucleic acid comprises at least 95% complementarity with a target sequence.

127. A method of deaminating a target nucleic acid in a subject, comprising, administering an effective amount of the ADAR recruiting molecule of any one of claims 2-63, and/or the RNA targeting molecule of any one of claims 64-126, wherein the ADAR recruiting molecule and/or the RNA targeting molecule comprises a single-stranded guide nucleic acid comprising a sequence which is sufficiently complementary to a target sequence to hybridize with the target sequence.

128. The method of claim 127, wherein the target sequence comprises a target adenosine.

129. A method of treating a subject, comprising administering the ADAR recruiting molecule of any one of claims 2-63, and/or the RNA targeting molecule of any one of claims 64-126, wherein the ADAR recruiting molecule and/or the RNA targeting molecule comprises a single-stranded guide nucleic acid comprising a sequence which is sufficiently complementary to a target sequence to hybridize with the target sequence.

130. The method of claim 129, wherein the target sequence comprises a target adenosine.

131. The method of claim 130, wherein the target adenosine is related to a disease or disorder, wherein the deamination of the target adenosine treats the disease or disorder.

132. The method of claim 131, wherein the disease or disorder is selected from: Cystic fibrosis, Hurler Syndrome, alpha-1-antitrypsin (A1AT) deficiency, Parkinson's disease, Alzheimer's disease, albinism, Amyotrophic lateral sclerosis, Asthma, beta-thalassemia (β-thalassemia), Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B, and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, Sturge-Weber Syndrome, and cancer.

133. An RNA targeting molecule comprising:
(a) a double-stranded RNA duplex comprising two RNA strands;
(b) a single-stranded guide nucleic acid; and
(c) a linker;
wherein the double-stranded RNA duplex is connected to the single-stranded guide nucleic acid via the linker.

134. The RNA targeting molecule of claim 133, wherein the linker is connected to the 5′ terminal nucleotide of one of the two strands of the RNA duplex.

135. The RNA targeting molecule of claim 133, wherein the linker is connected to the 3′ terminal nucleotide of one of the two strands of the RNA duplex.

136. The RNA targeting molecule of claim 133, wherein the linker is connected to a nucleotide located between the 5′ terminal nucleotide and the 3′ terminal nucleotide of one of the two strands of the RNA duplex.

137. The RNA targeting molecule of claim 133, wherein the linker is connected to the 5′ nucleotide of the guide nucleic acid.

138. The RNA targeting molecule of claim 133, wherein the linker is connected to the 3′ nucleotide of the guide nucleic acid.

139. The RNA targeting molecule of claim 133, wherein the linker is connected to a nucleotide located between the 5′ terminal nucleotide and the 3′ terminal nucleotide of the guide nucleic acid.

140. The RNA targeting molecule of claim 133, wherein the linker is connected to a sugar of the 5′ terminal nucleotide, or a 3′ hydroxyl or sugar of the 3′ terminal nucleotide of one strand of the RNA duplex.

141. The RNA targeting molecule of claim 133, wherein the linker is connected to a sugar of the 5′ terminal nucleotide, or a 3′ hydroxyl or sugar of the 3′ terminal nucleotide of the guide nucleic acid.

142. The RNA targeting molecule of claim 133, wherein the linker is connected to an internucleoside linkage of one RNA strand of the RNA duplex.

143. The RNA targeting molecule of claim 133, wherein the linker is connected to an internucleoside linkage of the guide nucleic acid.

144. The RNA targeting molecule of claim 133, wherein the linker is connected to a nucleoside sugar of one RNA strand of the RNA duplex.

145. The RNA targeting molecule of claim 133, wherein the linker is connected to a nucleoside sugar of the guide nucleic acid.

146. The RNA targeting molecule of claim 133, wherein the linker connects the 3′ end of the guide nucleic acid to the 5′ end of one RNA strand of the RNA duplex.

147. The RNA targeting molecule of claim 133, wherein the linker connects the 5′ end of the guide nucleic acid to the 3′ end of one RNA strand of the RNA duplex.

148. The RNA targeting molecule of claim 133, wherein the linker connects the 3′ end of the guide nucleic acid to the 3′ end of one RNA strand of the RNA duplex.

149. The RNA targeting molecule of claim 133, wherein the linker connects the 5′ end of the guide nucleic acid to the 5′ end of one RNA strand of the RNA duplex.

150. The RNA targeting molecule of claim 133, wherein the linker connects the 3′ end of the guide nucleic acid to a nucleotide located between the 5′ terminal nucleotide and the 3′ terminal nucleotide of one RNA strand of the RNA duplex.

151. The RNA targeting molecule of claim 150, wherein the linker is connected to an internucleoside linkage or a nucleoside sugar of one RNA strand of the RNA duplex.

152. The RNA targeting molecule of claim 133, wherein the linker connects the 5′ end of the guide nucleic acid to a nucleotide located between the 5′ terminal nucleotide and the 3′ terminal nucleotide of one RNA strand of the RNA duplex.

153. The RNA targeting molecule of claim 152, wherein the linker is connected to an internucleoside linkage or a nucleoside sugar of one RNA strand of the RNA duplex.

154. The RNA targeting molecule of claim 133, wherein the linker connects the 3′ end of one RNA strand of the RNA duplex to a nucleotide located between the 5′ terminal nucleotide and the 3′ terminal nucleotide of the guide nucleic acid.

155. The RNA targeting molecule of claim 154, wherein the linker is connected to an internucleoside linkage or a nucleoside sugar of the guide nucleic acid.

156. The RNA targeting molecule of claim 133, wherein the linker connects the 5′ end of one RNA strand of the RNA duplex to a nucleotide located between the 5′ terminal nucleotide and the 3′ terminal nucleotide of the guide nucleic acid.

157. The RNA targeting molecule of claim 156, wherein the linker is connected to an internucleoside linkage or a nucleoside sugar of the guide nucleic acid.

158. The RNA targeting molecule of claim 133, or any other prior claim, wherein the linker is an unbranched linker.

159. The RNA targeting molecule of claim 133, or any other prior claim, wherein the linker is a branched linker.

160. The RNA targeting molecule of claim 133, or any other prior claim, wherein the linker is a non-covalent linker comprising a first binding partner covalently attached to one strand of the double-stranded RNA duplex, and a second binding partner covalently attached to the single-stranded guide nucleic acid, wherein the first and second binding partners form a non-covalent complex connecting the double-stranded RNA duplex to the single-stranded guide nucleic acid.

161. The RNA targeting molecule of claim 160, wherein the first binding partner is a receptor and the second binding partner is a ligand specific for the receptor.

162. The RNA targeting molecule of claim 160, wherein the second binding partner is a receptor and the first binding partner is a ligand specific for the receptor.

163. The RNA targeting molecule of claim 160, wherein the first binding partner is biotin and the second binding partner is streptavidin.

164. The RNA targeting molecule of claim 160, wherein the first binding partner is streptavidin and the second binding partner is biotin.

165. The RNA targeting molecule of claim 133, or any other prior claim, wherein the linker is a covalent linker.

166. The RNA targeting molecule of claim 133, or any other prior claim, wherein the linker is greater than or equal to 4 atoms in length.

167. The RNA targeting molecule of claim 133, or any other prior claim, wherein the linker is fewer than or equal to 180 atoms in length.

168. The RNA targeting molecule of claim 165, wherein the linker comprises an alkyl, alkenyl, alkynyl, substituted alkyl, substituted alkenyl, substituted alkynyl, repeated ethylene glycol group, ether, thioether, urea, carbonate, amine, amide, maleimide-thioether, disulfide, phosphodiester, sulfonamide linkage, a product of a click reaction, a triazole from an azide-alkyne cycloaddition, carbamate, a cleavable linker, a redox cleavable linker, a reductively cleavable linker, a disulfide group, an acid cleavable linker, a hydrazone group, an ester group, an acetal group, a ketal group, an esterase cleavable linker, an ester group, a phosphatase cleavable linker, a phosphate group, a peptidase cleavable linker, a peptide bond, a bio-cleavable linker, DNA, RNA, disulfide, amide, functionalized monosaccharides, or oligosaccharides of galactosamine.

169. The RNA targeting molecule of claim 165, wherein the linker comprises a moiety derived from a click chemistry reaction.

170. The RNA targeting molecule of claim 169, wherein the moiety derived from a click chemistry reaction is a triazole, diazole, diazine, sulfide bond, maleimide ring, succinimide ring, ester, or amide.

171. The RNA targeting molecule of claim 165, wherein the linker comprises one or more amino acids.

172. The RNA targeting molecule of claim 165, wherein the linker comprises an organic molecule, group, polymer, or chemical domain.

173. The RNA targeting molecule of claim 172, wherein the chemical domain comprises an amide, urea, carbamate, carbonate, ester, acetal, ketal, phosphoramidite, hydrazone, imine, oxime, disulfide, silyl, hydrazine, hydrazone, thiol, imidazole, carbon-carbon bond, carbon-heteroatom bond, or azo domain.

174. The RNA targeting molecule of claim 165, wherein the linker is polymeric.

175. The RNA targeting molecule of claim 174, wherein the polymeric linker comprises polyethylene, polyethylene glycol, polyamide, polyester, or polyether.

176. The RNA targeting molecule of claim 133, or any other prior claim, wherein the linker comprises any one of Formula (I)-Formula (VII).

177. The RNA targeting molecule of claim 133, or any other prior claim, wherein the double-stranded RNA duplex comprises:
(a) an RNA strand comprising a sequence with at least 70% identity to Strand Ref.: 24 or 27; and
(b) an RNA strand comprising a sequence with at least 70% identity to Strand Ref.: 25 or 28;
and wherein the single-stranded guide nucleic acid comprises a sequence with at least 70% identity to Strand Ref.: 23 or 26.

178. The RNA targeting molecule of claim 133, or any other prior claim, wherein the double-stranded RNA duplex comprises:
(a) an RNA strand comprising a sequence according to Strand Ref.: 24 or 27; and
(b) an RNA strand comprising a sequence according to Strand Ref.: 25 or 28;
and wherein, the at least one single-stranded guide nucleic acid comprises a sequence according to Strand Ref.: 23 or 26.

179. The RNA targeting molecule of any one of claims 133-176, wherein the double-stranded RNA duplex comprises:
(a) an RNA strand comprising a sequence with at least 70% identity to Strand Ref.: 353 or 355; and
(b) an RNA strand comprising a sequence with at least 70% identity to Strand Ref.: 815 or 818.

180. The RNA targeting molecule of any one of claims 133-176, wherein the double-stranded RNA duplex comprises:
(a) an RNA strand comprising a sequence according to Strand Ref.: 353 or 355; and
(b) an RNA strand comprising a sequence according to Strand Ref.: 815 or 818.

181. The RNA targeting molecule of any one of claims 133-176, wherein the double-stranded RNA duplex comprises:
(a) an RNA strand comprising a sequence with at least 70% identity to Strand Ref.: 641 or 643; and
(b) an RNA strand comprising a sequence with at least 70% identity to Strand Ref.: 841 or 869.

182. The RNA targeting molecule of any one of claims 133-176, wherein the double-stranded RNA duplex comprises:
(a) an RNA strand comprising a sequence according to Strand Ref.: 641 or 643; and
(b) an RNA strand comprising a sequence according to Strand Ref.: 841 or 869.

183. The RNA targeting molecule of claim 133, or any other prior claim, comprising two or more double-stranded RNA duplexes.

184. The RNA targeting molecule of claim 133, or any other prior claims, comprising two or more single-stranded guide nucleic acids.

185. The RNA targeting molecule of claim 133, or any other prior claim, comprising 2-10 double-stranded RNA duplexes.

186. The RNA targeting molecule of claim 133, or any other prior claims, comprising 2-10 single-stranded guide nucleic acids.

187. The RNA targeting molecule of claim 133, or any other prior claim, comprising 2-5 double-stranded RNA duplexes.

188. The RNA targeting molecule of claim 133, or any other prior claims, comprising 2-5 single-stranded guide nucleic acids.

189. The RNA targeting molecule of any one of claims 133-188, wherein one strand of the double-stranded RNA duplex is not covalently connected to the other strand of the RNA duplex.

190. The RNA targeting molecule of any one of claims 133-189, wherein the double-stranded RNA duplex does not comprise a hairpin connecting one strand of the RNA duplex to the other strand of the RNA duplex.

191. The RNA targeting molecule of any one of claims 133-190, wherein the double-stranded RNA duplex comprises two RNA strands having an equal number of nucleotides.

192. The RNA targeting molecule of any one of claims 133-191, wherein the double-stranded RNA duplex comprises two RNA strands having a different number of nucleotides.

193. The RNA targeting molecule of any one of claims 133-192, wherein the linker does not comprise a nucleotide or nucleoside.

194. The RNA targeting molecule of any one of claims 133-193, wherein the linker is a non-nucleic acid linker.

195. An RNA targeting molecule comprising:
(a) a first double-stranded RNA duplex comprising two RNA strands;
(b) a second double-stranded RNA duplex comprising two RNA strands;
(b) a single-stranded guide nucleic acid; and
(c) a linker;
wherein the first double-stranded RNA duplex is connected to the second double-stranded RNA duplex via the linker.

196. An RNA targeting molecule comprising:
(a) a double-stranded RNA duplex comprising two RNA strands;
(b) a first single-stranded guide nucleic acid;
(c) a second single-stranded guide nucleic acid; and
(c) a linker;
wherein the first single-stranded guide nucleic acid is connected to the second single-stranded guide nucleic acid via the linker.

197. The RNA targeting molecule of claim 133, or any other prior claim, wherein the double-stranded RNA duplex comprises at least one mismatch.

198. The RNA targeting molecule of claim 133, or any other prior claim, wherein the single-stranded guide nucleic acid comprises at least two mismatches relative to a target sequence.
